(19) United States
(12) Patent Application Publication
(10) Pub. No.: US 2025/0026850 A1
(43) Pub. Date: Jan. 23, 2025

(54) ANTI-CD39 ANTIBODIES, COMPOSITIONS COMPRISING ANTI-CD39 ANTIBODIES AND METHODS OF USING ANTI-CD39 ANTIBODIES

(71) Applicant: Trishula Therapeutics, Inc., South San Francisco, CA (US)

(72) Inventors: Vanessa Soros, South San Francisco, CA (US); Maria Kovalenko, South San Francisco, CA (US); John Corbin, South San Francisco, CA (US); Courtney Beers, South San Francisco, CA (US); Paul Fredrick Widboom, Hanover, NH (US); Joseph Robert Warfield, White River Junction, VT (US)

(21) Appl. No.: 18/895,094

(22) Filed: Sep. 24, 2024

Related U.S. Application Data
(63) Continuation of application No. 17/725,142, filed on Apr. 20, 2022, now abandoned.
Application No. 17/725,142 is a continuation of application No. 16/635,526, filed on Jan. 30, 2020, now Pat. No. 11,345,757.
(60) Provisional application No. 62/539,527, filed on Jul. 31, 2017.

(51) Int. Cl. C07K 16/28; A61K 39/00; A61K 39/395; A61K 45/06; A61P 37/04; C12N 15/11; C12N 15/63; G01N 33/566

(52) U.S. Cl. CPC C07K 16/2896; A61K 39/3955; A61P 37/04; C12N 15/11; C12N 15/63; G01N 33/566; A61K 39/39541; A61K 2039/505; A61K 45/06; C07K 2317/21; C07K 2317/24; C07K 2317/31; C07K 2317/33; C07K 2317/56; C07K 2317/565; C07K 2317/74; C07K 2317/76; C07K 2317/77; C07K 2317/92; G01N 2333/70596; G01N 2500/04

(57) ABSTRACT

Provided herein are antibodies with binding specificity for CD39 and compositions comprising the antibodies, including pharmaceutical compositions, diagnostic compositions and kits. Also provided are methods of using anti-CD39 antibodies for therapeutic and diagnostic purposes.

### RELATED APPLICATION

This application claims priority to U.S. provisional application No. 62/539,527, filed Jul. 31, 2017, which is incorporated by reference herein in its entirety.

### FIELD

Provided herein are antibodies with binding specificity for CD39 and compositions comprising the antibodies, including pharmaceutical compositions, diagnostic compositions and kits. Also provided are methods of using anti-CD39 antibodies for therapeutic and diagnostic purposes.

### BACKGROUND

CD39 is an integral membrane protein that phosphohydrolyzes ATP to yield ADP and AMP. Human CD39 is a 510-amino acid protein with seven potential N-linked glycosylation sites, 11 cysteine residues, and two transmembrane regions. Structurally, it is characterized by two transmembrane domains, a small cytoplasmic domain comprising the NH₂- and COOH-terminal segments, and a large extracellular hydrophobic domain consisting of five highly conserved domains, known as apyrase conserved regions (ACR) 1-5, which are pivotal for the catabolic activity of the enzyme. CD39 becomes catalytically active upon its localization on the cell surface, and its glycosylation is important for protein folding, membrane targeting, and enzyme activity.

CD39 is constitutively expressed in spleen, thymus, lung, and placenta and in these tissues it is associated primarily with endothelial cells and immune cell populations, such as B cells, natural killer (NK) cells, dendritic cells. Langerhans cells, monocytes, macrophages, mesangial cells, neutrophils, and regulatory T cells (Tregs). Given that CD39, along with other enzymes, degrades ATP, ADP, and AMP to adenosine, CD39 can be viewed as an immunological switch that shifts ATP-driven pro-inflammatory immune cell activity toward an anti-inflammatory state mediated by adenosine.

Within a neoplastic milieu, cancer and immune cells can closely interact to generate an immunosuppressive environment by releasing immunomodulatory factors, which support neoplastic growth. The expression of CD) 39 is increased in many solid tumors (for example, colorectal cancer, head and neck cancer, pancreatic cancer (Kunzli et al., Am J Physiol. 2006, 292:223-230), bladder cancer, brain cancer, breast cancer, gastric cancer, hepatocellular carcinoma, lung cancer, non-small cell lung cancer (Li et al., Oncoimmunology, 2017, 6:6), chronic lymphocytic leukemia (Pulte et al., Clin Lymphoma Myeloma Leuk, 2011, 11 (4): 367-372) and lymphoma, melanoma (Dzhandzhugazyan et al., FEBS Letters, 1998, 430:227-230), ovarian cancer, and prostate cancer, among others) suggesting this enzyme is involved in the development and progression of malignancies. Modulators of CD39 may provide potential therapies for these types of cancers.

Interactions between tumor cells and their microenvironment are important for tumorigenesis. CD39 can participate in tumor immunoescape by inhibiting the activation, clonal expansion, and homing of tumor-specific T cells, impairing tumor cell killing by effector T lymphocytes. In addition to these immunoregulatory roles, CD39 can contribute directly to the modulation of cancer cell growth, differentiation, invasion, migration, metastasis, and angiogenesis. CD39 is important for both the initiation of angiogenesis and the progression of neovascularization. CD39 on vasculature mediates the angiogenic process in mouse models of melanoma, lung, and liver malignancy.

Modulators of CD39 activity may also provide potential therapeutics for the treatment of CD39 conditions including, but not limited to, autoimmune diseases and infections. In particular, modulators of CD39 activity may provide potential therapeutics for diseases such as, for example, without limitation, Celiac disease (Cook et al., American Academy of Allergy, Asthma & Immunology, 2017, Article in Press), colitis (Longhi et al., JCI Insight. 2017, 2 (9)), thrombotic disease (Marcus et al., Journal of Pharmacology and Experimental Therapeutics, 2003, 305, 1:9-16), HIV infection (zur Wiesch et al., Journal of Virology, 2011 February: 1287-1297), LIBV infection, NICV infection, and inflammatory bowel disease (Friedman et al. PNAS, 2009, 106, 39:16788-16793) and Crohn's disease (Bai et al., J Immunol, 2014, 3366-3377).

### SUMMARY

Provided herein are antibodies that selectively bind CD39. In some embodiments, the antibodies bind human CD39. In some embodiments, the antibodies also bind homologs of human CD39.

In some embodiments, the antibodies comprise at least one CDR sequence defined by a consensus sequence provided in this disclosure. In some embodiments, the antibodies comprise an illustrative CDR, V_{H}, or V_{L} sequence provided in this disclosure, heavy chain or light chain provided in the disclosure, or a variant thereof. In some aspects, the variant is a variant with one or more conservative amino acid substitutions.

Also provided are compositions and kits comprising the antibodies. In some embodiments, the compositions are pharmaceutical compositions. Any suitable pharmaceutical composition may be used. In some embodiments, the pharmaceutical composition is a composition for parenteral administration.

This disclosure also provides methods of using the anti-CD39 antibodies provided herein. In some embodiments, the method is a method of treatment. In some embodiments, the method is a diagnostic method. In some embodiments, the method is an analytical method. In some embodiments, the method is a method of purifying and/or quantifying CD39.

In some embodiments, the antibodies are used to treat a disease or condition. In some aspects, the disease or condition is selected from a cancer, autoimmune disease, and infection.

### BRIEF DESCRIPTION OF THE DRAWINGS

FIG. 1 provides a table showing monovalent affinity of anti-huCD39 antibodies to recombinant human CD39 extracellular domain. The table provides binding kinetics of anti-CD39 antibodies interacting with soluble recombinant human CD39 (ENTDP1) extracellular domain (ECD) by biolayer interferometry (ForteBio Octet). Anti-CD39 antibodies were captured on an anti-human Fc sensor and exposed to recombinant human CD39 ECD at concentration ranging from 10-300 nanomolar. The kinetic data was globally fit with a simple 1:1 Langmuir binding model to yield on-rate (kon) and off-rate (koff) values. The equilibrium dissociation constants (K_{D}) were calculated from the kon and koff values.

FIGS. 2A-E show inhibition of enzymatic catabolism of ATP and ADP to Pi by human CD39 extracellular domain (ECD). Recombinant human CD39 at a final concentration of either 10 nanomolar (A-D) or 5 nanomolar (E) was incubated with anti-CD39 IgGs at a final concentration of either 1 micromolar (A-D) or 0.25 micromolar (E) in 25 mM Tris, 5 mM CaCl₂), pH 7.5 at room temperature for 2 hours. ATP (500 micromolar) was added to the reaction and incubated at 37° C. for 60 minutes. Residual ATP levels in the reaction were measured using the CellTiter-Glo assay. Data values are the average of two replicates.

FIG. 2F shows antibodies that bind to CHO) cells expressing human or cyno CD39.

FIG. 3 shows evaluation of antibody binding to MEI-28 (A) and 721 (B) cells. Anti-CD39 antibodies (each antibody clone number indicated in the figure) were titrated from 15 to 0.001 ng/ml. EC₅₀s were calculated using Graph Pad Prism Software. The figure represents three independent experiments.

FIG. 4 provides evaluation of antibody driven inhibition of ATP hydrolysis of CD39 on MEL-28 cells in a short term ATP assay. FIG. 4A shows the results when anti-CD39 antibodies were compared to the non-specific small molecule inhibitors POM-1 and ARL. Inhibition is determined by decreased phosphate release (Pi). Data is representative of at least 10 independent experiments. Anti-CD39 antibodies (each antibody clone number is indicated in the figure) were titrated from 15 to 0.001 ng/ml. IC50 values were calculated using GraphPad Prism Software, as can be seen in FIG. 4B. Three independent experiments were performed.

FIG. 5 provides an illustration of quantification when MEL-28 cells are treated with CD39 enzymatic inhibitors. FIG. 5A shows evaluation of ATP levels after incubating MEL-28 cells with a dose titration of anti-CD39 antibodies that inhibit enzymatic activity. FIG. 5B provides IC50 values calculated using GrapPad Prism. At least three independent experiments were performed.

FIG. 6 shows that antibodies were evaluated for inhibiting CD39 enzymatic activity overnight to MEL-28 cells. FIG. 6A shows anti-CD39 antibodies (each number represents a unique clone indicated in the figure) were titrated from 100 nM to 0.000610 nM.

FIG. 6B provide IC50 values calculated using GraphPad Prism Software. Three independent experiments are represented.

FIG. 7 shows results of testing of antibodies for binding to CD39 on human and cyno primary B cells. In FIG. 7A, anti-CD39 antibodies were titrated on purified B cells from healthy donor and detected with anti-human IgG-PE secondary antibody. EC50 was calculated using GrapPad Prism software. In FIG. 7B, anti-CD39 antibodies were titrated on cyno PBMCs and detected using a-human IgG PE secondary antibody. B cells were gated using FlowJo software and ECsos were calculated using GraphPad Prism.

FIG. 8 sets forth antibodies that were evaluated for inhibition of CD39 activity on primary human B cells. Anti-CD39 antibodies (each unique antibody clone number indicated is in the figure) were titrated from 100 to 0.00013 nM.

FIG. 9 provides evaluation of inhibition of CD39 activity on primary human (a) and cyno (b) monocytes. Anti-CD39 antibodies (each number represents a unique antibody clone number as indicated in the figure) were titrated from 100 to 0.00013 nM and incubated with monocytes in presence of ATP. Phosphate release by CD39 processing of ATP was quantified using Malachite Green assay.

FIG. 10 shows binding of anti-CD39 antibodies on purified human CD4 CD25-CD127^{dim} Treg cells by FACS. Anti-CD39 antibodies were titrated on purified Treg from healthy donor and detected with anti-human IgG secondary antibody. EC₅₀s were calculated using GraphPad Prism software.

FIG. 11 shows results for antibody ability to inhibit primary Treg CD39 activity. CD24 CD25 CD127^{dim} T regulatory cells were incubated with serially diluted anti-CD39 antibodies and tested for ATPase activity after addition of exogenous ATP. Free phosphate (Pi) was used as a readout of CD39 activity.

FIG. 12 shows treatment with anti-CD39 antibodies increase the percent of IFN gamma producing CD8⁺ T cells that respond to CMV peptides in an antigen recall response assay.

FIG. 13 shows evaluation of antibodies for inhibition of CD39 activity on MEL-28 (FIG. 13A.) and human monocytes (FIG. 13B.) as compared to the anti-CD39 antibodies generated based on Innate/Orega (BY-40v9) and Igenica (9-8B) and variants thereof. Anti-CD39 antibodies were titrated as indicated and incubated in presence of ATP. Phosphate release by CD39 processing of ATP was quantified using Malachite Green assay.

FIGS. 14A-G provides examples of antibodies. FIG. 14A provides examples of antibodies that bind soluble recombinant CD39 ECD and cellular CD39 but do not inhibit ATPase activity and do not compete with cellular inhibitors for binding to ECD inhibitors. FIG. 14B provides examples of antibodies that have limited ability to inhibit the ATPase activity of both soluble recombinant and cellular CD39 and bin separately from other cellular CD39 inhibitors. FIG. 14C provides example of antibodies that inhibit the ATPase activity of soluble recombinant CD39 ECD but do not inhibit cellular CD39 and bin separately from other CD39 ECD inhibitors. FIG. 14D provides examples of antibodies that inhibit the ATPase activity of ECD and cellular CD39 and bin separately from other CD39 ECD and/or cellular inhibitors. FIG. 14E provides examples of inhibitory antibodies that make distinct contacts with CD39. FIG. 14E has two tables, Table 1 and Table 2. Table 1 provides examples of inhibitory antibodies that make distinct contacts with CD39. Table 2 provides examples of inhibitory antibodies that bind critical yet distinct contacts residues with CD39. FIG. 14F and FIG. 14G provide FACS plotting highlighting the importance of certain human CD39 residues.

FIG. 15 shows that anti-CD39 antibodies inhibit CD39 by 75-90%. Anti-CD39 antibodies (100 nanomolar), isotype control antibody (100 nanomolar), or ARL (200 micromolar) were incubated with MEL-28 cells endogenously expressing human CD39 for 2 hours. ATP was then added and the rate of ATP hydrolysis to Pi by CD39 was monitored using the EnzChek kinetic Pi detection assay. The initial enzyme velocity, ν0, was determined from the linear region of Pi vs. time curve over the first 15 minutes post-ATP addition. Each value is the mean of 3 replicates.

FIGS. 16A-B shows that the CD39 inhibitor 29872 is not a competitive inhibitor due to suppression of Vₘₐₓ suppression.

FIG. 17 shows that anti-CD39 antibodies can induce internalization of CD39 on cyno monocytes.

FIG. 18 provides a comparison of the Kabat and Chothia numbering systems for CDR-H1. Adapted from Martin A. C. R. (2010). Protein Sequence and Structure Analysis of Antibody Variable Domains. In R. Kontermann & S. Dübel (Eds.), Antibody Engineering vol. 2 (pp. 33-51). Springer-Verlag, Berlin Heidelberg.

FIG. 19 shows an anti-CD39 antibody increases proliferation of stimulated CD4⁺ and CD8⁺ T cells.

FIG. 20 shows anti-CD39 antibody increases stimulated PBMC secretion of INF-γ, TNF-α and IL-2.

FIG. 21 shows anti-CD39 antibody increases stimulated PBMC secretion of INF-γ, TNF-α, IL-2 and II-1β.

FIG. 22 shows CD39 inhibition leads to accumulation of ATP and blocks generation of adenosine.

### DETAILED DESCRIPTION

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, the term “about” indicates the designated value ±one standard deviation of that value.

The term “combinations thereof” includes every possible combination of elements to which the term refers.

The terms “CD39” and “CD39 antigen” and “Cluster of Differentiation 39” are used interchangeably herein. CD39 is also known as also known as ectonucleoside triphosphate diphosphohydrolase-1 (gene: ENTPD1; protein: NTPDase1, See www.ncbi.nlm.nih.gov/gene/953). CD39 has also been referred to as ATPDase and SPG64. Each of the terms set forth may be used interchangeably. Unless specified otherwise, the terms include any variants, isoforms and species homologs of human CD39 that are naturally expressed by cells, or that are expressed by cells transfected with a CD39 gene. In some embodiments, CD39 proteins include murine CD39. In some embodiments, CD39 proteins include cynomolgus CD39.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, C_{H1}, C_{H2}, and C_{H3}. Each light chain typically comprises a light chain variable region (VL) and a light chain constant region. The light chain constant region typically comprises one domain, abbreviated CL.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer. A “CD39 antibody,” “anti-CD39 antibody,” “CD39 Ab,” “CD39-specific antibody” or “anti-CD39 Ab” is an antibody, as described herein, which binds specifically to the antigen CD39. In some embodiments, the antibody binds the extracellular domain of CD39.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1 FR2-CDR2-FR3-CDR3-FR4. The CDRs are involved in antigen binding, and confer antigen specificity and binding affinity to the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun,. J. Mol. Biol., 2001, 309:657-70 (“AHo” numbering scheme), each of which is incorporated by reference in its entirety.

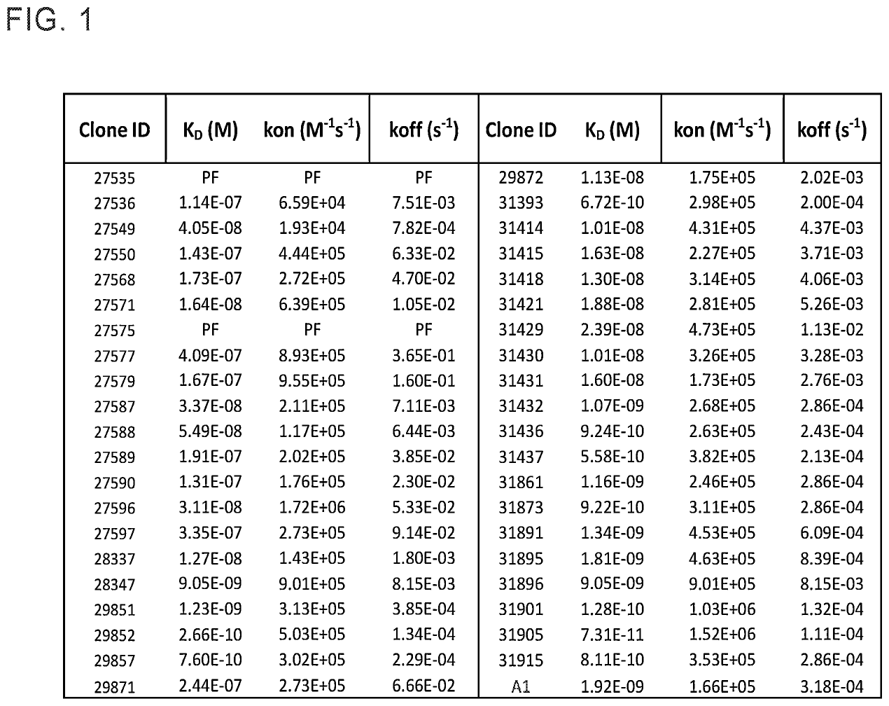

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes. FIG. 1 provides a comparison of the Kabat and Chothia numbering schemes for CDR-H1. See Martin (2010), supra.

Unless otherwise specified, the numbering scheme used for identification of a particular CDR herein is the Kabat/Chothia numbering scheme. Where the residues encompassed by these two numbering schemes diverge, the numbering scheme is specified as either Kabat or Chothia.

TABLE 1
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR, as illustrated in FIG. 1.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (CHI) of the heavy chain. Fab fragments may be generated, for example, by papain digestion of a full-length antibody.

“F(ab′)₂” fragments contain two Fab′ fragments joined, near the hinge region, by disulfide bonds. F(ab′)₂ fragments may be generated, for example, by pepsin digestion of an intact antibody. The F(ab′) fragments can be dissociated, for example, by treatment with β-mercaptoethanol.

“Single-chain Fv” or “sFv” or “scFy” antibody fragments comprise a V_{H} domain and a VL domain in a single polypeptide chain. The V_{H} and VL are generally linked by a peptide linker. See Pluckthun A. (1994). Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety. “scFv-Fc” fragments comprise an scFv attached to an Fc domain. For example, an Fc domain may be attached to the C-terminal of the scFv. The Fc domain may follow the V_{H} or VL, depending on the orientation of the variable domains in the scFv (i.e., V_{H}-V_{L} or V_{L}-V_{H}). Any suitable Fc domain known in the art or described herein may be used.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least 80%, 85%, 90%, 95%, or 99% by weight. In some embodiments, an isolated antibody is provided as a solution comprising at least 85%, 90%, 95%, 98%, 99% to 100% by weight of an antibody, the remainder of the weight comprising the weight of other solutes dissolved in the solvent.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Affinity can be determined, for example, using surface plasmon resonance (SPR) technology, such as a Biacore R instrument.

With regard to the binding of an antibody to a target molecule, the terms “specific binding.” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that is similar to the target, such as an excess of non-labeled target. In that case, specific binding is indicated if the binding of the labeled target to a probe is competitively inhibited by the excess non-labeled target.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ.

The term “K_{A}” (M⁻¹), as used herein, refers to the association equilibrium constant of a particular antibody-antigen interaction. K_{A}=kₐ/k_{d}.

An “affinity matured” antibody is one with one or more alterations in one or more CDRs or FRs that result in an improvement in the affinity of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more antibodies, the term “competes with” or “cross-competes with” indicates that the two or more antibodies compete for binding to an antigen (e.g., CD39). In one exemplary assay, CD39 is coated on a plate and allowed to bind a first antibody, after which a second, labeled antibody is added. If the presence of the first antibody reduces binding of the second antibody, then the antibodies compete. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 50%, at least 60%, at least 70%, at least 80%, or at least 90%.

The term “epitope” means a portion of an antigen capable of specific binding to an antibody. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to CD39 variants with different point-mutations.

Percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, or CLUSTAL OMEGA software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of one or more amino acids with one or more chemically or functionally similar amino acids. Conservative substitution tables providing similar amino acids are well known in the art. Polypeptide sequences having such substitutions are known as “conservatively modified variants.” Such conservatively modified variants are in addition to and do not exclude polymorphic variants, interspecies homologs, and alleles. By way of example, the following groups of amino acids are considered conservative substitutions for one another.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

| | |
|---|---|
| Alcohol Group-Containing Residues | S and T |
| Aliphatic Residues | I, L, V, and M |
| Cycloalkenyl-associated Residues | F, H, W, and Y |
| Hydrophobic Residues | A, C, F, G, H, I, L, M, |
| | R, T, V, W, and Y |
| Negatively Charged Residues | D and E |
| Polar Residues | C, D, E, H, K, N, Q, R, |
| | S, and T |
| Positively Charged Residues | H, K, and R |
| Small Residues | A, C, D, G, N, P, S, T, and V |
| Very Small Residues | A, G, and S |
| Residues Involved in Turn Formation | A, C, D, E, G, H, K, N, |
| | Q, R, S, P, and T |
| Flexible Residues | Q, T, K, S, G, P, D, E, and R |

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly: G); histidine (His; H), isoleucine (Ile; I), leucine (Leu; L.), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of an antibody or composition that when administered to a subject is effective to treat a disease or disorder.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has cancer, an autoimmune disease or condition, and/or an infection that can be treated with an antibody provided herein. In some embodiments, the subject is a human that is suspected to have cancer, an autoimmune disease or condition, and/or an infection.

### 2. Antibodies

Provided herein are antibodies that selectively bind human CD39, as well as the nucleic acids that encode the antibodies. In some aspects, the antibody selectively binds to the extracellular domain of human CD39.

In some embodiments, the antibody binds to homologs of human CD39. In some aspects, the antibody binds to a homolog of human CD39 from a species selected from monkeys, mice, dogs, cats, rats, cows, horses, goats, and sheep. In some aspects, the homolog is a cynomolgus monkey homolog. In some aspects, the homolog is a murine homolog.

In some embodiments, the antibody has one or more CDRs having particular lengths, in terms of the number of amino acid residues. In some embodiments, the Chothia CDR-H1 of the antibody is 6, 7, 8, or 9 residues in length. In some embodiments, the Kabat CDR-H1 of the antibody is 4, 5, 6, or 7 residues in length. In some embodiments, the Chothia CDR-H2 of the antibody is 5, 6, or 7 residues in length. In some embodiments, the Kabat CDR-H2 of the antibody is 15, 16, 17, or 18 residues in length. In some embodiments, the Kabat/Chothia CDR-H3 of the antibody is 5, 6, 7, 8, 9, 10, 11, or 12 residues in length.

In some aspects, the Kabat/Chothia CDR-L1 of the antibody is 9, 10, 11, 12, 13, 14, 15, or 16 residues in length. In some aspects, the Kabat/Chothia CDR-L2 of the antibody is 6, 7, or 8 residues in length. In some aspects, the Kabat/Chothia CDR-L3 of the antibody is 8, 9, 10, 11, or 12 residues in length.

In some embodiments, the antibody comprises a light chain. In some aspects, the light chain is a kappa light chain. In some aspects, the light chain is a lambda light chain.

In some embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

In some embodiments, the antibody is an affinity matured antibody. In some aspects, the antibody is an affinity matured antibody derived from an illustrative sequence provided in this disclosure.

In some aspects, the antibody inhibits conversion by CD39 of ATP to ADP and/or ADP to AMP. In some aspects, the antibody decreases the levels of phosphate, ADP, AMP, and/or adenosine and/or increases the levels of ATP.

In some embodiments, the antibody increases proliferation of stimulated CD4- and CD8⁺ T cells. In some embodiments, the antibody increases stimulated PBMC secretion of INF-γ, TNF-α, IL-2, and/or IL-1β.

In some embodiments, the antibody increases a T effector cell function. In some embodiments, the antibody decreases the number of regulatory T cells in tissues or in circulation. In some embodiments, the antibody suppresses a regulatory or T cell activity. In some embodiments, the antibody increase B cell function. In some embodiments, the antibody increases antigen presenting cell function. In some embodiments, the antibody decreases or prevents activation of phospho antigen specific T cells selected from MAIT cells and gamma delta T cells.

In some aspects, the decrease is about or less than a 10% decrease, about or less than a 20% decrease, about or less than a 30% decrease, about or less than a 40% decrease, about or less than a 50% decrease, about or less than a 60% decrease, about or less than a 70% decrease, about or less than an 80% decrease, about or less than a 90% decrease, or about a complete decrease. In some aspects, the increase is about or greater than a 10% increase, about or greater than a 20% increase, about or greater than a 30% increase, about or greater than a 40% increase, about or greater than a 50% increase, about or greater than a 60% increase, about or greater than a 70% increase, about or greater than an 80% increase, about or greater than a 90% increase, or a complete increase.

Given that CD39 degrades ATP and ADP to adenosine, CD39 can be viewed as an immunological switch that shifts ATP-driven pro-inflammatory immune cell activity toward an anti-inflammatory state mediated by adenosine. CD39 has a role in regulating the function of several immune cell types, including lymphocytes, neutrophils, monocytes/macrophages, dendritic cells, and endothelial cells and shifting the switch can have a significant impact on disease. For example, the generation of adenosine via CD39 is recognized as a major mechanism of regulatory T cell (Treg) immunosuppressive function.

The antibodies provided herein may be useful for the treatment of a variety of diseases and conditions, including cancers, autoimmune diseases, and infections. In some embodiments, the antibody inhibits CD39 function on tumor cells. In some embodiments, the antibody inhibits angiogenesis.

The frequency of CD39⁺ Tregs and the expression on the cell surface is increased in some human cancers, and the importance of CD39⁺ Tregs in promoting tumor growth and metastasis has been demonstrated using several in vivo models.

Immunohistochemical staining of normal and tumor tissues has revealed that CD39 expression is significantly higher in several types of human cancer than in normal tissues. In cancer specimens, CD39 is expressed by infiltrating lymphocytes, the tumor stroma, and/or tumor cells. CD39 in cancer cells displays ATPase activity and generates adenosine. CD39⁺ cancer cells inhibited the proliferation of CD4 and CD8 T cells and the generation of cytotoxic effector CD8 T cells (CTL.) in a CD39- and adenosine-dependent manner.

### 2.1. CDR-H3 Sequences

In some embodiments, the antibody comprises a CDR-H3 sequence comprising, consisting of, or consisting essentially of a sequence selected from SEQ ID NOs: 82-109. In some aspects, the antibody comprises a CDR-H3 sequence comprising, consisting of, or consisting essentially of SEQ ID NO: 82. In some aspects, the antibody comprises a CDR-H3 sequence comprising, consisting of, or consisting essentially of SEQ ID NO: 83. In some aspects, the antibody comprises a CDR-H3 sequence comprising, consisting of, or consisting essentially of SEQ ID NO: 84. In some aspects, the antibody comprises a CDR-H3 sequence comprising, consisting of, or consisting essentially of SEQ ID NO: 85. In some aspects, the antibody comprises a CDR-H3 sequence comprising, consisting of, or consisting essentially of SEQ ID NO: 86. In some aspects, the antibody comprises a CDR-H3 sequence comprising consisting of, or consisting essentially of SEQ ID NO: 87. In some aspects, the antibody comprises a CDR-H3 sequence comprising, consisting of, or consisting essentially of SEQ ID NO: 88. In some aspects, the antibody comprises a CDR-H3 sequence comprising, consisting of, or consisting essentially of SEQ ID NO: 89. In some aspects, the antibody comprises a CDR-13 sequence comprising, consisting of, or consisting essentially of SEQ ID NO: 90. In some aspects, the antibody comprises a CDR-H3 sequence comprising, consisting of, or consisting essentially of SEQ ID NO: 91. In some aspects, the antibody comprises a CDR-H3 sequence comprising, consisting of, or consisting essentially of SEQ ID NO: 92. In some aspects, the antibody comprises a CDR-13 sequence comprising, consisting of, or consisting essentially of SEQ ID NO: 93. In some aspects, the antibody comprises a CDR-H3 sequence comprising, consisting of, or consisting essentially of SEQ ID NO: 94. In some aspects, the antibody comprises a CDR-H3 sequence comprising, consisting of, or consisting essentially of SEQ ID NO: 95. In some aspects, the antibody comprises a CDR-H3 sequence comprising, consisting of, or consisting essentially of SEQ ID NO: 96. In some aspects, the antibody comprises a CDR-H3 sequence comprising, consisting of, or consisting essentially of SEQ ID NO: 97. In some aspects, the antibody comprises a CDR-H3 sequence comprising, consisting of, or consisting essentially of SEQ ID NO: 98. In some aspects, the antibody comprises a CDR-H3 sequence comprising, consisting of, or consisting essentially of SEQ ID NO: 99. In some aspects, the antibody comprises a CDR-H3 sequence comprising, consisting of, or consisting essentially of SEQ ID NO: 100. In some aspects, the antibody comprises a CDR-H3 sequence comprising, consisting of, or consisting essentially of SEQ ID NO: 101. In some aspects, the antibody comprises a CDR-H3 sequence comprising, consisting of, or consisting essentially of SEQ ID NO: 102. In some aspects, the antibody comprises a CDR-H3 sequence comprising, consisting of, or consisting essentially of SEQ ID NO: 103. In some aspects, the antibody comprises a CDR-H3 sequence comprising, consisting of, or consisting essentially of SEQ ID NO: 104. In some aspects, the antibody comprises a CDR-H3 sequence comprising, consisting of, or consisting essentially of SEQ ID NO: 105. In some aspects, the antibody comprises a CDR-H3 sequence comprising, consisting of, or consisting essentially of SEQ ID NO: 106. In some aspects, the antibody comprises a CDR-H3 sequence comprising, consisting of, or consisting essentially of SEQ ID NO: 107. In some aspects, the antibody comprises a CDR-H3 sequence comprising, consisting of, or consisting essentially of SEQ ID NO: 108. In some aspects, the antibody comprises a CDR-H3 sequence comprising, consisting of, or consisting essentially of SEQ ID NO: 109.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-13 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

### 2.2. V_{H} Sequences Comprising Illustrative CDRs

In some embodiments, the antibody comprises a V_{H} sequence comprising one or more CDR-H sequences comprising, consisting of, or consisting essentially of one or more illustrative CDR-H sequences provided in this disclosure, and variants thereof.

### 2.2.1. V_{H} Sequences Comprising Illustrative Kabat CDRs

In some embodiments, the antibody comprises a V_{H} sequence comprising one or more Kabat CDR-H sequences comprising, consisting of, or consisting essentially of one or more illustrative Kabat CDR-H sequences provided in this disclosure, and variants thereof.

### 2.2.1.1. KABAT CDR-H3

In some embodiments, the antibody comprises a V_{H} sequence comprising a Kabat CDR-H3 sequence comprising, consisting of, or consisting essentially of a sequence selected from SEQ ID NOs: 82-109. In some aspects, the antibody comprises a V_{H} sequence comprising a Kabat CDR-H3 sequence comprising, consisting of, or consisting essentially of SEQ ID NO: 82. In some aspects, the antibody comprises a V_{H} sequence comprising a Kabat CDR-H3 sequence comprising, consisting of, or consisting essentially of SEQ ID NO: 83. In some aspects, the antibody comprises a V_{H} sequence comprising a Kabat CDR-H3 sequence comprising, consisting of, or consisting essentially of SEQ ID NO: 84. In some aspects, the antibody comprises a V_{H} sequence comprising a Kabat CDR-H3 sequence comprising, consisting of, or consisting essentially of SEQ ID NO: 85. In some aspects, the antibody comprises a V_{H} sequence comprising a Kabat CDR-13 sequence comprising, consisting of, or consisting essentially of SEQ ID NO: 86. In some aspects, the antibody comprises a V_{H} sequence comprising a Kabat CDR-13 sequence comprising, consisting of, or consisting essentially of SEQ ID NO: 87. In some aspects, the antibody comprises a V_{H} sequence comprising a Kabat CDR-H3 sequence comprising, consisting of, or consisting essentially of SEQ ID NO: 88. In some aspects, the antibody comprises a V_{H} sequence comprising a Kabat CDR-H3 sequence comprising, consisting of, or consisting essentially of SEQ ID NO: 89. In some aspects, the antibody comprises a V_{H} sequence comprising a Kabat CDR-H3 sequence comprising, consisting of, or consisting essentially of SEQ ID NO: 90. In some aspects, the antibody comprises a V_{H} sequence comprising a Kabat CDR-H3 sequence comprising, consisting of, or consisting essentially of SEQ ID NO: 91. In some aspects, the antibody comprises a V_{H} sequence comprising a Kabat CDR-H3 sequence comprising, consisting of, or consisting essentially of SEQ ID NO: 92. In some aspects, the antibody comprises a V_{H} sequence comprising a Kabat CDR-H3 sequence comprising, consisting of, or consisting essentially of SEQ ID NO: 93. In some aspects, the antibody comprises a V_{H} sequence comprising a Kabat CDR-H3 sequence comprising, consisting of, or consisting essentially of SEQ ID NO: 94. In some aspects, the antibody comprises a V_{H} sequence comprising a Kabat CDR-H3 sequence comprising, consisting of, or consisting essentially of SEQ ID NO: 95. In some aspects, the antibody comprises a V_{H} sequence comprising a Kabat CDR-H3 sequence comprising, consisting of, or consisting essentially of SEQ ID NO: 96. In some aspects, the antibody comprises a V_{H} sequence comprising a Kabat CDR-H3 sequence comprising, consisting of, or consisting essentially of SEQ ID NO: 97. In some aspects, the antibody comprises a V_{H} sequence comprising a Kabat CDR-H3 sequence comprising, consisting of, or consisting essentially of SEQ ID NO: 98. In some aspects, the antibody comprises a Vu sequence comprising a Kabat CDR-H3 sequence comprising, consisting of, or consisting essentially of SEQ ID NO: 99. In some aspects, the antibody comprises a V_{H} sequence comprising a Kabat CDR-H3 sequence comprising, consisting of, or consisting essentially of SEQ ID NO: 100. In some aspects, the antibody comprises a V_{H} sequence comprising a Kabat CDR-H3 sequence comprising, consisting of, or consisting essentially of SEQ ID NO: 101. In some aspects, the antibody comprises a V_{H} sequence comprising a Kabat CDR-H3 sequence comprising, consisting of, or consisting essentially of SEQ ID NO: 102. In some aspects, the antibody comprises a V_{H} sequence comprising a Kabat CDR-H3 sequence comprising, consisting of, or consisting essentially of SEQ ID NO: 103. In some aspects, the antibody comprises a V_{H} sequence comprising a Kabat CDR-H3 sequence comprising, consisting of, or consisting essentially of SEQ ID NO: 104. In some aspects, the antibody comprises a V_{H} sequence comprising a Kabat CDR-H3 sequence comprising, consisting of, or consisting essentially of SEQ ID NO: 105. In some aspects, the antibody comprises a V_{H} sequence comprising a Kabat CDR-H3 sequence comprising, consisting of, or consisting essentially of SEQ ID NO: 106. In some aspects, the antibody comprises a V_{H} sequence comprising a Kabat CDR-H3 sequence comprising, consisting of, or consisting essentially of SEQ ID NO: 107. In some aspects, the antibody comprises a V_{H} sequence comprising a Kabat CDR-H3 sequence comprising, consisting of, or consisting essentially of SEQ ID NO: 108. In some aspects, the antibody comprises a V_{H} sequence comprising a Kabat CDR-H3 sequence comprising, consisting of, or consisting essentially of SEQ ID NO: 109.

### 2.2.1.2. Kabat CDR-H2

In some embodiments, the antibody comprises a V_{H} sequence comprising a Kabat CDR-H2 sequence comprising, consisting of, or consisting essentially of a sequence selected from SEQ ID NOs: 63-81. In some aspects, the antibody comprises a V_{H} sequence comprising a Kabat CDR-H2 sequence comprising, consisting of, or consisting essentially of SEQ ID NO: 63. In some aspects, the antibody comprises a V_{H} sequence comprising a Kabat CDR-H2 sequence comprising, consisting of, or consisting essentially of SEQ ID NO: 64. In some aspects, the antibody comprises a V_{H} sequence comprising a Kabat CDR-H2 sequence comprising, consisting of, or consisting essentially of SEQ ID NO: 65. In some aspects, the antibody comprises a V_{H} sequence comprising a Kabat CDR-H2 sequence comprising, consisting of, or consisting essentially of SEQ ID NO: 66. In some aspects, the antibody comprises a V_{H} sequence comprising a Kabat CDR-H2 sequence comprising, consisting of, or consisting essentially of SEQ ID NO: 67. In some aspects, the antibody comprises a V_{H} sequence comprising a Kabat CDR-H2 sequence comprising, consisting of, or consisting essentially of SEQ ID NO: 68. In some aspects, the antibody comprises a V_{H} sequence comprising a Kabat CDR-H2 sequence comprising, consisting of, or consisting essentially of SEQ ID NO: 69. In some aspects, the antibody comprises a V_{H} sequence comprising a Kabat CDR-H2 sequence comprising, consisting of, or consisting essentially of SEQ ID NO: 70. In some aspects, the antibody comprises a V_{H} sequence comprising a Kabat CDR-H2 sequence comprising, consisting of, or consisting essentially of SEQ ID NO: 71. In some aspects, the antibody comprises a V_{H} sequence comprising a Kabat CDR-H2 sequence comprising, consisting of, or consisting essentially of SEQ ID NO: 72. In some aspects, the antibody comprises a V_{H} sequence comprising a Kabat CDR-H2 sequence comprising, consisting of, or consisting essentially of SEQ ID NO: 73. In some aspects, the antibody comprises a V_{H} sequence comprising a Kabat CDR-H2 sequence comprising, consisting of, or consisting essentially of SEQ ID NO: 74. In some aspects, the antibody comprises a V_{H} sequence comprising a Kabat CDR-H2 sequence comprising, consisting of, or consisting essentially of SEQ ID NO: 75. In some aspects, the antibody comprises a V_{H} sequence comprising a Kabat CDR-H2 sequence comprising, consisting of, or consisting essentially of SEQ ID NO: 76. In some aspects, the antibody comprises a V_{H} sequence comprising a Kabat CDR-H2 sequence comprising, consisting of, or consisting essentially of SEQ ID NO: 77. In some aspects, the antibody comprises a V_{H} sequence comprising a Kabat CDR-H2 sequence comprising, consisting of, or consisting essentially of SEQ ID NO: 78. In some aspects, the antibody comprises a V_{H} sequence comprising a Kabat CDR-H2 sequence comprising, consisting of, or consisting essentially of SEQ ID NO: 79. In some aspects, the antibody comprises a V_{H} sequence comprising a Kabat CDR-H2 sequence comprising, consisting of, or consisting essentially of SEQ ID NO: 80. In some aspects, the antibody comprises a V_{H} sequence comprising a Kabat CDR-H2 sequence comprising, consisting of, or consisting essentially of SEQ ID NO: 81.

### 2.2.1.3. Kabat CDR-H1

In some embodiments, the antibody comprises a V_{H} sequence comprising a Kabat CDR-H1 sequence comprising, consisting of, or consisting essentially of a sequence selected from SEQ ID NOs: 25-45. In some aspects, the antibody comprises a V_{H} sequence comprising a Kabat CDR-H1 sequence comprising, consisting of, or consisting essentially of SEQ ID NO: 25. In some aspects, the antibody comprises a V_{H} sequence comprising a Kabat CDR-H1 sequence comprising, consisting of, or consisting essentially of SEQ ID NO: 26. In some aspects, the antibody comprises a V_{H} sequence comprising a Kabat CDR-H1 sequence comprising, consisting of, or consisting essentially of SEQ ID NO: 27. In some aspects, the antibody comprises a V_{H} sequence comprising a Kabat CDR-H1 sequence comprising, consisting of, or consisting essentially of SEQ ID NO: 28. In some aspects, the antibody comprises a V_{H} sequence comprising a Kabat CDR-H1 sequence comprising, consisting of, or consisting essentially of SEQ ID NO: 29. In some aspects, the antibody comprises a Vu sequence comprising a Kabat CDR-H1 sequence comprising, consisting of, or consisting essentially of SEQ ID NO: 30. In some aspects, the antibody comprises a V_{H} sequence comprising a Kabat CDR-H1 sequence comprising, consisting of, or consisting essentially of SEQ ID NO: 31. In some aspects, the antibody comprises a V_{H} sequence comprising a Kabat CDR-H1 sequence comprising, consisting of, or consisting essentially of SEQ ID NO: 32. In some aspects, the antibody comprises a V_{H} sequence comprising a Kabat CDR-H1 sequence comprising, consisting of, or consisting essentially of SEQ ID NO: 33. In some aspects, the antibody comprises a V_{H} sequence comprising a Kabat CDR-H1 sequence comprising, consisting of, or consisting essentially of SEQ ID NO: 34. In some aspects, the antibody comprises a V_{H} sequence comprising a Kabat CDR-H1 sequence comprising, consisting of, or consisting essentially of SEQ ID NO: 35. In some aspects, the antibody comprises a V_{H} sequence comprising a Kabat CDR-H1 sequence comprising, consisting of, or consisting essentially of SEQ ID NO: 36. In some aspects, the antibody comprises a V_{H} sequence comprising a Kabat CDR-H1 sequence comprising, consisting of, or consisting essentially of SEQ ID NO: 37. In some aspects, the antibody comprises a V_{H} sequence comprising a Kabat CDR-H1 sequence comprising, consisting of, or consisting essentially of SEQ ID NO: 38. In some aspects, the antibody comprises a V_{H} sequence comprising a Kabat CDR-H1 sequence comprising, consisting of, or consisting essentially of SEQ ID NO: 39. In some aspects, the antibody comprises a V_{H} sequence comprising a Kabat CDR-H1 sequence comprising, consisting of, or consisting essentially of SEQ ID NO: 40. In some aspects, the antibody comprises a V_{H} sequence comprising a Kabat CDR-H1 sequence comprising, consisting of, or consisting essentially of SEQ ID NO: 41. In some aspects, the antibody comprises a V_{H} sequence comprising a Kabat CDR-H1 sequence comprising, consisting of, or consisting essentially of SEQ ID NO: 42. In some aspects, the antibody comprises a V_{H} sequence comprising a Kabat CDR-H1 sequence comprising, consisting of, or consisting essentially of SEQ ID NO: 43. In some aspects, the antibody comprises a V_{H} sequence comprising a Kabat CDR-H1 sequence comprising, consisting of, or consisting essentially of SEQ ID NO: 44. In some aspects, the antibody comprises a V_{H} sequence comprising a Kabat CDR-H1 sequence comprising, consisting of, or consisting essentially of SEQ ID NO: 45.

### 2.2.1.4. Kabat CDR-H3+Kabat CDR-H2

In some embodiments, the antibody comprises a V_{H} sequence comprising a Kabat CDR-H3 sequence comprising, consisting of, or consisting essentially of a sequence selected from SEQ ID NOs: 82-109, and a Kabat CDR-H2 sequence comprising, consisting of, or consisting essentially of a sequence selected from SEQ ID NOs: 63-81. In some aspects, the Kabat CDR-H3 sequence and the Kabat CDR-H2 sequence are both from a single illustrative V_{H} sequence provided in this disclosure. For example, in some aspects, the Kabat CDR-H3 and Kabat CDR-H2 are both from a single illustrative V_{H} sequence selected from SEQ ID NOs: 179-218.

### 2.2.1.5. Kabat CDR-H3+Kabat CDR-H1

In some embodiments, the antibody comprises a V_{H} sequence comprising a Kabat CDR-H3 sequence comprising, consisting of, or consisting essentially of a sequence selected from SEQ ID NOs: 82-109, and a Kabat CDR-H1 sequence comprising, consisting of, or consisting essentially of a sequence selected from SEQ ID NOs: 25-45. In some aspects, the Kabat CDR-H3 sequence and the Kabat CDR-H1 sequence are both from a single illustrative V_{H} sequence provided in this disclosure. For example, in some aspects, the Kabat CDR-H3 and Kabat CDR-H1 are both from a single illustrative V_{H} sequence selected from SEQ ID NOs: 179-218.

### 2.2.1.6. Kabat CDR-H1+Kabat CDR-H2

In some embodiments, the antibody comprises a V_{H} sequence comprising a Kabat CDR-H1 sequence comprising, consisting of, or consisting essentially of a sequence selected from SEQ ID NOs: 25-45 and a Kabat CDR-H2 sequence comprising, consisting of, or consisting essentially of a sequence selected from SEQ ID NOs: 63-81. In some aspects, the Kabat CDR-H1 sequence and the Kabat CDR-H2 sequence are both from a single illustrative V_{H} sequence provided in this disclosure. For example, in some aspects, the Kabat CDR-H1 and Kabat CDR-H2 are both from a single illustrative V_{H} sequence selected from SEQ ID NOs: 179-218.

### 2.2.1.7. Kabat CDR-H1+Kabat CDR-H2+Kabat CDR-H3

In some embodiments, the antibody comprises a V_{H} sequence comprising a Kabat CDR-H1 sequence comprising, consisting of, or consisting essentially of a sequence selected from SEQ ID NOs: 25-45, a Kabat CDR-H2 sequence comprising, consisting of, or consisting essentially of a sequence selected from SEQ ID NOs: 63-81, and a Kabat CDR-H3 sequence comprising, consisting of, or consisting essentially of a sequence selected from SEQ ID NOs: 82-109. In some aspects, the Kabat CDR-H1 sequence, Kabat CDR-H2 sequence, and Kabat CDR-H3 sequence are all from a single illustrative V_{H} sequence provided in this disclosure. For example, in some aspects, the Kabat CDR-H1, Kabat CDR-H2, and Kabat CDR-H3 are all from a single illustrative V_{H} sequence selected from SEQ ID NOs: 179-218.

In some aspects, the antibody comprises a V_{H} sequence comprising a Kabat CDR-H1 sequence comprising SEQ ID NO: 25, a Kabat CDR-H2 sequence comprising SEQ ID NO: 63, and a Kabat CDR-H3 sequence comprising SEQ ID NO: 82. In some aspects, the antibody comprises a V_{H} sequence comprising a Kabat CDR-H1 sequence comprising SEQ ID NO: 26, a Kabat CDR-H2 sequence comprising SEQ ID NO: 64, and a Kabat CDR-H3 sequence comprising SEQ ID NO: 83. In some aspects, the antibody comprises a V_{H} sequence comprising a Kabat CDR-H1 sequence comprising SEQ ID NO: 27, a Kabat CDR-H2 sequence comprising SEQ ID NO: 65, and a Kabat CDR-H3 sequence comprising SEQ ID NO: 84. In some aspects, the antibody comprises a V_{H} sequence comprising a Kabat CDR-H1 sequence comprising SEQ ID NO: 26, a Kabat CDR-H2 sequence comprising SEQ ID NO: 64, and a Kabat CDR-H3 sequence comprising SEQ ID NO: 85. In some aspects, the antibody comprises a V_{H} sequence comprising a Kabat CDR-H1 sequence comprising SEQ ID NO: 26, a Kabat CDR-H2 sequence comprising SEQ ID NO: 64, and a Kabat CDR-H3 sequence comprising SEQ ID NO: 86. In some aspects, the antibody comprises a V_{H} sequence comprising a Kabat CDR-H1 sequence comprising SEQ ID NO: 25, a Kabat CDR-H2 sequence comprising SEQ ID NO: 66, and a Kabat CDR-H3 sequence comprising SEQ ID NO: 82. In some aspects, the antibody comprises a V_{H} sequence comprising a Kabat CDR-H1 sequence comprising SEQ ID NO): 26, a Kabat CDR-H2 sequence comprising SEQ ID NO: 65, and a Kabat CDR-H3 sequence comprising SEQ ID NO: 82. In some aspects, the antibody comprises a Vn sequence comprising a Kabat CDR-H1 sequence comprising SEQ ID NO: 27, a Kabat CDR-H2 sequence comprising SEQ ID NO: 65, and a Kabat CDR-H3 sequence comprising SEQ ID NO: 82. In some aspects, the antibody comprises a V_{H} sequence comprising a Kabat CDR-H1 sequence comprising SEQ ID NO: 28, a Kabat CDR-H2 sequence comprising SEQ ID NO: 67, and a Kabat CDR-H3 sequence comprising SEQ ID NO: 82. In some aspects, the antibody comprises a V_{H} sequence comprising a Kabat CDR-H1 sequence comprising SEQ ID NO: 29, a Kabat CDR-H2 sequence comprising SEQ ID NO: 68, and a Kabat CDR-H3 sequence comprising SEQ ID NO: 82. In some aspects, the antibody comprises a V_{H} sequence comprising a Kabat CDR-H1 sequence comprising SEQ ID NO: 26, a Kabat CDR-H2 sequence comprising SEQ ID NO: 64, and a Kabat CDR-H3 sequence comprising SEQ ID NO: 82. In some aspects, the antibody comprises a V_{H} sequence comprising a Kabat CDR-H1 sequence comprising SEQ ID NO: 30, a Kabat CDR-H2 sequence comprising SEQ ID NO: 69, and a Kabat CDR-H3 sequence comprising SEQ ID NO: 87. In some aspects, the antibody comprises a V_{H} sequence comprising a Kabat CDR-H1 sequence comprising SEQ ID NO: 31, a Kabat CDR-H2 sequence comprising SEQ ID NO: 70, and a Kabat CDR-H3 sequence comprising SEQ ID NO: 88. In some aspects, the antibody comprises a V_{H} sequence comprising a Kabat CDR-H1 sequence comprising SEQ ID NO: 32, a Kabat CDR-H2 sequence comprising SEQ ID NO: 71, and a Kabat CDR-H3 sequence comprising SEQ ID NO: 89. In some aspects, the antibody comprises a V_{H} sequence comprising a Kabat CDR-H1 sequence comprising SEQ ID NO: 33, a Kabat CDR-H2 sequence comprising SEQ ID NO: 70, and a Kabat CDR-H3 sequence comprising SEQ ID NO: 90. In some aspects, the antibody comprises a V_{H} sequence comprising a Kabat CDR-H1 sequence comprising SEQ ID NO: 34, a Kabat CDR-H2 sequence comprising SEQ ID NO: 72, and a Kabat CDR-H3 sequence comprising SEQ ID NO: 91. In some aspects, the antibody comprises a V_{H} sequence comprising a Kabat CDR-H1 sequence comprising SEQ ID NO: 30, a Kabat CDR-H2 sequence comprising SEQ ID NO: 71, and a Kabat CDR-H3 sequence comprising SEQ ID NO: 92. In some aspects, the antibody comprises a V_{H} sequence comprising a Kabat CDR-H1 sequence comprising SEQ ID NO: 35, a Kabat CDR-H2 sequence comprising SEQ ID NO: 70, and a Kabat CDR-H3 sequence comprising SEQ ID NO: 93. In some aspects, the antibody comprises a V_{H} sequence comprising a Kabat CDR-H1 sequence comprising SEQ ID NO: 33, a Kabat CDR-H2 sequence comprising SEQ ID NO: 70, and a Kabat CDR-H13 sequence comprising SEQ ID NO: 92. In some aspects, the antibody comprises a V_{H} sequence comprising a Kabat CDR-H1 sequence comprising SEQ ID NO: 36, a Kabat CDR-H2 sequence comprising SEQ ID NO: 72, and a Kabat CDR-H3 sequence comprising SEQ ID NO: 87. In some aspects, the antibody comprises a V_{H} sequence comprising a Kabat CDR-H1 sequence comprising SEQ ID NO: 30, a Kabat CDR-H2 sequence comprising SEQ ID NO: 73, and a Kabat CDR-H3 sequence comprising SEQ ID NO): 87. In some aspects, the antibody comprises a V_{H} sequence comprising a Kabat CDR-H1 sequence comprising SEQ ID NO: 37, a Kabat CDR-H2 sequence comprising SEQ ID NO: 74, and a Kabat CDR-H3 sequence comprising SEQ ID NO: 87. In some aspects, the antibody comprises a V_{H} sequence comprising a Kabat CDR-H1 sequence comprising SEQ ID NO: 38, a Kabat CDR-H2 sequence comprising SEQ ID NO: 75, and a Kabat CDR-H3 sequence comprising SEQ ID NO: 94. In some aspects, the antibody comprises a V_{H} sequence comprising a Kabat CDR-H1 sequence comprising SEQ ID NO: 39, a Kabat CDR-H2 sequence comprising SEQ ID NO: 76, and a Kabat CDR-H3 sequence comprising SEQ ID NO: 95. In some aspects, the antibody comprises a V_{H} sequence comprising a Kabat CDR-H1 sequence comprising SEQ ID NO: 40, a Kabat CDR-H2 sequence comprising SEQ ID NO: 76, and a Kabat CDR-H3 sequence comprising SEQ ID NO: 96. In some aspects, the antibody comprises a V_{H} sequence comprising a Kabat CDR-H1 sequence comprising SEQ ID NO: 39, a Kabat CDR-H2 sequence comprising SEQ ID NO: 76, and a Kabat CDR-H3 sequence comprising SEQ ID NO: 94. In some aspects, the antibody comprises a V_{H} sequence comprising a Kabat CDR-H1 sequence comprising SEQ ID NO: 38, a Kabat CDR-H2 sequence comprising SEQ ID NO: 69, and a Kabat CDR-H3 sequence comprising SEQ ID NO: 97. In some aspects, the antibody comprises a V_{H} sequence comprising a Kabat CDR-H1 sequence comprising SEQ ID NO: 41, a Kabat CDR-H2 sequence comprising SEQ ID NO: 69, and a Kabat CDR-H3 sequence comprising SEQ ID NO: 98. In some aspects, the antibody comprises a V_{H} sequence comprising a Kabat CDR-H1 sequence comprising SEQ ID NO: 41, a Kabat CDR-H2 sequence comprising SEQ ID NO: 69, and a Kabat CDR-H3 sequence comprising SEQ ID NO: 99. In some aspects, the antibody comprises a V_{H} sequence comprising a Kabat CDR-H1 sequence comprising SEQ ID NO: 41, a Kabat CDR-H2 sequence comprising SEQ ID NO: 69, and a Kabat CDR-H3 sequence comprising SEQ ID NO: 100. In some aspects, the antibody comprises a V_{H} sequence comprising a Kabat CDR-H1 sequence comprising SEQ ID NO: 38, a Kabat CDR-H2 sequence comprising SEQ ID NO: 75, and a Kabat CDR-H3 sequence comprising SEQ ID NO: 101. In some aspects, the antibody comprises a V_{H} sequence comprising a Kabat CDR-H1 sequence comprising SEQ ID NO: 42, a Kabat CDR-H2 sequence comprising SEQ ID NO: 78, and a Kabat CDR-H3 sequence comprising SEQ ID NO: 102. In some aspects, the antibody comprises a V_{H} sequence comprising a Kabat CDR-H1 sequence comprising SEQ ID NO: 38, a Kabat CDR-H2 sequence comprising SEQ ID NO: 75, and a Kabat CDR-H3 sequence comprising SEQ ID NO: 103. In some aspects, the antibody comprises a V_{H} sequence comprising a Kabat CDR-H1 sequence comprising SEQ ID NO: 43, a Kabat CDR-H2 sequence comprising SEQ ID NO: 79, and a Kabat CDR-H3 sequence comprising SEQ ID NO: 104. In some aspects, the antibody comprises a V_{H} sequence comprising a Kabat CDR-H1 sequence comprising SEQ ID NO: 38, a Kabat CDR-H2 sequence comprising SEQ ID NO: 69, and a Kabat CDR-H3 sequence comprising SEQ ID NO: 103. In some aspects, the antibody comprises a V_{H} sequence comprising a Kabat CDR-H1 sequence comprising SEQ ID NO: 38, a Kabat CDR-H2 sequence comprising SEQ ID NO: 69, and a Kabat CDR-H3 sequence comprising SEQ ID NO: 106. In some aspects, the antibody comprises a V_{H} sequence comprising a Kabat CDR-H1 sequence comprising SEQ ID NO: 38, a Kabat CDR-H2 sequence comprising SEQ ID NO: 69, and a Kabat CDR-H3 sequence comprising SEQ ID NO: 107. In some aspects, the antibody comprises a V_{H} sequence comprising a Kabat CDR-H1 sequence comprising SEQ ID NO: 44, a Kabat CDR-H2 sequence comprising SEQ ID NO: 80, and a Kabat CDR-H3 sequence comprising SEQ ID NO: 108. In some aspects, the antibody comprises a V_{H} sequence comprising a Kabat CDR-H1 sequence comprising SEQ ID NO: 45, a Kabat CDR-H2 sequence comprising SEQ ID NO: 81, and a Kabat CDR-H3 sequence comprising SEQ ID NO: 109.

In some aspects, the antibody comprises a V_{H} sequence comprising a Kabat CDR-H1 sequence comprising SEQ ID NO: 26, a Kabat CDR-H2 sequence comprising SEQ ID NO: 65, and a Kabat CDR-H3 sequence comprising SEQ ID NO: 83. In some aspects, the antibody comprises a V_{H} sequence comprising a Kabat CDR-H1 sequence comprising SEQ ID NO: 26, a Kabat CDR-H2 sequence comprising SEQ ID NO: 65, and a Kabat CDR-H3 sequence comprising SEQ ID NO: 84. In some aspects, the antibody comprises a V_{H} sequence comprising a Kabat CDR-H1 sequence comprising SEQ ID NO: 26, a Kabat CDR-H2 sequence comprising SEQ ID NO: 65, and a Kabat CDR-H3 sequence comprising SEQ ID NO: 85. In some aspects, the antibody comprises a V_{H} sequence comprising a Kabat CDR-H1 sequence comprising SEQ ID NO: 26, a Kabat CDR-H2 sequence comprising SEQ ID NO: 65, and a Kabat CDR-H3 sequence comprising SEQ ID NO: 86. In some aspects, the antibody comprises a V_{H} sequence comprising a Kabat CDR-H1 sequence comprising SEQ ID NO: 26, a Kabat CDR-H2 sequence comprising SEQ ID NO: 64, and a Kabat CDR-H3 sequence comprising SEQ ID NO: 84. In some aspects, the antibody comprises a V_{H} sequence comprising a Kabat CDR-H1 sequence comprising SEQ ID NO: 26, a Kabat CDR-H2 sequence comprising SEQ ID NO: 64, and a Kabat CDR-H3 sequence comprising SEQ ID NO: 86. In some aspects, the antibody comprises a V_{H} sequence comprising a Kabat CDR-H1 sequence comprising SEQ ID NO: 27, a Kabat CDR-H2 sequence comprising SEQ ID NO: 65, and a Kabat CDR-H3 sequence comprising SEQ ID NO: 83. In some aspects, the antibody comprises a V_{H} sequence comprising a Kabat CDR-H1 sequence comprising SEQ ID NO: 27, a Kabat CDR-H2 sequence comprising SEQ ID NO: 65, and a Kabat CDR-H3 sequence comprising SEQ ID NO: 86. In some aspects, the antibody comprises a V_{H} sequence comprising a Kabat CDR-H1 sequence comprising SEQ ID NO: 30, a Kabat CDR-H2 sequence comprising SEQ ID NO: 71, and a Kabat CDR-H3 sequence comprising SEQ ID NO: 93. In some aspects, the antibody comprises a V_{H} sequence comprising a Kabat CDR-H1 sequence comprising SEQ ID NO: 30, a Kabat CDR-H2 sequence comprising SEQ ID NO: 73, and a Kabat CDR-H13 sequence comprising SEQ ID NO: 87. In some aspects, the antibody comprises a V_{H} sequence comprising a Kabat CDR-H1 sequence comprising SEQ ID NO: 30, a Kabat CDR-H2 sequence comprising SEQ ID NO: 72, and a Kabat CDR-H3 sequence comprising SEQ ID NO: 87. In some aspects, the antibody comprises a V_{H} sequence comprising a Kabat CDR-H1 sequence comprising SEQ ID NO: 30, a Kabat CDR-H2 sequence comprising SEQ ID NO: 70, and a Kabat CDR-H3 sequence comprising SEQ ID NO: 87. In some aspects, the antibody comprises a V_{H} sequence comprising a Kabat CDR-H1 sequence comprising SEQ ID NO: 31, a Kabat CDR-H2 sequence comprising SEQ ID NO: 70, and a Kabat CDR-H3 sequence comprising SEQ ID NO: 87. In some aspects, the antibody comprises a V_{H} sequence comprising a Kabat CDR-H1 sequence comprising SEQ ID NO: 31, a Kabat CDR-H2 sequence comprising SEQ ID NO: 71, and a Kabat CDR-H3 sequence comprising SEQ ID NO: 87. In some aspects, the antibody comprises a V_{H} sequence comprising a Kabat CDR-H1 sequence comprising SEQ ID NO: 32, a Kabat CDR-H2 sequence comprising SEQ ID NO: 71, and a Kabat CDR-H3 sequence comprising SEQ ID NO: 87. In some aspects, the antibody comprises a V_{H} sequence comprising a Kabat CDR-H1 sequence comprising SEQ ID NO: 33, a Kabat CDR-H2 sequence comprising SEQ ID NO: 71, and a Kabat CDR-H3 sequence comprising SEQ ID NO: 87. In some aspects, the antibody comprises a V_{H} sequence comprising a Kabat CDR-H1 sequence comprising SEQ ID NO: 33, a Kabat CDR-H2 sequence comprising SEQ ID NO: 72, and a Kabat CDR-H3 sequence comprising SEQ ID NO: 87. In some aspects, the antibody comprises a V_{H} sequence comprising a Kabat CDR-H1 sequence comprising SEQ ID NO: 33, a Kabat CDR-H2 sequence comprising SEQ ID NO: 70, and a Kabat CDR-H3 sequence comprising SEQ ID NO: 87. In some aspects, the antibody comprises a V_{H} sequence comprising a Kabat CDR-H1 sequence comprising SEQ ID NO: 33, a Kabat CDR-H2 sequence comprising SEQ ID NO: 71, and a Kabat CDR-H3 sequence comprising SEQ ID NO: 93. In some aspects, the antibody comprises a V_{H} sequence comprising a Kabat CDR-H1 sequence comprising SEQ ID NO: 33, a Kabat CDR-H2 sequence comprising SEQ ID NO: 70, and a Kabat CDR-H3 sequence comprising SEQ ID NO: 93. In some aspects, the antibody comprises a V_{H} sequence comprising a Kabat CDR-H1 sequence comprising SEQ ID NO: 34, a Kabat CDR-H2 sequence comprising SEQ ID NO: 72, and a Kabat CDR-H3 sequence comprising SEQ ID NO: 87. In some aspects, the antibody comprises a V_{H} sequence comprising a Kabat CDR-H1 sequence comprising SEQ ID NO: 30, a Kabat CDR-H2 sequence comprising SEQ ID NO: 71, and a Kabat CDR-H3 sequence comprising SEQ ID NO: 87. In some aspects, the antibody comprises a V_{H} sequence comprising a Kabat CDR-H1 sequence comprising SEQ ID NO: 35, a Kabat CDR-H2 sequence comprising SEQ ID NO: 70, and a Kabat CDR-H3 sequence comprising SEQ ID NO: 87. In some aspects, the antibody comprises a V_{H} sequence comprising a Kabat CDR-H1 sequence comprising SEQ ID NO: 35, a Kabat CDR-H2 sequence comprising SEQ ID NO: 70, and a Kabat CDR-H3 sequence comprising SEQ ID NO: 90. In some aspects, the antibody comprises a V_{H} sequence comprising a Kabat CDR-H1 sequence comprising SEQ ID NO: 30, a Kabat CDR-H2 sequence comprising SEQ ID NO: 70, and a Kabat CDR-H3 sequence comprising SEQ ID NO: 87. In some aspects, the antibody comprises a V_{H} sequence comprising a Kabat CDR-H1 sequence comprising SEQ ID NO: 39, a Kabat CDR-H2 sequence comprising SEQ ID NO: 76, and a Kabat CDR-H3 sequence comprising SEQ ID NO: 94.

### 2.2.1.8. Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some aspects, the Kabat CDR-H3 sequence comprises, consists of, or consists essentially of a variant of an illustrative Kabat CDR-H3 sequence provided in this disclosure. In some aspects, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some aspects, the Kabat CDR-H13 sequence comprises, consists of, or consists essentially of any of the illustrative Kabat CDR-H3 sequences provided in this disclosure, with 1, 2, or 3 amino acid substitutions. In some aspects, the amino acid substitutions are conservative amino acid substitutions.

In some aspects, the Kabat CDR-H2 sequence comprises, consists of, or consists essentially of a variant of an illustrative Kabat CDR-H2 sequence provided in this disclosure. In some aspects, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some aspects, the Kabat CDR-H2 sequence comprises, consists of, or consists essentially of any of the illustrative Kabat CDR-H2 sequences provided in this disclosure, with 1, 2, or 3 amino acid substitutions. In some aspects, the amino acid substitutions are conservative amino acid substitutions.

In some aspects, the Kabat CDR-H1 sequence comprises, consists of, or consists essentially of a variant of an illustrative Kabat CDR-H1 sequence provided in this disclosure. In some aspects, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some aspects, the Kabat CDR-H1 sequence comprises, consists of, or consists essentially of any of the illustrative Kabat CDR-H1 sequences provided in this disclosure, with 1, 2, or 3 amino acid substitutions. In some aspects, the amino acid substitutions are conservative amino acid substitutions.

### 2.2.2. V_{H} Sequences Comprising Illustrative Chothia CDRs

In some embodiments, the antibody comprises a V_{H} sequence comprising one or more Chothia CDR-H sequences comprising, consisting of, or consisting essentially of one or more illustrative Chothia CDR-H sequences provided in this disclosure, and variants thereof.

### 2.2.2.1. Chothia CDR-H3

In some embodiments, the antibody comprises a V_{H} sequence comprising a Chothia CDR-H3 sequence comprising, consisting of, or consisting essentially of a sequence selected from SEQ ID NOs: 82-109. In some aspects, the antibody comprises a V_{H} sequence comprising a Chothia CDR-H3 sequence comprising, consisting of, or consisting essentially of SEQ ID NO: 82. In some aspects, the antibody comprises a V_{H} sequence comprising a Chothia CDR-H3 sequence comprising, consisting of, or consisting essentially of SEQ ID NO: 83. In some aspects, the antibody comprises a V_{H} sequence comprising a Chothia CDR-H3 sequence comprising, consisting of, or consisting essentially of SEQ ID NO: 84. In some aspects, the antibody comprises a V_{H} sequence comprising a Chothia CDR-H3 sequence comprising, consisting of, or consisting essentially of SEQ ID NO: 85. In some aspects, the antibody comprises a V_{H} sequence comprising a Chothia CDR-H3 sequence comprising, consisting of, or consisting essentially of SEQ ID NO: 86. In some aspects, the antibody comprises a V_{H} sequence comprising a Chothia CDR-H3 sequence comprising, consisting of, or consisting essentially of SEQ ID NO: 87. In some aspects, the antibody comprises a V_{H} sequence comprising a Chothia CDR-H3 sequence comprising, consisting of, or consisting essentially of SEQ ID NO: 88. In some aspects, the antibody comprises a V_{H} sequence comprising a Chothia CDR-H3 sequence comprising, consisting of, or consisting essentially of SEQ ID NO: 89. In some aspects, the antibody comprises a V_{H} sequence comprising a Chothia CDR-H3 sequence comprising, consisting of, or consisting essentially of SEQ ID NO: 90. In some aspects, the antibody comprises a V_{H} sequence comprising a Chothia CDR-H3 sequence comprising, consisting of, or consisting essentially of SEQ ID NO: 91. In some aspects, the antibody comprises a V_{H} sequence comprising a Chothia CDR-H3 sequence comprising, consisting of, or consisting essentially of SEQ ID NO: 92. In some aspects, the antibody comprises a V_{H} sequence comprising a Chothia CDR-H3 sequence comprising, consisting of, or consisting essentially of SEQ ID NO: 93. In some aspects, the antibody comprises a V_{H} sequence comprising a Chothia CDR-H3 sequence comprising, consisting of, or consisting essentially of SEQ ID NO: 94. In some aspects, the antibody comprises a V_{H} sequence comprising a Chothia CDR-H3 sequence comprising, consisting of, or consisting essentially of SEQ ID NO: 95. In some aspects, the antibody comprises a V_{H} sequence comprising a Chothia CDR-H3 sequence comprising, consisting of, or consisting essentially of SEQ ID NO: 96. In some aspects, the antibody comprises a V_{H} sequence comprising a Chothia CDR-H3 sequence comprising, consisting of, or consisting essentially of SEQ ID NO: 97. In some aspects, the antibody comprises a V_{H} sequence comprising a Chothia CDR-H3 sequence comprising, consisting of, or consisting essentially of SEQ ID NO: 98. In some aspects, the antibody comprises a V_{H} sequence comprising a Chothia CDR-H3 sequence comprising, consisting of, or consisting essentially of SEQ ID NO: 99. In some aspects, the antibody comprises a V_{H} sequence comprising a Chothia CDR-H3 sequence comprising, consisting of, or consisting essentially of SEQ ID NO: 100. In some aspects, the antibody comprises a V_{H} sequence comprising a Chothia CDR-H3 sequence comprising, consisting of, or consisting essentially of SEQ ID NO: 101. In some aspects, the antibody comprises a V_{H} sequence comprising a Chothia CDR-H3 sequence comprising, consisting of, or consisting essentially of SEQ ID NO: 102. In some aspects, the antibody comprises a V_{H} sequence comprising a Chothia CDR-H3 sequence comprising, consisting of, or consisting essentially of SEQ ID NO: 103. In some aspects, the antibody comprises a V_{H} sequence comprising a Chothia CDR-H3 sequence comprising, consisting of, or consisting essentially of SEQ ID NO: 104. In some aspects, the antibody comprises a V_{H} sequence comprising a Chothia CDR-13 sequence comprising, consisting of, or consisting essentially of SEQ ID NO: 105. In some aspects, the antibody comprises a V_{H} sequence comprising a Chothia CDR-H3 sequence comprising, consisting of, or consisting essentially of SEQ ID NO: 106. In some aspects, the antibody comprises a V_{H} sequence comprising a Chothia CDR-H3 sequence comprising, consisting of, or consisting essentially of SEQ ID NO: 107. In some aspects, the antibody comprises a V_{H} sequence comprising a Chothia CDR-H3 sequence comprising, consisting of, or consisting essentially of SEQ ID NO: 108. In some aspects, the antibody comprises a V_{H} sequence comprising a Chothia CDR-H3 sequence comprising, consisting of, or consisting essentially of SEQ ID NO: 109.

### 2.2.2.2. Chothia CDR-H2

In some embodiments, the antibody comprises a V_{H} sequence comprising a Chothia CDR-H2 sequence comprising, consisting of, or consisting essentially of a sequence selected from SEQ ID NOs: 46-62. In some aspects, the antibody comprises a V_{H} sequence comprising a Chothia CDR-H2 sequence comprising, consisting of, or consisting essentially of SEQ ID NO: 46. In some aspects, the antibody comprises a V_{H} sequence comprising a Chothia CDR-H2 sequence comprising, consisting of, or consisting essentially of SEQ ID NO: 47. In some aspects, the antibody comprises a V_{H} sequence comprising a Chothia CDR-H2 sequence comprising, consisting of, or consisting essentially of SEQ ID NO: 48. In some aspects, the antibody comprises a V_{H} sequence comprising a Chothia CDR-H2 sequence comprising, consisting of, or consisting essentially of SEQ ID NO: 49. In some aspects, the antibody comprises a V_{H} sequence comprising a Chothia CDR-H2 sequence comprising, consisting of, or consisting essentially of SEQ ID NO: 50. In some aspects, the antibody comprises a V_{H} sequence comprising a Chothia CDR-H2 sequence comprising, consisting of, or consisting essentially of SEQ ID NO: 51. In some aspects, the antibody comprises a V_{H} sequence comprising a Chothia CDR-H2 sequence comprising, consisting of, or consisting essentially of SEQ ID NO: 52. In some aspects, the antibody comprises a V_{H} sequence comprising a Chothia CDR-H2 sequence comprising, consisting of, or consisting essentially of SEQ ID NO: 53. In some aspects, the antibody comprises a V_{H} sequence comprising a Chothia CDR-H2 sequence comprising, consisting of, or consisting essentially of SEQ ID NO: 54. In some aspects, the antibody comprises a V_{H} sequence comprising a Chothia CDR-H2 sequence comprising, consisting of, or consisting essentially of SEQ ID NO: 55. In some aspects, the antibody comprises a V_{H} sequence comprising a Chothia CDR-H2 sequence comprising, consisting of, or consisting essentially of SEQ ID NO: 56. In some aspects, the antibody comprises a V_{H} sequence comprising a Chothia CDR-H2 sequence comprising, consisting of, or consisting essentially of SEQ ID NO: 57. In some aspects, the antibody comprises a V_{H} sequence comprising a Chothia CDR-H2 sequence comprising, consisting of, or consisting essentially of SEQ ID NO: 58. In some aspects, the antibody comprises a V_{H} sequence comprising a Chothia CDR-H2 sequence comprising, consisting of, or consisting essentially of SEQ ID NO: 59. In some aspects, the antibody comprises a V_{H} sequence comprising a Chothia CDR-H2 sequence comprising, consisting of, or consisting essentially of SEQ ID NO: 60. In some aspects, the antibody comprises a V_{H} sequence comprising a Chothia CDR-H2 sequence comprising, consisting of, or consisting essentially of SEQ ID NO: 61. In some aspects, the antibody comprises a V_{H} sequence comprising a Chothia CDR-H2 sequence comprising, consisting of, or consisting essentially of SEQ ID NO: 62.

### 2.2.2.3. Chothia CDR-H1

In some embodiments, the antibody comprises a V_{H} sequence comprising a Chothia CDR-H1 sequence comprising, consisting of, or consisting essentially of a sequence selected from SEQ ID NOs: 1-24. In some aspects, the antibody comprises a V_{H} sequence comprising a Chothia CDR-H1 sequence comprising, consisting of, or consisting essentially of SEQ ID NO: 1. In some aspects, the antibody comprises a V_{H} sequence comprising a Chothia CDR-H1 sequence comprising, consisting of, or consisting essentially of SEQ ID NO: 2. In some aspects, the antibody comprises a V_{H} sequence comprising a Chothia CDR-H1 sequence comprising, consisting of, or consisting essentially of SEQ ID NO: 3. In some aspects, the antibody comprises a V_{H} sequence comprising a Chothia CDR-H1 sequence comprising, consisting of, or consisting essentially of SEQ ID NO: 4. In some aspects, the antibody comprises a V_{H} sequence comprising a Chothia CDR-H1 sequence comprising, consisting of, or consisting essentially of SEQ ID NO: 5. In some aspects, the antibody comprises a Vu sequence comprising a Chothia CDR-H1 sequence comprising, consisting of, or consisting essentially of SEQ ID NO: 6. In some aspects, the antibody comprises a V_{H} sequence comprising a Chothia CDR-H1 sequence comprising, consisting of, or consisting essentially of SEQ ID NO: 7. In some aspects, the antibody comprises a V_{H} sequence comprising a Chothia CDR-H1 sequence comprising, consisting of, or consisting essentially of SEQ ID NO: 8. In some aspects, the antibody comprises a V_{H} sequence comprising a Chothia CDR-H1 sequence comprising, consisting of, or consisting essentially of SEQ ID NO: 9. In some aspects, the antibody comprises a V_{H} sequence comprising a Chothia CDR-H1 sequence comprising, consisting of, or consisting essentially of SEQ ID NO: 10. In some aspects, the antibody comprises a V_{H} sequence comprising a Chothia CDR-H1 sequence comprising, consisting of, or consisting essentially of SEQ ID NO: 11. In some aspects, the antibody comprises a V_{H} sequence comprising a Chothia CDR-H1 sequence comprising, consisting of, or consisting essentially of SEQ ID NO: 12. In some aspects, the antibody comprises a V_{H} sequence comprising a Chothia CDR-H1 sequence comprising, consisting of, or consisting essentially of SEQ ID NO: 13. In some aspects, the antibody comprises a V_{H} sequence comprising a Chothia CDR-H1 sequence comprising, consisting of, or consisting essentially of SEQ ID NO: 14. In some aspects, the antibody comprises a V_{H} sequence comprising a Chothia CDR-H1 sequence comprising, consisting of, or consisting essentially of SEQ ID NO: 15. In some aspects, the antibody comprises a V_{H} sequence comprising a Chothia CDR-H1 sequence comprising, consisting of, or consisting essentially of SEQ ID NO: 16. In some aspects, the antibody comprises a V_{H} sequence comprising a Chothia CDR-H1 sequence comprising, consisting of, or consisting essentially of SEQ ID NO: 17. In some aspects, the antibody comprises a V_{H} sequence comprising a Chothia CDR-H1 sequence comprising, consisting of, or consisting essentially of SEQ ID NO: 18. In some aspects, the antibody comprises a V_{H} sequence comprising a Chothia CDR-H1 sequence comprising, consisting of, or consisting essentially of SEQ ID NO: 19. In some aspects, the antibody comprises a V_{H} sequence comprising a Chothia CDR-H1 sequence comprising, consisting of, or consisting essentially of SEQ ID NO: 20. In some aspects, the antibody comprises a V_{H} sequence comprising a Chothia CDR-H1 sequence comprising, consisting of, or consisting essentially of SEQ ID NO: 21. In some aspects, the antibody comprises a V_{H} sequence comprising a Chothia CDR-H1 sequence comprising, consisting of, or consisting essentially of SEQ ID NO: 22. In some aspects, the antibody comprises a V_{H} sequence comprising a Chothia CDR-H1 sequence comprising, consisting of, or consisting essentially of SEQ ID NO: 23. In some aspects, the antibody comprises a V_{H} sequence comprising a Chothia CDR-H1 sequence comprising, consisting of, or consisting essentially of SEQ ID NO: 24.

### 2.2.2.4. Chothia CDR-H3+Chothia CDR-H2

In some embodiments, the antibody comprises a V_{H} sequence comprising a Chothia CDR-H13 sequence comprising, consisting of, or consisting essentially of a sequence selected from SEQ ID NOs: 82-109, and a Chothia CDR-H2 sequence comprising, consisting of, or consisting essentially of a sequence selected from SEQ ID NOs: 46-62. In some aspects, the Chothia CDR-H3 sequence and the Chothia CDR-H2 sequence are both from a single illustrative V_{H} sequence provided in this disclosure. For example, in some aspects, the Chothia CDR-H3 and Chothia CDR-H2 are both from a single illustrative V_{H} sequence selected from SEQ ID NOs: 179-218.

### 2.2.2.5. Chothia CDR-H3+Chothia CDR-H1

In some embodiments, the antibody comprises a V_{H} sequence comprising a Chothia CDR-H3 sequence comprising, consisting of, or consisting essentially of a sequence selected from SEQ ID NOs: 82-109, and a Chothia CDR-H1 sequence comprising, consisting of, or consisting essentially of a sequence selected from SEQ ID NOs: 1-24. In some aspects, the Chothia CDR-H3 sequence and the Chothia CDR-H1 sequence are both from a single illustrative V_{H} sequence provided in this disclosure. For example, in some aspects, the Chothia CDR-H3 and Chothia CDR-H1 are both from a single illustrative V_{H} sequence selected from SEQ ID NOs: 179-218.

### 2.2.2.6. Chothia CDR-H1+Chothia CDR-H2

In some embodiments, the antibody comprises a V_{H} sequence comprising a Chothia CDR-H1 sequence comprising, consisting of, or consisting essentially of a sequence selected from SEQ ID NOs: 1-24 and a Chothia CDR-H2 sequence comprising, consisting of, or consisting essentially of a sequence selected from SEQ ID NOs: 46-62. In some aspects, the Chothia CDR-H1 sequence and the Chothia CDR-H2 sequence are both from a single illustrative V_{H} sequence provided in this disclosure. For example, in some aspects, the Chothia CDR-H1 and Chothia CDR-H2 are both from a single illustrative V_{H} sequence selected from SEQ ID NOs: 179-218.

### 2.2.2.7. Chothia CDR-H1+Chothia CDR-H2+Chothia CDR-H3

In some embodiments, the antibody comprises a V_{H} sequence comprising a Chothia CDR-H1 sequence comprising, consisting of, or consisting essentially of a sequence selected from SEQ ID NOs: 1-24, a Chothia CDR-H2 sequence comprising, consisting of, or consisting essentially of a sequence selected from SEQ ID NOs: 46-62, and a Chothia CDR-113 sequence comprising, consisting of, or consisting essentially of a sequence selected from SEQ ID NOs: 82-109. In some aspects, the Chothia CDR-H1 sequence, Chothia CDR-H2 sequence, and Chothia CDR-H3 sequence are all from a single illustrative V_{H} sequence provided in this disclosure. For example, in some aspects, the Chothia CDR-H1, Chothia CDR-H2, and Chothia CDR-H3 are all from a single illustrative V_{H} sequence selected from SEQ ID NOs: 179-218.

In some embodiments, the antibody comprises a V_{H} sequence comprising a Chothia CDR-H1 sequence comprising SEQ ID NO: 1, a Chothia CDR-H2 sequence comprising SEQ ID NO: 46, and a Chothia CDR-H3 sequence comprising SEQ ID NO: 82. In some embodiments, the antibody comprises a V_{H} sequence comprising a Chothia CDR-H1 sequence comprising SEQ ID NO: 2, a Chothia CDR-H2 sequence comprising SEQ ID NO: 47, and a Chothia CDR-H3 sequence comprising SEQ ID NO: 83. In some embodiments, the antibody comprises a V_{H} sequence comprising a Chothia CDR-H1 sequence comprising SEQ ID NO: 1, a Chothia CDR-H2 sequence comprising SEQ ID NO: 46, and a Chothia CDR-H3 sequence comprising SEQ ID NO: 84. In some embodiments, the antibody comprises a V_{H} sequence comprising a Chothia CDR-H1 sequence comprising SEQ ID NO: 2, a Chothia CDR-H2 sequence comprising SEQ ID NO: 47, and a Chothia CDR-H3 sequence comprising SEQ ID NO: 85. In some embodiments, the antibody comprises a V_{H} sequence comprising a Chothia CDR-H1 sequence comprising SEQ ID NO: 3, a Chothia CDR-H2 sequence comprising SEQ ID NO: 47, and a Chothia CDR-H3 sequence comprising SEQ ID NO: 86. In some embodiments, the antibody comprises a V_{H} sequence comprising a Chothia CDR-H1 sequence comprising SEQ ID NO: 4, a Chothia CDR-H2 sequence comprising SEQ ID NO: 46, and a Chothia CDR-H3 sequence comprising SEQ ID NO: 82. In some embodiments, the antibody comprises a V_{H} sequence comprising a Chothia CDR-H1 sequence comprising SEQ ID NO: 2, a Chothia CDR-H2 sequence comprising SEQ ID NO: 46, and a Chothia CDR-H3 sequence comprising SEQ ID NO: 82. In some embodiments, the antibody comprises a V_{H} sequence comprising a Chothia CDR-H1 sequence comprising SEQ ID NO: 5, a Chothia CDR-H2 sequence comprising SEQ ID NO: 49, and a Chothia CDR-H3 sequence comprising SEQ ID NO: 82. In some embodiments, the antibody comprises a V_{H} sequence comprising a Chothia CDR-H1 sequence comprising SEQ ID NO: 6, a Chothia CDR-H2 sequence comprising SEQ ID NO: 50, and a Chothia CDR-H3 sequence comprising SEQ ID NO: 82. In some embodiments, the antibody comprises a V_{H} sequence comprising a Chothia CDR-H1 sequence comprising SEQ ID NO: 2, a Chothia CDR-H2 sequence comprising SEQ ID NO: 47, and a Chothia CDR-H3 sequence comprising SEQ ID NO: 82. In some embodiments, the antibody comprises a V_{H} sequence comprising a Chothia CDR-H1 sequence comprising SEQ ID NO: 7, a Chothia CDR-H2 sequence comprising SEQ ID NO: 51, and a Chothia CDR-H3 sequence comprising SEQ ID NO: 87. In some embodiments, the antibody comprises a V_{H} sequence comprising a Chothia CDR-H1 sequence comprising SEQ ID NO: 8, a Chothia CDR-H2 sequence comprising SEQ ID NO: 52, and a Chothia CDR-H3 sequence comprising SEQ ID NO: 88. In some embodiments, the antibody comprises a V_{H} sequence comprising a Chothia CDR-H1 sequence comprising SEQ ID NO: 9, a Chothia CDR-H2 sequence comprising SEQ ID NO: 53, and a Chothia CDR-H3 sequence comprising SEQ ID NO: 89. In some embodiments, the antibody comprises a V_{H} sequence comprising a Chothia CDR-H1 sequence comprising SEQ ID NO: 10, a Chothia CDR-H2 sequence comprising SEQ ID NO: 52, and a Chothia CDR-H3 sequence comprising SEQ ID NO: 90. In some embodiments, the antibody comprises a V_{H} sequence comprising a Chothia CDR-H1 sequence comprising SEQ ID NO: 11, a Chothia CDR-H2 sequence comprising SEQ ID NO: 54, and a Chothia CDR-H3 sequence comprising SEQ ID NO: 91. In some embodiments, the antibody comprises a V_{H} sequence comprising a Chothia CDR-H1 sequence comprising SEQ ID NO: 12, a Chothia CDR-H2 sequence comprising SEQ ID NO: 53, and a Chothia CDR-H3 sequence comprising SEQ ID NO: 92. In some embodiments, the antibody comprises a V_{H} sequence comprising a Chothia CDR-H1 sequence comprising SEQ ID NO: 13, a Chothia CDR-H2 sequence comprising SEQ ID NO: 52, and a Chothia CDR-H3 sequence comprising SEQ ID NO: 93. In some embodiments, the antibody comprises a V_{H} sequence comprising a Chothia CDR-H1 sequence comprising SEQ ID NO: 10, a Chothia CDR-H2 sequence comprising SEQ ID NO: 52, and a Chothia CDR-H3 sequence comprising SEQ ID NO: 92. In some embodiments, the antibody comprises a V_{H} sequence comprising a Chothia CDR-H1 sequence comprising SEQ ID NO: 14, a Chothia CDR-H2 sequence comprising SEQ ID NO: 54, and a Chothia CDR-H3 sequence comprising SEQ ID NO: 87. In some embodiments, the antibody comprises a V_{H} sequence comprising a Chothia CDR-H1 sequence comprising SEQ ID NO: 15, a Chothia CDR-H2 sequence comprising SEQ ID NO: 55, and a Chothia CDR-H3 sequence comprising SEQ ID NO: 87. In some embodiments, the antibody comprises a V_{H} sequence comprising a Chothia CDR-H1 sequence comprising SEQ ID NO: 16, a Chothia CDR-H2 sequence comprising SEQ ID NO: 56, and a Chothia CDR-H3 sequence comprising SEQ ID NO: 87. In some embodiments, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 94. In some embodiments, the antibody comprises a V_{H} sequence comprising a Chothia CDR-H1 sequence comprising SEQ ID NO: 18, a Chothia CDR-H2 sequence comprising SEQ ID NO: 57, and a Chothia CDR-H3 sequence comprising SEQ ID NO: 95. In some embodiments, the antibody comprises a V_{H} sequence comprising a Chothia CDR-H1 sequence comprising SEQ ID NO: 19, a Chothia CDR-H2 sequence comprising SEQ ID NO: 57, and a Chothia CDR-H3 sequence comprising SEQ ID NO: 96. In some embodiments, the antibody comprises a V_{H} sequence comprising a Chothia CDR-H1 sequence comprising SEQ ID NO: 21, a Chothia CDR-H2 sequence comprising SEQ ID NO: 57, and a Chothia CDR-H3 sequence comprising SEQ ID NO: 94. In some embodiments, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 97. In some embodiments, the antibody comprises a V_{H} sequence comprising a Chothia CDR-H1 sequence comprising SEQ ID NO: 22, a Chothia CDR-H2 sequence comprising SEQ ID NO: 51, and a Chothia CDR-H3 sequence comprising SEQ ID NO: 98. In some embodiments, the antibody comprises a V_{H} sequence comprising a Chothia CDR-H1 sequence comprising SEQ ID NO: 22, a Chothia CDR-H2 sequence comprising SEQ ID NO: 51, and a Chothia CDR-H3 sequence comprising SEQ ID NO: 99. In some embodiments, the antibody comprises a V_{H} sequence comprising a Chothia CDR-H1 sequence comprising SEQ ID NO: 22, a Chothia CDR-H2 sequence comprising SEQ ID NO: 51, and a Chothia CDR-H3 sequence comprising SEQ ID NO: 100. In some embodiments, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 101. In some embodiments, the antibody comprises a V_{H} sequence comprising a Chothia CDR-H1 sequence comprising SEQ ID NO: 17, a Chothia CDR-H2 sequence comprising SEQ ID NO: 59, and a Chothia CDR-H3 sequence comprising SEQ ID NO: 102. In some embodiments, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 103. In some embodiments, the antibody comprises a V_{H} sequence comprising a Chothia CDR-H1 sequence comprising SEQ ID NO: 23, a Chothia CDR-H2 sequence comprising SEQ ID NO: 60, and a Chothia CDR-H3 sequence comprising SEQ ID NO: 104. In some embodiments, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 105. In some embodiments, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 106. In some embodiments, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 107. In some embodiments, the antibody comprises a V_{H} sequence comprising a Chothia CDR-H1 sequence comprising SEQ ID NO: 17, a Chothia CDR-H2 sequence comprising SEQ ID NO: 61, and a Chothia CDR-H3 sequence comprising SEQ ID NO: 108. In some embodiments, the antibody comprises a V_{H} sequence comprising a Chothia CDR-H1 sequence comprising SEQ ID NO: 17, a Chothia CDR-H2 sequence comprising SEQ ID NO: 62, and a Chothia CDR-H3 sequence comprising SEQ ID NO: 109.

In some embodiments, the antibody comprises a V_{H} sequence comprising a Chothia CDR-H1 sequence comprising SEQ ID NO: 2, a Chothia CDR-H2 sequence comprising SEQ ID NO: 46, and a Chothia CDR-H3 sequence comprising SEQ ID NO: 83. In some embodiments, the antibody comprises a VA sequence comprising a Chothia CDR-H1 sequence comprising SEQ ID NO: 2, a Chothia CDR-H2 sequence comprising SEQ ID NO: 46, and a Chothia CDR-H3 sequence comprising SEQ ID NO: 84. In some embodiments, the antibody comprises a V_{H} sequence comprising a Chothia CDR-H1 sequence comprising SEQ ID NO: 2, a Chothia CDR-H2 sequence comprising SEQ ID NO: 46, and a Chothia CDR-H3 sequence comprising SEQ ID NO: 85. In some embodiments, the antibody comprises a V_{H} sequence comprising a Chothia CDR-H1 sequence comprising SEQ ID NO: 2, a Chothia CDR-H2 sequence comprising SEQ ID NO: 46, and a Chothia CDR-H3 sequence comprising SEQ ID NO: 86. In some embodiments, the antibody comprises a V_{H} sequence comprising a Chothia CDR-H1 sequence comprising SEQ ID NO: 2, a Chothia CDR-H2 sequence comprising SEQ ID NO: 47, and a Chothia CDR-H3 sequence comprising SEQ ID NO: 84. In some embodiments, the antibody comprises a V_{H} sequence comprising a Chothia CDR-H1 sequence comprising SEQ ID NO: 2, a Chothia CDR-H2 sequence comprising SEQ ID NO: 47, and a Chothia CDR-H3 sequence comprising SEQ ID NO: 86. In some embodiments, the antibody comprises a V_{H} sequence comprising a Chothia CDR-H1 sequence comprising SEQ ID NO: 1, a Chothia CDR-H2 sequence comprising SEQ ID NO: 46, and a Chothia CDR-H3 sequence comprising SEQ ID NO: 83. In some embodiments, the antibody comprises a V_{H} sequence comprising a Chothia CDR-H1 sequence comprising SEQ ID NO: 1, a Chothia CDR-H2 sequence comprising SEQ ID NO: 46, and a Chothia CDR-H3 sequence comprising SEQ ID NO: 86. In some embodiments, the antibody comprises a V_{H} sequence comprising a Chothia CDR-H1 sequence comprising SEQ ID NO: 10, a Chothia CDR-H2 sequence comprising SEQ ID NO: 53, and a Chothia CDR-H3 sequence comprising SEQ ID NO: 93. In some embodiments, the antibody comprises a V_{H} sequence comprising a Chothia CDR-H1 sequence comprising SEQ ID NO: 10, a Chothia CDR-H2 sequence comprising SEQ ID NO: 55, and a Chothia CDR-H3 sequence comprising SEQ ID NO: 87. In some embodiments, the antibody comprises a V_{H} sequence comprising a Chothia CDR-H1 sequence comprising SEQ ID NO: 10, a Chothia CDR-H2 sequence comprising SEQ ID NO: 54, and a Chothia CDR-H3 sequence comprising SEQ ID NO: 87. In some embodiments, the antibody comprises a V_{H} sequence comprising a Chothia CDR-H1 sequence comprising SEQ ID NO: 10, a Chothia CDR-H2 sequence comprising SEQ ID NO: 52, and a Chothia CDR-H3 sequence comprising SEQ ID NO: 87. In some embodiments, the antibody comprises a V_{H} sequence comprising a Chothia CDR-H1 sequence comprising SEQ ID NO: 8, a Chothia CDR-H2 sequence comprising SEQ ID NO: 52, and a Chothia CDR-H3 sequence comprising SEQ ID NO: 87. In some embodiments, the antibody comprises a V_{H} sequence comprising a Chothia CDR-H1 sequence comprising SEQ ID NO: 8, a Chothia CDR-H2 sequence comprising SEQ ID NO: 53, and a Chothia CDR-H3 sequence comprising SEQ ID NO: 87. In some embodiments, the antibody comprises a V_{H} sequence comprising a Chothia CDR-H1 sequence comprising SEQ ID NO: 9, a Chothia CDR-H2 sequence comprising SEQ ID NO: 53, and a Chothia CDR-H3 sequence comprising SEQ ID NO: 87. In some embodiments, the antibody comprises a V_{H} sequence comprising a Chothia CDR-H1 sequence comprising SEQ ID NO: 10, a Chothia CDR-H2 sequence comprising SEQ ID NO: 53, and a Chothia CDR-H3 sequence comprising SEQ ID NO: 87. In some embodiments, the antibody comprises a V_{H} sequence comprising a Chothia CDR-H1 sequence comprising SEQ ID NO: 10, a Chothia CDR-H2 sequence comprising SEQ ID NO: 54, and a Chothia CDR-H3 sequence comprising SEQ ID NO: 87. In some embodiments, the antibody comprises a V_{H} sequence comprising a Chothia CDR-H1 sequence comprising SEQ ID NO: 10, a Chothia CDR-H2 sequence comprising SEQ ID NO: 52, and a Chothia CDR-H3 sequence comprising SEQ ID NO: 87. In some embodiments, the antibody comprises a V_{H} sequence comprising a Chothia CDR-H1 sequence comprising SEQ ID NO: 10, a Chothia CDR-H2 sequence comprising SEQ ID NO: 53, and a Chothia CDR-H3 sequence comprising SEQ ID NO: 93. In some embodiments, the antibody comprises a V_{H} sequence comprising a Chothia CDR-H1 sequence comprising SEQ ID NO: 10, a Chothia CDR-H2 sequence comprising SEQ ID NO: 52, and a Chothia CDR-H3 sequence comprising SEQ ID NO: 93. In some embodiments, the antibody comprises a V_{H} sequence comprising a Chothia CDR-H1 sequence comprising SEQ ID NO: 11, a Chothia CDR-H2 sequence comprising SEQ ID NO: 54, and a Chothia CDR-H3 sequence comprising SEQ ID NO: 87. In some embodiments, the antibody comprises a V_{H} sequence comprising a Chothia CDR-H1 sequence comprising SEQ ID NO: 12, a Chothia CDR-H2 sequence comprising SEQ ID NO: 53, and a Chothia CDR-H3 sequence comprising SEQ ID NO: 87. In some embodiments, the antibody comprises a V_{H} sequence comprising a Chothia CDR-H1 sequence comprising SEQ ID NO: 13, a Chothia CDR-H2 sequence comprising SEQ ID NO: 52, and a Chothia CDR-13 sequence comprising SEQ ID NO: 87. In some embodiments, the antibody comprises a V_{H} sequence comprising a Chothia CDR-H1 sequence comprising SEQ ID NO: 13, a Chothia CDR-H2 sequence comprising SEQ ID NO: 52, and a Chothia CDR-H3 sequence comprising SEQ ID NO: 90. In some embodiments, the antibody comprises a V_{H} sequence comprising a Chothia CDR-H1 sequence comprising SEQ ID NO: 15, a Chothia CDR-H2 sequence comprising SEQ ID NO: 52, and a Chothia CDR-H3 sequence comprising SEQ ID NO: 87. In some embodiments, the antibody comprises a V_{H} sequence comprising a Chothia CDR-H1 sequence comprising SEQ ID NO: 18, a Chothia CDR-H2 sequence comprising SEQ ID NO: 57, and a Chothia CDR-H3 sequence comprising SEQ ID NO: 94.

### 2.2.2.8. Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some aspects, the Chothia CDR-H3 sequence comprises, consists of, or consists essentially of a variant of an illustrative Chothia CDR-H3 sequence provided in this disclosure. In some aspects, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some aspects, the Chothia CDR-H3 sequence comprises, consists of, or consists essentially of any of the illustrative Chothia CDR-H3 sequences provided in this disclosure, with 1, 2, or 3 amino acid substitutions. In some aspects, the amino acid substitutions are conservative amino acid substitutions.

In some aspects, the Chothia CDR-H2 sequence comprises, consists of, or consists essentially of a variant of an illustrative Chothia CDR-H2 sequence provided in this disclosure. In some aspects, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some aspects, the Chothia CDR-H2 sequence comprises, consists of, or consists essentially of any of the illustrative Chothia CDR-H2 sequences provided in this disclosure, with 1, 2, or 3 amino acid substitutions. In some aspects, the amino acid substitutions are conservative amino acid substitutions.

In some aspects, the Chothia CDR-H1 sequence comprises, consists of, or consists essentially of a variant of an illustrative Chothia CDR-H1 sequence provided in this disclosure. In some aspects, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some aspects, the Chothia CDR-H1 sequence comprises, consists of, or consists essentially of any of the illustrative Chothia CDR-H1 sequences provided in this disclosure, with 1, 2, or 3 amino acid substitutions. In some aspects, the amino acid substitutions are conservative amino acid substitutions.

### 2.3. V_{H} Sequences

In some embodiments, the antibody comprises a V_{H} sequence comprising, consisting of, or consisting essentially of a sequence selected from SEQ ID NOs: 179-218. In some aspects, the antibody comprises a V_{H} sequence comprising, consisting of, or consisting essentially of SEQ ID NO: 179. In some aspects, the antibody comprises a V_{H} sequence comprising, consisting of, or consisting essentially of SEQ ID NO: 180. In some aspects, the antibody comprises a V_{H} sequence comprising, consisting of, or consisting essentially of SEQ ID NO: 181. In some aspects, the antibody comprises a V_{H} sequence comprising, consisting of, or consisting essentially of SEQ ID NO: 182. In some aspects, the antibody comprises a Vu sequence comprising, consisting of, or consisting essentially of SEQ ID NO: 183. In some aspects, the antibody comprises a V_{H} sequence comprising, consisting of, or consisting essentially of SEQ ID NO: 184. In some aspects, the antibody comprises a V_{H} sequence comprising, consisting of, or consisting essentially of SEQ ID NO: 185. In some aspects, the antibody comprises a V_{H} sequence comprising, consisting of, or consisting essentially of SEQ ID NO: 186. In some aspects, the antibody comprises a V_{H} sequence comprising, consisting of, or consisting essentially of SEQ ID NO: 187. In some aspects, the antibody comprises a V_{H} sequence comprising, consisting of, or consisting essentially of SEQ ID NO: 188. In some aspects, the antibody comprises a Vn sequence comprising, consisting of, or consisting essentially of SEQ ID NO: 189. In some aspects, the antibody comprises a V_{H} sequence comprising, consisting of, or consisting essentially of SEQ ID NO: 190. In some aspects, the antibody comprises a V_{H} sequence comprising, consisting of, or consisting essentially of SEQ ID NO: 191. In some aspects, the antibody comprises a V_{H} sequence comprising, consisting of, or consisting essentially of SEQ ID NO: 192. In some aspects, the antibody comprises a V_{H} sequence comprising, consisting of, or consisting essentially of SEQ ID NO: 193. In some aspects, the antibody comprises a V_{H} sequence comprising, consisting of, or consisting essentially of SEQ ID NO: 194. In some aspects, the antibody comprises a V_{H} sequence comprising, consisting of, or consisting essentially of SEQ ID NO: 195. In some aspects, the antibody comprises a V_{H} sequence comprising, consisting of, or consisting essentially of SEQ ID NO: 196. In some aspects, the antibody comprises a V_{H} sequence comprising, consisting of, or consisting essentially of SEQ ID NO: 197. In some aspects, the antibody comprises a V_{H} sequence comprising, consisting of, or consisting essentially of SEQ ID NO: 198. In some aspects, the antibody comprises a V_{H} sequence comprising, consisting of, or consisting essentially of SEQ ID NO: 199. In some aspects, the antibody comprises a V_{H} sequence comprising, consisting of, or consisting essentially of SEQ ID NO: 200. In some aspects, the antibody comprises a V_{H} sequence comprising, consisting of, or consisting essentially of SEQ ID NO: 201. In some aspects, the antibody comprises a V_{H} sequence comprising, consisting of, or consisting essentially of SEQ ID NO: 202. In some aspects, the antibody comprises a V_{H} sequence comprising, consisting of, or consisting essentially of SEQ ID NO: 203. In some aspects, the antibody comprises a V_{H} sequence comprising, consisting of, or consisting essentially of SEQ ID NO: 204. In some aspects, the antibody comprises a V_{H} sequence comprising, consisting of, or consisting essentially of SEQ ID NO: 205. In some aspects, the antibody comprises a V_{H} sequence comprising, consisting of, or consisting essentially of SEQ ID NO: 206. In some aspects, the antibody comprises a V_{H} sequence comprising, consisting of, or consisting essentially of SEQ ID NO: 207. In some aspects, the antibody comprises a V_{H} sequence comprising, consisting of, or consisting essentially of SEQ ID NO: 208. In some aspects, the antibody comprises a V_{H} sequence comprising, consisting of, or consisting essentially of SEQ ID NO: 209. In some aspects, the antibody comprises a V_{H} sequence comprising, consisting of, or consisting essentially of SEQ ID NO: 210. In some aspects, the antibody comprises a V_{H} sequence comprising, consisting of, or consisting essentially of SEQ ID NO: 211. In some aspects, the antibody comprises a V_{H} sequence comprising, consisting of, or consisting essentially of SEQ ID NO: 212. In some aspects, the antibody comprises a V_{H} sequence comprising, consisting of, or consisting essentially of SEQ ID NO: 213. In some aspects, the antibody comprises a V_{H} sequence comprising, consisting of, or consisting essentially of SEQ ID NO: 214. In some aspects, the antibody comprises a V_{H} sequence comprising, consisting of, or consisting essentially of SEQ ID NO: 215. In some aspects, the antibody comprises a V_{H} sequence comprising, consisting of, or consisting essentially of SEQ ID NO: 216. In some aspects, the antibody comprises a V_{H} sequence comprising, consisting of, or consisting essentially of SEQ ID NO: 217. In some aspects, the antibody comprises a V_{H} sequence comprising, consisting of, or consisting essentially of SEQ ID NO: 218.

### 2.3.1. Variants of V_{H} Sequences

In some embodiments, the V_{H} sequences provided herein comprise, consist of, or consist essentially of a variant of an illustrative V_{H} sequence provided in this disclosure.

In some aspects, the V_{H} sequence comprises, consists of, or consists essentially of a variant of an illustrative V_{H} sequence provided in this disclosure. In some aspects, the Vu sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.4. CDR-L3 Sequences

In some embodiments, the antibody comprises a CDR-L3 sequence comprising, consisting of, or consisting essentially of a sequence selected from SEQ ID NOs: 141-166. In some aspects, the antibody comprises a CDR-L3 sequence comprising, consisting of, or consisting essentially of SEQ ID NO: 141. In some aspects, the antibody comprises a CDR-L3 sequence comprising, consisting of, or consisting essentially of SEQ ID NO: 142. In some aspects, the antibody comprises a CDR-L3 sequence comprising, consisting of, or consisting essentially of SEQ ID NO: 143. In some aspects, the antibody comprises a CDR-L3 sequence comprising, consisting of, or consisting essentially of SEQ ID NO: 144. In some aspects, the antibody comprises a CDR-L3 sequence comprising, consisting of, or consisting essentially of SEQ ID NO: 145. In some aspects, the antibody comprises a CDR-L3 sequence comprising, consisting of, or consisting essentially of SEQ ID NO: 146. In some aspects, the antibody comprises a CDR-L3 sequence comprising, consisting of, or consisting essentially of SEQ ID NO): 147. In some aspects, the antibody comprises a CDR-L3 sequence comprising, consisting of, or consisting essentially of SEQ ID NO: 148. In some aspects, the antibody comprises a CDR-L3 sequence comprising, consisting of, or consisting essentially of SEQ ID NO: 149. In some aspects, the antibody comprises a CDR-L3 sequence comprising, consisting of, or consisting essentially of SEQ ID NO: 150. In some aspects, the antibody comprises a CDR-L3 sequence comprising, consisting of, or consisting essentially of SEQ ID NO: 151. In some aspects, the antibody comprises a CDR-L3 sequence comprising, consisting of, or consisting essentially of SEQ ID NO: 152. In some aspects, the antibody comprises a CDR-L3 sequence comprising, consisting of, or consisting essentially of SEQ ID NO: 153. In some aspects, the antibody comprises a CDR-L3 sequence comprising, consisting of, or consisting essentially of SEQ ID NO: 154. In some aspects, the antibody comprises a CDR-L3 sequence comprising, consisting of, or consisting essentially of SEQ ID NO: 155. In some aspects, the antibody comprises a CDR-L3 sequence comprising, consisting of, or consisting essentially of SEQ ID NO: 156. In some aspects, the antibody comprises a CDR-L3 sequence comprising, consisting of, or consisting essentially of SEQ ID NO: 157. In some aspects, the antibody comprises a CDR-L3 sequence comprising, consisting of, or consisting essentially of SEQ ID NO: 158. In some aspects, the antibody comprises a CDR-L3 sequence comprising, consisting of, or consisting essentially of SEQ ID NO: 159. In some aspects, the antibody comprises a CDR-L3 sequence comprising, consisting of, or consisting essentially of SEQ ID NO: 160. In some aspects, the antibody comprises a CDR-L3 sequence comprising, consisting of, or consisting essentially of SEQ ID NO: 161. In some aspects, the antibody comprises a CDR-L3 sequence comprising, consisting of, or consisting essentially of SEQ ID NO: 162. In some aspects, the antibody comprises a CDR-L3 sequence comprising, consisting of, or consisting essentially of SEQ ID NO: 163. In some aspects, the antibody comprises a CDR-L3 sequence comprising, consisting of, or consisting essentially of SEQ ID NO: 164. In some aspects, the antibody comprises a CDR-L3 sequence comprising, consisting of, or consisting essentially of SEQ ID NO: 165. In some aspects, the antibody comprises a CDR-L3 sequence comprising, consisting of, or consisting essentially of SEQ ID NO: 166.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-13 sequences provided in this disclosure, with 1, 2, or 3 amino acid substitutions. In some aspects, the amino acid substitutions are conservative amino acid substitutions.

### 2.5. V_{L} Sequences Comprising Illustrative CDRs

In some embodiments, the antibody comprises a V_{L} sequence comprising one or more CDR-L sequences comprising, consisting of, or consisting essentially of one or more illustrative CDR-L sequences provided in this disclosure, and variants thereof.

### 2.5.1. CDR-L3

In some embodiments, the antibody comprises a V_{L} sequence comprising a CDR-L3 sequence comprising, consisting of, or consisting essentially of a sequence selected from SEQ ID NOs: 141-166. In some aspects, the antibody comprises a V_{L} sequence comprising a CDR-L3 sequence comprising, consisting of, or consisting essentially of SEQ ID NO: 141. In some aspects, the antibody comprises a V_{L} sequence comprising a CDR-13 sequence comprising, consisting of, or consisting essentially of SEQ ID NO: 142. In some aspects, the antibody comprises a V_{L} sequence comprising a CDR-L3 sequence comprising, consisting of, or consisting essentially of SEQ ID NO: 143. In some aspects, the antibody comprises a V_{L} sequence comprising a CDR-L3 sequence comprising, consisting of, or consisting essentially of SEQ ID NO: 144. In some aspects, the antibody comprises a V_{L} sequence comprising a CDR-L3 sequence comprising, consisting of, or consisting essentially of SEQ ID NO: 145. In some aspects, the antibody comprises a V_{L} sequence comprising a CDR-L3 sequence comprising, consisting of, or consisting essentially of SEQ ID NO: 146. In some aspects, the antibody comprises a V_{L} sequence comprising a CDR-L3 sequence comprising, consisting of, or consisting essentially of SEQ ID NO: 147. In some aspects, the antibody comprises a V_{L} sequence comprising a CDR-L3 sequence comprising, consisting of, or consisting essentially of SEQ ID NO: 148. In some aspects, the antibody comprises a V_{L} sequence comprising a CDR-L3 sequence comprising, consisting of, or consisting essentially of SEQ ID NO: 149. In some aspects, the antibody comprises a V_{L} sequence comprising a CDR-L3 sequence comprising, consisting of, or consisting essentially of SEQ ID NO: 150. In some aspects, the antibody comprises a V_{L} sequence comprising a CDR-L3 sequence comprising, consisting of, or consisting essentially of SEQ ID NO: 151. In some aspects, the antibody comprises a V_{L} sequence comprising a CDR-L3 sequence comprising, consisting of, or consisting essentially of SEQ ID NO: 152. In some aspects, the antibody comprises a V_{L} sequence comprising a CDR-L3 sequence comprising, consisting of, or consisting essentially of SEQ ID NO: 153. In some aspects, the antibody comprises a V_{L} sequence comprising a CDR-L3 sequence comprising, consisting of, or consisting essentially of SEQ ID NO: 154. In some aspects, the antibody comprises a V_{L} sequence comprising a CDR-L3 sequence comprising, consisting of, or consisting essentially of SEQ ID NO: 155. In some aspects, the antibody comprises a V_{L} sequence comprising a CDR-L3 sequence comprising, consisting of, or consisting essentially of SEQ ID NO: 156. In some aspects, the antibody comprises a V_{L} sequence comprising a CDR-L3 sequence comprising, consisting of, or consisting essentially of SEQ ID NO: 157. In some aspects, the antibody comprises a V_{L} sequence comprising a CDR-L3 sequence comprising, consisting of, or consisting essentially of SEQ ID NO: 158. In some aspects, the antibody comprises a V_{L} sequence comprising a CDR-L3 sequence comprising, consisting of, or consisting essentially of SEQ ID NO: 159. In some aspects, the antibody comprises a V_{L} sequence comprising a CDR-L3 sequence comprising, consisting of, or consisting essentially of SEQ ID NO: 160. In some aspects, the antibody comprises a V_{L} sequence comprising a CDR-L3 sequence comprising, consisting of, or consisting essentially of SEQ ID NO: 161. In some aspects, the antibody comprises a V_{L} sequence comprising a CDR-L3 sequence comprising, consisting of, or consisting essentially of SEQ ID NO: 162. In some aspects, the antibody comprises a V_{L} sequence comprising a CDR-L3 sequence comprising, consisting of, or consisting essentially of SEQ ID NO: 163. In some aspects, the antibody comprises a V_{L} sequence comprising a CDR-L3 sequence comprising, consisting of, or consisting essentially of SEQ ID NO: 164. In some aspects, the antibody comprises a V_{L} sequence comprising a CDR-L3 sequence comprising, consisting of, or consisting essentially of SEQ ID NO: 165. In some aspects, the antibody comprises a V_{L} sequence comprising a CDR-L3 sequence comprising, consisting of, or consisting essentially of SEQ ID NO: 166.

### 2.5.2. CDR-L2

In some embodiments, the antibody comprises a V_{L} sequence comprising a CDR-L2 sequence comprising, consisting of, or consisting essentially of a sequence selected from SEQ ID NOs: 125-140. In some aspects, the antibody comprises a V_{L} sequence comprising a CDR-L2 sequence comprising, consisting of, or consisting essentially of SEQ ID NO: 125. In some aspects, the antibody comprises a V_{L} sequence comprising a CDR-L2 sequence comprising, consisting of, or consisting essentially of SEQ ID NO: 126. In some aspects, the antibody comprises a V_{L} sequence comprising a CDR-L2 sequence comprising, consisting of, or consisting essentially of SEQ ID NO: 127. In some aspects, the antibody comprises a V_{L} sequence comprising a CDR-L2 sequence comprising, consisting of, or consisting essentially of SEQ ID NO: 128. In some aspects, the antibody comprises a V_{L} sequence comprising a CDR-L2 sequence comprising, consisting of, or consisting essentially of SEQ ID NO: 129. In some aspects, the antibody comprises a V_{L} sequence comprising a CDR-L2 sequence comprising, consisting of, or consisting essentially of SEQ ID NO: 130. In some aspects, the antibody comprises a V_{L} sequence comprising a CDR-H2 sequence comprising, consisting of, or consisting essentially of SEQ ID NO: 131. In some aspects, the antibody comprises a V_{L} sequence comprising a CDR-L2 sequence comprising, consisting of, or consisting essentially of SEQ ID NO: 132. In some aspects, the antibody comprises a V_{L} sequence comprising a CDR-1.2 sequence comprising, consisting of, or consisting essentially of SEQ ID NO: 133. In some aspects, the antibody comprises a V_{L} sequence comprising a CDR-L2 sequence comprising, consisting of, or consisting essentially of SEQ ID NO: 134. In some aspects, the antibody comprises a V_{L} sequence comprising a CDR-L2 sequence comprising, consisting of, or consisting essentially of SEQ ID NO: 135. In some aspects, the antibody comprises a V_{L} sequence comprising a CDR-L2 sequence comprising, consisting of, or consisting essentially of SEQ ID NO: 136. In some aspects, the antibody comprises a V_{L} sequence comprising a CDR-L2 sequence comprising, consisting of, or consisting essentially of SEQ ID NO: 137. In some aspects, the antibody comprises a V_{L} sequence comprising a CDR-L2 sequence comprising, consisting of, or consisting essentially of SEQ ID NO: 138. In some aspects, the antibody comprises a V_{L} sequence comprising a CDR-L2 sequence comprising, consisting of, or consisting essentially of SEQ ID NO: 139. In some aspects, the antibody comprises a V_{L} sequence comprising a CDR-1.2 sequence comprising, consisting of, or consisting essentially of SEQ ID NO: 140.

### 2.5.3. CDR-L1

In some embodiments, the antibody comprises a V_{L} sequence comprising a CDR-L1 sequence comprising, consisting of, or consisting essentially of a sequence selected from SEQ ID NOs: 110-124. In some aspects, the antibody comprises a V_{L} sequence comprising a CDR-L1 sequence comprising, consisting of, or consisting essentially of SEQ ID NO: 110. In some aspects, the antibody comprises a V_{L} sequence comprising a CDR-L1 sequence comprising, consisting of, or consisting essentially of SEQ ID NO: 111. In some aspects, the antibody comprises a V_{L} sequence comprising a CDR-L1 sequence comprising, consisting of, or consisting essentially of SEQ ID NO: 112. In some aspects, the antibody comprises a V_{L} sequence comprising a CDR-L1 sequence comprising, consisting of, or consisting essentially of SEQ ID NO: 113. In some aspects, the antibody comprises a V_{L} sequence comprising a CDR-L1 sequence comprising, consisting of, or consisting essentially of SEQ ID NO: 114. In some aspects, the antibody comprises a V_{L} sequence comprising a CDR-L1 sequence comprising, consisting of, or consisting essentially of SEQ ID NO: 115. In some aspects, the antibody comprises a V_{L} sequence comprising a CDR-L1 sequence comprising, consisting of, or consisting essentially of SEQ ID NO: 116. In some aspects, the antibody comprises a V_{L} sequence comprising a CDR-L1 sequence comprising, consisting of, or consisting essentially of SEQ ID NO: 117. In some aspects, the antibody comprises a V_{L} sequence comprising a CDR-L1 sequence comprising, consisting of, or consisting essentially of SEQ ID NO: 118. In some aspects, the antibody comprises a V_{L} sequence comprising a CDR-L1 sequence comprising, consisting of, or consisting essentially of SEQ ID NO: 119. In some aspects, the antibody comprises a V_{L} sequence comprising a CDR-L1 sequence comprising, consisting of, or consisting essentially of SEQ ID NO: 120. In some aspects, the antibody comprises a V_{L} sequence comprising a CDR-L1 sequence comprising, consisting of, or consisting essentially of SEQ ID NO: 121. In some aspects, the antibody comprises a V_{L} sequence comprising a CDR-L1 sequence comprising, consisting of, or consisting essentially of SEQ ID NO: 122. In some aspects, the antibody comprises a V_{L} sequence comprising a CDR-L1 sequence comprising, consisting of, or consisting essentially of SEQ ID NO: 123. In some aspects, the antibody comprises a V_{L} sequence comprising a CDR-L1 sequence comprising, consisting of, or consisting essentially of SEQ ID NO: 124.

### 2.5.4. CDR-L3+CDR-L2

In some embodiments, the antibody comprises a V_{L} sequence comprising a CDR-L3 sequence comprising, consisting of, or consisting essentially of a sequence selected from SEQ ID NOs: 141-166 and a CDR-L2 sequence comprising, consisting of, or consisting essentially of a sequence selected from SEQ ID NOs: 125-140. In some aspects, the CDR-L3 sequence and the CDR-L2 sequence are both from a single illustrative V_{L} sequence provided in this disclosure. For example, in some aspects, the CDR-L3 and CDR-L2 are both from a single illustrative V_{L} sequence selected from SEQ ID NOs: 219-248.

### 2.5.5. CDR-13+CDR-L1

In some embodiments, the antibody comprises a V_{L} sequence comprising a CDR-L3 sequence comprising, consisting of, or consisting essentially of a sequence selected from SEQ ID NOs: 141-166 and a CDR-L1 sequence comprising, consisting of, or consisting essentially of a sequence selected from SEQ ID NOs: 110-124. In some aspects, the CDR-L3 sequence and the CDR-L1 sequence are both from a single illustrative V_{L} sequence provided in this disclosure. For example, in some aspects, the CDR-L3 and CDR-L1 are both from a single illustrative V_{L} sequence selected from SEQ ID NOs: 219-248.

### 2.5.6. CDR-L1+CDR-L2

In some embodiments, the antibody comprises a V_{L} sequence comprising a CDR-L1 sequence comprising, consisting of, or consisting essentially of a sequence selected from SEQ ID NOs: 110-124 and a CDR-L2 sequence comprising, consisting of, or consisting essentially of a sequence selected from SEQ ID NOs: 125-140. In some aspects, the CDR-L1 sequence and the CDR-L2 sequence are both from a single illustrative V_{L} sequence provided in this disclosure. For example, in some aspects, the CDR-L1 and CDR-L2 are both from a single illustrative V_{L} sequence selected from SEQ ID NOs: 219-248.

### 2.5.7. CDR-L1+CDR-L2+CDR-L3

In some embodiments, the antibody comprises a V_{L} sequence comprising a CDR-L1 sequence comprising, consisting of, or consisting essentially of a sequence selected from SEQ ID NOs: 110-124, a CDR-L2 sequence comprising, consisting of, or consisting essentially of a sequence selected from SEQ ID NOs: 125-140, and a CDR-L3 sequence comprising, consisting of, or consisting essentially of a sequence selected from SEQ ID NOs: 141-166. In some aspects, the CDR-L1 sequence, CDR-L2 sequence, and CDR-L3 sequence are all from a single illustrative V_{L} sequence provided in this disclosure. For example, in some aspects, the CDR-L1, CDR-L2, and CDR-L3 are all from a single illustrative V_{L} sequence selected from SEQ ID NOs: 219-248.

In some aspects, the antibody comprises a V_{L} sequence comprising a CDR-L1 sequence comprising SEQ ID NO: 110, a CDR-L2 sequence comprising SEQ ID NO: 125, and a CDR-L3 sequence SEQ ID NO: 141. In some aspects, the antibody comprises a VL sequence comprising a CDR-L1 sequence comprising SEQ ID NO: 111, a CDR-L2 sequence comprising SEQ ID NO: 126, and a CDR-L3 sequence comprising SEQ ID NO: 142. In some aspects, the antibody comprises a VL sequence comprising a CDR-L1 sequence comprising SEQ ID NO: 110, a CDR-L2 sequence comprising SEQ ID NO: 127, and a CDR-L3 sequence comprising sequence selected from SEQ ID NO: 142. In some aspects, the antibody comprises a VL sequence comprising a CDR-L1 sequence comprising SEQ ID NO: 110, a CDR-L2 sequence comprising SEQ ID NO: 125, and a CDR-L3 sequence comprising SEQ ID NO: 143. In some aspects, the antibody comprises a VL sequence comprising a CDR-L1 sequence comprising SEQ ID NO: 112, a CDR-L2 sequence comprising SEQ ID NO: 128, and a CDR-L3 sequence comprising SEQ ID NO: 144. In some aspects, the antibody comprises a VL sequence comprising a CDR-L1 sequence comprising SEQ ID NO: 111, a CDR-L2 sequence comprising SEQ ID NO: 126, and a CDR-L3 sequence comprising SEQ ID NO: 145. In some aspects, the antibody comprises a VL sequence comprising a CDR-L1 sequence comprising SEQ ID NO: 113, a CDR-L2 sequence comprising SEQ ID NO: 129, and a CDR-L3 sequence comprising SEQ ID NO: 146. In some aspects, the antibody comprises a VL sequence comprising a CDR-L1 sequence comprising SEQ ID NO: 114, a CDR-L2 sequence comprising SEQ ID NO: 130, and a CDR-L3 sequence comprising SEQ ID NO: 147. In some aspects, the antibody comprises a VL sequence comprising a CDR-L1 sequence comprising SEQ ID NO: 115, a CDR-L2 sequence comprising SEQ ID NO: 131, and a CDR-L3 sequence comprising SEQ ID NO: 148. In some aspects, the antibody comprises a VL sequence comprising a CDR-L1 sequence comprising SEQ ID NO: 115, a CDR-L2 sequence comprising SEQ ID NO: 131, and a CDR-L3 sequence comprising SEQ ID NO: 148. In some aspects, the antibody comprises a VL sequence comprising a CDR-L1 sequence comprising SEQ ID NO: 116, a CDR-L2 sequence comprising SEQ ID NO: 131, and a CDR-L3 sequence comprising SEQ ID NO: 149. In some aspects, the antibody comprises a VL sequence comprising a CDR-L1 sequence comprising SEQ ID NO: 115, a CDR-L2 sequence comprising SEQ ID NO: 131, and a CDR-L3 sequence comprising SEQ ID NO: 148. In some aspects, the antibody comprises a VL sequence comprising a CDR-L1 sequence comprising SEQ ID NO: 115, a CDR-L2 sequence comprising SEQ ID NO: 132, and a CDR-L3 sequence comprising SEQ ID NO: 149. In some aspects, the antibody comprises a VL sequence comprising a CDR-L1 sequence comprising SEQ ID NO: 117, a CDR-L2 sequence comprising SEQ ID NO: 133, and a CDR-L3 sequence comprising SEQ ID NO: 150. In some aspects, the antibody comprises a VL sequence comprising a CDR-L1 sequence comprising SEQ ID NO: 115, a CDR-L2 sequence comprising SEQ ID NO: 134, and a CDR-L3 sequence comprising SEQ ID NO: 148. In some aspects, the antibody comprises a VL sequence comprising a CDR-L1 sequence comprising SEQ ID NO: 118, a CDR-L2 sequence comprising SEQ ID NO: 135, and a CDR-L3 sequence comprising SEQ ID NO: 151. In some aspects, the antibody comprises a V_{L} sequence comprising a CDR-L1 sequence comprising SEQ ID NO: 118, a CDR-L2 sequence comprising SEQ ID NO: 135, and a CDR-L3 sequence comprising SEQ ID NO: 152. In some aspects, the antibody comprises a VL sequence comprising a CDR-L1 sequence comprising SEQ ID NO: 118, a CDR-1.2 sequence comprising SEQ ID NO: 136, and a CDR-L3 sequence comprising SEQ ID NO: 152. In some aspects, the antibody comprises a VL sequence comprising a CDR-L1 sequence comprising SEQ ID NO: 115, a CDR-L2 sequence comprising SEQ ID NO: 131, and a CDR-L3 sequence comprising SEQ ID NO: 153. In some aspects, the antibody comprises a VL sequence comprising a CDR-L1 sequence comprising SEQ ID NO: 119, a CDR-L2 sequence comprising SEQ ID NO: 131, and a CDR-L3 sequence comprising SEQ ID NO: 154. In some aspects, the antibody comprises a VL sequence comprising a CDR-L1 sequence comprising SEQ ID NO: 120, a CDR-L2 sequence comprising SEQ ID NO: 137, and a CDR-L3 sequence comprising SEQ ID NO: 155. In some aspects, the antibody comprises a VL sequence comprising a CDR-L1 sequence comprising SEQ ID NO: 121, a CDR-L2 sequence comprising SEQ ID NO: 138, and a CDR-L3 sequence comprising SEQ ID NO: 156. In some aspects, the antibody comprises a VL sequence comprising a CDR-L1 sequence comprising SEQ ID NO: 121, a CDR-L2 sequence comprising SEQ ID NO: 138, and a CDR-L3 sequence comprising SEQ ID NO: 157. In some aspects, the antibody comprises a VL sequence comprising a CDR-L1 sequence comprising SEQ ID NO: 122, a CDR-L2 sequence comprising SEQ ID NO: 138, and a CDR-13 sequence comprising SEQ ID NO: 158. In some aspects, the antibody comprises a VL sequence comprising a CDR-L1 sequence comprising SEQ ID NO: 121, a CDR-1.2 sequence comprising SEQ ID NO: 138, and a CDR-L3 sequence comprising SEQ ID NO: 159. In some aspects, the antibody comprises a VL sequence comprising a CDR-L1 sequence comprising SEQ ID NO: 118, a CDR-L2 sequence comprising SEQ ID NO: 135, and a CDR-L3 sequence comprising SEQ ID NO: 160. In some aspects, the antibody comprises a VL sequence comprising a CDR-L1 sequence comprising SEQ ID NO: 121, a CDR-L2 sequence comprising SEQ ID NO: 139, and a CDR-L3 sequence comprising SEQ ID NO: 161. In some aspects, the antibody comprises a VL sequence comprising a CDR-1.1 sequence comprising SEQ ID NO: 123, a CDR-L2 sequence comprising SEQ ID NO: 140, and a CDR-L3 sequence comprising SEQ ID NO: 162. In some aspects, the antibody comprises a VL sequence comprising a CDR-L1 sequence comprising SEQ ID NO: 121, a CDR-L2 sequence comprising SEQ ID NO: 138, and a CDR-L3 sequence comprising SEQ ID NO: 163. In some aspects, the antibody comprises a VL sequence comprising a CDR-L1 sequence comprising SEQ ID NO: 121, a CDR-L2 sequence comprising SEQ ID NO: 139, and a CDR-L3 sequence comprising SEQ ID NO: 164. In some aspects, the antibody comprises a VL sequence comprising a CDR-L1 sequence comprising SEQ ID NO: 120, a CDR-L2 sequence comprising SEQ ID NO: 137, and a CDR-L3 sequence comprising SEQ ID NO: 165. In some aspects, the antibody comprises a VL sequence comprising a CDR-L1 sequence comprising SEQ ID NO: 124, a CDR-L2 sequence comprising SEQ ID NO: 125, and a CDR-L3 sequence comprising SEQ ID NO: 166.

In some aspects, the antibody comprises a VL sequence comprising a CDR-L1 sequence comprising SEQ ID NO: 110, a CDR-L2 sequence comprising SEQ ID NO: 128, and a CDR-L3 sequence SEQ ID NO: 144. In some aspects, the antibody comprises a VL sequence comprising a CDR-L1 sequence comprising SEQ ID NO: 110, a CDR-L2 sequence comprising SEQ ID NO: 125, and a CDR-L3 sequence comprising SEQ ID NO: 144. In some aspects, the antibody comprises a VL sequence comprising a CDR-L1 sequence comprising SEQ ID NO: 110, a CDR-H2 sequence comprising SEQ ID NO: 126, and a CDR-L3 sequence comprising sequence selected from SEQ ID NO: 144. In some aspects, the antibody comprises a VL sequence comprising a CDR-L1 sequence comprising SEQ ID NO: 116, a CDR-L2 sequence comprising SEQ ID NO: 131, and a CDR-L3 sequence comprising SEQ ID NO: 150. In some aspects, the antibody comprises a VL sequence comprising a CDR-L1 sequence comprising SEQ ID NO: 117, a CDR-L2 sequence comprising SEQ ID NO: 134, and a CDR-L3 sequence comprising SEQ ID NO: 150.

### 2.5.8. Variants of V1 Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

### 2.6. V_{L} Sequences

In some embodiments, the antibody comprises a V_{L} sequence comprising, consisting of, or consisting essentially of a sequence selected from SEQ ID NOs: 219-248. In some aspects, the antibody comprises a V_{L} sequence comprising, consisting of, or consisting essentially of SEQ ID NO: 219. In some aspects, the antibody comprises a V_{L} sequence comprising, consisting of, or consisting essentially of SEQ ID NO: 220. In some aspects, the antibody comprises a V_{L} sequence comprising, consisting of, or consisting essentially of SEQ ID NO: 221. In some aspects, the antibody comprises a V_{L} sequence comprising, consisting of, or consisting essentially of SEQ ID NO: 222. In some aspects, the antibody comprises a V_{L} sequence comprising, consisting of, or consisting essentially of SEQ ID NO: 223. In some aspects, the antibody comprises a V_{L} sequence comprising, consisting of, or consisting essentially of SEQ ID NO: 224. In some aspects, the antibody comprises a V_{L} sequence comprising, consisting of, or consisting essentially of SEQ ID NO: 225. In some aspects, the antibody comprises a V_{L} sequence comprising, consisting of, or consisting essentially of SEQ ID NO: 226. In some aspects, the antibody comprises a V_{L} sequence comprising, consisting of, or consisting essentially of SEQ ID NO: 227. In some aspects, the antibody comprises a V_{L} sequence comprising, consisting of, or consisting essentially of SEQ ID NO: 228. In some aspects, the antibody comprises a V_{L} sequence comprising, consisting of, or consisting essentially of SEQ ID NO: 229. In some aspects, the antibody comprises a V_{L} sequence comprising, consisting of, or consisting essentially of SEQ ID NO: 230. In some aspects, the antibody comprises a V_{L} sequence comprising, consisting of, or consisting essentially of SEQ ID NO: 231. In some aspects, the antibody comprises a V_{L} sequence comprising, consisting of, or consisting essentially of SEQ ID NO: 232. In some aspects, the antibody comprises a V_{L} sequence comprising, consisting of, or consisting essentially of SEQ ID NO: 233. In some aspects, the antibody comprises a V_{L} sequence comprising, consisting of, or consisting essentially of SEQ ID NO: 234. In some aspects, the antibody comprises a V_{L} sequence comprising, consisting of, or consisting essentially of SEQ ID NO: 235. In some aspects, the antibody comprises a V_{L} sequence comprising, consisting of, or consisting essentially of SEQ ID NO: 236. In some aspects, the antibody comprises a V_{L} sequence comprising, consisting of, or consisting essentially of SEQ ID NO: 237. In some aspects, the antibody comprises a V_{L} sequence comprising, consisting of, or consisting essentially of SEQ ID NO: 238. In some aspects, the antibody comprises a V_{L} sequence comprising, consisting of, or consisting essentially of SEQ ID NO: 239. In some aspects, the antibody comprises a V_{L} sequence comprising, consisting of, or consisting essentially of SEQ ID NO: 240. In some aspects, the antibody comprises a V_{L} sequence comprising, consisting of, or consisting essentially of SEQ ID NO: 241. In some aspects, the antibody comprises a V_{L} sequence comprising, consisting of, or consisting essentially of SEQ ID NO: 242. In some aspects, the antibody comprises a V_{L} sequence comprising, consisting of, or consisting essentially of SEQ ID NO: 243. In some aspects, the antibody comprises a V_{L} sequence comprising, consisting of, or consisting essentially of SEQ ID NO: 244. In some aspects, the antibody comprises a V_{L} sequence comprising, consisting of, or consisting essentially of SEQ ID NO: 245. In some aspects, the antibody comprises a V_{L} sequence comprising, consisting of, or consisting essentially of SEQ ID NO: 246. In some aspects, the antibody comprises a V_{L} sequence comprising, consisting of, or consisting essentially of SEQ ID NO: 247. In some aspects, the antibody comprises a V_{L} sequence comprising, consisting of, or consisting essentially of SEQ ID NO: 248.

### 2.6.1. Variants of V_{L} Sequences

In some embodiments, the V_{L} sequences provided herein comprise, consist of, or consist essentially of a variant of an illustrative V_{L} sequence provided in this disclosure.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer. 3 or fewer. 2 or fewer, or 1 or fewer amino acid substitutions. In some aspects, the amino acid substitutions are conservative amino acid substitutions.

### 2.7. Pairs

### 2.7.1. CDR-H3-CDR-L3 Pairs

In some embodiments, the antibody comprises a CDR-H3 sequence and a CDR-L3 sequence. In some aspects, the CDR-H3 sequence is part of a V_{H} and the CDR-L3 sequence is part of a V_{L}.

In some aspects, the CDR-H3 sequence is a CDR-H3 sequence comprising, consisting of, or consisting essentially of SEQ ID NOs: 82-109, and the CDR-L3 sequence is a CDR-L3 sequence comprising, consisting of, or consisting essentially of SEQ ID NOs: 141-166.

In some aspects, the CDR-H3 sequence is SEQ ID NO: 82 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-13 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-13 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 83 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 84 and the CDR-L3 sequence is selected from SEQ ID NOs: 141-166. In some aspects, the CDR-L3 sequence is SEQ ID NO: 141. In some aspects, the CDR-13 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-13 sequence is SEQ ID NO: 85 and the CDR-13 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-13 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-13 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 86 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-13 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 87 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 88 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 89 and the CDR-L3 sequence is selected from SEQ ID NOs: 141-166. In some aspects, the CDR-L3 sequence is SEQ ID NO: 141. In some aspects, the CDR-13 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 90 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-13 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-13 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 91 and the CDR-L3 sequence is selected from SEQ ID NOs: 141-166. In some aspects, the CDR-13 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 92 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 93 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 94 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-13 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-13 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 95 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-13 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-13 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 96 and the CDR-L3 sequence is selected from SEQ ID NOs: 141-166. In some aspects, the CDR-13 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H13 sequence is SEQ ID NO: 97 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 98 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 99 and the CDR-L3 sequence is selected from SEQ ID NOs: 141-166. In some aspects, the CDR-L3 sequence is SEQ ID NO: 141. In some aspects, the CDR-13 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-13 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-13 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 100 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-13 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 101 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 102 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 103 and the CDR-13 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-13 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 104 and the CDR-13 sequence is selected from SEQ ID NOs: 141-166. In some aspects, the CDR-L3 sequence is SEQ ID NO: 141. In some aspects, the CDR-13 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 105 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 106 and the CDR-L3 sequence is selected from SEQ ID NOs: 141-166. In some aspects, the CDR-13 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 107 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 108 and the CDR-13 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-13 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-13 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 109 and the CDR-L3 sequence is selected from SEQ ID NOs: 141-166. In some aspects, the CDR-L3 sequence is SEQ ID NO: 141. In some aspects, the CDR-L3 sequence is SEQ ID NO: 142. In some aspects, the CDR-L3 sequence is SEQ ID NO: 143. In some aspects, the CDR-L3 sequence is SEQ ID NO: 144. In some aspects, the CDR-L3 sequence is SEQ ID NO: 145. In some aspects, the CDR-L3 sequence is SEQ ID NO: 146. In some aspects, the CDR-L3 sequence is SEQ ID NO: 147. In some aspects, the CDR-L3 sequence is SEQ ID NO: 148. In some aspects, the CDR-L3 sequence is SEQ ID NO: 149. In some aspects, the CDR-L3 sequence is SEQ ID NO: 150. In some aspects, the CDR-13 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-13 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

### 2.7.1.1. Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 2.7.2. V_{H}-V_{L} Pairs

In some embodiments, the antibody comprises a V_{H} sequence and a V_{L} sequence.

In some aspects, the V_{H} sequence is a V_{H} sequence comprising, consisting of, or consisting essentially of SEQ ID NOs: 179-218 and the V_{L} sequence is a V_{L} sequence comprising, consisting of, or consisting essentially of SEQ ID NOs: 219-248.

In some aspects, the V_{H} sequence is SEQ ID NO: 179 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 180 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO): 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 181 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 182 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 183 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO): 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 184 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 185 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 186 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 187 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 188 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 189 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 190 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 191 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 192 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 193 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 194 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 195 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 196 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 197 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 198 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 199 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 200 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 201 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO): 246. In some aspects, the V_{L} sequence is SEQ ID NO: 247. In some aspects, the V_{L} sequence is SEQ ID NO: 248.

In some aspects, the V_{H} sequence is SEQ ID NO: 202 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 203 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 204 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 205 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 206 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 207 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 208 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 209 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 210 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 211 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 212 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 213 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO): 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 214 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 215 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 216 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO): 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 217 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

In some aspects, the V_{H} sequence is SEQ ID NO: 218 and the V_{L} sequence is selected from SEQ ID NOs: 219-248. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228. In some aspects, the V_{L} sequence is SEQ ID NO: 229. In some aspects, the V_{L} sequence is SEQ ID NO: 230. In some aspects, the V_{L} sequence is SEQ ID NO: 231. In some aspects, the V_{L} sequence is SEQ ID NO: 232. In some aspects, the V_{L} sequence is SEQ ID NO: 233. In some aspects, the V_{L} sequence is SEQ ID NO: 234. In some aspects, the V_{L} sequence is SEQ ID NO: 235. In some aspects, the V_{L} sequence is SEQ ID NO: 236. In some aspects, the V_{L} sequence is SEQ ID NO: 237. In some aspects, the V_{L} sequence is SEQ ID NO: 238. In some aspects, the V_{L} sequence is SEQ ID NO: 239. In some aspects, the V_{L} sequence is SEQ ID NO: 240. In some aspects, the V_{L} sequence is SEQ ID NO: 241. In some aspects, the V_{L} sequence is SEQ ID NO: 242. In some aspects, the V_{L} sequence is SEQ ID NO: 243. In some aspects, the V_{L} sequence is SEQ ID NO: 244. In some aspects, the V_{L} sequence is SEQ ID NO: 245. In some aspects, the V_{L} sequence is SEQ ID NO: 246. In some aspects, the V_{L} sequence is SEQ ID NO: 247. In some aspects, the V_{L} sequence is SEQ ID NO: 248.

### 2.7.3. CDR-H1+CDR-H2+CDR-H3+CDR-L1+CDR-H2+CDR-L3

In some aspects, the antibody comprises a V_{H} sequence comprising a Kabat CDR-H1 sequence comprising SEQ ID NO: 25, a Kabat CDR-H2 sequence comprising SEQ ID NO: 63, and a Kabat CDR-H3 sequence comprising SEQ ID NO: 82 and a VL sequence comprising a CDR-L1 sequence comprising SEQ ID NO: 110, a CDR-L2 sequence comprising SEQ ID NO: 125, and a CDR-L3 sequence SEQ ID NO: 141. In some aspects, the antibody comprises a V_{H} sequence comprising a Kabat CDR-H1 sequence comprising SEQ ID NO: 26, a Kabat CDR-H2 sequence comprising SEQ ID NO: 64, and a Kabat CDR-H3 sequence comprising SEQ ID NO: 83 and a VL sequence comprising a CDR-L1 sequence comprising SEQ ID NO: 111, a CDR-L2 sequence comprising SEQ ID NO: 126, and a CDR-L3 sequence SEQ ID NO: 142. In some aspects, the antibody comprises a V_{H} sequence comprising a Kabat CDR-H1 sequence comprising SEQ ID NO: 27, a Kabat CDR-H2 sequence comprising SEQ ID NO: 65, and a Kabat CDR-H3 sequence comprising SEQ ID NO: 84 and a VL sequence comprising a CDR-L1 sequence comprising SEQ ID NO: 110, a CDR-L2 sequence comprising SEQ ID NO: 125, and a CDR-L3 sequence SEQ ID NO: 141. In some aspects, the antibody comprises a V_{H} sequence comprising a Kabat CDR-H1 sequence comprising SEQ ID NO: 26, a Kabat CDR-H2 sequence comprising SEQ ID NO: 64, and a Kabat CDR-H3 sequence comprising SEQ ID NO: 85 and a VL sequence comprising a CDR-L1 sequence comprising SEQ ID NO: 110, a CDR-L2 sequence comprising SEQ ID NO: 125, and a CDR-L3 sequence SEQ ID NO: 141. In some aspects, the antibody comprises a V_{H} sequence comprising a Kabat CDR-H1 sequence comprising SEQ ID NO: 26, a Kabat CDR-H2 sequence comprising SEQ ID NO: 64, and a Kabat CDR-H3 sequence comprising SEQ ID NO: 86 and a VL sequence comprising a CDR-L1 sequence comprising SEQ ID NO: 110, a CDR-L2 sequence comprising SEQ ID NO: 127, and a CDR-L3 sequence SEQ ID NO: 142. In some aspects, the antibody comprises a V_{H} sequence comprising a Kabat CDR-H1 sequence comprising SEQ ID NO: 25, a Kabat CDR-H2 sequence comprising SEQ ID NO: 66, and a Kabat CDR-H3 sequence comprising SEQ ID NO: 82 and a VL sequence comprising a CDR-L1 sequence comprising SEQ ID NO: 110, a CDR-L2 sequence comprising SEQ ID NO: 125, and a CDR-L3 sequence SEQ ID NO: 141. In some aspects, the antibody comprises a V_{H} sequence comprising a Kabat CDR-H1 sequence comprising SEQ ID NO: 26, a Kabat CDR-H2 sequence comprising SEQ ID NO: 65, and a Kabat CDR-H3 sequence comprising SEQ ID NO: 82 and a VL sequence comprising a CDR-L1 sequence comprising SEQ ID NO: 110, a CDR-L2 sequence comprising SEQ ID NO: 125, and a CDR-L3 sequence SEQ ID NO: 143. In some aspects, the antibody comprises a V_{H} sequence comprising a Kabat CDR-H1 sequence comprising SEQ ID NO: 26, a Kabat CDR-H2 sequence comprising SEQ ID NO: 65, and a Kabat CDR-H3 sequence comprising SEQ ID NO: 82 and a VL sequence comprising a CDR-L1 sequence comprising SEQ ID NO: 112, a CDR-L2 sequence comprising SEQ ID NO: 128, and a CDR-L3 sequence SEQ ID NO: 144. In some aspects, the antibody comprises a V_{H} sequence comprising a Kabat CDR-H1 sequence comprising SEQ ID NO: 27, a Kabat CDR-H2 sequence comprising SEQ ID NO: 65, and a Kabat CDR-H3 sequence comprising SEQ ID NO: 82 and a V_{L} sequence comprising a CDR-L1 sequence comprising SEQ ID NO: 111, a CDR-L2 sequence comprising SEQ ID NO: 126, and a CDR-L3 sequence SEQ ID NO: 145. In some aspects, the antibody comprises a V_{H} sequence comprising a Kabat CDR-H1 sequence comprising SEQ ID NO: 28, a Kabat CDR-H2 sequence comprising SEQ ID NO: 67, and a Kabat CDR-H3 sequence comprising SEQ ID NO: 82 and a VL sequence comprising a CDR-L1 sequence comprising SEQ ID NO: 113, a CDR-H2 sequence comprising SEQ ID NO: 129, and a CDR-L3 sequence SEQ ID NO: 146. In some aspects, the antibody comprises a V_{H} sequence comprising a Kabat CDR-H1 sequence comprising SEQ ID NO: 29, a Kabat CDR-H2 sequence comprising SEQ ID NO: 68, and a Kabat CDR-H3 sequence comprising SEQ ID NO: 82 and a VL sequence comprising a CDR-L1 sequence comprising SEQ ID NO: 114, a CDR-L2 sequence comprising SEQ ID NO: 130, and a CDR-L3 sequence SEQ ID NO: 147. In some aspects, the antibody comprises a V_{H} sequence comprising a Kabat CDR-H1 sequence comprising SEQ ID NO: 26, a Kabat CDR-H2 sequence comprising SEQ ID NO: 64, and a Kabat CDR-H3 sequence comprising SEQ ID NO: 82 and a VL sequence comprising a CDR-L1 sequence comprising SEQ ID NO: 110, a CDR-H2 sequence comprising SEQ ID NO: 125, and a CDR-L3 sequence SEQ ID NO: 141. In some aspects, the antibody comprises a V_{H} sequence comprising a Kabat CDR-H1 sequence comprising SEQ ID NO: 30, a Kabat CDR-H2 sequence comprising SEQ ID NO: 69, and a Kabat CDR-H3 sequence comprising SEQ ID NO: 87 and a VL sequence comprising a CDR-L1 sequence comprising SEQ ID NO: 115, a CDR-H2 sequence comprising SEQ ID NO: 131, and a CDR-L3 sequence SEQ ID NO: 148. In some aspects, the antibody comprises a V_{H} sequence comprising a Kabat CDR-H1 sequence comprising SEQ ID NO: 31, a Kabat CDR-H2 sequence comprising SEQ ID NO: 70, and a Kabat CDR-H3 sequence comprising SEQ ID NO: 88 and a VL sequence comprising a CDR-L1 sequence comprising SEQ ID NO: 116, a CDR-L2 sequence comprising SEQ ID NO: 131, and a CDR-L3 sequence SEQ ID NO: 149. In some aspects, the antibody comprises a V_{H} sequence comprising a Kabat CDR-H1 sequence comprising SEQ ID NO: 32, a Kabat CDR-H2 sequence comprising SEQ ID NO: 71, and a Kabat CDR-H3 sequence comprising SEQ ID NO: 89 and a VL sequence comprising a CDR-L1 sequence comprising SEQ ID NO: 115, a CDR-L2 sequence comprising SEQ ID NO: 131, and a CDR-L3 sequence SEQ ID NO: 148. In some aspects, the antibody comprises a V_{H} sequence comprising a Kabat CDR-H1 sequence comprising SEQ ID NO: 33, a Kabat CDR-H2 sequence comprising SEQ ID NO: 70, and a Kabat CDR-H3 sequence comprising SEQ ID NO: 90 and a VL sequence comprising a CDR-L1 sequence comprising SEQ ID NO: 115, a CDR-L2 sequence comprising SEQ ID NO: 132, and a CDR-L3 sequence SEQ ID NO: 149. In some aspects, the antibody comprises a V_{H} sequence comprising a Kabat CDR-H1 sequence comprising SEQ ID NO: 34, a Kabat CDR-H2 sequence comprising SEQ ID NO: 72, and a Kabat CDR-H3 sequence comprising SEQ ID NO: 91 and a V_{L} sequence comprising a CDR-L1 sequence comprising SEQ ID NO: 115, a CDR-L2 sequence comprising SEQ ID NO: 131, and a CDR-L3 sequence SEQ ID NO: 148. In some aspects, the antibody comprises a V_{H} sequence comprising a Kabat CDR-H1 sequence comprising SEQ ID NO: 30, a Kabat CDR-H2 sequence comprising SEQ ID NO: 71, and a Kabat CDR-H3 sequence comprising SEQ ID NO: 92 and a VL sequence comprising a CDR-L1 sequence comprising SEQ ID NO: 115, a CDR-L2 sequence comprising SEQ ID NO: 131, and a CDR-L3 sequence SEQ ID NO: 148. In some aspects, the antibody comprises a V_{H} sequence comprising a Kabat CDR-H1 sequence comprising SEQ ID NO: 35, a Kabat CDR-H2 sequence comprising SEQ ID NO: 70, and a Kabat CDR-H3 sequence comprising SEQ ID NO: 93 and a VL sequence comprising a CDR-L1 sequence comprising SEQ ID NO: 117, a CDR-L2 sequence comprising SEQ ID NO: 133, and a CDR-L3 sequence SEQ ID NO: 150. In some aspects, the antibody comprises a V_{H} sequence comprising a Kabat CDR-H1 sequence comprising SEQ ID NO: 33, a Kabat CDR-H2 sequence comprising SEQ ID NO: 70, and a Kabat CDR-H3 sequence comprising SEQ ID NO: 92 and a VL sequence comprising a CDR-L1 sequence comprising SEQ ID NO: 115, a CDR-L2 sequence comprising SEQ ID NO: 134, and a CDR-L3 sequence SEQ ID NO: 148. In some aspects, the antibody comprises a V_{H} sequence comprising a Kabat CDR-H1 sequence comprising SEQ ID NO: 36, a Kabat CDR-H2 sequence comprising SEQ ID NO: 72, and a Kabat CDR-H3 sequence comprising SEQ ID NO: 87 and a VL sequence comprising a CDR-L1 sequence comprising SEQ ID NO: 115, a CDR-L2 sequence comprising SEQ ID NO: 131, and a CDR-L3 sequence SEQ ID NO: 148. In some aspects, the antibody comprises a V_{H} sequence comprising a Kabat CDR-H1 sequence comprising SEQ ID NO: 30, a Kabat CDR-H2 sequence comprising SEQ ID NO: 73, and a Kabat CDR-H3 sequence comprising SEQ ID NO: 87 and a VL sequence comprising a CDR-L1 sequence comprising SEQ ID NO: 115, a CDR-L2 sequence comprising SEQ ID NO: 131, and a CDR-L3 sequence SEQ ID NO: 148. In some aspects, the antibody comprises a V_{H} sequence comprising a Kabat CDR-H1 sequence comprising SEQ ID NO: 37, a Kabat CDR-H2 sequence comprising SEQ ID NO: 74, and a Kabat CDR-H3 sequence comprising SEQ ID NO: 87 and a VL sequence comprising a CDR-L1 sequence comprising SEQ ID NO: 115, a CDR-L2 sequence comprising SEQ ID NO: 131, and a CDR-L3 sequence SEQ ID NO: 148. In some aspects, the antibody comprises a V_{H} sequence comprising a Kabat CDR-H1 sequence comprising SEQ ID NO: 38, a Kabat CDR-H2 sequence comprising SEQ ID NO: 75, and a Kabat CDR-H3 sequence comprising SEQ ID NO: 94 and a V_{L} sequence comprising a CDR-L1 sequence comprising SEQ ID NO: 118, a CDR-L2 sequence comprising SEQ ID NO: 135, and a CDR-L3 sequence SEQ ID NO: 151. In some aspects, the antibody comprises a V_{H} sequence comprising a Kabat CDR-H1 sequence comprising SEQ ID NO: 39, a Kabat CDR-H2 sequence comprising SEQ ID NO: 76, and a Kabat CDR-H3 sequence comprising SEQ ID NO: 95 and a VL sequence comprising a CDR-L1 sequence comprising SEQ ID NO: 118, a CDR-L2 sequence comprising SEQ ID NO: 135, and a CDR-L3 sequence SEQ ID NO: 152. In some aspects, the antibody comprises a V_{H} sequence comprising a Kabat CDR-H1 sequence comprising SEQ ID NO: 40, a Kabat CDR-H2 sequence comprising SEQ ID NO: 76, and a Kabat CDR-H3 sequence comprising SEQ ID NO: 96 and a VL sequence comprising a CDR-L1 sequence comprising SEQ ID NO: 118, a CDR-L2 sequence comprising SEQ ID NO: 136, and a CDR-L3 sequence SEQ ID NO: 152. In some aspects, the antibody comprises a V_{H} sequence comprising a Kabat CDR-H1 sequence comprising SEQ ID NO: 39, a Kabat CDR-H2 sequence comprising SEQ ID NO: 76, and a Kabat CDR-H3 sequence comprising SEQ ID NO: 94 and a VL sequence comprising a CDR-L1 sequence comprising SEQ ID NO: 118, a CDR-L2 sequence comprising SEQ ID NO: 135, and a CDR-L3 sequence SEQ ID NO: 152. In some aspects, the antibody comprises a V_{H} sequence comprising a Kabat CDR-H1 sequence comprising SEQ ID NO: 38, a Kabat CDR-H2 sequence comprising SEQ ID NO: 69, and a Kabat CDR-H3 sequence comprising SEQ ID NO: 97 and a VL sequence comprising a CDR-L1 sequence comprising SEQ ID NO: 115, a CDR-L2 sequence comprising SEQ ID NO: 131, and a CDR-L3 sequence SEQ ID NO: 153. In some aspects, the antibody comprises a V_{H} sequence comprising a Kabat CDR-H1 sequence comprising SEQ ID NO: 41, a Kabat CDR-H2 sequence comprising SEQ ID NO: 69, and a Kabat CDR-H3 sequence comprising SEQ ID NO: 98 and a VL sequence comprising a CDR-L1 sequence comprising SEQ ID NO: 119, a CDR-L2 sequence comprising SEQ ID NO: 131, and a CDR-L3 sequence SEQ ID NO: 154. In some aspects, the antibody comprises a V_{H} sequence comprising a Kabat CDR-H1 sequence comprising SEQ ID NO: 41, a Kabat CDR-H2 sequence comprising SEQ ID NO: 69, and a Kabat CDR-H3 sequence comprising SEQ ID NO: 99 and a V_{L} sequence comprising a CDR-L1 sequence comprising SEQ ID NO: 120, a CDR-L2 sequence comprising SEQ ID NO: 137, and a CDR-L3 sequence SEQ ID NO: 155. In some aspects, the antibody comprises a V_{H} sequence comprising a Kabat CDR-H1 sequence comprising SEQ ID NO: 41, a Kabat CDR-H2 sequence comprising SEQ ID NO: 69, and a Kabat CDR-H3 sequence comprising SEQ ID NO: 100 and a VL sequence comprising a CDR-L1 sequence comprising SEQ ID NO: 121, a CDR-L2 sequence comprising SEQ ID NO: 138, and a CDR-L3 sequence SEQ ID NO: 156. In some aspects, the antibody comprises a V_{H} sequence comprising a Kabat CDR-H1 sequence comprising SEQ ID NO: 38, a Kabat CDR-H2 sequence comprising SEQ ID NO: 75, and a Kabat CDR-H3 sequence comprising SEQ ID NO: 101 and a VL sequence comprising a CDR-L1 sequence comprising SEQ ID NO: 121, a CDR-1.2 sequence comprising SEQ ID NO: 138, and a CDR-L3 sequence SEQ ID NO: 157. In some aspects, the antibody comprises a VL sequence comprising a Kabat CDR-H1 sequence comprising SEQ ID NO: 42, a Kabat CDR-H2 sequence comprising SEQ ID NO: 78, and a Kabat CDR-H3 sequence comprising SEQ ID NO: 102 and a VL sequence comprising a CDR-L1 sequence comprising SEQ ID NO: 122, a CDR-L2 sequence comprising SEQ ID NO: 138, and a CDR-L3 sequence SEQ ID NO: 158. In some aspects, the antibody comprises a V_{H} sequence comprising a Kabat CDR-H1 sequence comprising SEQ ID NO: 38, a Kabat CDR-H2 sequence comprising SEQ ID NO: 75, and a Kabat CDR-H3 sequence comprising SEQ ID NO: 103 and a VL sequence comprising a CDR-L1 sequence comprising SEQ ID NO: 121, a CDR-H2 sequence comprising SEQ ID NO: 138, and a CDR-13 sequence SEQ ID NO: 159. In some aspects, the antibody comprises a V_{H} sequence comprising a Kabat CDR-H1 sequence comprising SEQ ID NO: 43, a Kabat CDR-H2 sequence comprising SEQ ID NO: 79, and a Kabat CDR-H3 sequence comprising SEQ ID NO: 104 and a VL sequence comprising a CDR-L1 sequence comprising SEQ ID NO: 118, a CDR-L2 sequence comprising SEQ ID NO: 135, and a CDR-L3 sequence SEQ ID NO: 160. In some aspects, the antibody comprises a V_{H} sequence comprising a Kabat CDR-H1 sequence comprising SEQ ID NO: 38, a Kabat CDR-H2 sequence comprising SEQ ID NO: 75, and a Kabat CDR-H3 sequence comprising SEQ ID NO: 103 and a VL sequence comprising a CDR-L1 sequence comprising SEQ ID NO: 121, a CDR-L2 sequence comprising SEQ ID NO: 139, and a CDR-L3 sequence SEQ ID NO: 161. In some aspects, the antibody comprises a V_{H} sequence comprising a Kabat CDR-H1 sequence comprising SEQ ID NO: 38, a Kabat CDR-H2 sequence comprising SEQ ID NO: 69, and a Kabat CDR-H3 sequence comprising SEQ ID NO: 105 and a VL sequence comprising a CDR-L1 sequence comprising SEQ ID NO: 123, a CDR-L2 sequence comprising SEQ ID NO: 140, and a CDR-L3 sequence SEQ ID NO: 162. In some aspects, the antibody comprises a V_{H} sequence comprising a Kabat CDR-H1 sequence comprising SEQ ID NO: 38, a Kabat CDR-H2 sequence comprising SEQ ID NO: 69, and a Kabat CDR-H3 sequence comprising SEQ ID NO: 106 and a VL sequence comprising a CDR-L1 sequence comprising SEQ ID NO: 121, a CDR-L2 sequence comprising SEQ ID NO: 138, and a CDR-L3 sequence SEQ ID NO: 163. In some aspects, the antibody comprises a V_{H} sequence comprising a Kabat CDR-H1 sequence comprising SEQ ID NO: 38, a Kabat CDR-H2 sequence comprising SEQ ID NO: 69, and a Kabat CDR-H3 sequence comprising SEQ ID NO: 107 and a VL sequence comprising a CDR-L1 sequence comprising SEQ ID NO: 121, a CDR-L2 sequence comprising SEQ ID NO: 139, and a CDR-L3 sequence SEQ ID NO: 164. In some aspects, the antibody comprises a V_{H} sequence comprising a Kabat CDR-H1 sequence comprising SEQ ID NO: 44, a Kabat CDR-H2 sequence comprising SEQ ID NO: 80, and a Kabat CDR-H3 sequence comprising SEQ ID NO: 108 and a VL sequence comprising a CDR-L1 sequence comprising SEQ ID NO: 120, a CDR-L2 sequence comprising SEQ ID NO: 137, and a CDR-L3 sequence SEQ ID NO: 165. In some aspects, the antibody comprises a V_{H} sequence comprising a Kabat CDR-H1 sequence comprising SEQ ID NO: 45, a Kabat CDR-H2 sequence comprising SEQ ID NO: 81, and a Kabat CDR-H3 sequence comprising SEQ ID NO: 109 and a VL sequence comprising a CDR-L1 sequence comprising SEQ ID NO: 124, a CDR-L2 sequence comprising SEQ ID NO: 125, and a CDR-L3 sequence SEQ ID NO: 166.

In some aspects, the antibody comprises a V_{H} sequence comprising a Chothia CDR-H1 sequence comprising SEQ ID NO: 1, a Chothia CDR-H2 sequence comprising SEQ ID NO: 46, and a Chothia CDR-H3 sequence comprising SEQ ID NO): 82 and a VL sequence comprising a CDR-L1 sequence comprising SEQ ID NO: 110, a CDR-L2 sequence comprising SEQ ID NO: 125, and a CDR-L3 sequence SEQ ID NO: 141. In some aspects, the antibody comprises a V_{H} sequence comprising a Chothia CDR-H1 sequence comprising SEQ ID NO: 2, a Chothia CDR-H2 sequence comprising SEQ ID NO: 47, and a Chothia CDR-H3 sequence comprising SEQ ID NO: 83 and a VL sequence comprising a CDR-L1 sequence comprising SEQ ID NO: 111, a CDR-L2 sequence comprising SEQ ID NO: 126, and a CDR-L3 sequence SEQ ID NO: 142. In some aspects, the antibody comprises a V_{H} sequence comprising a Chothia CDR-H1 sequence comprising SEQ ID NO: 1, a Chothia CDR-H2 sequence comprising SEQ ID NO: 46, and a Chothia CDR-H3 sequence comprising SEQ ID NO: 84 and a VL sequence comprising a CDR-L1 sequence comprising SEQ ID NO: 110, a CDR-L2 sequence comprising SEQ ID NO: 125, and a CDR-L3 sequence SEQ ID NO: 141. In some aspects, the antibody comprises a V_{H} sequence comprising a Chothia CDR-H1 sequence comprising SEQ ID NO: 2, a Chothia CDR-H2 sequence comprising SEQ ID NO: 47, and a Chothia CDR-H3 sequence comprising SEQ ID NO: 85 and a VL sequence comprising a CDR-L1 sequence comprising SEQ ID NO: 110, a CDR-L2 sequence comprising SEQ ID NO: 125, and a CDR-L3 sequence SEQ ID NO: 141. In some aspects, the antibody comprises a V_{H} sequence comprising a Chothia CDR-H1 sequence comprising SEQ ID NO: 3, a Chothia CDR-H2 sequence comprising SEQ ID NO: 47, and a Chothia CDR-H3 sequence comprising SEQ ID NO: 86 and a VL sequence comprising a CDR-L1 sequence comprising SEQ ID NO: 110, a CDR-L2 sequence comprising SEQ ID NO: 127, and a CDR-L3 sequence SEQ ID NO: 142. In some aspects, the antibody comprises a V_{H} sequence comprising a Chothia CDR-H1 sequence comprising SEQ ID NO: 4, a Chothia CDR-H2 sequence comprising SEQ ID NO: 46, and a Chothia CDR-H3 sequence comprising SEQ ID NO: 82 and a VL sequence comprising a CDR-L1 sequence comprising SEQ ID NO: 110, a CDR-L2 sequence comprising SEQ ID NO: 125, and a CDR-L3 sequence SEQ ID NO: 141. In some aspects, the antibody comprises a V_{H} sequence comprising a Chothia CDR-H1 sequence comprising SEQ ID NO: 2, a Chothia CDR-H2 sequence comprising SEQ ID NO: 46, and a Chothia CDR-H3 sequence comprising SEQ ID NO: 82 and a VL sequence comprising a CDR-L1 sequence comprising SEQ ID NO: 110, a CDR-L2 sequence comprising SEQ ID NO: 125, and a CDR-L3 sequence SEQ ID NO: 143. In some aspects, the antibody comprises a V_{H} sequence comprising a Chothia CDR-H1 sequence comprising SEQ ID NO: 2, a Chothia CDR-H2 sequence comprising SEQ ID NO: 46, and a Chothia CDR-H3 sequence comprising SEQ ID NO: 82 and a VL sequence comprising a CDR-L1 sequence comprising SEQ ID NO: 112, a CDR-L2 sequence comprising SEQ ID NO: 128, and a CDR-L3 sequence SEQ ID NO: 144. In some aspects, the antibody comprises a V_{H} sequence comprising a Chothia CDR-H1 sequence comprising SEQ ID NO: 1, a Chothia CDR-H2 sequence comprising SEQ ID NO: 46, and a Chothia CDR-H3 sequence comprising SEQ ID NO: 82 and a VL sequence comprising a CDR-L1 sequence comprising SEQ ID NO: 111, a CDR-L2 sequence comprising SEQ ID NO: 126, and a CDR-13 sequence SEQ ID NO: 145. In some aspects, the antibody comprises a V_{H} sequence comprising a Chothia CDR-H1 sequence comprising SEQ ID NO: 5, a Chothia CDR-H2 sequence comprising SEQ ID NO: 49, and a Chothia CDR-H3 sequence comprising SEQ ID NO: 82 and a VL sequence comprising a CDR-L1 sequence comprising SEQ ID NO: 113, a CDR-1.2 sequence comprising SEQ ID NO: 129, and a CDR-L3 sequence SEQ ID NO: 146. In some aspects, the antibody comprises a V_{H} sequence comprising a Chothia CDR-H1 sequence comprising SEQ ID NO: 6, a Chothia CDR-H2 sequence comprising SEQ ID NO: 50, and a Chothia CDR-H3 sequence comprising SEQ ID NO: 82 and a VL sequence comprising a CDR-L1 sequence comprising SEQ ID NO: 114, a CDR-H2 sequence comprising SEQ ID NO: 130, and a CDR-L3 sequence SEQ ID NO: 147. In some aspects, the antibody comprises a V_{H} sequence comprising a Chothia CDR-H1 sequence comprising SEQ ID NO: 2, a Chothia CDR-H2 sequence comprising SEQ ID NO: 47, and a Chothia CDR-H3 sequence comprising SEQ ID NO: 82 and a VL sequence comprising a CDR-L1 sequence comprising SEQ ID NO: 110, a CDR-L2 sequence comprising SEQ ID NO: 125, and a CDR-L3 sequence SEQ ID NO: 141. In some aspects, the antibody comprises a V_{H} sequence comprising a Chothia CDR-H1 sequence comprising SEQ ID NO: 7, a Chothia CDR-H2 sequence comprising SEQ ID NO: 51, and a Chothia CDR-H3 sequence comprising SEQ ID NO: 87 and a VL sequence comprising a CDR-L1 sequence comprising SEQ ID NO: 115, a CDR-L2 sequence comprising SEQ ID NO: 131, and a CDR-L3 sequence SEQ ID NO: 148. In some aspects, the antibody comprises a V_{H} sequence comprising a Chothia CDR-H1 sequence comprising SEQ ID NO: 8, a Chothia CDR-H2 sequence comprising SEQ ID NO: 52, and a Chothia CDR-H3 sequence comprising SEQ ID NO: 88 and a VL sequence comprising a CDR-L1 sequence comprising SEQ ID NO: 116, a CDR-L2 sequence comprising SEQ ID NO: 131, and a CDR-L3 sequence SEQ ID NO: 149. In some aspects, the antibody comprises a V_{H} sequence comprising a Chothia CDR-H1 sequence comprising SEQ ID NO: 9, a Chothia CDR-112 sequence comprising SEQ ID NO: 53, and a Chothia CDR-13 sequence comprising SEQ ID NO: 89 and a VL sequence comprising a CDR-L1 sequence comprising SEQ ID NO: 115, a CDR-L2 sequence comprising SEQ ID NO: 131, and a CDR-L3 sequence SEQ ID NO: 148. In some aspects, the antibody comprises a V_{H} sequence comprising a Chothia CDR-H1 sequence comprising SEQ ID NO: 10, a Chothia CDR-H2 sequence comprising SEQ ID NO: 52, and a Chothia CDR-H3 sequence comprising SEQ ID NO: 90 and a VL sequence comprising a CDR-L1 sequence comprising SEQ ID NO: 115, a CDR-L2 sequence comprising SEQ ID NO: 132, and a CDR-L3 sequence SEQ ID NO: 149. In some aspects, the antibody comprises a V_{H} sequence comprising a Chothia CDR-H1 sequence comprising SEQ ID NO: 11, a Chothia CDR-H2 sequence comprising SEQ ID NO: 54, and a Chothia CDR-H3 sequence comprising SEQ ID NO: 91 and a VL sequence comprising a CDR-L1 sequence comprising SEQ ID NO: 115, a CDR-L2 sequence comprising SEQ ID NO: 131, and a CDR-L3 sequence SEQ ID NO: 148. In some aspects, the antibody comprises a V_{H} sequence comprising a Chothia CDR-H1 sequence comprising SEQ ID NO: 12, a Chothia CDR-H2 sequence comprising SEQ ID NO: 53, and a Chothia CDR-H3 sequence comprising SEQ ID NO: 92 and a VL sequence comprising a CDR-L1 sequence comprising SEQ ID NO: 115, a CDR-L2 sequence comprising SEQ ID NO: 131, and a CDR-L3 sequence SEQ ID NO: 148. In some aspects, the antibody comprises a V_{H} sequence comprising a Chothia CDR-H1 sequence comprising SEQ ID NO: 13, a Chothia CDR-H2 sequence comprising SEQ ID NO: 52, and a Chothia CDR-H3 sequence comprising SEQ ID NO: 93 and a VL sequence comprising a CDR-L1 sequence comprising SEQ ID NO: 117, a CDR-L2 sequence comprising SEQ ID NO: 133, and a CDR-L3 sequence SEQ ID NO: 150. In some aspects, the antibody comprises a Vu sequence comprising a Chothia CDR-H1 sequence comprising SEQ ID NO: 10, a Chothia CDR-H2 sequence comprising SEQ ID NO: 52, and a Chothia CDR-H3 sequence comprising SEQ ID NO: 92 and a VL sequence comprising a CDR-1.1 sequence comprising SEQ ID NO: 115, a CDR-L2 sequence comprising SEQ ID NO: 134, and a CDR-L3 sequence SEQ ID NO: 148. In some aspects, the antibody comprises a V_{H} sequence comprising a Chothia CDR-H1 sequence comprising SEQ ID NO: 14, a Chothia CDR-H2 sequence comprising SEQ ID NO: 54, and a Chothia CDR-H3 sequence comprising SEQ ID NO: 87 and a VL sequence comprising a CDR-L1 sequence comprising SEQ ID NO: 115, a CDR-H2 sequence comprising SEQ ID NO: 131, and a CDR-L3 sequence SEQ ID NO: 148. In some aspects, the antibody comprises a V_{H} sequence comprising a Chothia CDR-H1 sequence comprising SEQ ID NO: 15, a Chothia CDR-H2 sequence comprising SEQ ID NO: 55, and a Chothia CDR-H3 sequence comprising SEQ ID NO: 87 and a VL sequence comprising a CDR-L1 sequence comprising SEQ ID NO: 115, a CDR-L2 sequence comprising SEQ ID NO: 131, and a CDR-L3 sequence SEQ ID NO: 148. In some aspects, the antibody comprises a V_{H} sequence comprising a Chothia CDR-H1 sequence comprising SEQ ID NO: 16, a Chothia CDR-H2 sequence comprising SEQ ID NO: 56, and a Chothia CDR-H3 sequence comprising SEQ ID NO: 87 and a VL sequence comprising a CDR-L1 sequence comprising SEQ ID NO: 115, a CDR-L2 sequence comprising SEQ ID NO: 131, and a CDR-L3 sequence SEQ ID NO: 148. In some aspects, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 94 and a VL sequence comprising a CDR-L1 sequence comprising SEQ ID NO: 118, a CDR-L2 sequence comprising SEQ ID NO: 135, and a CDR-L3 sequence SEQ ID NO: 151. In some aspects, the antibody comprises a V_{H} sequence comprising a Chothia CDR-H1 sequence comprising SEQ ID NO: 18, a Chothia CDR-H2 sequence comprising SEQ ID NO: 57, and a Chothia CDR-H3 sequence comprising SEQ ID NO: 95 and a VL sequence comprising a CDR-L1 sequence comprising SEQ ID NO: 118, a CDR-L2 sequence comprising SEQ ID NO: 135 and a CDR-L3 sequence SEQ ID NO: 152. In some aspects, the antibody comprises a V_{H} sequence comprising a Chothia CDR-H1 sequence comprising SEQ ID NO: 19, a Chothia CDR-H2 sequence comprising SEQ ID NO: 57, and a Chothia CDR-H3 sequence comprising SEQ ID NO: 96 and a VL sequence comprising a CDR-L1 sequence comprising SEQ ID NO: 118, a CDR-L2 sequence comprising SEQ ID NO: 136, and a CDR-L3 sequence SEQ ID NO: 152. In some aspects, the antibody comprises a V_{H} sequence comprising a Chothia CDR-H1 sequence comprising SEQ ID NO: 21, a Chothia CDR-H2 sequence comprising SEQ ID NO: 57, and a Chothia CDR-H3 sequence comprising SEQ ID NO: 94 and a VL sequence comprising a CDR-L1 sequence comprising SEQ ID NO: 118, a CDR-H2 sequence comprising SEQ ID NO: 135, and a CDR-13 sequence SEQ ID NO: 152. In some aspects, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 97 and a VL sequence comprising a CDR-L1 sequence comprising SEQ ID NO: 115, a CDR-L2 sequence comprising SEQ ID NO: 131, and a CDR-13 sequence SEQ ID NO: 153. In some aspects, the antibody comprises a V_{H} sequence comprising a Chothia CDR-H1 sequence comprising SEQ ID NO: 22, a Chothia CDR-H2 sequence comprising SEQ ID NO: 52, and a Chothia CDR-H3 sequence comprising SEQ ID NO: 98 and a VL sequence comprising a CDR-L1 sequence comprising SEQ ID NO: 119, a CDR-H2 sequence comprising SEQ ID NO: 131, and a CDR-L3 sequence SEQ ID NO: 154. In some aspects, the antibody comprises a Vu sequence comprising a Chothia CDR-H1 sequence comprising SEQ ID NO: 22, a Chothia CDR-H2 sequence comprising SEQ ID NO: 51, and a Chothia CDR-H3 sequence comprising SEQ ID NO: 99 and a VL sequence comprising a CDR-L1 sequence comprising SEQ ID NO: 120, a CDR-L2 sequence comprising SEQ ID NO: 137, and a CDR-L3 sequence SEQ ID NO: 155. In some aspects, the antibody comprises a V_{H} sequence comprising a Chothia CDR-H1 sequence comprising SEQ ID NO: 22, a Chothia CDR-H2 sequence comprising SEQ ID NO: 51, and a Chothia CDR-H3 sequence comprising SEQ ID NO: 100 and a VL sequence comprising a CDR-L1 sequence comprising SEQ ID NO: 121, a CDR-L2 sequence comprising SEQ ID NO: 138, and a CDR-L3 sequence SEQ ID NO: 156. In some aspects, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 101 and a VL sequence comprising a CDR-L1 sequence comprising SEQ ID NO: 121, a CDR-L2 sequence comprising SEQ ID NO: 138, and a CDR-L3 sequence SEQ ID NO: 157. In some aspects, the antibody comprises a V_{H} sequence comprising a Chothia CDR-H1 sequence comprising SEQ ID NO: 17, a Chothia CDR-H2 sequence comprising SEQ ID NO: 59, and a Chothia CDR-H3 sequence comprising SEQ ID NO: 102 and a VL sequence comprising a CDR-L1 sequence comprising SEQ ID NO: 122, a CDR-L2 sequence comprising SEQ ID NO: 138, and a CDR-L3 sequence SEQ ID NO: 158. In some aspects, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 103 and a VL sequence comprising a CDR-L1 sequence comprising SEQ ID NO: 121, a CDR-L2 sequence comprising SEQ ID NO: 138, and a CDR-L3 sequence SEQ ID NO: 159. In some aspects, the antibody comprises a V_{H} sequence comprising a Chothia CDR-H1 sequence comprising SEQ ID NO: 23, a Chothia CDR-H2 sequence comprising SEQ ID NO: 60, and a Chothia CDR-H3 sequence comprising SEQ ID NO: 104 and a VL sequence comprising a CDR-L1 sequence comprising SEQ ID NO: 118, a CDR-L2 sequence comprising SEQ ID NO: 135, and a CDR-L3 sequence SEQ ID NO: 160. In some aspects, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 103 and a VL sequence comprising a CDR-L1 sequence comprising SEQ ID NO: 121, a CDR-L2 sequence comprising SEQ ID NO: 139, and a CDR-L3 sequence SEQ ID NO: 161. In some aspects, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 105 and a VL sequence comprising a CDR-L1 sequence comprising SEQ ID NO: 123, a CDR-H2 sequence comprising SEQ ID NO: 140, and a CDR-13 sequence SEQ ID NO: 162. In some aspects, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 106 and a VL sequence comprising a CDR-L1 sequence comprising SEQ ID NO: 121, a CDR-H2 sequence comprising SEQ ID NO: 138, and a CDR-L3 sequence SEQ ID NO: 163. In some aspects, the antibody comprises a V_{H} sequence comprising a Chothia CDR-H1 sequence comprising SEQ ID NO: 17, a Chothia CDR-H2 sequence comprising SEQ ID NO: 51, and a Chothia CDR-H3 sequence comprising SEQ ID NO: 107 and a VL sequence comprising a CDR-L1 sequence comprising SEQ ID NO: 121, a CDR-L2 sequence comprising SEQ ID NO: 139, and a CDR-L3 sequence SEQ ID NO: 164. In some aspects, the antibody comprises a V_{H} sequence comprising a Chothia CDR-H1 sequence comprising SEQ ID NO: 17, a Chothia CDR-H2 sequence comprising SEQ ID NO: 61, and a Chothia CDR-H3 sequence comprising SEQ ID NO: 108 and a VL sequence comprising a CDR-L1 sequence comprising SEQ ID NO: 120, a CDR-L2 sequence comprising SEQ ID NO: 137, and a CDR-L3 sequence SEQ ID NO: 165. In some aspects, the antibody comprises a V_{H} sequence comprising a Chothia CDR-H1 sequence comprising SEQ ID NO: 17, a Chothia CDR-H2 sequence comprising SEQ ID NO: 62, and a Chothia CDR-H3 sequence comprising SEQ ID NO: 109 and a VL sequence comprising a CDR-L1 sequence comprising SEQ ID NO: 124, a CDR-L2 sequence comprising SEQ ID NO: 125, and a CDR-L3 sequence SEQ ID NO: 166.

### 2.7.3.1. Variants of V_{H}-V_{L} Pairs

In some embodiments, the V_{H} V_{L} pairs provided herein comprise a variant of an illustrative V_{H} and/or V_{L} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.7.4 HC+LC

In some embodiments, the antibody comprises or consists of one or more heavy chains consisting of an HC sequence and one or more light chains consisting of an LC sequence. In some embodiments, the antibody comprises or consists of two identical heavy chains consisting of an HC sequence and two identical light chains consisting of an LC sequence.

In some embodiments, the HC sequence is an HC sequence comprising, consisting of, or consisting essentially of SEQ ID NO: 255, SEQ ID NO: 257, SEQ ID NO: 259, SEQ ID NO: 261, SEQ ID NO: 263, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289. SEQ ID NO: 291. SEQ ID NO: 293. SEQ ID NO: 295, or SEQ ID NO: 297 and the LC sequence is a LC sequence comprising, consisting of, or consisting essentially of SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276. SEQ ID NO: 278. SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, or SEQ ID NO: 298. In some embodiments, the HC sequence is an HC sequence consisting of SEQ ID NO: 255, SEQ ID NO: 257, SEQ ID NO: 259, SEQ ID NO: 261, SEQ ID NO: 263, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, or SEQ ID NO: 297 and the LC sequence is an LC sequence consisting of SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, or SEQ ID NO: 298.

In some embodiments, the HC sequence is an HC sequence consisting of SEQ ID NO: 255 and the LC sequence is an LC sequence consisting of SEQ ID NO: 256. In some embodiments, the HC sequence is an HC sequence consisting of SEQ ID NO: 257 and the LC sequence is an LC sequence consisting of SEQ ID NO: 258. In some embodiments, the HC sequence is an HIC sequence consisting of SEQ ID NO: 259 and the LC sequence is an LC sequence consisting of SEQ ID NO: 260. In some embodiments, the HC sequence is an HC sequence consisting of SEQ ID NO: 261 and the LC sequence is an LC sequence consisting of SEQ ID NO: 262. In some embodiments, the HC sequence is an HC sequence consisting of SEQ ID NO: 263 and the LC sequence is an LC sequence consisting of SEQ ID NO: 264. In some embodiments, the HC sequence is an HC sequence consisting of SEQ ID NO: 265 and the LC sequence is an LC sequence consisting of SEQ ID NO: 266. In some embodiments, the HC sequence is an HC sequence consisting of SEQ ID NO: 267 and the LC sequence is an LC sequence consisting of SEQ ID NO: 268. In some embodiments, the HC sequence is an HC sequence consisting of SEQ ID NO: 269 and the LC sequence is an LC sequence consisting of SEQ ID NO: 270. In some embodiments, the HC sequence is an HC sequence consisting of SEQ ID NO: 271 and the LC sequence is an LC sequence consisting of SEQ ID NO: 272. In some embodiments, the HC sequence is an HC sequence consisting of SEQ ID NO: 273 and the LC sequence is an LC sequence consisting of SEQ ID NO: 274. In some embodiments, the HC sequence is an HC sequence consisting of SEQ ID NO: 275 and the LC sequence is an LC sequence consisting of SEQ ID NO: 276. In some embodiments, the HC sequence is an HC sequence consisting of SEQ ID NO: 277 and the LC sequence is an LC sequence consisting of SEQ ID NO: 278. In some embodiments, the HC sequence is an HC sequence consisting of SEQ ID NO: 279 and the LC sequence is an LC sequence consisting of SEQ ID NO: 280. In some embodiments, the HC sequence is an HC sequence consisting of SEQ ID NO: 281 and the LC sequence is an LC sequence consisting of SEQ ID NO: 282. In some embodiments, the HC sequence is an HC sequence consisting of SEQ ID NO: 283 and the LC sequence is an LC sequence consisting of SEQ ID NO: 284. In some embodiments, the HC sequence is an HC sequence consisting of SEQ ID NO: 285 and the LC sequence is an LC sequence consisting of SEQ ID NO: 286. In some embodiments, the HC sequence is an HC sequence consisting of SEQ ID NO: 287 and the LC sequence is an LC sequence consisting of SEQ ID NO: 288. In some embodiments, the HC sequence is an HC sequence consisting of SEQ ID NO: 289 and the LC sequence is an LC sequence consisting of SEQ ID NO: 290. In some embodiments, the HIC sequence is an HC sequence consisting of SEQ ID NO: 291 and the LC sequence is an LC sequence consisting of SEQ ID NO: 292. In some embodiments, the HC sequence is an HC sequence consisting of SEQ ID NO: 293 and the LC sequence is an LC sequence consisting of SEQ ID NO: 294. In some embodiments, the HC sequence is an HC sequence consisting of SEQ ID NO: 295 and the LC sequence is an LC sequence consisting of SEQ ID NO: 296. In some embodiments, the HC sequence is an HC sequence consisting of SEQ ID NO: 297 and the LC sequence is an LC sequence consisting of SEQ ID NO: 298.

### 2.8. Consensus Sequences

In some embodiments, provided herein are anti-CD39 antibodies comprising one or more sequences defined by consensus sequences. Each consensus sequence is based, at least in part, on one or more alignments of two or more useful anti-CD39 CDR sequences provided in this disclosure. Based on such alignments, a person of skill in the art would recognize that different amino acid residues may useful in certain positions of the CDRs. Accordingly, each consensus sequence encompasses two or more useful anti-CD39 CDR sequences.

### 2.8.1. CDR-H3 Consensus Sequences

In some embodiments, the antibody comprises a CDR-H3 sequence defined by the consensus sequence G-K-R-E-G-G-T-E-Y-L-R-γ₁₂ (SEQ ID NOS: 82-86), where γ₁₂ is H, K, S, N, or V.

In some aspects, γ₁₂ is H. In some aspects, γ₁₂ is K. In some aspects, γ₁₂ is S. In some aspects, γ₁₂ is N. In some aspects, γ₁₂ is V.

In some embodiments, the antibody comprises a CDR-H3 sequence defined by the consensus sequence E-S-G-Φ₄-Y-R-D-H-R-L-Φ₁₁-V (SEQ ID NOS: 94-96), where Φ₄ is G or T and Øn is D or G.

In some aspects, Φ₄ is G when Φ₁₁ is D or G. In some aspects, Φ₄ is D when Φ₄ is G or T.

In some aspects, Φ₄ is G and Φ₁₁ is D. In some aspects, Φ₄ is T and Φ₁₁ is D. In some aspects, Φ₄ is G and Φ₁₁ is G.

In some embodiments, the antibody comprises a CDR-H3 sequence defined by the consensus sequence G-G-A-K-Y-A-∃₇-∃₈-∃₉-G-M-D-V (SEQ ID NOS: 87-93), where ∃₇ is S, V, G, or R; ∃₈ is T, Q, K, G, or R; and ∃₉ is Y, H, L, or W.

In some aspects, ∃₇ is S when ∃₈ is T, Q, or K and ∃₉ is Y, H, L, or W. In some aspects, ∃₈ is T when ∃₇ is S or R and ∃₉ is Y or H. In some aspects, ∃₉ is Y when ∃₇ is S, V, G, or R and ∃₈ is T, G, or R.

In some aspects, ∃₇ is S when ∃₈ is T and ∃₉ is Y. In some aspects, ∃₇ is S when ∃₈ is T and ∃₉ is H. In some aspects, ∃₇ is S when ∃₈ is Q and ∃₉ is L. In some aspects, ∃₇ is S when ∃₈ is K and ∃₉ is W. In some aspects, ∃₇ is V when ∃₈ is G and ∃₉ is Y. In some aspects, ∃₇ is G when ∃₈ is R and ∃₉ is Y. In some aspects, ∃₇ is R when ∃₈ is T and ∃₉ is Y.

### 2.8.2. Chothia CDR-H2 Consensus Sequences

In some embodiments, the antibody comprises a Chothia CDR-H2 sequence defined by the consensus sequence N-P-ε₅-ε₆-G-S-T (SEQ ID NOS: 46-48), where ε₅ is L, R, or S and ε₆ is G or V.

In some aspects, when ε₅ is S, ε₆ is G or V. In some aspects, when ε₆ is G, ε₅ is S, L, or R.

In some aspects, when ε₅ is L, ε₆ is G. In some aspects, when ε₅ is S, ∃₆ is G. In some aspects, when ε₅ is S, ε₆ is V. In some aspects, when ε₅ is R, ε₆ is G.

In some embodiments, the antibody comprises a Chothia CDR-H2 sequence defined by the consensus sequence α₃-α₄-α₅-α₆-G-T-A (SEQ ID NOS: 51-54), where α₃ is I or L or is absent; α₄ is P or is absent; and α₅ is 1, G, or R; and α₆ is A, F, or G.

In some aspects, when α₃, is I, α₄ is P; α₅ is I or R; and α₆ is F or G. In some aspects, when α₃ is L, α₄ is P; α₅ is I; and α₆ is A or G. In some aspects, when α₄, is P, α₃ is I or L; α₅ is I or R; and α₆ is A, F, or G. In some aspects, when α₅, is I, α₃ is I or L; α₄ is P; and α₆ is A, F, or G. In some aspects, when α₆ is F, α₃ is I or is absent; α₄ is P or is absent; and α₅ is I or G. In some aspects, when α₆, is G, α₃ is I or L; α₄ is P; and α₅ is I or R.

In some aspects, when α₃ is L, α₄ is P; α₅ is I; and α₆ is A. In some aspects, when α₃ is I, α₄ is P; α₅ is I; and α₆ is F. In some aspects, when α₃ is absent, α₄ is absent; α₅ is G; and α₆ is F. In some aspects, when α₃ is L, α₄ is P; α₅ is I; and α₆ is G. In some aspects, when α₃ is I, α₄ is P; α₅ is R; and α₆ is G.

In some embodiments, the antibody comprises a Chothia CDR-H2 sequence defined by the consensus sequence I-P-β₅-β₆-G-β₈-A (SEQ ID NOS: 56-60), where β₅ is I, E, S, or T; β₆ is F, I, or S; and β₈ is I or T.

In some aspects, when β₅ is I, β₆ is F or S and β₈ is T. In some aspects, when β6 is F, β₅ is E, I, or T and β₈ is I or T. In some aspects, when β₈ is T, β₅ is I, S, or T and β₆ is F, I, or S.

In some aspects, when β₅ is I, β₆ is F and β₈ is T. In some aspects, when β₅ is E, β₆ is F and β₈ is 1. In some aspects, when β₅ is S, β₆ is 1 and β₈ is T. In some aspects, when β5 is I, β₆ is S and β₈ is T. In some aspects, when β₅ is T, β₆ is S and β₈ is T.

### 2.8.3. Chothia CDR-H1 Consensus Sequences

In some embodiments, the antibody comprises a Chothia CDR-H1 sequence defined by the consensus sequence G-Y-T-F-Ω₅-S-Y (SEQ ID NOS: 1-2 and 4-6), where Ω₅ is T, K, Q, F, or V.

In some aspects, Ω₅ is T. In some aspects, Ω₅ is K. In some aspects, Ω₅ is Q. In some aspects, Ω₅ is F. In some aspects, Ω₅ is V.

In some embodiments, the antibody comprises a Chothia CDR-H1 sequence defined by the consensus sequence G-G-T-F-ν₅-ν₆-Y (SEQ ID NOS: 17-22 and 24), where ν₅ is S, G, or E and ν₆ is S, K, R, or S.

In some aspects, ν₅ is S when ν₆ is S or K. In some aspects, ν₆ is S when ν₅ is S or E.

In some aspects, ν₅ is S when ν₆ is S. In some embodiments, ν₅ is S when ν₆ is K. In some aspects, ν₅ is G when ν₆ is R. In some aspects ν₅ is E when ν₆ is S.

In some embodiments, the antibody comprises a Chothia CDR-H1 sequence defined by the consensus sequence G-G-T-F-κ₅-κ₆-κ₇ (SEQ ID NOS: 7-16), where κ₅ is S, Q, P, or A; κ₆ is S, K, H, L, A, or W; and κ₇ is Y, L, T, N, or M.

In some aspects, when κ₅ is S, κ₆ is S, K, H, L, A, or W and κ₇ is Y, L, T, or M. In some aspects, when κ₆ is S, κ₅ is S, Q, P, or A and κ₇ is Y, L, or N. In some aspects, when κ₇ is L, κ₅ is S, Q, or A and κ₆ is S, K, L, or W.

In some aspects, when κ₅ is S, κ₆ is S and κ₇ is Y. In some aspects, when κ₅ is S, κ₆ is S and κ₇ is L. In some aspects, when κ₅ is S, κ₆ is K and κ₇ is L. In some aspects, when κ₅ is S, κ₆ is H and κ₇ is T. In some aspects, when κ₅ is S, κ₆ is L and κ₇ is L. In some aspects, when κ₅ is Q, κ₆ is S and κ₇ is L. In some aspects, when κ₅ is P, κ₆ is S and κ₇ is N. In some aspects, when κ₅ is S, κ₆ is A and κ₇ is M. In some aspects, when κ₅ is A, κ₆ is S and κ₇ is L. In some aspects, when κ₅ is S, κ₆ is W and Kris L.

### 2.8.4. Kabat CDR-H2 Consensus Sequences

In some embodiments, the antibody comprises a Kabat CDR-H2 sequence defined by the consensus sequence ε₁-I-N-P-ε₅-ε₆-G-S-T-ε₁₀-Y-A-Q-K-F-Q-G (SEQ ID NOS: 63-66 and 68), where ε₁ is K, S, R, or V; ε₅ is L, R, or S; ε₆ is G or V; and ε₁₀ is S or W.

In some aspects, when ε₁ is V; ε₅ is L or S; ε₆ is G; and ε₁₀ is S. In some aspects, when ε₁ is R; ε₅ is S; ε₆ is V or G; and ε₁₀ is W. In some aspects, when ε₅ is S; ε₁ is R or V; ε₆ is G or V; and ε₁₀ is S or W. In some aspects, when ε₆ is G; ε₁ is R or V; ε₅ is S; and ε₁₀ is S or W. In some aspects, when ε₁₀ is S; ε₁ is V or S; ε₅ is L, R, or S; and ε₆ is G. In some aspects, when ε₁₀ is W; ε₁ is K or R; ε₅ is S; and ε₆ is G or V.

In some aspects, when ε₁ is V; ε₅ is L; ε₆ is G; and ε₁₀ is S. In some aspects, when ε₁ is V; ε₅ is S; ε₆ is G; and ε₁₀ is S. In some aspects, when ε₁ is R; ε₅ is S; ε₆ is V; and ε₁₀ is W. In some aspects, when ε₁ is R; ε₅ is S; ε₆ is G; and ε₁₀ is W. In some aspects, when ε₁ is K; ε₅ is S; ε₆ is G; and ε₁₀ is W. In some aspects, when ε₁ is S; ε₅ is R; ε₆ is G; and ε₁₀ is S.

In some embodiments, the antibody comprises a Kabat CDR-H2 sequence defined by the consensus sequence G-I-α₃-α₄-α₅-α₆-G-T-A-N-Y-A-Q-K-F-Q-G (SEQ ID NOS: 69-72), where α₃ is I or L or is absent; α₄ is P or is absent; and α₅ is I, G, or R; and α₆ is A, F, or G.

In some aspects, when α₃, is I, α₄ is P; α₅ is I or R; and α₆ is F or G. In some aspects, when α₃ is L, α₄ is P; α₅ is I; and α₆ is A or G. In some aspects, when α₄, is P, α₃ is I or L; α₅ is I or R; and α₆ is A, F, or G. In some aspects, when α₅, is I, α₃ is I or L; α₄ is P; and α₆ is A, F, or G. In some aspects, when α₆, is F, α₃ is I or is absent; α₄ is P or is absent; and α₅ is I or G. In some aspects, when α₆, is G, α₃ is I or L; α₄ is P; and α₅ is I or R.

In some aspects, when α₃ is L, α₄ is P; α₅ is I; and α₆ is A. In some aspects, when α₃ is I, α₄ is P; α₅ is I; and α₆ is F. In some aspects, when α₃ is absent, α₄ is absent; α₅ is G; and α₆ is F. In some aspects, when α₃ is L, α₄ is P; α₅ is I; and α₆ is G. In some aspects, when α₃ is I, α₄ is P; α₅ is R; and α₆ is G.

In some embodiments, the antibody comprises a Kabat CDR-H2 sequence defined by the consensus sequence β₁-I-I-P-β₅-β₆-G-β₈-A-N-Y-A-Q-K-F-G-Q (SEQ ID NOS: 74 and 76-79) where β₁ is S or G; β₅ is I, E, S, or T; β₆ is F, I, or S; and β₈ is I or T.

In some aspects, when β₁ is S, β₅ is E, I, or S; β₆ is I or F; and β₈ is I or T. In some aspects, when β₁ is G, β₅ is I or T; β₆ is F or S; and β₈ is T. In some aspects, when β₅ is I, β₁ is G or S; β₆ is F or S; and β₈ is T. In some aspects, when β₆ is F, β₁ is G or S; β₅ is E, I, or T; and β₈ is I or T. In some aspects, when β₈ is T, β₁ is G or S; β₅ is I, S, or T; and β₆ is F, I, or S.

In some aspects, when β₁ is S, β₅ is 1; β₅ is F; and β₈ is T. In some aspects, when β₁ is S, β₅ is E; β₅ is F; and β₈ is I. In some aspects, when β₁ is S, β₅ is S; β₅ is I; and β₈ is T. In some aspects, when β₁ is G, β₅ is I; β₅ is F; and β₈ is T. In some aspects, when β₁ is G, β₅ is I; β₅ is S; and β₈ is T. In some aspects, when β₁ is G, β₅ is T; β₅ is F; and β₈ is T.

### 2.8.5. Kabat CDR-H1 Consensus Sequences

In some embodiments, the antibody comprises a Kabat CDR-H1 sequence defined by the consensus sequence S-Y-Δ₃-M-Δ₅ (SEQ ID NOS: 25-29 and 44-45), where Δ₃ is E, F, Q, or Y and Δ₅ is H or Y.

In some aspects, when Δ₃ is Y, Δ₅ is II or Y. In some aspects, when Δ₅ is II, Δ₃ is E, F, Q, or Y.

In some aspects, Δ₃ is Y when Δ₅ is II. In some aspects, Δ₃ is Y when Δ₅ is Y. In some aspects, Δ₃ is E when Δ₅ is H. In some aspects, Δ₃ is Q when Δ₅ is H. In some aspects, Δ₃ is F when Δ₅ is H.

In some embodiments, the antibody comprises a Kabat CDR-H1 sequence defined by the consensus sequence θ₁-θ₂-θ₃-I-S(SEQ ID NOS: 30-37), where θ₁ is A, H, K, L, S, or W; θ₂ is L, M, N, or T; and θ₃ is A or P.

In some aspects, when θ₁ is S, θ₂ is L or N and θ₃ is A or P. In some aspects, when θ₂ is L, θ₁ is K, L, S, or W and θ₃ is A or P. In some aspects, when θ₃ is A, θ₁ is A, H, K, L, S, or W and θ₂ is L, M, N, or T.

In some aspects, θ₁ is S, when θ₂ is L and θ₃ is A. In some aspects, θ₁ is K, when θ₂ is L and θ₃ is A. In some aspects, θ₁ is H when θ₂ is T and θ₃ is A. In some aspects, θ₁ is S when θ₂ is L and θ₃ is P. In some aspects, θ₁ is L when θ₂ is L and θ₃ is A. In some aspects, θ₁ is S when θ₂ is N and θ₃ is A. In some aspects, θ₁ is A when θ₂ is M and θ₃ is A. In some aspects, θ₁ is W when θ₂ is L and θ₃ is A.

In some embodiments, the antibody comprises a Kabat CDR-H1 sequence defined by the consensus sequence η₁-Y-η₃-I-S SEQ ID NOS: 38-41), where η₁ is S, K, N, or R and η₃ is A or G.

In some aspects, η₁ is S where η₃ is A or G. In some aspects, η₃ is A where η₁ is N or S. In some aspects, η₃ is G where η₁ is K, R, or S.

In some aspects, when η₁ is S, η₃ is A. In some aspects, when η₁ is S, η₃ is G. In some aspects, when η₁ is K, η₃ is G. In some aspects, when η₁ is R, η₃ is G. In some aspects, when η₁ is N, η₃ is A.

### 2.8.6. CDR-L3 Consensus Sequences

In some embodiments, the antibody comprises a CDR-L3 sequence defined by the consensus sequence Q-Q-Y-π₄-π₅-π₆-π₇-T (SEQ ID NOS: 141-147), where π₄ is G, H, or Y; π₅ is S, N, F, G, or R; π₆ is S, Y, A, G, or R; and π₇ is P, I, or L.

In some aspects, π₄ is H when π₅ is S, N, G, or R; π₆ is Y, A, G, or R; and π₇ is I or L. In some aspects, π₅ is S, when π₄ is G or H; π₅ is S, Y, or A; and π₇ is P, I or L. In some aspects, π₆ is Y, when π₄ is H or Y; π₅ is S or F; and π₇ is I. In some aspects, π₆ is A when π₄ is II; π₅ is N or S; and π₇ is I or L. In some aspects, π₇ is I when π₄ is II or Y; π₅ is S, N, F, G, or R; and π₆ is Y, A, G, or R.

In some aspects, π₄ is G when π₅ is S; π₆ is S; and π₇ is P. In some aspects, π₇ is H when π₅ is S; π₆ is Y; and π₇ is I. In some aspects, π₄ is H when π₅ is N; π₆ is I; and π₇ is A. In some aspects, π₄ is Y when π₅ is F; π₆ is Y; and π₇ is I. In some aspects, π₄ is H when π₅ is S; π₆ is A; and π₇ is L. In some aspects, π₄ is H when π₅ is G; π₆ is G; and π₇ is I. In some aspects, π₄ is H when π₅ is R; π₆ is R; and π₇ is I.

In some embodiments, the antibody comprises a CDR-L3 sequence defined by consensus sequence Q-Q-λ₃-λ₄-λ₅-λ₆-P-T (SEQ ID NOS: 148-150), where λ₃ is R, F, II, S, L, D, Y, or V; λ₄ is S, V, T, G, L, Y, or N; λ₅ is N, L, F, K, or V; and λ₆ is W, F, Y, or L.

In some aspects, λ₃ is R, when λ₄ is S or N; λ₅ is N or F; and λ₆ is W or Y. In some aspects, λ₃ is H when λ₄ is V or T; λ₅ is N or V; and Δ₆ is F or W. In some aspects, λ₃ is S when λ₄ is V or Y; λ₅ is F; and λ₆ is W or L. In some aspects, λ₄ is V when λ₃ is F, H, S, or D; λ₅ is L, N, or F; and λ₆ is W or F. In some aspects, λ₄ is T when λ₃ is L or H; λ₅ is K or V; and λ₆ is W. In some aspects, λ₅ is N when λ₃ is R, H, or V; λ₄ is S, V, or L; and λ₆ is W, F, or Y. In some aspects, λ₅ is L when λ₃ is F, D, or Y; λ₄ is V or G; and λ₆ is W or F. In some aspects, λ₅ is F when λ₃ is S or R; λ₄ is V, Y, or N; and λ₆ is W, L, or Y. In some aspects, λ₆ is W when λ₃ is R, F, S, L, D, or H; λ₄ is S, V, or T; and λ₅ is N, L, F, K, or V. In some aspects, λ₆ is F when λ₃ is H or Y; λ₄ is V or G; and λ₅ is N or L. In some aspects, λ6 is Y when λ₃ is V or R; λ₄ is L or N; and λ₅ is N or F.

In some aspects, λ₃ is R when λ₄ is S; λ₅ is N; and λ₆ is W. In some aspects, λ₃ is F when λ₄ is V; λ₅ is L; and λ₆ is W. In some aspects, λ₃ is H when λ₄ is V; λ₅ is N; and λ₆ is F. In some aspects, λ₃ is S when λ₄ is V; λ₅ is F; and λ₆ is W. In some aspects, λ₃ is L when λ₄ is T; λ₅ is K; and λ₆ is W. In some aspects, λ₃ is D when λ₄ is V; λ₅ is L; and λ₆ is W. In some aspects, λ₃ is Y when λ₄ is G; λ₅ is L; and λ₆ is F. In some aspects, λ₃ is H when λ₄ is T; λ₅ is V; and λ₆ is W. In some aspects, λ₃ is V when λ₄ is L; λ₅ is N; and λ₆ is Y. In some aspects, λ₃ is S when λ₄ is Y; λ₅ is F; and λ₆ is L. In some aspects, λ₃ is R when λ₄ is N; λ₅ is F; and λ₆ is Y.

In some embodiments, the antibody comprises a CDR-L3 sequence defined by the consensus sequence Q-Q-Y-ρ₃-ρ₄-W-P-L-T (SEQ ID NOS: 151 and 152), where ρ₃ is N or L and ρ₄ is N or L.

In some aspects, ρ₃ is N when ρ₄ is L. In some aspects, ρ₃ is L when ρ₄ is L.

In some embodiments, the antibody comprises a CDR-L3 sequence defined by the consensus sequence Q-Q-ω₃-ω₄-ω₅-ω₆-P-ω₈-T (SEQ ID NOS: 153-156), where ω₃ is Y or F; ω₄ is Y or W; ω₅ is S, L, T, or F; ω₆ is T, Y, or F; and ω₈ is L or P.

In some aspects, ω₃ is Y when ω₄ is Y or W; ω₅ is S, L, or T; ψ₆ is T or Y; and ω₈ is L. In some aspects, ω₄ is Y when ω₃ is Y or I; ω₅ is S, L, or F; ω₆ is T or Y; and ω₆ is L or P. In some aspects, ω₆ is Y when ω₃ is Y; ω₄ is Y or W; ω₅ is L or T; and ω₈ is L. In some aspects, is L when ω₈ is Y; ω₄ is Y or W; ω₅ is S, L, or T; and ω₆ is T or Y.

In some aspects, ω₃ is Y when ω₄ is Y; ω₅ is S; ω₆ is T; and ω₈ is L. In some aspects, ω₃ is Y when ψ₄ is Y; ω₅ is L; ω₆ is Y; and ω₈ is L. In some aspects, ω₃ is Y when ω₄ is W; ω₅ is T; ω₆ is Y; and ω₈ is L. In some aspects, ω₃ is F when ω₄ is Y; ω₅ is F; ω₆ is F; and ω₈ is P.

### 2.8.7. CDR-L2 Consensus Sequences

In some embodiments, the antibody comprises a CDR-L2 sequence defined by the consensus sequence ψ₁-A-S-ψ₄-R-ψ₆-ψ₇ (SEQ ID NOS: 125-136), where ψ₁ is G or Y, ψ₄ is S or N; Ω₆ is A or H; and ψ₇ is T, Y, or N.

In some aspects, ψ₁ is G when ψ₄ is S or N; ψ₆ is A or H; and ψ₇ is T or N. In some aspects, ψ₁ is Y when is S or N; ψ₆ is A; and ψ₇ is Y or T. In some aspects, ψ₄ is S when ψ₁ is G or Y; ψ₆ is A; and ψ₇ is T, Y, or N. In some aspects, ψ₄ is N when ψ₁ is G or Y; ψ₆ is H or A; and ψ₇ is T. In some aspects, ψ₆ is A when ψ₁ is G or Y; ψ₄ is S or N; and ω₇ is T, Y, or N. In some aspects, ψ₇ is T when ψ₁ is G or Y; ψ₄ is S or N; and ψ₆ is A or H.

In some aspects, ω₁ is G when ψ₄ is S; ψ₆ is A; and ψ₇ is T. In some aspects, ψ₁ is G when ψ₄ is N; ψ₆ is H; and ψ₇ is T. In some aspects, ψ₁ is Y when ψ₄ is S; ψ₆ is A; and ψ₇ is Y. In some aspects, ψ₁ is G when ψ₄ is S; ψ₆ is A; and ψ₇ is N. In some aspects, ψ₁ is Y when ψ₄ is N; ψ₆ is A; and ψ₇ is T.

In some embodiments, the antibody comprises a CDR-L2 sequence defined by the consensus sequence D-A-S-χ₄-R-A-T (SEQ ID NOS: 138 and 139), where £₄ is N or K.

In some aspects, χ₄ is N. In some aspects, χ₄ is K.

In some embodiments, the antibody comprises a CDR-H2 sequence defined by the consensus sequence W-A-S-T-R-σ₆-S(SEQ ID NOS: 131 and 133-134), where σ₆ is A, E, or Q.

In some aspects, σ₆ is A. In some aspects, σ₆ is E. In some aspects, σ₆ is Q.

### 2.8.8. CDR-L1 Consensus Sequences

In some embodiments, the antibody comprises a CDR-L1 sequence defined by the consensus sequence ϕ₁-A-S-ϕ₄-ϕ₅-V-ϕ₇-ϕ₈-ϕ₉-Y-L-A (SEQ ID NOS: 1101-114), where ϕ₁ is E, K, or R; ϕ_{b 4} is Q or E; ϕ₅ is S or Y; ϕ₇ is S or A; ϕ₈ is S or Y; and ϕ₉ is D or S.

In some aspects, ϕ₁ is R when ϕ₄ is Q or E; ϕ₅ is S or Y: ϕ₇ is S or A; ϕ₈ is S or Y; and ϕ₉ is S or D. In some aspects, ϕ₄ is E when ϕ₁ is K or R; ϕ₅ is S; ϕ₇ is S; ϕ₈ is S; and ϕ₉ is S. In some aspects, ϕ₄ is Q when ϕ₁ is E or R; ϕ₅ is S or Y; ϕ₇ is S or A; ϕ₈ is S or Y; and ϕ₉ is S or D. In some aspects, ϕ₅ is S when ϕ₁ is E, K, or R; ϕ₄ is E or Q; ϕ₇ is S or A; ϕ₈ is S or Y; and ϕ₉ is S or D. In some aspects, ϕ₇ is S when ϕ₁ is E, K, or R: ϕ₄ is E or Q; ϕ₅ is S or Y; ϕ₈ is S or Y; and ϕ₉ is S or D. In some aspects, ϕ₈ is S when ϕ₁ is K or R; ϕ₄ is E or Q; ϕ₅ is S or 5; ϕ₇ is A or S; and ϕ₉ is S or D. In some aspects, ϕ₈ is R when ϕ₁ is E or R; ϕ₄ is Q; ϕ₅ is S; ϕ₇ is S; and ϕ₉ is S. In some aspects, ϕ₉ is S when ϕ₁ is E, K, or R; ϕ₄ is E or Q; ϕ₅ is S or Y; ϕ₇ is A or S; and ϕ₈ is S or Y.

In some aspects, ϕ₁ is K when ϕ₄ is E; ϕ₅ is S; ϕ₇ is S; ϕ₈ is S; and ϕ₉ is S. In some aspects, ϕ₁ is E, when ϕ₄ is Q; ϕ₅ is S; ϕ₇ is S; ϕ₈ is Y; and ϕ₉ is S. In some aspects, ϕ₁ is R when ϕ₄ is Q; ϕ₅ is S; ϕ₇ is S; ϕ₈ is S; and ϕ₉ is D. In some aspects, ϕ₁ is R when ϕ₄ is Q; ϕ₅ is S; ϕ₇ is S; ϕ₈ is S; and ϕ₉ is S. In some aspects, ϕ₁ is R when ϕ₄ is Q; ϕ₅ is S; ϕ₇ is A; ϕ₈ is S; and ϕ₉ is S. In some aspects, ϕ₁ is R when ϕ₄ is Q; ϕ₅ is S; ϕ₇ is S; ϕ₈ is Y; and ϕ₉ is S. In some aspects, ϕ₁ is R when ϕ₄ is E; ϕ₅ is S; ϕ₇ is S; ϕ₈ is S; and ϕ₉ is S. In some aspects, ϕ₁ is R when ϕ₄ is Q; ϕ₅ is Y; ϕ₇ is S; ϕ₈ is S; and ϕ₉ is S.

In some embodiments, the antibody comprises a CDR-L1 sequence defined by the consensus sequence σ₁-A-S-Q-σ₅-σ₆-σ₇-σ₈-σ₉-I-σ₁₁ (SEQ ID NOS: 118 and 120-123), where σ₁ is Q or R1; σ₅ is D or S; σ₆ is I or V; σ₇ is G or S; σ₈ is N, R, or S; σ₉ is N, Y, or W; and σ₁₁ is A or N.

In some aspects, when σ₁ is R, σ₅ is S; σ₆ is I or V; σ₇ is G or S; σ₈ is R or S; σ₉ is N, Y, or W; and σ₁₁ is A. In some aspects, when σ₅ is S, σ₁ is R; σ₆ is I or V; σ₇ is G or S; σ₈ is R or S; σ₉ is N, Y, or W; and σ₁₁ is A. In some aspects, when σ₆ is I, σ₁ is Q or R; σ₅ is D or S; σ₇ is S; σ₈ is N or S; σ₉ is Y or W; and σ₁₁ is A or N. In some aspects, when σ₆ is V, σ₁ is R; σ₅ is S; σ₇ is G or S; σ₈ is R or S; σ₉ is N or W; and σ₁₁ is A. In some aspects, when σ₇ is S, σ₁ is Q or R; σ₅ is D or S; σ₆ is I or V; σ₈ is N, S, or R; σ₉ is Y or W; and σ₁₁ is A or N. In some aspects, when σ₈ is S, σ₁ is R; σ₅ is S; σ₆ is I or V; σ₇ is S; σ₉ is N, Y, or W; and σ₁₁ is A. In some aspects, when σ₉ is Y, σ₁ is Q or R; σ₅ is D or S; σ₆ is I or V; σ₇ is S; σ₈ is N, S, or R; and σ₁₁ is A or N. In some aspects, when σ₁₁ is A, σ₁ is R; σ₅ is S; σ₆ is I or V; σ₇ is S or G; σ₈ is S, or R; and σ₉ is N, W, or Y.

In some aspects, when σ₁ is R, σ₅ is S; σ₆ is V; σ₇ is S; σ₈ is S; σ₉ is Y; and σ₁₁ is A. In some aspects, when σ₁ is Q, σ₅ is D; σ₆ is I; σ₇ is S; σ₈ is N; σ₉ is Y; and σ₁₁ is N. In some aspects, when σ₁ is R, σ₅ is S; σ₆ is V; σ₇ is S; σ₈ is R; σ₉ is Y; and σ₁₁ is A. In some aspects, when σ₁ is R, σ₅ is S; σ₆ is V; σ₇ is G; σ₈ is S; σ₉ is N; and σ₁₁ is A. In some aspects, when σ₁ is R, σ₅ is S; σ₆ is I; σ₇ is S; σ₈ is S; σ₉ is W; and σ₁₁ is A.

In some embodiments, the antibody comprises a CDR-L1 sequence defined by the consensus sequence K—S-S-Γ₄-S-V-L-Γ₈-S-Γ₁₀-N—N-K-N-Y-L-A (SEQ ID NOS: 115-117), where Γ₄ is Q, R or K; Γ₈ is F or Y; and Γ₁₀ is S or N.

In some aspects, Γ₄ is Q when Γ₈ is F or Y and Γ₁₀ is S. In some aspects, Γ₈ is F when Γ₄ is Q or R and Γ₁₀ is S. In some aspects, Γ₈ is Y when Γ₄ is K or Q and Γ₁₀ is S or N. In some aspects, Γ₁₀ is S when Γ₄ is R or Q and Γ₈ is F or Y.

In some aspects, Γ₄ is Q when Γ₈ is Y and Γ₁₀ is S. In some aspects, Γ₄ is K when Γ₄ is Y and Γ₁₀ is N. In some aspects, Γ₄ is Q when Γ₈ is F and Γ₁₀ is S. In some aspects, Γ₄ is R when Γ₈ is F and Γ₁₀ is S.

### 3. Germline

In some embodiments, the antibody that specifically binds CD39 is an antibody comprising a variable region that is encoded by a particular germline gene, or a variant thereof. The illustrative antibodies provided herein comprise variable regions that are encoded by the heavy chain variable region germline genes V_{H}1-46, V_{H}1-69, 1-69, and V_{H}1-46, or variants thereof; and the light chain variable region germline genes VK3-20, VK3-11, VK4-01, VK3, and VK3-15, or variants thereof. One of skill in the art would recognize that the CDR sequences provided herein may also be useful when combined with variable regions encoded by other variable region germline genes, or variants thereof. In particular, the CDR sequences provided herein may be useful when combined with variable regions encoded by variable region germline genes, or variants thereof, that are structurally similar to the variable region germline genes recited above. For example, in some embodiments, a CDR-H sequence provided herein may be combined with a variable region encoded by a variable region germline gene selected from the VH1 or V_{H}3 family, or a variant thereof. In some embodiments, a CDR-L sequence provided herein may be combined with a variable region encoded by a variable region germline gene selected from the Vλ3, Vκ1, Vκ3, and Vκ4 families, or a variant thereof.

### 4. Affinity

In some embodiments, the affinity of the antibody for CD39, as indicated by K_{D}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸ M and 10⁻¹¹ M. In some embodiments, the affinity of the antibody is between about 10⁻⁸ M and 10⁻¹⁰ M. In some embodiments, the affinity of the antibody is between about 10⁻⁹ M and 10⁻¹¹ M. In some embodiments, the affinity of the antibody is between about 10⁻¹⁰ M and 10⁻¹¹ M.

In some embodiments, the affinity of the antibody for human CD39 is between about 4.09×10⁻⁷ M and 7.31×10⁻¹¹ M. In some embodiment, the affinity of the antibody for human CD39 is about 1.14×10⁻⁷ M, about 1.31×10⁻⁷ M, about 1.67×10⁻⁷ M, about 1.43×10⁻⁷ M, about 1.30×10⁻⁸ M, about 1.27×10⁻⁸ M, about 1.13×10⁻⁸ M, about 1.60×10⁻⁸ M, about 1.34×10⁻⁹ M, about 1.16×10⁻⁹ M, about 7.31×10⁻¹¹ M, about 7.60×10⁻¹⁰ M, about 2.66×10⁻¹⁰ M, about 9.22×10⁻¹⁰ M, about 6.72×10⁻¹⁰ M, about 9.24×10⁻¹⁰ M, about 5.58×10⁻¹⁰ M, about 5.48×10⁻⁸ M, about 3.37×10⁻⁸ M, about 3.11×10⁻⁸ M, about 1.88×10⁻⁸ M, about 1.63×10⁻⁸ M about 1.64×10⁻⁸ M, about 1.01×10⁻⁸ M, about 2.44×10⁻⁸ M, about 4.09×10⁻⁷ M, about 3.35×10⁻⁸ M, about 1.91×10⁻⁸ M, about 1.73×10⁻⁸ M, or about 2.39×10⁻⁸ M.

In some embodiments the antibody has a kon when associating with human CD39 of between about 1.93×10⁴ M⁻¹×sec⁻¹ and about 1.72×10⁶ M⁻¹×sec⁻¹. In some embodiments the antibody has a kₐ when associating with human CD39 of about 6.59×10⁴ M⁻¹×sec⁻¹, about 1.93×10+M⁻¹×sec⁻¹, about 4.44×10⁵ M⁻¹×sec⁻¹, about 2.72×10⁵ M⁻¹×sec⁻¹, about 6.39×10⁵ M⁻¹×sec⁻¹, about 8.93×10⁵ M⁻¹×sec⁻¹, about 9.55×10⁵ M⁻¹×sec⁻¹, about 2.11×10⁵ M⁻¹×sec⁻¹, about 1.17×10⁵ M⁻¹×sec⁻¹, about 2.02×10⁵ M⁻¹×sec⁻¹, about 1.76×10⁵ M⁻¹×sec⁻¹, about 1.72×10⁵ M⁻¹×sec⁻¹, about 2.73×10⁵ M⁻¹×sec⁻¹, about 1.43×10⁵ M⁻¹×sec⁻¹, about 9.01×10⁵ M⁻¹×sec⁻¹, about 3.13×10⁵ M⁻¹×sec⁻¹, about 5.03×10⁵ M⁻¹×sec⁻¹, about 3.02×10⁵ M⁻¹×sec⁻¹, about 2.73×10⁵ M⁻¹×sec⁻¹, about 1.78×10⁵ M⁻¹×sec⁻¹, about 2.98×10⁵ M⁻¹×sec⁻¹, about 4.31×10⁵ M⁻¹×sec⁻¹, about 2.27×10⁵ M⁻¹×sec⁻¹, about 3.14×10⁵ M⁻¹×sec⁻¹, about 2.81×10⁵ M⁻¹×sec⁻¹, about 4.73×10⁵ M⁻¹×sec⁻¹, about 3.26×10⁵ M⁻¹×sec⁻¹, about 1.73×10⁵ M⁻¹×sec⁻¹, about 2.68×10⁵ M⁻¹×sec⁻¹, about 2.63×10⁵ M⁻¹×sec⁻¹, about 3.82×10⁵ M⁻¹×sec⁻¹, about 2.46×10⁵ M⁻¹×sec⁻¹, about 3.11×10⁵ M⁻¹×sec⁻¹, about 4.53×10⁵ M⁻¹×sec⁻¹, about 4.63×10⁵ M⁻¹×sec⁻¹, about 9.01×10⁵ M⁻¹×sec⁻¹, about 1.03×10⁶ M⁻¹×sec⁻¹, about 1.52×10⁶ M⁻¹×sec⁻¹, or about 3.53×10⁵ M⁻¹×sec⁻¹.

In some embodiments the antibody has a k_{off} of about 7.51×10⁻³ sec⁻¹, about 6.33×10⁻² sec⁻¹, about 4.70×10⁻² sec⁻¹, about 7.82×10⁻⁴ sec⁻¹, about 4.70×10⁻² sec⁻¹, about 1.05×10⁻² sec⁻¹, about 3.65×10⁻¹ sec⁻¹, about 1.60×10⁻¹ sec⁻¹, about 7.11×10⁻³ sec⁻¹, about 6.44×10⁻³ sec⁻¹, about 3.85×10⁻² sec⁻¹, about 2.30×10⁻² sec⁻¹, about 5.33×10⁻² sec⁻¹, about 9.14×10⁻² sec⁻¹, about 1.80×10⁻³ sec⁻¹, about 8.15×10⁻³ sec⁻¹, about 3.85×10⁻⁴ sec⁻¹, about 1.34×10⁻⁴ sec⁻¹, about 2.29×10⁻⁴ sec⁻¹, about 4.37×10⁻³ sec⁻¹, about 3.71×10⁻³ sec⁻¹, about 4.06×10⁻³ sec⁻¹, about 6.66×10⁻² sec⁻¹, about 2.02×10⁻³ sec⁻¹, about 2.00×10⁻⁴ sec⁻¹, about 5.26×10⁻³ sec⁻¹, about 1.13×10⁻² sec⁻¹, about 3.28×10⁻³ sec⁻¹, about 2.76×10⁻³ sec⁻¹, about 2.86×10⁻⁴ sec⁻¹, about 2.43×10⁻⁴ sec⁻¹, about 2.13×10⁻⁴ sec⁻¹, about 6.09×10⁻⁴ sec⁻¹, about 8.39×10⁻⁴ sec⁻¹, about 8.15×10⁻³ sec⁻¹, about 1.32×10⁻⁴ sec⁻¹, about 1.11×10⁻⁴ sec⁻¹, about 2.43×10⁻⁴ sec⁻¹, about 2.13×10⁻⁴ sec⁻¹, about 6.09×10⁻⁴ sec⁻¹, about 8.15×10⁻³ sec⁻¹, about 1.32×10⁻⁴ sec⁻¹, or about 1.11×10⁻⁴ sec⁻¹.

In some aspects, the K_{D}, kₐ, and k_{d} are determined at 25° C. In some embodiments, the K_{D} kₐ, and k_{d} are determined by surface plasmon resonance. In some embodiments, the K_{D}, kₐ, and k_{d} are determined according to the methods described in the examples.

### 5. Inhibition of CD39

In some aspects, the antibody decreases affinity of CD39 to its substrate. In some aspects, the antibody inhibits CD39 function on tumor cells. In some aspects, the antibody inhibits or impedes the release of ADP or AMP from CD39. In some aspects, the antibody inhibits or impedes CD39 processivity.

In some aspects, the antibody binds CD39 but does not inhibit ATPase. In some aspects, the antibody binds CD39 and inhibits extracellular CD39 activity but not cellular ATPase activity. In some aspects, the antibody binds both the extracellular domain of CD39 and cellular CD39 and can inhibit both the extracellular domain of CD39 and cellular CD39. In some aspects, the antibodies do not compete with A1 and/or others in binding to the extracellular domain.

### 6. CD39 Assays

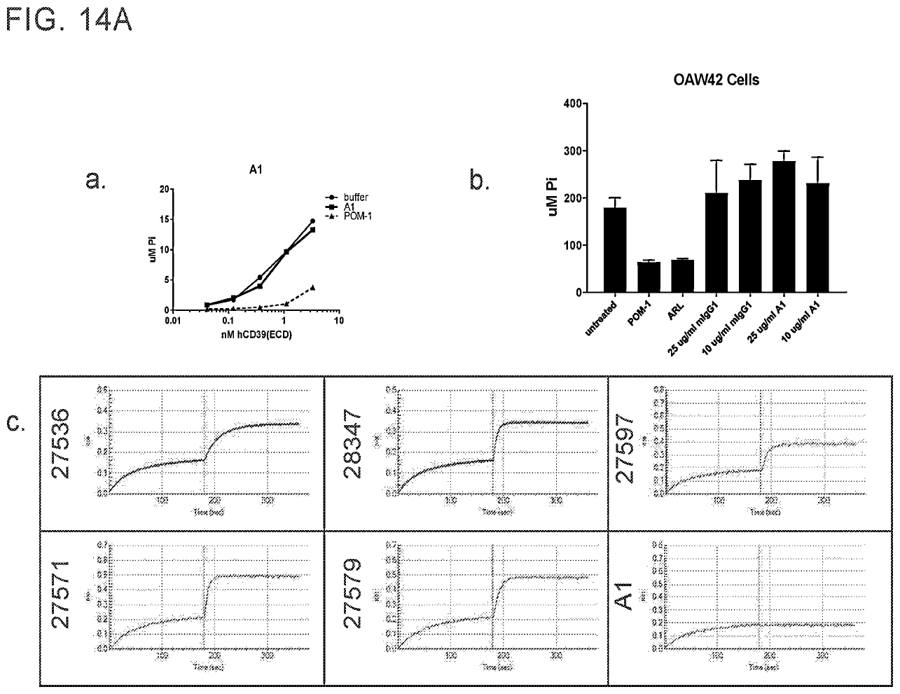

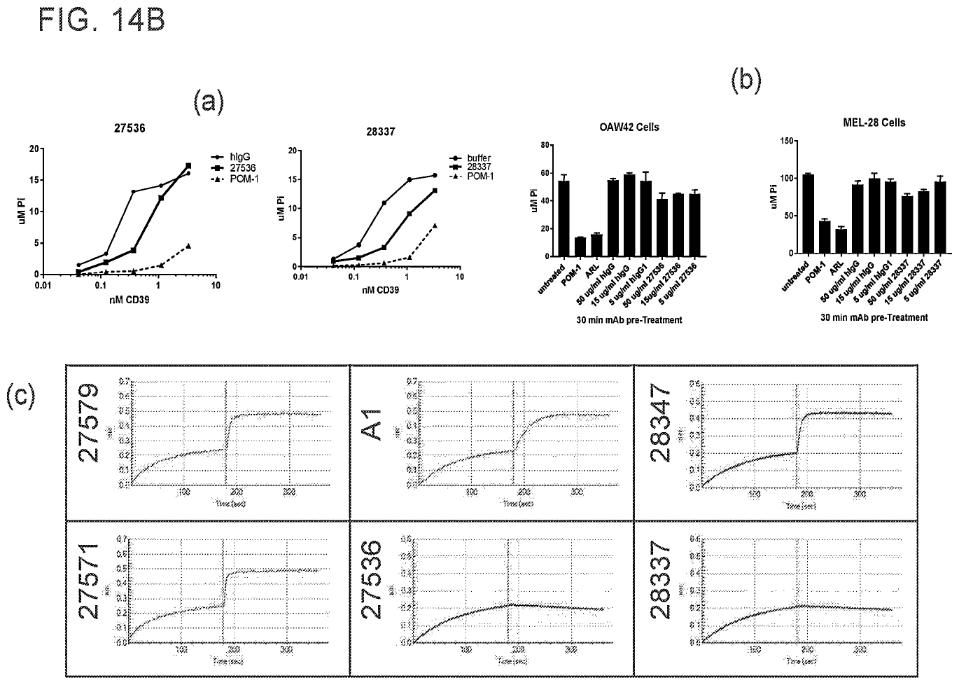

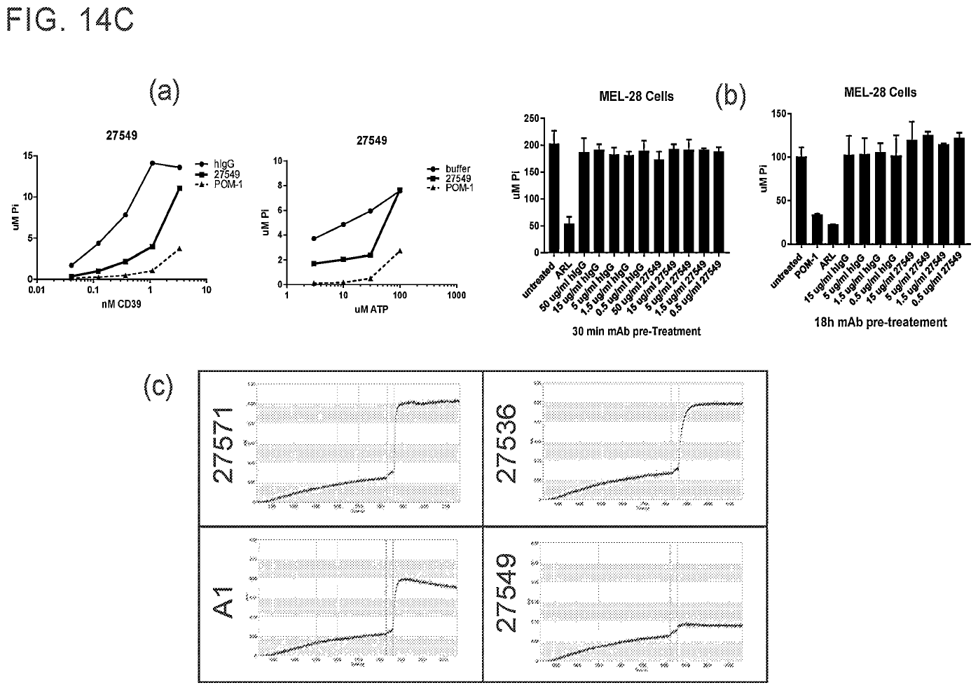

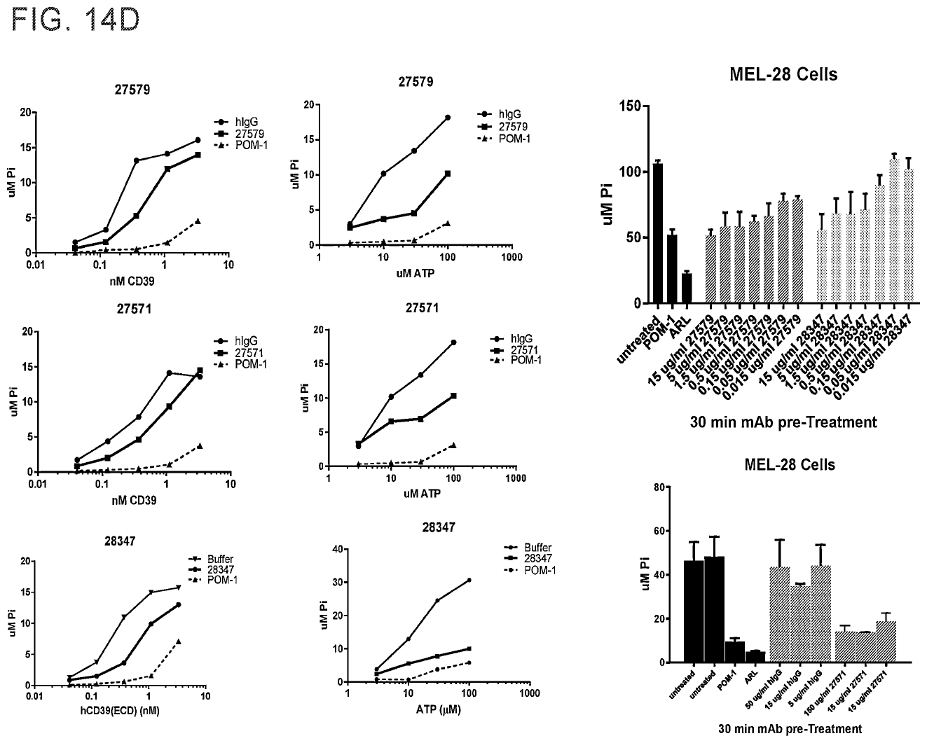

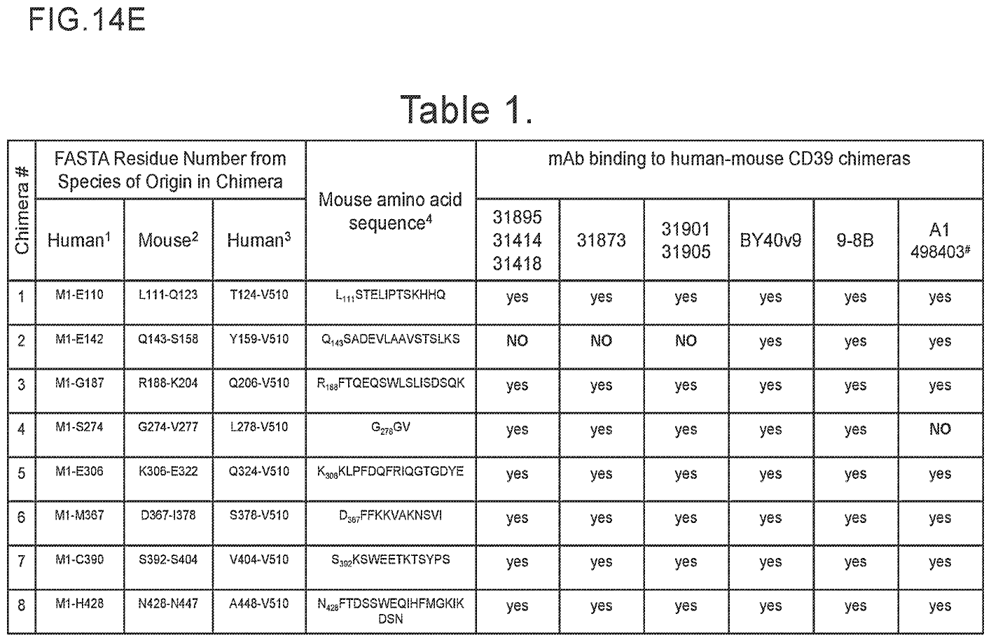

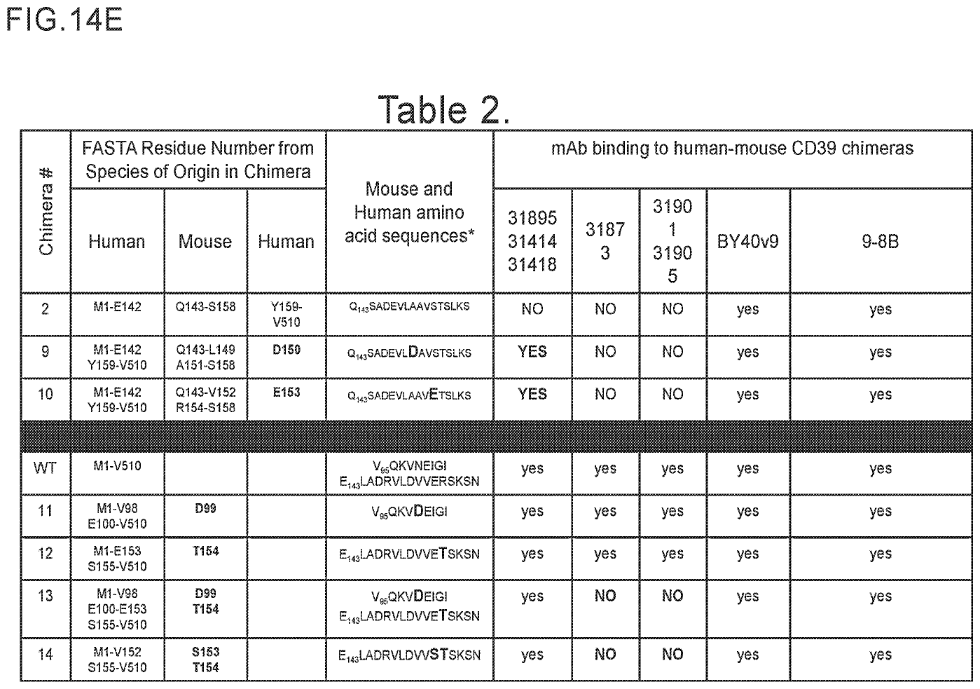

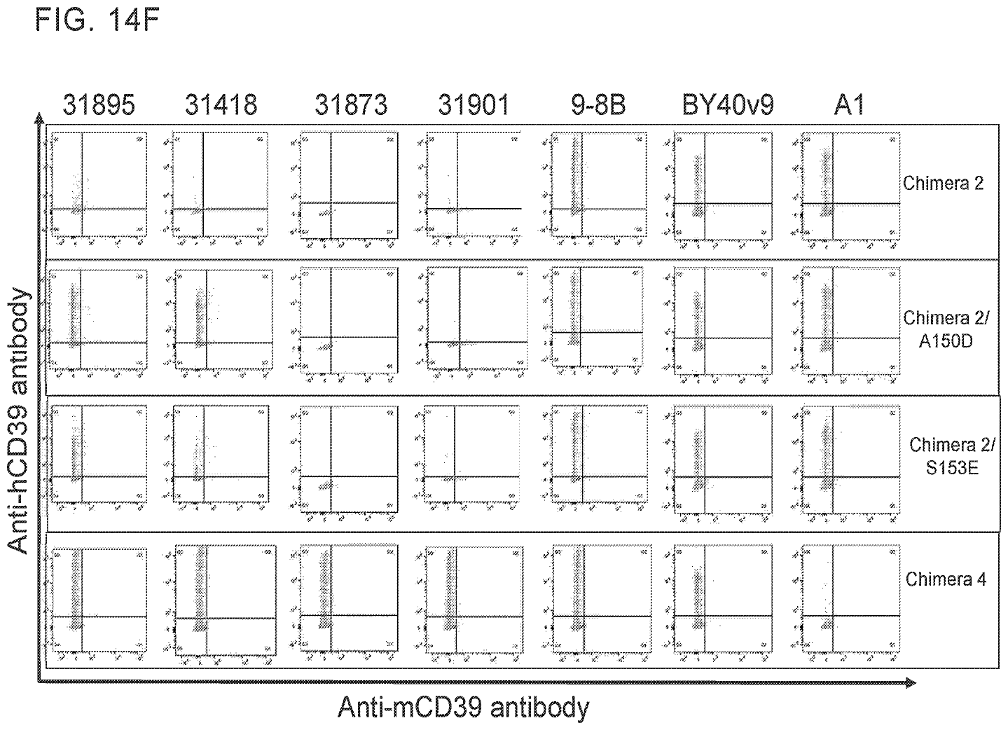

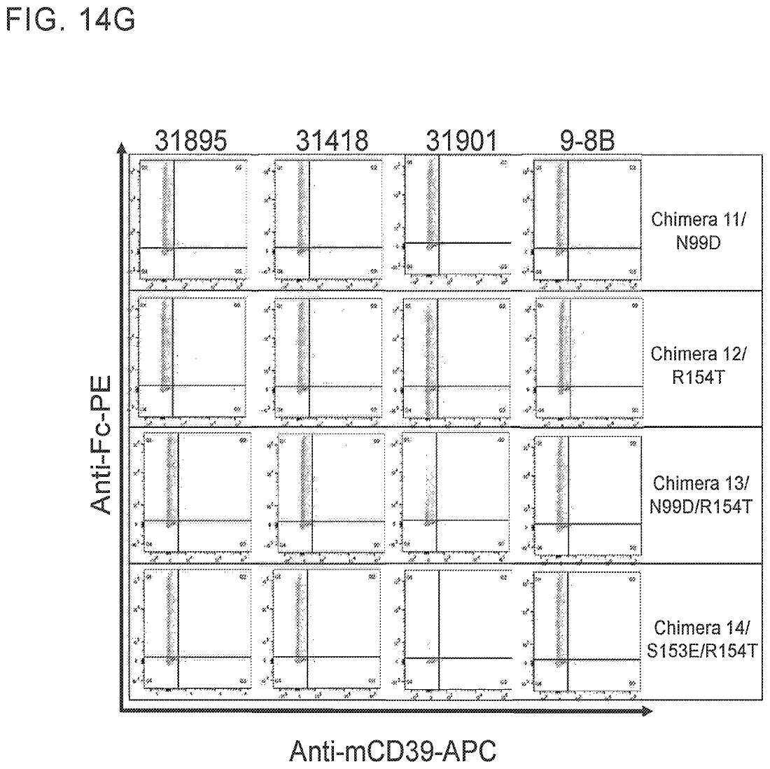

In some embodiments, the antibody binds to an epitope of CD39. In some aspects, CD39 has a sequence identical to the amino acid sequence set forth in SEQ ID NO: 249. In some aspects, the epitope has an amino acid sequence that is identical to the amino acid sequence set forth in SEQ ID NO: 249. In some aspects, the epitope is in an extracellular domain of CD39. In some aspects, the extracellular domain corresponds to all or at least a portion of amino acids 38-478 of SEQ ID NO: 249. In some aspects, the epitope has an amino acid sequence that is 60%, 65%, 70%, 75%, 80%, 85%, 90%, or 95% identical to the sequence set forth in SEQ ID NO: 249 or all or a portion of the extracellular domain. In some aspects, the epitope has a sequence that is identical or corresponds to residues 143-158 and/or residues 274-277 of SEQ ID NO: 249. In some aspects, the epitope is in the region of E143 to N158 on the human CD39 polypeptide having the sequence set forth in SEQ ID NO: 249. In some aspects, the epitope has a sequence that has a 60%, 65%, 70%, 75%, 80%, 85%, 90%, or 95% identity to residues 143-158 or 274-277 of the sequence set forth in SEQ ID NO: 249. In some aspects, the epitope has 1, 2, 3, 4, 5, 6, 7, 8, or 9 substitutions from residues 143-158 of the sequence forth in SEQ ID NO: 249. In some aspects, the epitope has 1, 2 or 3 substitutions from residues 274-277 of SEQ ID NO: 249. In some aspects, the antibody makes contact with any of the residues set forth in FIG. 14E, Table 1. In some aspects, the antibody makes contact with any of the residues set forth in FIG. 14E, Table 2. In some aspects, the antibody binds to D150, E153, and/or R154 or to N99 and none, one, two, or three of D150, E153, and R154 or to any of the above alone or in combination.

In some aspects, the antibody competes with 1, 2, 3, 4, or 5 of antibodies 27536, 27571, 28347, 27579, or 27597 as set forth in FIG. 14A. In some aspects, the antibody competes with 1, 2, 3, 4, or 5 antibodies 27536, 27571, 28347, 27579, or 27597 as set forth in FIG. 14A. In some aspects, the antibody competes with 1, 2, or 3 of antibodies 25571, 27536, or 27549 as set forth in FIG. 14C. In some aspects, the antibody competes with 1, 2, or 3 of antibodies 25571, 27536, or 27549 set forth in FIG. 14C.

In some aspects, the antibody inhibits conversion by CD39 of ATP to ADP and/or ADP to AMP. In some aspects, the antibody inhibits platelet aggregation. In some aspects, the antibody decreases or prevents activation of phospho antigen specific T cells selected from MAIT cells and γδ T cells. In some aspects, the antibody inhibits angiogenesis. In some aspects, the antibody decreases levels of phosphate, ADP, AMP, and/or adenosine and/or increasing levels of ATP. In some aspects, the antibody increases T effector cell function. In some aspects, the antibody decreases the number of regulatory T cells in tissues or in circulation. In some aspects, the antibody decreases the regulatory T cells or regulatory T cell activity. In some aspects, the antibody increases B cell function. In some aspects, the antibody increases antigen presenting cell function. In some aspects, the antibody inhibits processing of at least one of phospho-antigen from phosphorylated isoprenoid, phosphorylated vitamin B metabolite, and/or phosphorylated riboflavin.

In some aspects, the antibody has limited ability to limit ATPase of the soluble or extracellular domain. In some aspects, the antibody has limited ability to inhibit ATPase of the cellular and/or extracellular domain of CD39.

### 7. Glycosylation Variants

In certain embodiments, an antibody may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

In certain embodiments, the antibody is glycosylated. In certain embodiments, the antibody is deglycosylated. Carbohydrates may be removed by standard techniques. In certain embodiments, the antibody is aglycosylated, for instance by expression in a system that does not glycosylate.

### 8. Fc Variants

In certain embodiments, amino acid modifications may be introduced into the Fc region of an antibody provided herein to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

An alteration in in CDC and/or ADCC activity can be confirmed using in vitro and/or in vivo assays. For example, Fc receptor (FcR) binding assays can be conducted to measure FcγR binding. The primary cells for mediating ADCC, NK cells, express FcγRIII only, whereas monocytes express FcγRI, FcγRII and FcγRIII. FcR expression on hematopoietic cells is summarized in Ravetch and Kinet, Ann. Rev. Immunol., 1991, 9:457-492.

Non-limiting examples of in vitro assays to assess ADCC activity of a molecule of interest are provided in U.S. Pat. Nos. 5,500,362 and 5,821,337; Hellstrom et al., Proc. Natl. Acad. Sci. U.S.A., 1986, 83:7059-7063; Hellstrom et al., Proc. Natl. Acad. Sci. U.S.A., 1985, 82:1499-1502; and Bruggemann et al., J. Exp. Med., 1987, 166:1351-1361. Useful effector cells for such assays include peripheral blood mononuclear cells (PBMC) and Natural Killer (NK) cells. Alternatively, or additionally, ADCC activity of the molecule of interest may be assessed in vivo, using an animal model such as that disclosed in Clynes et al. Proc. Natl. Acad. Sci. U.S.A., 1998, 95:652-656.

C1q binding assays may also be carried out to confirm that the antibody is unable to bind C1q and hence lacks CDC activity. Examples of C1q binding assays include those described in WO 2006/029879 and WO 2005/100402.

Complement activation assays include those described, for example, in Gazzano-Santoro et al., J. Immunol. Methods, 1996, 202:163-171; Cragg et al., Blood, 2003, 101:1045-1052; and Cragg and Glennie, Blood, 2004, 103:2738-2743.

FcRn binding and in vivo clearance (half-life determination) can also be measured, for example, using the methods described in Petkova et al., Intl. Immunol., 2006, 18:1759-1769.

### 9. Preparation of Antibodies

### 9.1. Antigen Preparation

The CD39 antigen to be used for production of antibodies may be intact CD39 or a fragment of CD39. The intact CD39, or fragment of CD39, may be in the form of an isolated protein or expressed by a cell. Other forms of CD39 useful for generating antibodies will be apparent to those skilled in the art.

### 9.2. Monoclonal Antibodies

Monoclonal antibodies may be obtained, for example, using the hybridoma method first described by Kohler et al., Nature, 1975, 256:495-497, and/or by recombinant DNA methods (see e.g., U.S. Pat. No. 4,816,567). Monoclonal antibodies may also be obtained, for example, using phage or yeast-based libraries. See e.g., U.S. Pat. Nos. 8,258,082 and 8,691,730.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, CA.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

### 9.3. Humanized Antibodies

Humanized antibodies may be generated by replacing most, or all, of the structural portions of a monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370.

### 9.4. Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905). Human antibodies may also be generated by in vitro activated B cells (see e.g., U.S. Pat. Nos. 5,567,610 and 5,229,275). Human antibodies may also be derived from yeast-based libraries (see e.g., U.S. Pat. No. 8,691,730).

### 10. Vectors, Host Cells, and Recombinant Methods

The invention also provides isolated nucleic acids encoding anti-CD39 antibodies, vectors and host cells comprising the nucleic acids, and recombinant techniques for the production of the antibodies.

For recombinant production of the antibody, the nucleic acid encoding it may be isolated and inserted into a replicable vector for further cloning (i.e., amplification of the DNA) or expression. In some aspects, the nucleic acid may be produced by homologous recombination, for example as described in U.S. Pat. No. 5,204,244.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615.

Illustrative examples of suitable host cells are provided below. these host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for anti-CD39 antibody-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida ((. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO)); mouse sertoli cells; African green monkey kidney cells (VERO-76), and the like.

The host cells used to produce the anti-CD) 39 antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 minutes. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth., 1983, 62:1-13). Protein G is useful for all mouse isotypes and for human γ3 (Guss et al., EMBO J., 1986, 5:1567-1575).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 11. Pharmaceutical Compositions and Methods of Administration

Any of the antibodies provided herein can be provided in any appropriate pharmaceutical composition and be administered by any suitable route of administration. Suitable routes of administration include, but are not limited to, the inhalation, intraarterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a cosolvent. Illustrative examples of cosolvents include ethanol, poly(ethylene)glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene (glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes. In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies.

Further encompassed herein are anhydrous pharmaceutical compositions and dosage forms comprising an antibody, since water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

### 11.1. Parenteral Dosage Forms

In certain embodiments, provided are parenteral dosage forms. Parenteral dosage forms can be administered to subjects by various routes including, but not limited to, subcutancous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

### 11.2. Dosage and Unit Dosage Forms

In human therapeutics, the doctor will determine the posology which he considers most appropriate according to a preventive or curative treatment and according to the age, weight, condition and other factors specific to the subject to be treated.

The amount of the antibody or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the antibody provided herein, based on weight of the antibody, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

The dose can be administered according to a suitable schedule, for example, once, two times, three times, or for times weekly. It may be necessary to use dosages of the antibody outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an antibody or composition provided herein followed by one or more maintenance doses.

In certain embodiments, a dose of an antibody or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least 1 day, 2 days, 3 days, 5 days, 10 days, 15 days, 30 days, 45 days, 2 months, 75 days, 3 months, or 6 months.

### 12. Therapeutic Applications

For therapeutic applications, the antibodies of the invention are administered to a mammal, generally a human, in a pharmaceutically acceptable dosage form such as those known in the art and those discussed above. For example, the antibodies of the invention may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibodies also are suitably administered by peritumoral, intralesional, or perilesional routes, to exert local as well as systemic therapeutic effects. The intraperitoneal route may be particularly useful, for example, in the treatment of ovarian tumors.

The antibodies provided herein may be useful for the treatment of any disease or condition involving CD39, such as cancer, autoimmune disease, and infection.

Any suitable cancer may be treated with the antibodies provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

Any suitable autoimmune disease may be treated with the antibodies provided herein. Illustrative suitable autoimmune diseases, or diseases with an autoimmune component, include, for example, acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner car disease (AIED), autoimmune myocarditis, autoimmune oophoritis, autoimmune pancreatitis, autoimmune retinopathy, autoimmune thrombocytopenia purpura (ATP), autoimmune thyroid disease, autoimmune urticarial, axonal & neuronal neuropathies, Balo disease. Behcet's disease, bullous pemphigoid, cardiomyopathy, Castleman disease, Celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid. Crohn's disease, Cogans syndrome, cold agglutinin disease, colitis, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis. Evans syndrome, fibromyalgia, fibrosing alveolitis, giant cell arteritis (temporal arteritis), giant cell myocarditis, glomerulonephritis, Goodpasture's syndrome, granulomatosis with polyangiitis (GPA) (formerly called Wegener's Granulomatosis), Graves' disease, Guillain-Barre syndrome, Hashimoto's encephalitis. Hashimoto's thyroiditis, hemolytic anemia, Henoch-Schonlein purpura, herpes gestationis, hypogammaglobulinemia, idiopathic thrombocytopenia purpura (ITP), IgA nephropathy, IgG4-related sclerosing disease, immunoregulatory lipoproteins, inclusion body myositis, inflammatory bowel disease, interstitial cystitis, juvenile arthritis, juvenile diabetes (Type 1 diabetes), juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (SLE), Lyme disease (chronic), Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, rimary sclerosing cholangitis, psoriasis, psoriatic arthritis, idiopathic pulmonary fibrosis, pyoderma gangrenosum, pure red cell aplasia, Raynauds phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma. Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/giant cell arteritis, thrombotic disease, thrombocytopeniaurpura (TTP), Tolosa-Hunt syndrome, transverse myelitis, type 1 diabetes, ulcerative colitis, undifferentiated connective tissue disease (UCTD), uveitis, vasculitis, vesiculobullous dermatosis, vitiligo, and Wegener's granulomatosis (now termed Granulomatosis with Polyangiitis (GPA).

Any suitable infection may be treated with the antibodies provided herein. Illustrative suitable infections include, for example, hepatitis A virus, hepatitis B virus, hepatitis C virus (HCV), human immunodeficiency virus (HIV), and other viral infections.

### 13. Diagnostic Applications

In some embodiments, the antibodies provided herein are used in diagnostic applications. For example, an ant-CD39 antibody may be useful in assays for CD39 protein. In some aspects, the antibody can be used to detect the expression of CD39 in various cells and tissues. These assays may be useful, for example, evaluating cancer and autoimmune disease.

In some diagnostic applications, the antibody may be labeled with a detectable moiety. Suitable detectable moieties include, but are not limited to radioisotopes, fluorescent labels, and enzyme-substrate labels. In another embodiment of the invention, the anti-CD39 antibody need not be labeled, and the presence thereof can be detected using a labeled antibody which specifically binds to the anti-CD39 antibody.

### 14. Affinity Purification Reagents

The antibodies of the invention may be used as affinity purification agents. In this process, the antibodies may be immobilized on a solid phase such a resin or filter paper, using methods well known in the art. The immobilized antibody is contacted with a sample containing the CD39 protein (or fragment thereof) to be purified, and thereafter the support is washed with a suitable solvent that will remove substantially all the material in the sample except the CD39 protein, which is bound to the immobilized antibody. Finally, the support is washed with another suitable solvent, such as glycine buffer, pH 5.0, that will release the CD39 protein from the antibody.

### 15. Kits

In some embodiments, an anti-CD39 antibody provided herein is provided in the form of a kit, i.e., a packaged combination of reagents in predetermined amounts with instructions for performing a procedure. In some embodiments, the procedure is a diagnostic assay. In other embodiments, the procedure is a therapeutic procedure.

In some embodiments, the kit further comprises a solvent for the reconstitution of the anti-CD39 antibody. In some embodiments, the anti-CD39 antibody is provided in the form of a pharmaceutical composition.

### EXAMPLES

### Example 1: Selection of CD39 Antigen-Binding Proteins

CD39 ABPs were selected from a synthetic library of human antibodies presented on the surface of yeast cells in IgG format, as generally described, e.g., in WO2009036379; WO2010105256; WO2012009568; and Xu et al., Protein Eng. Des. Sel., 2013, 26:663-670 (each incorporated by reference in its entirety), and more specifically as provided below. The sequences and characteristics of the ABPs isolated from the recombinant library are provided in Table S.

Eight naïve human synthetic yeast libraries each of ˜10E+09 diversity were propagated as described in WO009036379; WO010105256; WO2012009568; and Xu et al., Protein Eng. Des. Sel., 2013, 26:663-670; each incorporated by reference in its entirety. For the first two rounds of selection, a magnetic bead sorting technique utilizing the Miltenyi MACS® system was performed, as described in Siegel et al., J. Immunol. Meth., 2004, 286:141-153. The following rounds of selection were performed using flow cytometry based sorting. For all round of selection, the antigen was biotinylated human CD39 extracellular domain (heretofore “ECD”), and decreasing concentrations of antigen were used in each subsequent round of selection. In addition to selection on antigen, some rounds of selection were employed in order to reduce the number of non-specific binders utilizing soluble membrane proteins from CHO cells (see WO2014179363 and Xu et al., Protein Eng. Des. Sel., 2013, 26:663-670, each incorporated by reference in its entirety). After the final round of sorting, yeast were plated and individual colonies were picked for characterization and for nomination of clones for affinity maturation.

Antibody variable domains of interest were synthesized, with codon optimization to maximize transient expression in host cells. The variable regions were cloned in to expression vectors containing human immunoglobulin constant domains and their sequence confirmed. Antibody heavy and light chain vector pairings were transfected into Expi293 cells using the Expifectamine system (Invitrogen). Transient cultures were harvested on day 4 and clarified cell culture supernatant IgGì titer was estimated using Bio-Layer Interferometry (BLI) using Octet (ForteBio) alongside standards. Antibodies were subsequently purified on a Protein A column and eluted using low pH glycine. Purified antibody samples were then buffer-exchanged or dialyzed into downstream assay-compatible buffers.

Antibody purity was assessed by running samples on SDS-PAGE and on an analytical size exclusion chromatography column.

Light Chain Shuffling: Heavy chain plasmids were extracted from naïve outputs (described herein) and transformed into a pre-made naïve light chain library with a diversity of 10E+06. Selections were performed as described above with one round of MACS sorting and three rounds of FACS sorting using decreasing amounts of biotinylated ECD antigen for respective rounds. Selected individual heavy chains from the primary discovery process were also independently transformed into separate pre-made light chain libraries with a diversity of 10E+06 and selections performed as described above with one round of MACS sorting and three rounds of FACS sorting using decreasing amount of biotinylated ECD antigen for respective rounds.

### Example 2: Affinity Maturation

Optimization of naïve clones was carried out utilizing three maturation strategies; diversification of CDR-H1 and CDR-H2; diversification of CDR-H3; diversification of CDR-L1, L2 and L3; shuffling of diversified heavy and light chains.

CDR-H1 and CDR-H2 Selection: The CDR-H3s from clones selected from each of the light chain batch diversification, light chain diversification, and naive discovery efforts were independently recombined into premade libraries with CDR-H1 and CDR-H2 variants of a diversity of >10E+8 and selections were performed using ECD antigen. Affinity pressures were applied by using decreasing concentrations of antigen.

CDR-H3 Selection: Clones obtained from the CDR-H1 and CDR-H2 selection procedure were subject to additional rounds of affinity maturation via walking dimer mutagenesis of the heavy chain. Selections were performed using ECD as antigen generally as described above but with the addition of employing FACS sorting for all selection rounds.

CDR-L1, L2, L3 Selection: Clones obtained from the CDR-H1 and CDR-H2 selection procedure were subject to additional rounds of affinity maturation via mutagenesis of the light chain. The CDR-L1 and CDR-H2 diversity derived from a pre-made library while CDR-L3 diversity derived from walking dimer mutagenesis. Selections were performed using ECD as antigen generally as described above but with the addition of employing FACS sorting for all selection rounds, with one round of MACS followed by three rounds of FACS in the CDR-L1, L2, L3 process described here.

Diversified Heavy Chain and Light Chain Shuffling: Outputs from heavy chain diversification and light diversification described above were recombined and selections were performed using ECD as antigen generally as described above but with the addition of employing FACS sorting for all selection rounds.

### Example 3: Monovalent Affinity of Anti-hCD39 Antibodies to Recombinant CD39 Extracellular Domain

Binding kinetics were measured using the Octet Red96 system (ForteBio) at 25° C. in running buffer (1× Pall ForteBio Kinetics Buffer diluted into PBS or Tris pH 7.4). In brief, 1.25 mg/ml of unlabeled anti-hCD39 antibodies were immobilized onto anti-human Fc sensors. After a short baseline step in running buffer, the sensors were exposed to varying concentrations (10-300 nM) of rhCD39-ECD-His (R&D Systems) for the association step. Dissociation of the complex was monitored upon exposure of the sensors to running buffer once again. Data was processed using ForteBio Octet software with baseline subtraction, global fit and 1:1 binding model to obtain association and dissociation rates. K_{D} was calculated from the ratio of k_{d} to kₐ.

Data shown in FIG. 1 had R²>0.980. PF=poor fit. The association and dissociation time course data was globally fit with a simple 1:1 Langmuir binding model to yield on-rate (kon) and off-rate (koff) values. The equilibrium dissociation constants (K_{D}) were calculated from the kon and koff values. The kon values ranged from 1.93E+04 to 1.72E+06 M⁻¹s⁻¹ and the off rate values ranges from 3.65E-01 to 1.11E-04 s⁻¹. The K_{D} values ranged from 4.09E-07 to 7.31E-011 molar indicating that all of the antibodies bound with moderate or high affinity to human CD39 ECD.

The paralog specificity of the anti-CD39 antibodies was assessed by biolayer interferometry using soluble recombinant human ENTDP2 and soluble recombinant human ENTDP3 (both from R&D Systems). ENTDP2 and ENTDP3 are enzymes with functions similar to CD39. None of the antibodies exhibited detectable binding to ENTDP2 or ENTDP3 (data not shown). Thus all of the antibodies exhibit specific binding to human CD39.

### Example 4: Inhibition of Recombinant Human CD39 Extracellular Domain

The inhibition of recombinant human CD39 ECD by anti-CD39 antibodies was measured as follows. Recombinant human CD39/ENTPD1 (4397-EN from R&D systems), (either 5 or 10 nM final concentration) was combined with anti-CD39 IgGs (0.25 or 1 micromolar final concentration) in 25 mM Tris, 5 mM CaCl₂), pH 7.5 in a 96-well plate and incubated at room temperature for 2 hrs. ATP (Sigma A1852-1VL) was then added to a final concentration of 500 micromolar and incubated at 37° C. for 60 minutes. The plate was then placed at room temperature and CellTiter-Glo Luminescent Cell Viability Assay solution was added to each well of the assay plate, mixed and read on a microplate reader using “CellTiter-Glo luminescent” preset. Control reactions consisting of negative control IgG, IgG only (no ATP), ATP only (no CD39) were run using the same method. Data values are the average of 2 replicates.

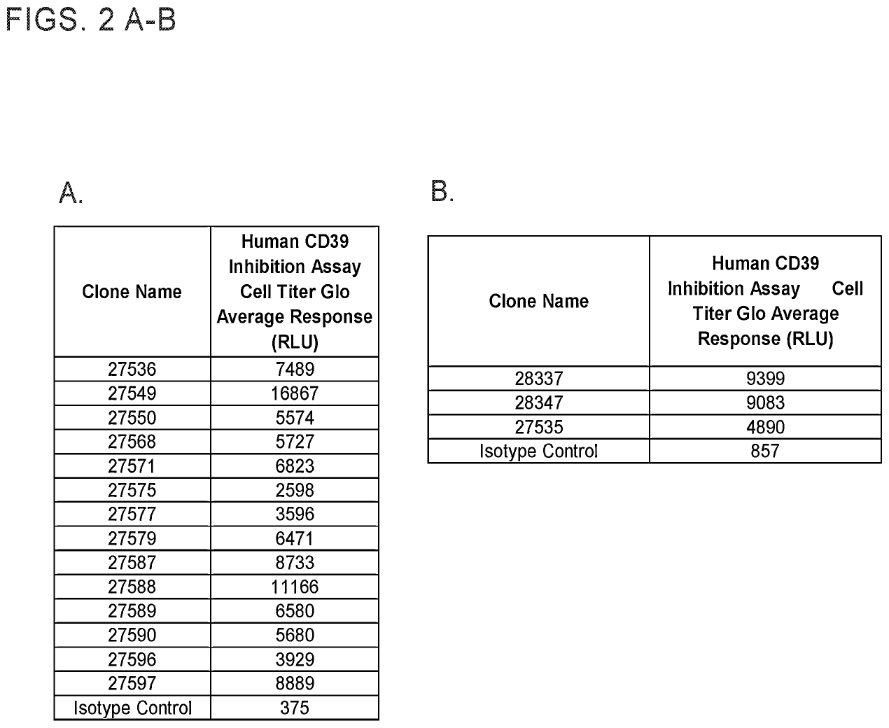

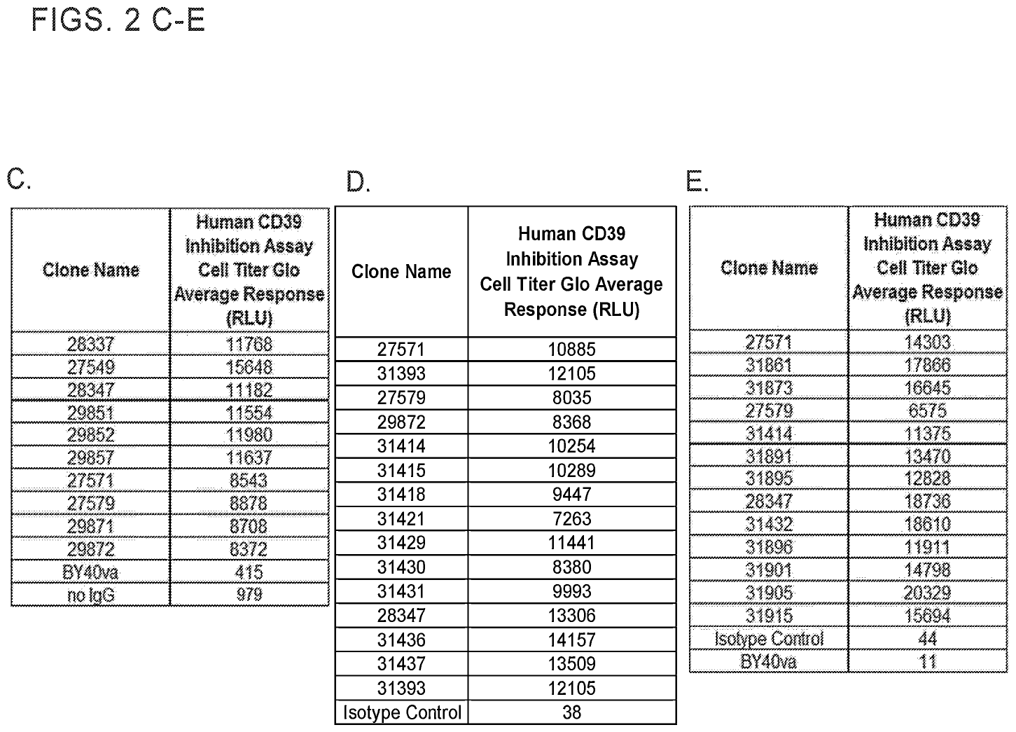

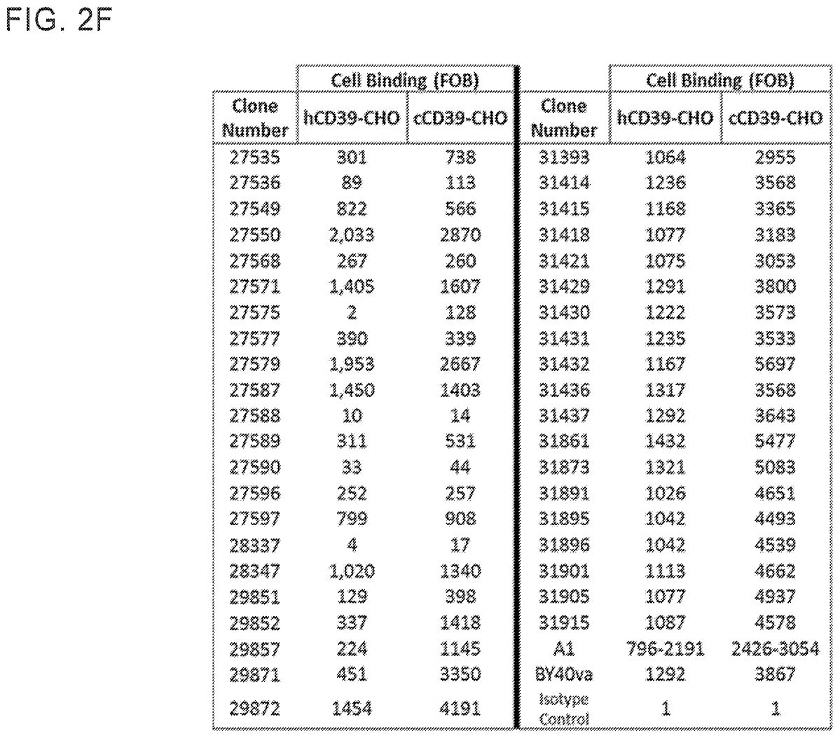

Inhibition of human CD39 ECD enzymatic activity by anti-CD39 antibodies was determined by measuring ATP levels using the CellTiter-Glo assay (FIGS. 2A-E). The enzymatic catabolism of ATP by CD39 ECD was observed in the presence of an isotype control antibody or no IgG with average RLU values ranging from 38 to 857. All of the anti-CD39 antibodies showed marked inhibition ATP catabolism by CD39, having much higher average RLU values than the isotype control antibody (average RLU values range from 4890 to 20329). In contrast, Benchmark antibody BY40va did not show significant inhibition of CC39 ECD in this assay having RLU values similar to the isotype control antibody (average RLU values 11 and 415).

### Example 5: Antibodies Bind to CHO Cells Expressing Human and Cyno CD39

Binding of anti-CD39 IgGs to Chinese Hamster Ovary K1 (CHO) CD39 cells. 100 nanomolar IgGs (each antibody is indicated as a unique clone number in the FIG.) were incubated at 25° C. for 30 minutes on ice in phosphate buffered saline (PBS) with parental CHO cells or CHO cells engineered to express either human or cynomolgus macaque (Macaca fascicularis) CD39 (CHO CD39 cells). Cells were then washed with ice cold PBS and incubated with a fluorescently labeled goat-anti-human IgG for 20 minutes on ice. Cells were washed and resuspended in ice cold PBS prior to analysis by flow cytrometry. Fold over background binding levels represent the ratio of median fluorescence intensity (MFI) values for anti-CD39 antibodies binding to CHO CD39 to MFI values for anti-CD39 antibodies binding to the parental CHO cells.

The anti-CD39 antibodies bound to CHO cells expressing cellular human CD39 (CHO) CD39 cells) and did not exhibit significant binding to parental CHO cells (FIGS. 2F-J). The binding of these antibodies to CHO CD39 cells ranged from 10 to 2033-fold over background. Antibodies 28337 and 27575 did not show significant binding to CHO CD39 cells (only 2 to 4-fold over background) (FIGS. 2F-J) indicating that these antibodies do not have low affinity for the cellular form of human CD39.

The ortholog specificity of the anti-CD39 antibodies was assessed with flow cytrometry using CHO) cells engineered to express either cynomolgus macaque or mouse CD39. The anti-CD39 antibodies bound to cynomolgus macaque CD39 to a similar extent as human CD39 (FIGS. 2F-J). None of the anti-CD39 antibodies showed detectable binding to mouse CD39. Thus, the anti-CD39 antibodies are cross reactive to cynomolgus macaque CD39 but not to mouse CD39.

### Example 6: Binding of Antibodies to Cell Surface CD39 in MEL-28 or 721 Cells and Antibodies That Inhibit CD39 On MEL-28 Cells A Short Term ATPAse Assay

Cells were incubated with serially diluted anti-CD39 antibodies for 30 minutes at 4 degrees C. Cells were washed 3 times in FACS buffer (PS, 2% FBS, and 2 mM EDTA) and next incubated with secondary antibody (goat anti-human IgG Southern Biotech) at 1:100 for 30 minutes at 4 degrees C. Cells were washed, resuspended in FACS buffer, and analyzed for binding by flow cytometry analysis on BD Celesta.

3.5×10⁴ MEL-28 cells/well were washed with Tris buffer and incubated with serially diluted (100-0.00013 nM) antibody for 30 minutes at 37 degrees C. 50 μM ATP was added to each well and incubated with cells for 15 minutes. The supernatants were collected and analyzed in Malachite Green Assay (R&D) according to manufacturer's protocol. Phosphate released from CD39 processing of ATP was used as a readout of enzyme activity. Palivizumab was used as an isotype control and ARL (Tocris) and POM-1 (Alpha Aesar), non-specific small molecule inhibitors of CD39, were used as positive controls at 100 μm.

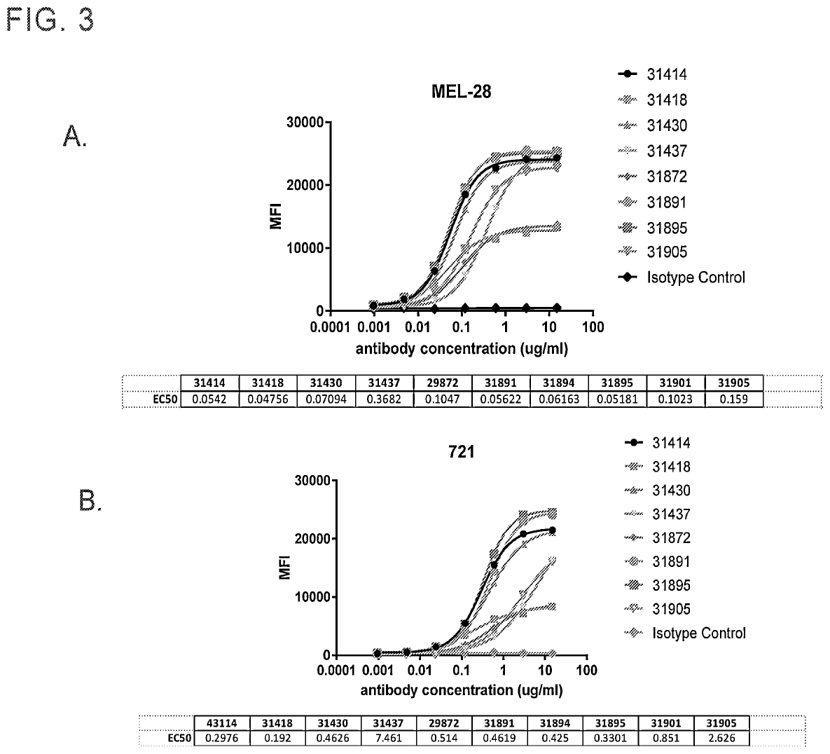

All antibodies bound to endogenously expressed CD39 on both cell lines with similar affinity with EC50 ranges from 0.05-0.28 μg/ml on MEL-28 (see FIG. 3A) and with EC50 ranges of 0.2-7.5 μg/ml on 721.22 cell line (see FIG. 3B). Maximum signal (MFI) differed between antibodies tested even when EC50 values were similar (FIGS. 3A-B).

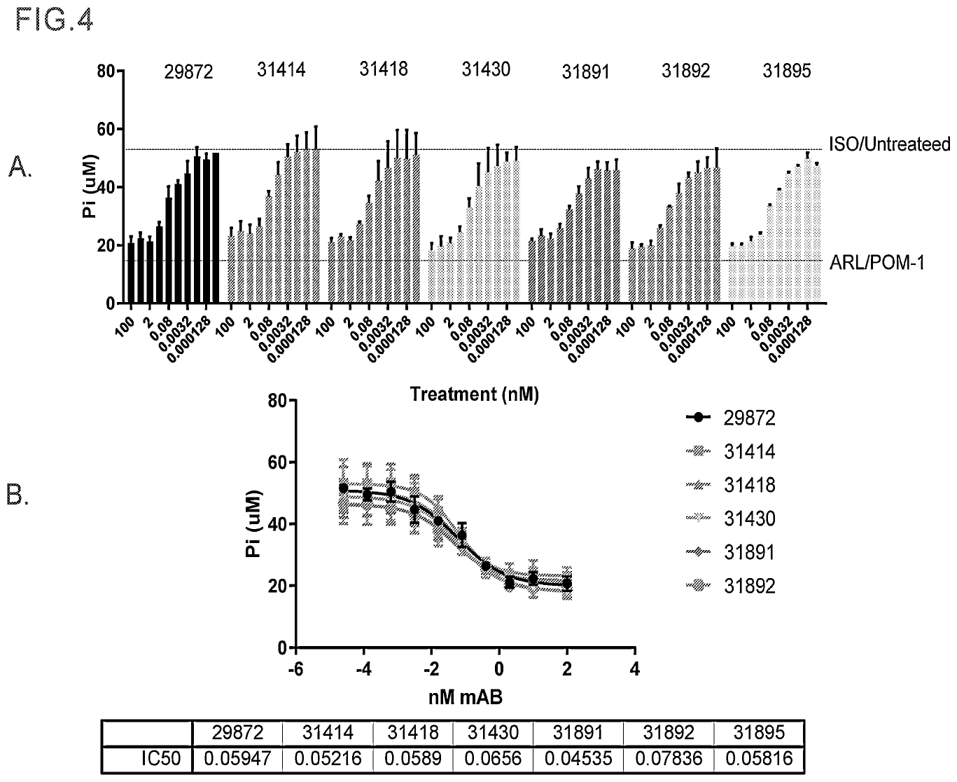

After confirmation of cellular binding, anti-CD39 antibodies were evaluated for inhibition of ATPase activity on MEL-28 cell in short term 30 minute Malachite Green phosphate readout assay. Isotype control was used to establish maximum possible signal from ATP processing in MEL-28 cells-50 μm ATP addition to cells typically resulted in 55-60 μM phosphate signal in this assay (see FIGS. 4A and B). Anti-CD39 antibodies inhibited ATPase activity in MEL-28 cells by 60-80% at the highest concentration of antibodies tested (100 nM) this level of inhibition was similar to non-specific ATPase inhibitors ARL and POM1 (see FIG. 4A). IC50 values for anti-CD39 antibodies in MEL-28 malachite green assay were all in sub-nanomolar range (see FIG. 4B).

### Example 7: ATP Preservation Quantified when MEL-28 Cells are Treated with CD39 Enzymatic Inhibitors

3.5×10⁴ MEL-28 cells were plated overnight at 37 degrees C. Cells were washed with Tris assay buffer to remove phosphate. 100 nM titrated down to 0.005 μM of monoclonal antibodies were incubated with cells for 30 minutes at 37 degrees C. 50 μM ATP was added and incubated for 15 minutes. Supernates were harvested and frozen. Supernates were thawed and evaluated for ATP using the EnzyLight (EnzyLight ATP Assay Kit, BioAssay Systems). Palivizumab was used as an isotype control and ARL (Tocris) and POM-1 (Alpha Aesar) used at a concentration of 100 μM are non-specific small molecule inhibitors of CD39 as positive controls.

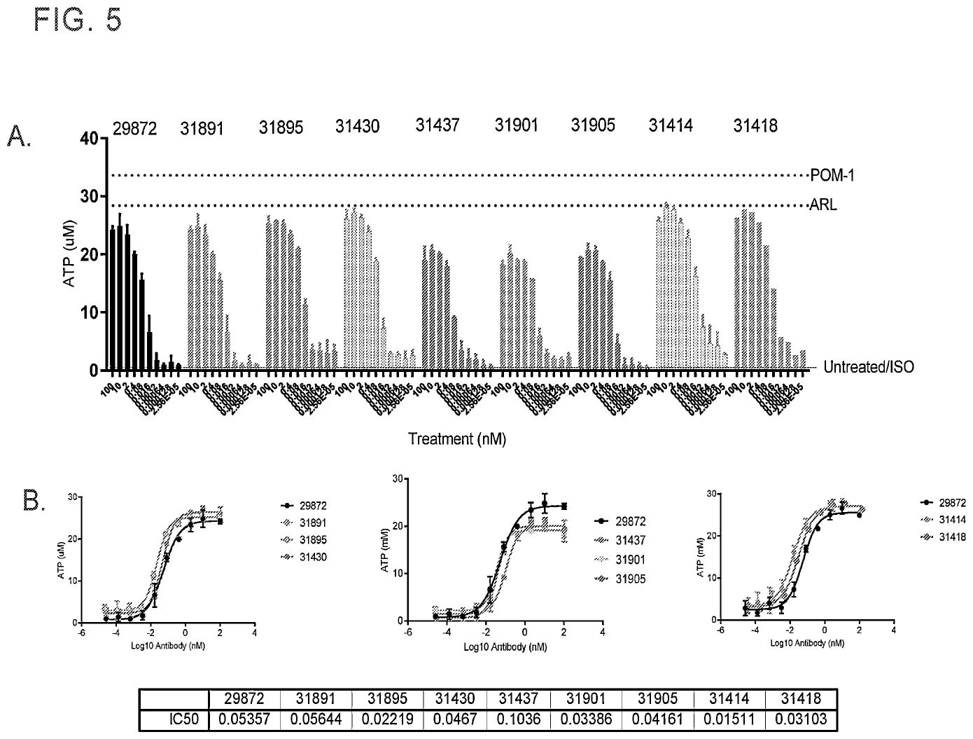

ATP was almost undetectable after 30 minutes post ATP addition to the cells in untreated and/or isotype treated samples (see FIG. 5A) while all of the anti-CD39 antibodies tested prevented processing of ATP in dose dependent manner (see FIG. 5A, B). Most of the anti-CD39 antibodies tested in this assay prevented ATP processing by CD39 to a similar extent as ARL (see FIG. 5A). IC50s of anti-CD39 antibodies in ATP preservation assay ranged from 0.02-0. InM. Overall potency of antibodies in this assay was consistent with what was observed in Malachite Green phosphate readout assay.

### Example 8: Antibodies Inhibit CD39 Activity On MEL-28 In An Overnight Assay

3.5×10⁴ MEL-28 cells/well were plated and incubated with antibodies overnight at 37 degrees C. Cells were washed to remove FBS. Cells next were pre-treated with antibodies in X-VIVO 15 FBS free media overnight at 37 degrees C. ATP was then spiked in at 50 μM for 15 minutes. Supernatants were collected and analyzed using AMP-Glo kit according to manufacturer's instructions (Promega). Palivizumab was used as an isotype control and POM-1 (Alpha Aesar), a non-specific small molecule inhibitor of CD39, was used as positive control at 100 μM.

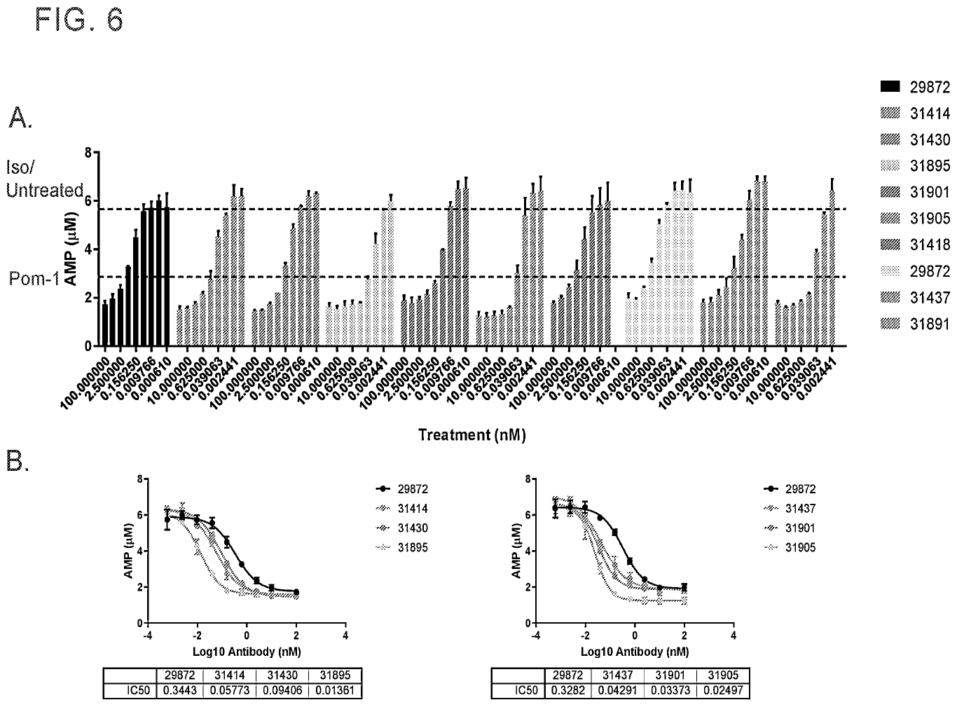

Anti-CD39 antibodies tested in overnight AMPGlo assay in MEL-28 cells demonstrated sustained inhibition of ATPase activity as indicated by decreased AMP levels present in the supernatants (see FIG. 6A). Inhibition of CD39 activity by antibodies was equivalent to or more potent compared to POM-1 treatment. The data is consistent with results obtained in CD39 short-term Malachite Green assay in MEL-28 (see FIG. 4). Antibodies tested in an overnight assay had IC50 values in an AMPGlo CD39 inhibition assay ranging from 0.01 to 0.3 nM. (see FIG. 6B)

### Example 9: Anti-CD 39 Antibodies Bind to Primary Human and Cyno B Cells

B cells were isolated from human donor leukopak using EasySep B cell isolation kit (STEMCELL Technologies). Cyno monocytes were purified from fresh cyno blood using NHP CD14 positive selection kit (Miltenyi) and flow through was collected and stained with CD4, CD8, CD20, CD16, and CD3 antibodies (BD). Human B cells or cyno cells were incubated with serially diluted anti-CD39 antibodies (15 μg/ml 7.5 fold serial dilution, 8-point) for 30 minutes at 4 degrees C. Cell were washed 3 times in FACS buffer (PS, 2% FBS, and 2 mM EDTA) and incubated with secondary antibody (mouse anti-human IgG southern biotech) at 1:100 for 30 minutes at 4 degrees C. Cells were washed 2 times in FACS buffer and resuspended in FACS buffer and analyzed on BD Celesta.

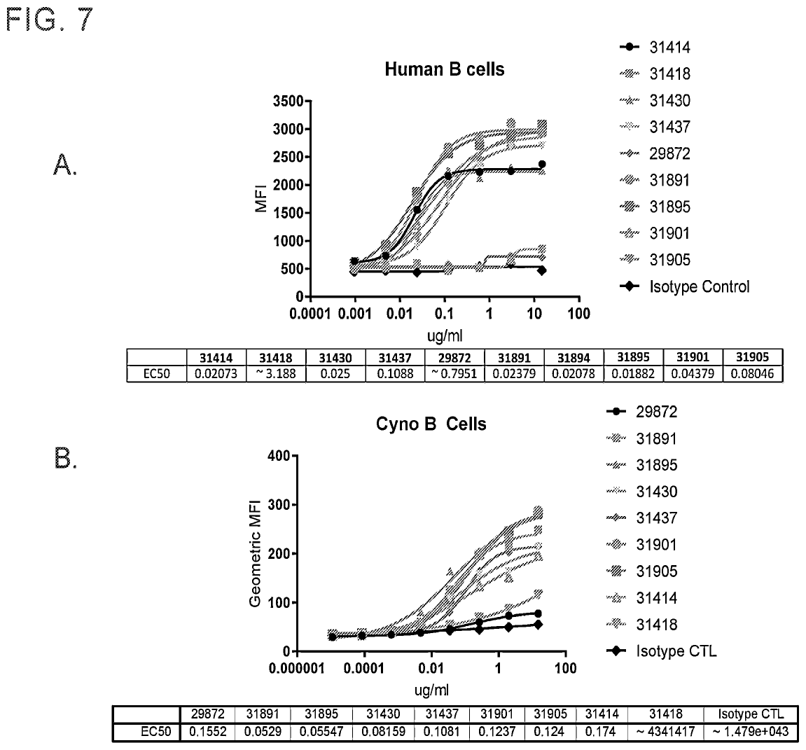

Detection of antibody binding is as described in FIG. 7 where B cells were incubated with serially diluted antibodies and detected using a fluorescently tagged antibody and analyzed by flow cytometry. The results are shown in FIG. 7 and appear to indicate that the antibodies bind specifically to both human and cyno B cells with EC50s that range from 0.02 μg/ml to 3.18 μg/ml (human) and 0.03 μg/ml to 0.17 μg/ml (cyno). Similar binding was observed on human tumor cells lines (FIG. 3) where subset of the antibodies had a low maximal MFI and a subset had a high MFI to both the human and cyno B cells.

### Example 10: Antibodies Inhibit CD39 Activity On Human B Cells

B cells were isolated from human leukopak using EasySep B cell isolation kit (STEMCELL Technologies). 5×10⁴ B cells/well were washed with Tris buffer and incubated with serially diluted (100-0.00013 nM) antibodies for 30 minutes at 37 degrees C. 50 μM ATP was added to each well and incubated with cells for 2 hrs. The supernatants were collected and analyzed in Malachite Green Assay (R&D) according to manufacturer's protocol. Phosphate released from CD39 processing of ATP was used as a readout of enzyme activity. Palivizumab was used as an isotype control and ARL (Tocris) and POM-1 (Alpha Aesar), non-specific small molecule inhibitors of CD39, were used as positive controls at 100 μM.

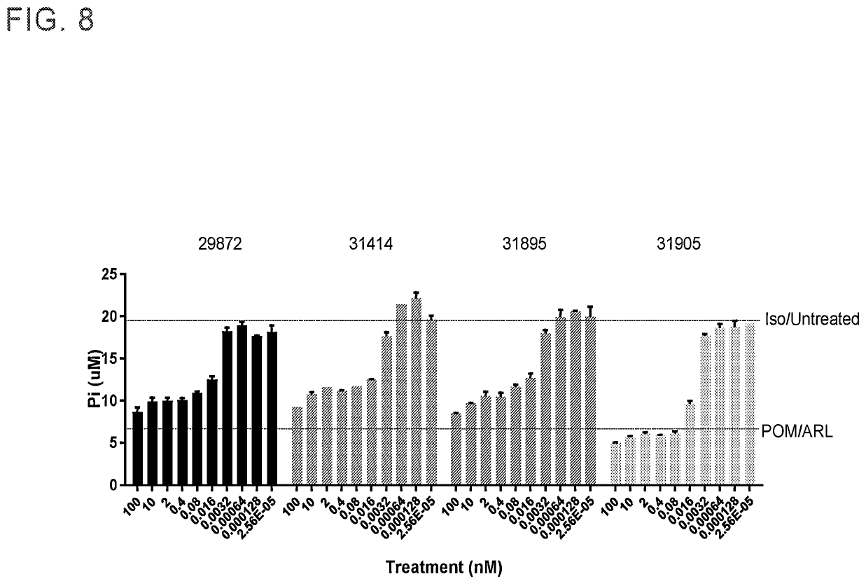

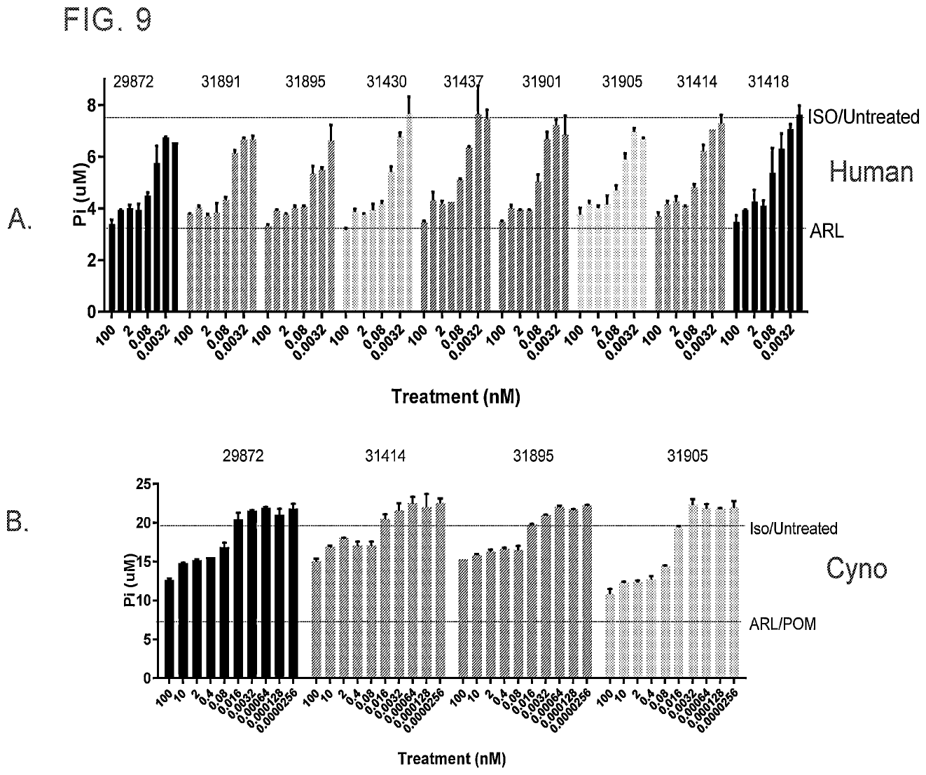

The antibodies were demonstrated to bind to primary human and cyno B cells (see FIG. 7) and the next step was to evaluate the inhibition of ATP hydrolysis by detection of free phosphate (Pi) using a malachite green assay. The results are shown in FIG. 8 and indicate the anti-CD39 antibodies inhibit the enzymatic inhibition/dephosphorylation of ATP by primary human B cells. The ability of the antibodies to inhibit enzymatic activity was comparable regardless of high vs. low max MFI detected in the binding to human B cells (FIG. 7).

### Example 11: Anti-CD39 Antibodies Inhibit ATPAse Activity On Human And Cyano Monocytes

Human monocytes were purified from leukopak using EasySep Human monocytes isolation kit (STEMCELL). Cyno monocytes were isolated from whole cyno blood using NHP CD14 positive selection kit (Miltenyi). Monocytes at 5×10⁴ cells/well were washed with Tris buffer and incubated with serially diluted (100-0.00013 nM) anti-CD39 antibodies for 30 minutes at 37C. 50 μM ATP was added to the cells for 15 minutes at 37C and supernatants were harvested and analyzed in Malachite Green Assay (R&D) for phosphate levels. Palivizumab was used as an isotype control and ARL (Tocris) and POM-1 (Alpha Aesar), non-specific small molecule inhibitors of CD39, were used as positive controls at 100 μM.

CD39 expression has been detected on human leukocytes with the highest expression detected on monocytes (Thromb Res. 2007; 121 (3): 309-17). Because of this information, it was important to evaluate the ability of the anti-CD39 antibodies to inhibit ATPase activity on the cell surface. As demonstrated in FIG. 7, anti-CD39 antibodies bind to both human and cyno B cells. It is appropriate to evaluate the inhibition of enzymatic activity on human and cyno monocytes. The results indicate that all the antibodies are able to inhibit ATPase activity of CD39 on human and cyno monocytes with similar potencies.

### Example 12: Anti-CD39 Antibodies Bind To Primary Human TRegs And Inhibit CD39 Enzymatic Activity

Treg cells were isolated from human donor leukopak using CD4⁺CD25⁺CD127^{dim} regulatory T cell isolation kit II (Miltenyi). Human Treg cells were incubated with serially diluted anti-CD39 antibodies (100 nM-0.00064 nM) for 30 minutes at 4 degrees C. Cell were washed 3 times in FACS buffer (PS, 2% FBS, and 2 mM EDTA) and incubated with secondary antibody (mouse anti-human IgG southern biotech) at 1:100 for 30 minutes at 4 degrees C. Cells were washed 2 times in FACS buffer, resuspended in FACS buffer and analyzed on BD Fortessa.

CD4⁺CD25⁺CD127^{dim} human Treg cells were washed 3× with Tris buffer. Cells were incubated with anti-CD39 antibodies (100 nM-0.00064 nM) for 30 minutes at 37C. The cells were spiked with 50 μM ATP and supernatants were collected after 15 minutes incubation at 37C. Supernatants were analyzed for phosphate levels in Malachite Green Assay kit (R&D). Palivizumab was used as an isotype control and ARL (Tocris) and POM-1 (Alpha Aesar), non-specific small molecule inhibitors of CD39, were used as positive controls at 100 μM.

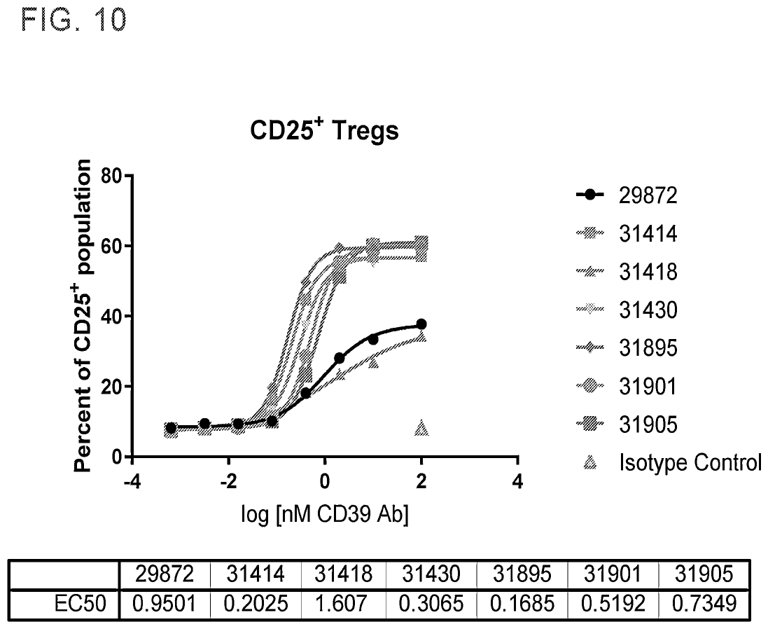

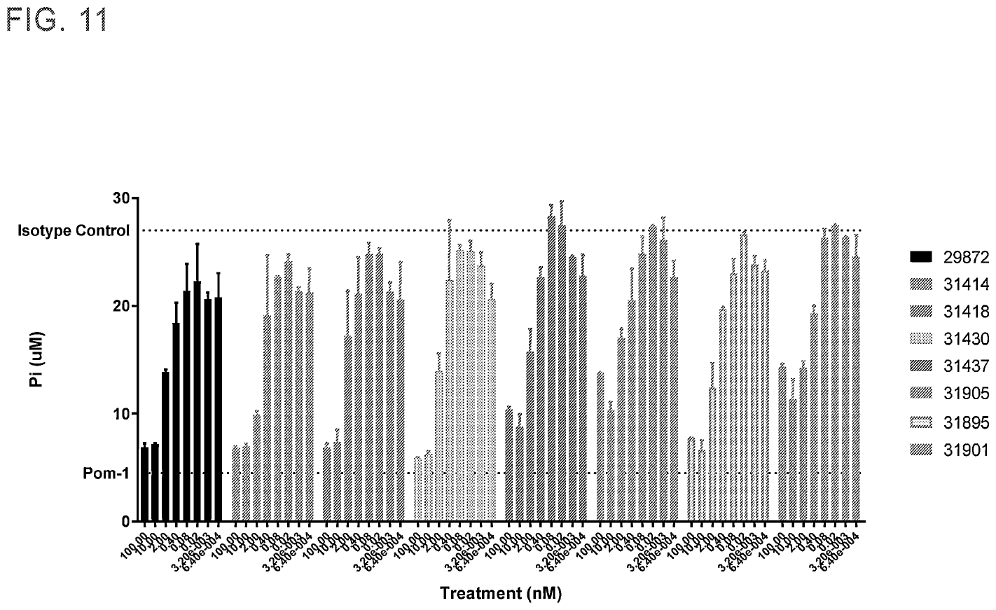

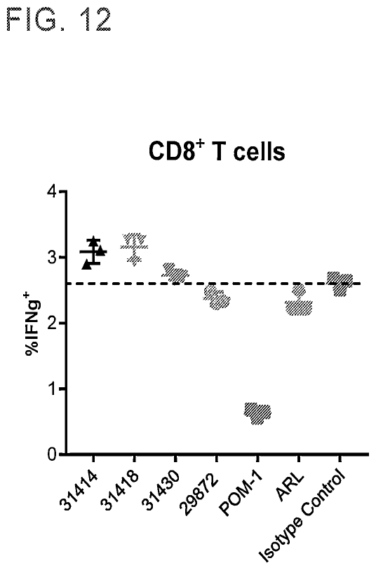

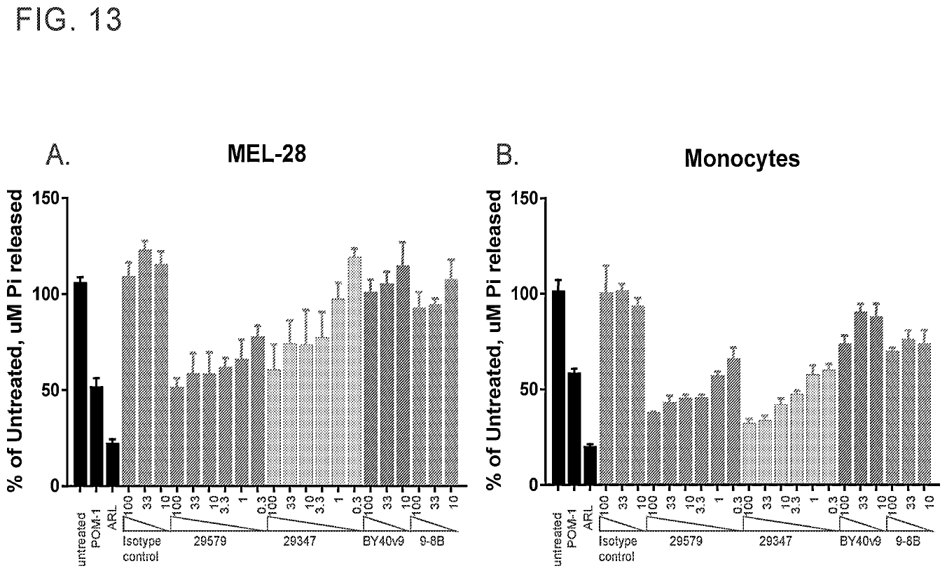

CD39 had been shown to be expressed on human regulatory T cells (Treg) and important for their suppressive function by hydrolysis of ATP to immune suppressive adenosine. (Blood. 2007 Aug. 15; 110 (4): 1225-32, Cellular Molecular Immunology (2017) 14, 521-528; doi: 10.1038/cmi.2016.30). In order to determine whether the anti-CD39 antibodies were capable of inhibiting CD39 enzymatic activity, it was important to evaluate the binding to human Tregs. Tregs were isolated from human PBMCs and the Tregs were purified and the anti-bodies were evaluated for binding by flow cytometry. The result indicate that the anti-CD39 antibodies bind to human Tregs (FIG. 10). Of note, both the high maximum MFI and low MFI profiles were observed similar to the human B cell staining (see FIG. 8). The ability of the antibodies to inhibit the ATPase activity on human Tregs was also evaluated. All the antibodies inhibit Treg CD39 enzymatic activity (see FIG. 11) equally well regardless of the maximal MFI staining observed.

### Example 13: Anti-CD39 Antibodies Increase CD8⁺ T Cell Response in a CMV Recall Response Assay

Frozen PBMCs are thawed and resuspended at 3×10⁶/ml and cultured in presence of CMV peptides (Miltenyi, PeptTivator CMV pp65) for 3 days in complete (10% FBS) media at 37 degrees C. T cells were then purified (STEMCELL, EasyStep) and rested for 24 hours at 37 degrees C. APCs were generated by depleting CD2 positive cells (STEMCELL, Easystep) from PBMC's from the same donor and plated at 5×10⁴/well overnight at 37 degrees C. The following day 5×10⁴ rested T cells were added to the APCs. Antibodies were added at 25 μg/ml plus 100 μM ATP+1 μM EHNA and Golgi plug/stop with CMV peptides and incubated at 37 degrees C. for 5 hours. T cells were stained and analyzed for intracellular IFN gamma on a Fortessa (Becton Dickinson) flow cytometer.

Adenosine has been shown to inhibit T cell activation. (Int J Oncol. 2008 March; 32 (3): 527-35). As the rate-limiting enzyme in ATP/ADP-AMP-adenosine pathway, inhibiting CD39 would diminish the levels of immune suppressive adenosine and increase immune activating ATP resulting in enhanced T cell activity. In order to evaluate the role of anti-CD39 antibodies inhibiting ATP/ADP-AMP hydrolysis, preventing the generation of adenosine which could result in an increased T cell response, a CMV recall assay was used. PBMCs were cultured in the presence of a pool of CMV peptides for 3 days and then the T cells were purified cultured with autologous PBMC plus CMV peptides and ATP+EHNA. After 5 hours the T cells were evaluated for the production of IFN gamma. The results indicate that some of the anti-CD39 antibodies were able to increase CD8⁺ T cells activity in a CMV recall responses assay. This demonstrates that inhibiting the enzymatic activity of CD39 prevents the generation of adenosine and preserves ATP levels and allowing for a robust T cell response to a peptide: MHC complex. In addition, not all antibodies that bind to CD39 and inhibit cell surface enzymatic activity are capable of increasing T cell response and the specific interaction of the antibody to CD39 is important.

### Example 14: Evaluation of Antibodies for Inhibition of CD39 Activity on Mel-28 and Human Monocytes

3.5×10⁴ MEL-28 cells/well were incubated with 100 nM down to 0.32 pM of monoclonal antibodies for 30 minutes. 50 μM ATP was added and incubated for 15 minutes. Supernate was evaluated for free phosphate (Pi) using the Malachite Green Phosphate Detection Kit (R&D Systems cat #DY996). Palivizumab was used as an isotype control and ARL (Tocris) and POM-1 (Alpha Aesar) used at a concentration of 100 μM as non-specific small molecule inhibitors of CD39 as positive controls. Cyno monocytes were isolated from whole cyno blood using NHP CD14 positive selection kit (Miltenyi). Monocytes at 5×10⁴ cells/well were washed with Tris buffer and incubated as described above.

When comparing anti-CD39 antibodies for enzymatic inhibition in short term assays, differences were observed. Some were able to block the release of free phosphate (Pi) measured in a malachite green assay and others demonstrated very little activity. For example, BY40v9 and 9-8B have little to no enzymatic inhibition at concentrations of 100 nM using both the MEL-28 cell or primary human monocyte compared to other anti-CD39 antibodies (e.g., 29579 and 28347) that are able to inhibit ATPase activity at low nM concentrations.

9-8B was produced as described in US 2017/0335007 A1, using SEQ ID Nos. 22 and 23, as hlgG4. BY40v9 is an engineered variant of the antibody BY40 described in WO 2009/095478 A1 SEQ ID Nos. 1 and 5. We tried to express BY40 as described as a human IgG4 but repeated attempts failed. The VH described in SEQ No. 1 appears to be missing several N-terminal residues compared to germline V_{H}'s, so we engineered in the missing residues with closest germline sequence from IMGT (http://imgt.org/), resulting in BY40-v9, which expressed and was determined to specifically bind rhCD39-ECD and cellular ECD.

### Example 15: Design of Chimera and Region Of Distinct Binding

(a) Examples of Antibodies that Bind Soluble Recombinant CD39 ECD and Cellular CD39 but do not Inhibit ATPase Activity and do not Compete with Cellular Inhibitors for Binding to ECD Inhibitors

0.04-3.3 nM rhCD39-ECD (R&D Systems) was incubated with buffer, 50 μg/ml antibody or 100 μM POM-1 (Alpha Aesar) for 1 hr at 37° C. in assay buffer (25 mM Tris pH 7.5, 5 mM CaCl₂) at which point ATP (Sigma) was spiked in to a final concentration of 10 μM, and the reaction further incubated for 30 min 37° C. Production of free phosphate (Pi) was subsequently measured using Malachite Green Phosphate Detection kit (R&D Systems).

Indicated cells were incubated in assay buffer with 10-25 μg/ml antibody, 100 μM POM-1 or 100 μM ARL for 30 min at 37° C. 5% CO₂. ATP was added to a final concentration of 50 μM and the incubation continued for 15 min. Supernatant was evaluated for free phosphate (Pi) using the Malachite Green Phosphate Detection kit. Palivizumab is used as an isotype control and POM-1 and ARL are non-specific small molecule inhibitors of CD39.

A1 antibody was immobilized onto an Anti-Mouse IgG Fc Capture (AMC) biosensor (ForteBio). Association of hCD39-ECD was then monitored for 180 seconds via Bio-Layer Interferometry (BLI) using the Octet system (ForteBio), at which point the biosensor was dipped into competitor antibody and monitored for another 180 seconds. Association of the second antibody was recorded as an upward shift in the interference pattern and indicates that the antibodies bind to different epitopes on CD39. No change in the interference pattern indicated that A1 blocks the second antibody from binding to CD39.

Although A1 does bind hCD39 ECD (FIG. 1), it does not inhibit its ATPase activity. Although A1 does bind cellular CD39 (FIG. 2), it does not appreciably directly inhibit the ATPase activity of CD) 39 expressed on OAW42. Capture of hCD39 ECD by A1 blocks subsequent binding by A1 (FIG. 14A, bottom right hand sensorgram), but does not block binding of inhibitory antibodies such as 27536, 27571, 27579, 27597, or 38347.

The commercially available A1 antibody represents a group of anti-human CD39 monoclonal antibodies that do not directly inhibit the ATPase activity of CD39 and do not bin with any of the other anti-CD39 antibodies described here. More than 30 antibodies were discovered that do not inhibit ECD ATPase activity, do not inhibit cellular CD39 ATPase activity, but do compete with A1 for binding to ECD. These antibodies may be considered to bin with anti-hCD39 antibody A1.

(b) Example of Antibodies that have Limited Ability to Inhibit the ATPase Activity of Both Soluble Recombinant and Cellular CD39 and Bin Separately from Other Cellular CD39 Inhibitors

The methods are the same as set forth above except that instead of A1, the representative antibody was immobilized using anti-human IgG Fc Capture (AHC) biosensor.

Anti-hCD39 antibodies exemplified by 27536 and 28337 represent a group of antibodies that bind both soluble ECD and cellular CD39 and have the ability to inhibit their ATPase activity yet do not compete with other inhibitors for CD39 binding. Both 27536 and 28337 are able to inhibit the hydrolysis of ATP by hCD39 ECD compared to isotype or buffer controls as shown by μM Pi, as described herein (top left panel). 27536 and 28337 inhibit the hydrolysis of CD39 expressed on MEL-28 and OAW42 cells, as shown, compared to isotype control. Antibodies 27536 and 28337 represent a distinct bin group of anti-hCD39 inhibitory antibodies since they are blocked from binding CD39 ECD by another bin member that does not block other inhibitory antibodies such as 27571, 27579, 27597, or 38347 (see insert (c) at the bottom of FIG. 14B).

(c) Example of Antibodies that Inhibit the ATPase Activity of Soluble Recombinant CD39ECD but do not Inhibit Cellular CD39 and Bin Separately from Other CD39 ECD Inibitors

The methods are as provided above, with the addition of experimental design where inhibition of 0.37 nM CD39 by 50 μg/ml antibody was challenged by ATP concentrations of 3-100 μM. Also, an 18 hour incubation was used with the antibody or isotype control. Finally, an additional 30 second baseline dip in buffer between antigen capture and dipping into a second competitor antibody for 300 seconds was used.

Anti-hCD39 antibodies exemplified by 27549 represent a group of antibodies that can bind ECD and cellular CD39 but can only inhibit ECD ATPase activity and not cellular CD39 ATPase activity. As can be seen in FIG. 14C (top left), 27549 is able to inhibit the hydrolysis of ATP by hCD39 ECD compared to isotype or buffer controls. Also from FIG. 14C (top right), 27549 is unable to inhibit the hydrolysis of CD39 expressed on MEL-28 cells compared to isotype control. Antibodies 27536 and 28337 represent a distinct bin group of anti-hCD39 inhibitory antibodies since they are blocked from binding CD39 ECD by another bin member that does not block other inhibitory antibodies such as 27571, 27579, 27597, or 38347 (see FIG. 14C, bottom).

Antibody 27549 represents a group of anti-human CD39 monoclonal antibodies that directly inhibit the ATPase activity of sol CD39 ECD yet does inhibit cellular CD39 despite being able to bind to CD39 expressed on cells (FIG. 2). It does not compete with A1 or with any of the other inhibitory anti-CD39 antibodies described here for binding to ECD, so 27549 represents another bin of anti-hCD39 antibodies (see FIG. 14C).

(d) Example of Antibodies that Inhibit the ATPase Activity of ECD and Cellular CD39 and Bin Separately from Other CD39 ECD and/or Cellular Inhibitors

The methods are as provided above.

Antibodies 27571, 27579 and 28347 represent a group of anti-human CD39 antibodies that directly inhibit the ATPase activity of soluble CD39 ECD and inhibit cellular CD39 and yet do not compete with A1, cellular inhibitors 27536 or 28337, or ECD inhibitor 27549 (FIGS. 14A-C) for binding to ECD. 27571, 27579, and 28347 are able to inhibit the hydrolysis of ATP by hCD39 ECD compared to isotype or buffer controls (see FIG. 14D, left). 27571, 27579, and 28347 also inhibit the hydrolysis of CD39 expressed on MEL-28 cells compared to isotype control (see FIG. 14D, right). These cellular inhibitors represent another bin of anti-hCD39 antibodies (see FIG. 14D).

(e) Examples of Inhibitory Antibodies that Make Distinct Contacts with CD39

Chimeras were generated by replacing human CD39 with mouse CD39 sequence in mammalian expression vectors. The chimeras were expressed in CHO cells and the ability of anti-human CD39 antibodies to bind the chimeras tested via FACS. In brief, cells were washed and blocked in FACS buffer (PBS/2% FBS) for 30 min on ice. They were then incubated with 15 μg/ml anti-human CD39 antibodies diluted in FACS buffer×1 h on ice. After 2 washes, cells were incubated with fluorescently labeled anti-human Fc antibodies, anti-mouse Fc antibodies, or anti-mCD39 antibody (R&D) Systems 495826) diluted in FACS buffer per manufacturers' instructions×30 min on ice. After 2 washes, cells were resuspended in FACS buffer and analyzed on BD Fortessa. Data was processed with FlowJo. Positive binding was scored as “yes” and lack of binding was scored as “NO” and examples of FACS plots used to generate this table are in FIGS. 14.F and 14.G.

Chimeras formed between human and mouse CD39 distinguish regions critical for antibody contact that are unique to inhibitory antibodies such as 31414, 31895, 31873, 31901, 31905, reference antibodies such as BY40v9 and 9-8B, and commercial antibodies such as A1 and 498403. Columns 1 and 3 describe the human CD39 sequences flanking the mouse CD39 sequence described in column 2. Column 4 lists the exact mouse amino sequence in the chimera.

The antibodies described thus far have no/minimal cross-reactivity to mouse CD39, which shares 78% percent identity with human CD39 in the extracellular domain. Chimeras were therefore made between human and mouse CD39 in order to identify those region(s) critical to antibody recognition and binding. To that end, 8 chimeras were generated with the sequence swaps chosen based on sequence diversity between human and mouse CD39 and potential for surface exposure based on rat ENTPD1 and rat ENTPD2 crystal structures. Thus, these 8 chimeras do not comprehensively interrogate all potential contact residues. All tested antibodies were able to bind Chimeras #1,3,5,6,7, and 8, suggesting that these chimeras maintained overall global structural integrity. A1 and 498403 lost the ability to bind Chimera #4, indicating that residues critical to their contact with human CD39 had been lost. A1, 498403, BY40v9 and 9-8B were able to bind Chimera #2, suggesting that this chimera maintained overall global structural integrity. In contrast, inhibitory antibodies 31414, 31895, 31901, 31905, and 31873 were able to bind all chimeras but Chimera #2. Thus, Chimera #2 has lost residues critical to making contacts with these antibodies and therefore E143-N158 constitute part or all of the human CD39 epitope for these antibodies. Notably, these antibodies belong to the bin group exemplified by 27571, 27579 and 28347 in FIG. 14D, i.e. they do not bin with anti-CD39 antibody bin groups represented by antibodies A1, 27536, or 27549.

Chimeras were generated by mutagenesis of Chimera #2 or WT hCD39 expression vectors. Residues for mutagenesis were chosen based on divergence between human and mouse CD39. The chimeras were expressed in CHO cells and the ability of anti-human CD39 antibodies to bind the chimeras tested via FACS, as described in FIG. 14E. Table 2. Positive binding was scored as “Yes” and lack of binding was scored as “No” and examples of FACS plots used to generate this table are in FIGS. 14.F and 14.G.

Specific residues critical for antibody contact can distinguish inhibitory antibodies 31414, 31418, and 31895 from 31901, 31905, and 31873. Individual amino acids in the mouse sequence of human-mouse Chimera #2, when reverted back to the respective human residue (resulting in Chimera #9,10), restore the ability of antibodies 31895, 31414, 31418 to bind to CD39 but not the ability of 31901, 31905, or 31873 to bind CD39. Mutation of individual residues in the context of otherwise fully human CD39 (Chimeras #11, 12) did not impair the binding ability of any of the tested antibodies. Co-mutation of two human residues to the cognate mouse residues (Chimeras #13, 14) did not impair binding by antibodies 31414, 31418 or 31895. In stark contrast, antibodies 31901, 31905, and 31873 completely lost their ability to bind Chimeras #13 and 14. The ability of indicated antibodies to bind a given chimera was determined by FACS. In particular, FIG. 14F and FIG. 14G show representative FACS plots used to populate this table and FIG. 14G shows the importance of human CD39 residues N99, R154, and/or S153 in recognition by antibodies represented by 31901, 31905, and 31873.

As demonstrated in FIG. 14E, Table 1, inhibitory antibodies 31414, 31418, 31895, 31901, 31905 and 31873 can be distinguished from other monoclonal anti-CD39 antibodies based on their inability to recognize Chimera #2. In Table 2, these antibodies are further distinguished by the gain or loss of the ability to bind specific point mutants of hCD39 or Chimera #2. Thus, these antibodies, while binding the same general region of human CD39, form distinct contacts with different critical residues in hCD39. Since these antibodies share the requirement for residues E143-N158, they may share some common contact residues yet can be differentiated based on the demonstrated unique points of contact.

(f) FACS Plots Highlighting the Importance of Human CD39 Residue D150 and/or E153 in Recognition of Antibodies Represented by 31414, 31418, and 31895 and FACS Plots Highlighting the Importance of Human CD39 Residues N99, R154, and/or E153 in Recognition by Antibodies Represented by 31901, 31905, and 31873

The methods are as provided above for the examples of inhibitory antibodies that make distinct contacts with CD39.

The ability of indicated antibodies to bind to a given chimera was determined by FACS.

While antibodies 31414, 31418, 31895, 31873, 31901, and 31905 all share a common requirement for human CD39 residues E143-N158, these antibodies make different critical contacts with residues within and outside of this region. Thus, antibodies, which belong to the bin group represented, by antibodies 27571, 27579, and 28347 (FIG. 14. D) can be further distinguished into at least two more groups based on sensitivity to distinct residues. There are at least 5 bin groups: (1) A1-like antibodies that compete each other for ECD binding yet do not inhibit ECD activity, but do not compete with antibodies in bin groups 2,3,4, or 5 for ECD binding and make critical contacts with N275-1277 as evidenced by loss of binding to Chimera #4; (2) 27536/28337-like antibodies that compete each other for ECD binding and can inhibit both ECD and cellular CD39 ATPase activity, but do not compete with antibodies in bin groups 1,3,4 or 5 for ECD binding; (3) 27549-like antibodies that compete each other for ECD binding and can inhibit CD39 ECD but not cellular CD39, and do not compete with antibodies in bin groups 1,2,4 or 5 for ECD binding; (4) 31414/31418/31895-like antibodies that make critical contacts with E143-N158 in cellular CD39 including (but not limited to) D150 and E153, inhibit the ATPase activity of both ECD and cellular CD39, compete with each other for ECD binding, but do not compete with antibodies in bins 1, 2 or 3 for ECD binding; and (5) 31873/31901/31905-like antibodies that make critical contacts with E143-N158 in cellular CD39 including (but not limited to) E153 and R154, as well as a sensitivity to residue N99, inhibit the ATPase activity of both ECD and cellular CD39, compete with each other for ECD binding, but do not compete with antibodies in bins 1, 2, or 3 for ECD binding. Thus, there are anti-human CD39 antibodies that can be distinguished based on their affinity for ECD, affinity for cells, ability to inhibit ECD ATPase activity, ability to inhibit cellular ATPase activity, and points of contact with CD39. For example, 27549 binds both ECD (FIG. 1) and cells well (FIG. 2) yet can only inhibit ECD ATPase activity. 27579 binds ECD weakly but is a potent cellular CD39 inhibitor.

### Example 16: Anti-CD39 Antibodies are Reversible Allosteric, not Competitive, Inhibitors Due to Vₘₐₓ Suppression of Vₘₐₓ

Anti-CD39 antibodies or isotype control antibody (100 nanomolar final concentration) were incubated with MEL-28 cells (35,000 MEL-28 cells/well) endogenously expressing human CD39 at 37° C. for 20 minutes in the presence of EnzChek reagents (PNP & MESG). Immediately following the addition of ATP (final concentrations ranging from 0-450 micromolar), the rate of ATP hydrolysis to free phosphate (Pi) by CD39 was monitored over time at Abs360 nm using SpectraMax i3× plate reader. The initial enzyme velocity, v0, was determined from the linear region of Pi vs. time curve for each ATP concentration. The plot of v0 vs. [ATP] was curve fit using non-linear regression of the Michaelis-Menten kinetic model.

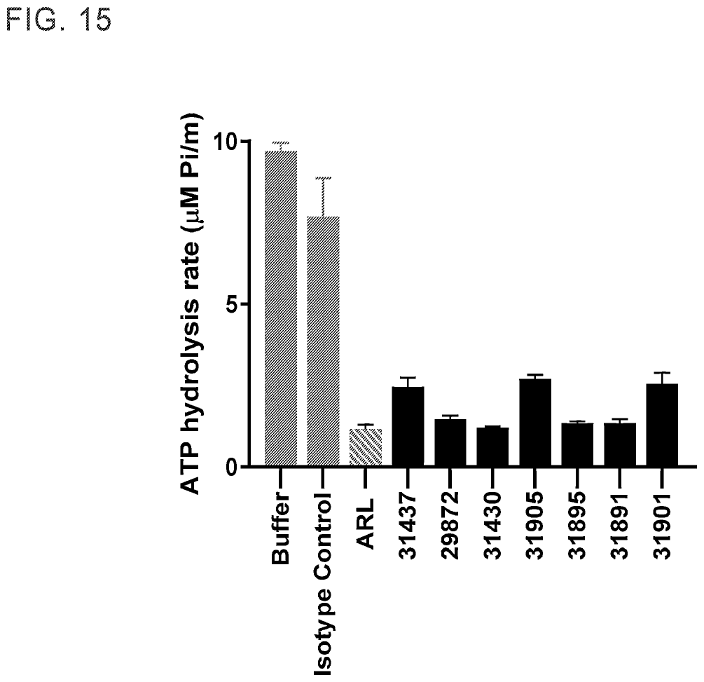

The ATP hydrolysis rate by CD39 expressed on MEL-28 cells can be markedly reduced by the anti-CD39 antibodies (see FIG. 15). The ATP hydrolysis rate in the presence of the anti-CD39 antibodies ranges from 1.2 to 2.7 micromolar Pi per minute, which is much less than the ATP hydrolysis rate observed in the presence of an isotype control antibody (7.7 micromolar Pi per minute) and similar in magnitude to the pan ATPase inhibitor ARL (1.2 micromolar Pi per minute).

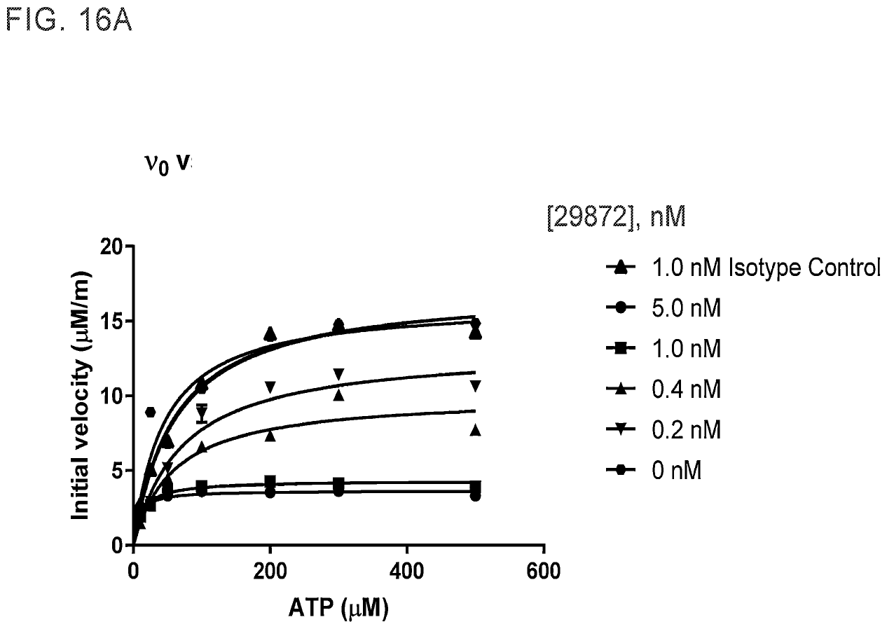

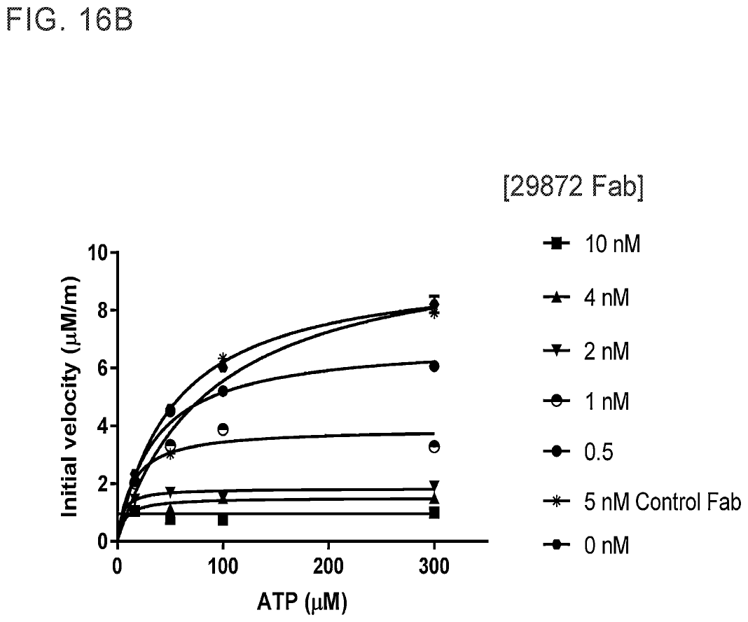

The plot of initial velocity versus ATP concentration for multiple concentrations of the anti-CD39 antibody 29872 indicates that the mechanism of inhibition of this IgG antibody is not competitive (see FIG. 16A.). The reduction in initial velocity is constant for ATP concentrations above 100 micromolar (100, 200, 300 and 500 micromolar) in the presence of 5 nanomolar 29872, 29872 could be a non-competitive inhibitor, an un-competitive inhibitor, or a mixed inhibitor of CD39 (having properties of both non-competitive and un-competitive inhibition). The monovalent Fab form of anti-CD39 antibody 29872 showed a very similar inhibition profile (see FIG. 16B.), indicating that the CD39 inhibition activity of 29872 is not dependent on the bivalent structure of the IgG and the potential properties that could result from IgG bivalency such as CD39 crosslinking.

Many of the other anti-CD39 antibodies tested in this assay showed a similar profile for CD39 enzymatic inhibition (data not shown), indicating that they also may be non-competitive inhibitors, un-competitive inhibitors, or mixed inhibitors of CD39. The mechanism of enzymatic inhibition of these antibodies suggests that they retain their full inhibition of CD39 enzymatic inhibition at high ATP concentrations. The anti-CD39 antibodies appear to be reversible inhibitors.

### Example 17: Anti-CD39 can Induce Internalization of CD39 on Cyno Monocytes

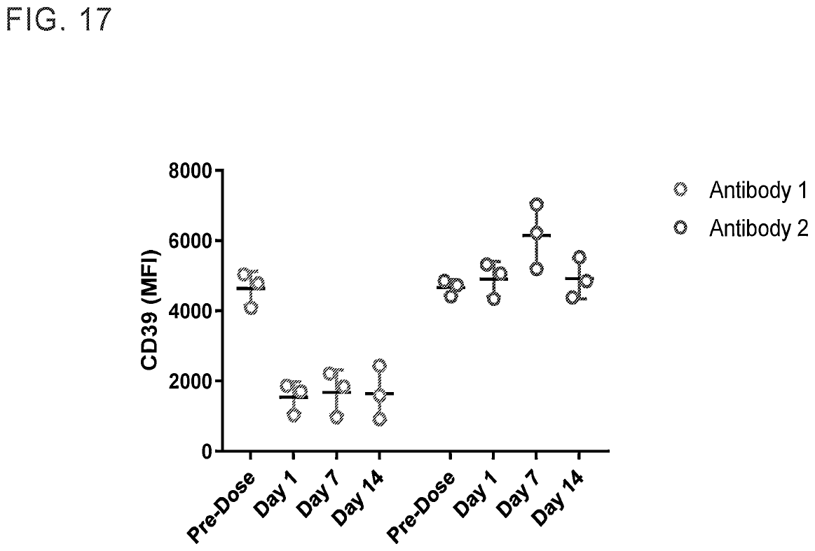

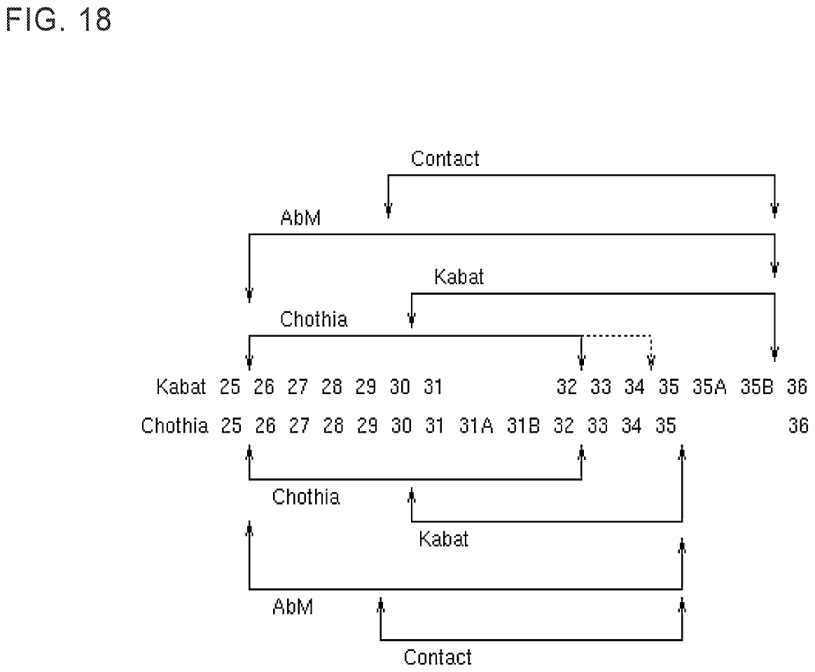

Anti-CD39 antibodies were injected into cynomolgus monkeys to test for ability to downregulate CD39 on cell surface of cyno monocytes. CD39 internalization was assessed by FACS of whole blood samples collected pre-dose, Day 1, Day 7, and Day 14 after antibody treatment. Non-competing anti-CD39-PE antibody (clone A1) was used to measure CD39 levels on CD14+ gated monocytes. Data is shown as MFI of A1-PF on gated cyno monocytes in FIG. 17.

Cynomolgus monkeys were injected with anti-CD39 antibodies at 10 mg/kg. Blood samples were collected prior to injection and on Day 1, Day 7, and Day 14 after treatment. For each time-point, 50 uL of cynomolgus whole blood was incubated with 30 uL of staining buffer (PBS, 2% FBS, 2 mM EDTA, 3% mouse serum, 5% goat serum) containing CD14 and CD20 antibodies (eBioscience) and an Fc blocking reagent (BD) for 30 minutes at 4C. CD39 antibody (clone eBioA1, eBioscience) was added to the sample and incubated for an additional 40 minutes at 4 degrees C. Following incubation, the samples were treated with ACK lysis buffer (ThermoFisher) for 10 minutes at room temperature to lyse red blood cells. Samples were washed several times with staining buffer and fixed with 1% PFA (Sigma) before acquisition on a BD Fortessa X-20 flow cytometer. Total CD39 receptor expression on CD14 positive monocytes was determined by mean fluorescence intensity.

CD39 was highly expressed on cyno monocytes prior to antibody treatment in all blood samples tested (pre-dose). Anti-CD39 antibodies tested in vivo had distinct internalization profiles where antibody 1 treatment led to downregulation of CD39 on cell surface of monocytes and antibody 2 had no effect on overall CD39 levels. Apparent decrease of CD39 levels after antibody 1 treatment was not due to A1 antibody competing with antibody 1 for binding to CD39 as the epitopes for A1 and antibody 1 are distinct from each other.

### Example 18: Anti-CD39 Antibody Increases Stimulated Human CD4+ and CD8⁺ T Cell in the Presence of Exogenous ATP

Anti-CD3+anti-CD28 stimulated PBMCs were treated with anti-CD39 antibody 31895 or isotype control in the presence of 50 μM ATP for 96 hours. Proliferation of CD4⁺ and CD8⁺ T cells was measured by Cell Trace Violet by flow cytometry.

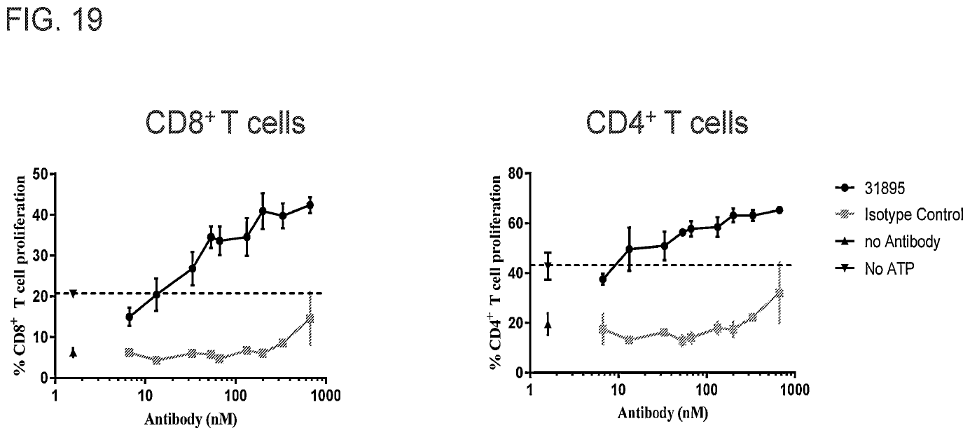

FIG. 19 shows that anti-CD39 antibody increases proliferation of stimulated human CD4⁺ and CD8⁺ T cell in the presence of exogenous ATP. The left side shows CD8⁺ T cells, with the x-axis showing antibody (nM) and the y-axis showing % CD8⁺ T cell proliferation. The right side shows CD4⁺ T cells, with the x-axis showing antibody (nM) and the y-axis showing % CD4⁺ T cell proliferation. The inset to the right shows symbols for the respective antibodies and controls.

### Example 19: Anti-CD39 Antibody Increase Stimulated PBMC Secretion of INF-γ, TNF-α and IL-2

Human PBMCs were treated with anti-CD3+anti-CD28 and incubated with anti-CD39 antibody 31895 or isotype control in presence (B) or absence (A) of exogenous ATP (50 μM). Supernatants were harvested after 96 hours and cytokines were measured using a Meso Scale Discovery human cytokine kit.

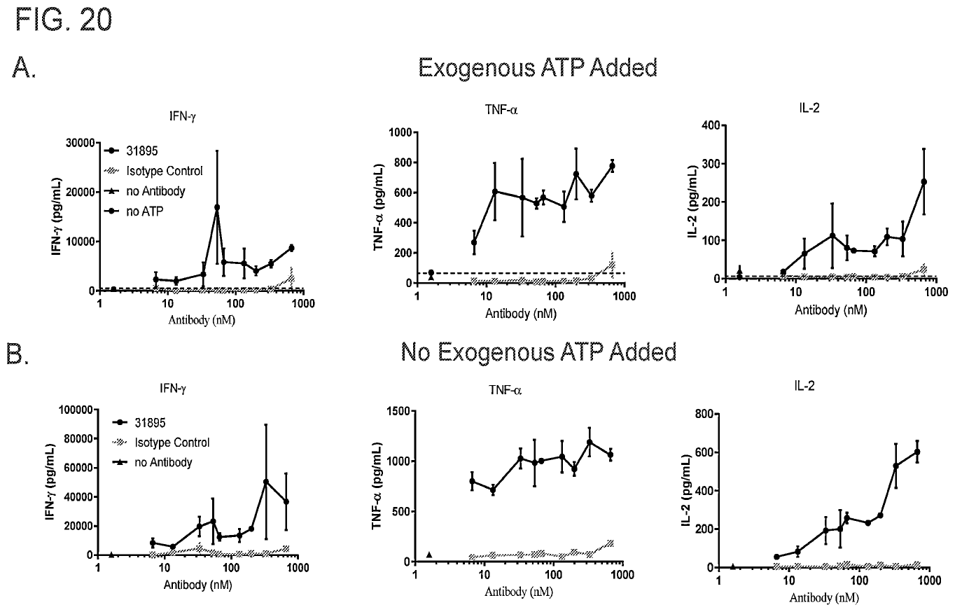

FIG. 20 shows anti-CD39 antibody 31895 increases cytokine secretion by anti-CD3+anti-CD28 activated PBMC in absence (A) or presence (B) of exogenous ATP in a dose dependent manner. The top row shows results with exogenous ATP added and the bottom row shows results with no exogeonous ATP added. The x-axis shows antibody (n M) and the y-axis shows secretion of INF-γ, TNF-α and IL-2, respectively.

### Example 20: Anti-CD39 Antibody Increases Stimulated PBMC Secretion of INF-γ, TNF-α, IL-2 and IL-1β

Human PBMCs were stimulated with anti-CD3+anti-CD28 and incubated with anti-CD39 antibodies 31895, HAO-391 (See, SEQ ID 10/SEQ ID 11 from WO 2017/089334), HAO mAb4 (See SEQ ID 12/SEQ ID 13 from WO2017157948) or isotype control at a fixed concentration of 50 g/ml in presence of ATP. Supernatants were harvested after 96 hours and analyzed by Meso Scale Discovery human cytokine kit.

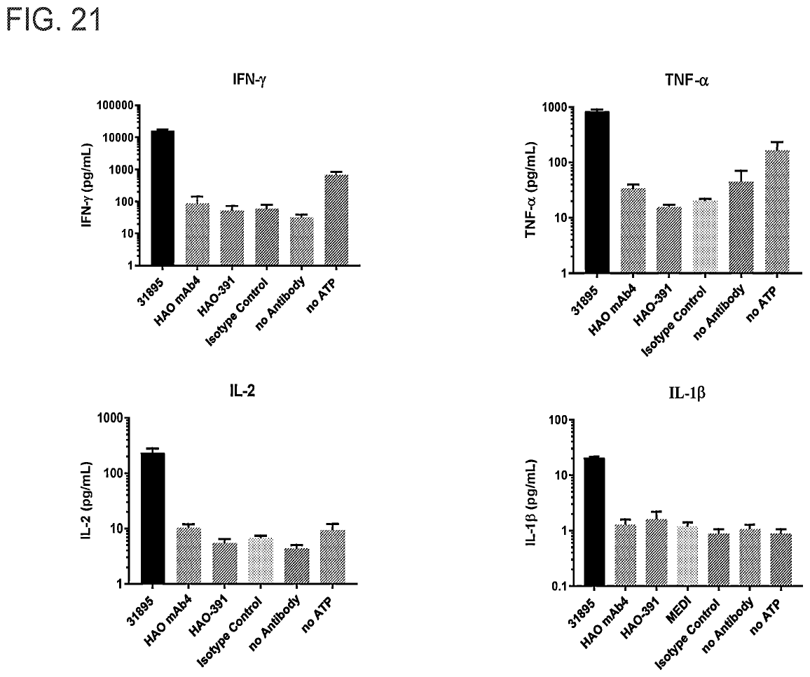

FIG. 21 shows anti-CD39 antibody 31895 increased cytokine release by activated PBMCs to a higher degree compared to anti-CD39 antibodies HAO-391 (VL SEQ ID No. 10; VH SEQ ID No. 11 from WO 2017/089334) and HAO mAb4 (VL SEQ ID No. 12; VH SEQ ID No. 13 from WO2017157948). The x-axis indicates the andtibody and/or conditions and the y-axis shows INF-γ, TNF-α, IL-2 and IL-1β, respectively.

### Example 21: Anti-39 Antibody 31895 Increase Extracellular ATP Accumulation and Reduces Adenosine Generation by CD39⁺CD73⁺ SK-MEL-28 Cells

SK-MEL-28 cells were treated with 31895, isotype control, or small molecule inhibitors (EHNA, ARL, or POM-1) for 1 hour and 50 μM ATP was added for 15 min prior to harvesting of the supernatants. The supernatants were analyzed for ATP levels using AmpGlo Kit (A) and for adenosine levels using LC/MS analysis (B). % Adenosine levels were normalized to isotype control (100%) and SK-MEL-28 CD39 KO cells (0%).

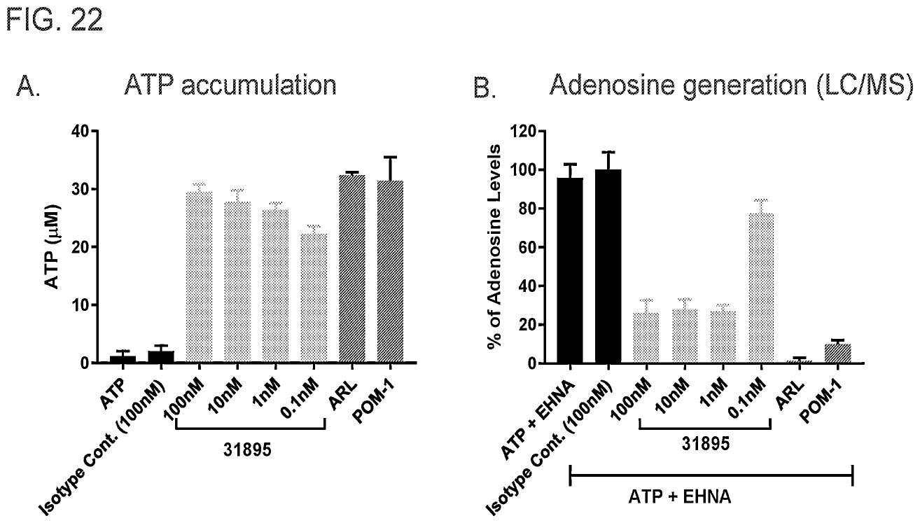

The results are shown in FIG. 22. The left graph shows ATP accumulation and the right graph shows adenosine generation (LC/MC). The x-axis for each graph shows conditions. The y-axis for the left graph shows ATP (μM) and the y-axis for the right graph shows % of adenosine levels.

### Example S: Sequences

Table S provides sequences referred to herein.

TABLE S
Sequences.

| SEQ ID | | | |
|---|---|---|---|
| NO: | Region | Scheme/Clone | Sequence |
| 1 | CDR-H1 | Chothia | GYTFTSY |
| 2 | CDR-H1 | Chothia | GYTFKSY |
| 3 | CDR-H1 | Chothia | GYIFKSY |
| 4 | CDR-H1 | Chothia | GYTFQSY |
| 5 | CDR-H1 | Chothia | GYTFFSY |
| 6 | CDR-H1 | Chothia | GYTFVSY |
| 7 | CDR-H1 | Chothia | GGTFSSLAIS |
| 8 | CDR-H1 | Chothia | GGTFSKLAIS |
| 9 | CDR-H1 | Chothia | GGTFSHT |
| 10 | CDR-H1 | Chothia | GGTFSSL |
| 11 | CDR-H1 | Chothia | GGTFSLL |
| 12 | CDR-H1 | Chothia | GGTFQSL |
| 13 | CDR-H1 | Chothia | GGTFPSN |
| 14 | CDR-H1 | Chothia | GGTFSAM |
| 15 | CDR-H1 | Chothia | GGTFASL |
| 16 | CDR-H1 | Chothia | GGTFSWL |
| 17 | CDR-H1 | Chothia | GGTFSSY |
| 18 | CDR-H1 | Chothia | GGTFGSY |
| 19 | CDR-H1 | Chothia | GGTFSKY |
| 20 | CDR-H1 | Chothia | GGTFGRY |
| 21 | CDR-H1 | Chothia | GGTFESY |
| 22 | CDR-H1 | Chothia | GGTFSNY |
| 23 | CDR-H1 | Chothia | GGAFSSY |
| 24 | CDR-H1 | Chothia | GFTFSSY |
| 25 | CDR-H1 | Kabat | SYYMH |
| 26 | CDR-H1 | Kabat | SYEMH |
| 27 | CDR-H1 | Kabat | SYQMH |
| 28 | CDR-H1 | Kabat | SYYMY |
| 29 | CDR-H1 | Kabat | SYFMH |
| 30 | CDR-H1 | Kabat | SLAIS |
| 31 | CDR-H1 | Kabat | KLAIS |
| 32 | CDR-H1 | Kabat | HTAIS |
| 33 | CDR-H1 | Kabat | SLPIS |
| 34 | CDR-H1 | Kabat | LLAIS |
| 35 | CDR-H1 | Kabat | SNAIS |
| 36 | CDR-H1 | Kabat | AMAIS |
| 37 | CDR-H1 | Kabat | WLAIS |
| 38 | CDR-H1 | Kabat | SYAIS |
| 39 | CDR-H1 | Kabat | SYGIS |
| 40 | CDR-H1 | Kabat | KYGIS |
| 41 | CDR-H1 | Kabat | NYAIS |
| 42 | CDR-H1 | Kabat | SYATS |
| 43 | CDR-H1 | Kabat | SYAIG |
| 44 | CDR-H1 | Kabat | SYSMN |
| 45 | CDR-H1 | Kabat | SYGMN |
| 46 | CDR-H2 | Chothia | NPSGGST |
| 47 | CDR-H2 | Chothia | NPSVGS |
| 48 | CDR-H2 | Chothia | NPSGGS |
| 49 | CDR-H2 | Chothia | NPLGGG |
| 50 | CDR-H2 | Chothia | NPRGGS |
| 51 | CDR-H2 | Chothia | IPIFGT |
| 52 | CDR-H2 | Chothia | GFGT |
| 53 | CDR-H2 | Chothia | LPIGGT |
| 54 | CDR-H2 | Chothia | LPIAGT |
| 55 | CDR-H2 | Chothia | LPIFGE |
| 56 | CDR-H2 | Chothia | IPRGGT |
| 57 | CDR-H2 | Chothia | IPEFGI |
| 58 | CDR-H2 | Chothia | IPSIGT |
| 59 | CDR-H2 | Chothia | IPISGT |
| 60 | CDR-H2 | Chothia | IPTFGT |
| 61 | CDR-H2 | Chothia | SSSSSY |
| 62 | CDR-H2 | Chothia | WYDGSN |
| 63 | CDR-H2 | Kabat | VINPSGGSTSYAQKFQG |
| 64 | CDR-H2 | Kabat | RINPSVGSTWYAQKFQG |
| 65 | CDR-H2 | Kabat | RINPSGGSTWYAQKFQG |
| 66 | CDR-H2 | Kabat | KINPSGGSTWYAQKFQG |
| 67 | CDR-H2 | Kabat | VINPLGGGTSYAQKFQG |
| 68 | CDR-H2 | Kabat | SINPRGGSTSYAQKFQG |
| 69 | CDR-H2 | Kabat | GIIPIFGTANYAQKFQG |
| 70 | CDR-H2 | Kabat | GI--GFGTANYAQKFQG |
| 71 | CDR-H2 | Kabat | GILPIGGTANYAQKFQG |
| 72 | CDR-H2 | Kabat | GILPIAGTANYAQKFQG |
| 73 | CDR-H2 | Kabat | GILPIFGEANYAQKFQG |
| 74 | CDR-H2 | Kabat | GIIPRGGTANYAQKFQG |
| 75 | CDR-H2 | Kabat | SIIPIFGTANYAQKFRG |
| 76 | CDR-H2 | Kabat | SIIPEFGIANYAQKFQG |
| 77 | CDR-H2 | Kabat | SIIPIFGTANYAQKFQG |
| 78 | CDR-H2 | Kabat | GIIPISGTANYAQEFQG |
| 79 | CDR-H2 | Kabat | GIIPTFGTANYAQKFQG |
| 80 | CDR-H2 | Kabat | SISSSSSYIYYADSVKG |
| 81 | CDR-H2 | Kabat | VIWYDGSNKYYADSVKG |
| 82 | CDR-H3 | | GKREGGTEYLRH |
| 83 | CDR-H3 | | GKREGGTEYLRK |
| 84 | CDR-H3 | | GKREGGTEYLRS |
| 85 | CDR-H3 | | GKREGGTEYLRN |
| 86 | CDR-H3 | | GKREGGTEYLRV |
| 87 | CDR-H3 | | GGAKYASTYGMDV |
| 88 | CDR-H3 | | GGAKYASTHGMDV |
| 89 | CDR-H3 | | GGAKYASQLGMDV |
| 90 | CDR-H3 | | GGAKYASKWGMDV |
| 91 | CDR-H3 | | GGAKYAVGYGMDV |
| 92 | CDR-H3 | | GGAKYAGRYGMDV |
| 93 | CDR-H3 | | GGAKYARTYGMDV |
| 94 | CDR-H3 | | ESGGYRDHRLDV |
| 95 | CDR-H3 | | ESGTYRDHRLDV |
| 96 | CDR-H3 | | ESGGYRDHRLGV |
| 97 | CDR-H3 | | DFTDYSSGYSSGWTY |
| 98 | CDR-H3 | | DTLYSSGAYYGYNV |
| 99 | CDR-H3 | | AKRGYDSYGGVYFDY |
| 100 | CDR-H3 | | GPTVTATTSIGTHNWFDP |
| 101 | CDR-H3 | | EGRGYDSSRYYKFWFDPWGQGTLVTVSS |
| 102 | CDR-H3 | | DGGGYRHHYFDL |
| 103 | CDR-H3 | | ESGGYRDHKLDV |
| 104 | CDR-H3 | | DGGGYQHHYFDL |
| 105 | CDR-H3 | | DSGYHRHYSDY |
| 106 | CDR-H3 | | DPLGIRKHWFDP |
| 107 | CDR-H3 | | DTPRWRYHYFDY |
| 108 | CDR-H3 | | ERRGSLALGMDV |
| 109 | CDR-H3 | | DLGGYSYGEPYYYYYGMDV |
| 110 | CDR-L1 | | RASQSVSSSYLA |
| 111 | CDR-L1 | | RASQSVASSYLA |
| 112 | CDR-L1 | | EASQSVSYSYLA |
| 113 | CDR-L1 | | KASESVSSSYLA |
| 114 | CDR-L1 | | RASQYVSSSYLA |
| 115 | CDR-L1 | | KSSQSVLFSSNNKNYLA |
| 116 | CDR-L1 | | KSSRSVLFSSNNKNYLA |
| 117 | CDR-L1 | | KSSKSVLYSNNNKNYLA |
| 118 | CDR-L1 | | RASQSVGSNLA |
| 119 | CDR-L1 | | KSSQSVLYSSNNKNYLA |
| 120 | CDR-L1 | | QASQDISNYLN |
| 121 | CDR-L1 | | RASQSVSSYLA |
| 122 | CDR-L1 | | RASQSVSRYLA |
| 123 | CDR-L1 | | RASQSISSWLA |
| 124 | CDR-L1 | | RASQSVSSDYLA |
| 125 | CDR-L2 | | GASSRAT |
| 126 | CDR-L2 | | GASNRHT |
| 127 | CDR-L2 | | YASSRAY |
| 128 | CDR-L2 | | GASSRAN |
| 129 | CDR-L2 | | YASSRAT |
| 130 | CDR-L2 | | YASNRAT |
| 131 | CDR-L2 | | WASTRES |
| 132 | CDR-L2 | | WASSRES |
| 133 | CDR-L2 | | WASTRQS |
| 134 | CDR-L2 | | WASTRAS |
| 135 | CDR-L2 | | GASTRAT |
| 136 | CDR-L2 | | GASTRAS |
| 137 | CDR-L2 | | DASNLET |
| 138 | CDR-L2 | | DASNRAT |
| 139 | CDR-L2 | | DASKRAT |
| 140 | CDR-L2 | | KASSLES |
| 141 | CDR-L3 | | QQYHSYIT |
| 142 | CDR-L3 | | QQYHNAIT |
| 143 | CDR-L3 | | QQYYFYIT |
| 144 | CDR-L3 | | QQYHSALT |
| 145 | CDR-L3 | | QQYHGGIT |
| 146 | CDR-L3 | | QQYHRRIT |
| 147 | CDR-L3 | | QQYHSGIT |
| 148 | CDR-L3 | | QQYYLYPLT |
| 149 | CDR-L3 | | QQYWTYPLT |
| 150 | CDR-L3 | | QQYLLYPLT |
| 151 | CDR-L3 | | QQYLIWPLT |
| 152 | CDR-L3 | | QQYLLWPLT |
| 153 | CDR-L3 | | QQFYFFPPT |
| 154 | CDR-L3 | | QQAYTFPPT |
| 155 | CDR-L3 | | QQYYIFPPT |
| 156 | CDR-L3 | | QQRNFYPPT |
| 157 | CDR-L3 | | QQFVLWPRT |
| 158 | CDR-L3 | | QQHVNFPLT |
| 159 | CDR-L3 | | QQSVFWPIT |
| 160 | CDR-L3 | | QQLTKWPLT |
| 161 | CDR-L3 | | QQDVLWPLT |
| 162 | CDR-L3 | | QQYGLFPIT |
| 163 | CDR-L3 | | QQHTVWPIT |
| 164 | CDR-L3 | | QQVLNYPLT |
| 165 | CDR-L3 | | QQSYFLPPT |
| 166 | CDR-L3 | | QQAHSSPYT |
| 167 | Leader for | Leader | MKYLLPTAAAGLLLLAAQPAMA |
| | scFV, scFv-Fc | | |
| 168 | Linker for | Linker | GGGGSGGGGSGGGGS |
| | scFV, scFV-FC | | |
| 169 | C-Term Tag for | C-Term Tag | GPGGQHHHHHH |
| | scFV, scFV-FC | | |
| 170 | C-Term Tag for | C-Term Tag | PKSCDKTHTCPPCPAPELLGGPSVFLFPPKPKD |
| | scFv, scFV-FC | | TLMISRTPEVTCVVVDVSHEDPEVKFNWYVDGV |
| | | | EVHNAKTKPREEQYNSTYRVVSVLTVLHQDWLN |
| | | | GKEYKCKVSNKALPAPIEKTISKAKGQPREPQV |
| | | | YTLPPSREEMTKNQVSLTCLVKGFYPSDIAVEW |
| | | | ESNGQPENNYKTTPPVLDSDGSFFLYSKLTVDK |
| | | | SRWQQGNVFSCSVMHEALHNHYTQKSLSLSPGK |
| 171 | scFv | 29872 | MKYLLPTAAAGLLLLAAQPAMAQVQLVQSGAEV |
| | | | KEPGASVKVSCKAPGYTFTSYYMHWVRQAPGQG |
| | | | LEWMGVINPSGGSTSYAQKFQGRVTMTRDTSTS |
| | | | TVYMELSSLRSEDTAVYYCARGKREGGTEYLRH |
| | | | WGQGTLVTVSSGGGGSGGGGSGGGGSEIVLTQS |
| | | | PGTLSLSPGERATLSCRASQSVSSSYLAWYQQK |
| | | | PGQAPRLLIYGASSRATGIPDRFSGSGSGTDFT |
| | | | LTISRLEPEDFAVYYCQQYHSYITFGGGTKVEI |
| | | | KGPGGQHHHHHH |
| 172 | scFv | 31895 | MKYLLPTAAAGLLLLAAQPAMAQVQLVQSGAEV |
| | | | KKPGASVKVSCKASGYTFKSYEMHWVRQAPGQG |
| | | | LEWMGRINPSVGSTWYAQKFQGRVTMTRDTSTS |
| | | | TVYMELSSLRSEDTAVYYCARGKREGGTEYLRK |
| | | | WGQGTLVTVSSGGGGSGGGGSGGGGSEIVLTQS |
| | | | PGTLSLSPGERATLSCRASQSVASSYLAWYQQK |
| | | | PGQAPRLLIYGASNRHTGIPDRFSGSGSGTDFT |
| | | | LTISRLEPEDFAVYYCQQYHNAIYFGGGTKVEI |
| | | | K GPGGQHHHHHH |
| 173 | scFv | 31414 | MKYLLPTAAAGLLLLAAQPAMAQVQLVQSGAEV |
| | | | KKPGASVKVSCKASGYTFKSYEMHWVRQAPGQG |
| | | | LEWMGRINPSVGSTWYAQKFQGRVTMTRDTSTS |
| | | | TVYMELSSLRSEDTAVYYCARGKREGGTEYLRN |
| | | | WGQGTLVTVSSGGGGSGGGGSGGGGSEIVLTQS |
| | | | PGTLSLSPGERATLSCRASQSVSSSYLAWYQQK |
| | | | PGQAPRLLIYGASSRATGIPDRFSGSGSGTDFT |
| | | | LTISRLEPEDFAVYYCQQYHSYIYFGGGTKVEI |
| | | | KGPGGQHHHHHH |
| 174 | scFv | 31905 | MKYLLPTAAAGLLLLAAQPAMAQVQLVQSGAEV |
| | | | KKPGSSVKVSCHASGGTEPSNAISWVRQAPGQG |
| | | | LEWMGGIGFGTANYAQKFQGRVTITADESTSTA |
| | | | YMELSSLRSEDTAVYYCARGGAKYARTYGMDVW |
| | | | GQGTTVTVSSGGGGSGGGGSGGGGSDIVMTQSP |
| | | | DSLAVSLGERATINCKSSKSVLYSNNNKNYLAW |
| | | | YQQKPGQPPKLLIYWASTRQSGVPDRFSGSGSG |
| | | | TDFTLTISSLQAEDVAVYYCQQYLLYPLTEGGG |
| | | | TKVEIKGPGGQHHHHHH |
| 175 | scFv-Fc | 29872 | MKYLLPTAAAGLLLLAAQPAMAQVQLVQSGAEV |
| | | | KEPGASVKVSCHAPGYTFTSYYMHWVRQAPGQG |
| | | | LEWMGVINPSGGSTSYAQKFQGRVTMTRDTSTS |
| | | | TVYMELSSLRSEDTAVYYCARGKREGGIEYLRH |
| | | | WGQGTLVTVSSGGGGSGGGGSGGGGSEIVLTQS |
| | | | PGTLSLSPGERATLSCRASQSVSSSYLAWYQQK |
| | | | PGQAPRLLIYGASSRATGIPDRFSGSGSGTDFT |
| | | | LTISRLEPEDFAVYYCQQYHSYITFGGGTKVEI |
| | | | KPKSCDKTHTCPPCPAPELLGGPSVFLFPPKPK |
| | | | DTLMISRTPEVTCVVVDVSHEDPEVKFNWYVDG |
| | | | VEVHNAKTKPREEQYNSTYRVVSVLTVLHQDWL |
| | | | NGKEYKCKVSNKALPAPIEKTISKAKGQPREPQ |
| | | | VYTLPPSREEMTKNQVSLTCLVKGFYPSDIAVE |
| | | | WESNGQPENNYHTTPPVLDSDGSFFLYSKLTVD |
| | | | KSRWQQGNVFSCSVMHEALHNHYTQKSLSLSPG |
| | | | K |
| 176 | scFv-Fc | 31895 | MKYLLPTAAAGLLLLAAQPAMAQVQLVQSGAEV |
| | | | KKPGASVKVSCKASGYTFKSYEMHWVRQAPGQG |
| | | | LEWMGRINPSVGSTWYAQKFQGRVTMTRDTSTS |
| | | | TVYMELSSLRSEDTAVYYCARGKREGGTEYLRK |
| | | | WGQGTLVTVSSGGGGSGGGGSGGGGSEIVLTQS |
| | | | PGTLSLSPGERATLSCRASQSVASSYLAWYQQK |
| | | | PGQAPRLLIYGASNRHTGIPDRFSGSGSGTDFT |
| | | | LTISRLEPEDFAVYYCQQYHNAITFGGGTKVEI |
| | | | KPKSCDKTHTCPPCPAPELLGGPSVFLFPPKPK |
| | | | DTLMISRTPEVTCVVVDVSHEDPEVKFNWYVDG |
| | | | VEVHNAKTKPREEQYNSTYRVVSVLTVLHQDWL |
| | | | NGKEYKCKVSNKALPAPIEKTISKAKGQPREPQ |
| | | | VYTLPPSREEMTKNQVSLTCLVKGFYPSDIAVE |
| | | | WESNGQPENNYKTIPPVLDSDGSFFLYSKLTVD |
| | | | KSRWQQGNVFSCSVMHEALHNHYTQKSLSLSPG |
| | | | K |
| 177 | scFv-Fc | 31414 | MKYLLPTAAAGLLLLAAQPAMAQVQLVQSGAEV |
| | | | KKPGASVKVSCKASGYTFKSYEMHWVRQAPGQG |
| | | | LEWMGRINPSVGSTWYAQKYQGRVTMTRDTSTS |
| | | | TVYMELSSLRSEDTAVYYCARGKREGGTEYLRN |
| | | | WGQGTLVTVSSGGGGSGGGGSGGGGSEIVLTQS |
| | | | PGTLSLSPGERATLSCRASQSVSSSYLAWYQQK |
| | | | PGQAPRLLIYGASSRATGIPDRFSGSGSGTDFT |
| | | | LTISRLEPEDFAVYYCQQYHSYITFGGGTKVEI |
| | | | KPKSCDKTHTCPPCPAPELLGGPSVFLFPPKPK |
| | | | DTLMISRTPEVTCVVVDVSHEDPEVKFNWYVDG |
| | | | VEVHNAKTKPREEQYNSTYRVVSVLTVLHQDWL |
| | | | NGKEYKCKVSNKALPAPIEKTISKAKGQPREPQ |
| | | | VYTLPPSREEMTKNQVSLTCLVKGFYPSDIAVE |
| | | | WESNGQPENNYKTTPPVLDSDGSFFLYSKLTVD |
| | | | KSRWQQGNVFSCSVMHEALHNHYTQKSLSLSPG |
| | | | K |
| 178 | scFv-Fc | 31905 | MKYLLPTAAAGLLLLAAQPAMAQVQLVQSGAEV |
| | | | KKPGSSVKVSCKASGGTFPSNAISWVRQAPGQG |
| | | | LEWMGGIGFGTANYAQKFQGRVTITADESTSTA |
| | | | YMELSSLRSEDTAVYYCARGGAKYARTYGMDVW |
| | | | GQGTTVTVSSGGGGSGGGGSGGGGSDIVMTQSP |
| | | | DSLAVSLGERATINCKSSKSVLYSNNNKNYLAW |
| | | | YQQKPGQPPKLLIYWASTRQSGVPDRFSGSGSG |
| | | | TDFTLTISSLQAEDVAVYYCQQYLLYPLTFGGG |
| | | | TKVEIKPKSCDKTHTCPPCPAPELLGGPSVFLF |
| | | | PPKPKDTLMISRTPEVTCVVVDVSHEDPEVKFN |
| | | | WYVDGVEVHNAKTKPREEQYNSTYRVVSVLTVL |
| | | | HQDWLNGKEYKCKVSNKALPAPIEKTISKAKGQ |
| | | | PREPQVYTLPPSREEMTKNQVSLTCLVKGFYPS |
| | | | DIAVEWESNGQPENNYKTTPPVLDSDGSFELYS |
| | | | KLTVDKSRWQQGNVFSCSVMHEALHNHYTQKSL |
| | | | SLSPGK |
| 179 | VH | 27579 | QVQLVQSGAEVHEPGASVKVSCKAPGYTFTSYY |
| | | | MHWVRQAPGQGLEWMGVINPSGGSTSYAQKFQG |
| | | | RVTMTRDTSTSTVYMELSSLRSEDTAVYYCARG |
| | | | KREGGTEYLRHWGQGTLVTVSS |
| 180 | VH | 31895 | QVQLVQSGAEVKKPGASVKVSCKASGYTFKSYE |
| | | | MHWVRQAPGQGLEWMGRINPSVGSTWYAQKFQG |
| | | | RVTMTRDTSTSTVYMELSSLRSEDTAVYYCARG |
| | | | KREGGTEYLRKWGQGTLVTVSS |
| 181 | VH | 31415 | QVQLVQSGAEVKKPGASVKVSCKASGYTFTSYQ |
| | | | MHWVRQAPGQGLEWMGRINPSGGSTWYAQKFQG |
| | | | RVTMTRDTSTSTVYMELSSLRSEDTAVYYCARG |
| | | | KREGGTEYLRSWGQGTLVTVSS |
| 182 | VH | 31414 | QVQLVQSGAEVKKPGASVKVSCKASGYTFTSYQ |
| | | | MHWVRQAPGQGLEWMGRINPSVGSTWYAQKFQG |
| | | | RVTMTRDTSTSTVYMELSSLRSEDTAVYYCARG |
| | | | KREGGTEYLRNWGQGTLVTVSS |
| 183 | VH | 31891 | QVQLVQSGAEVKKPGASVKVSCKASGYTFKSYE |
| | | | MHWVRQAPGQGLEWMGRINPSVGSTWYAQKFQG |
| | | | RVTMTRDTSTSTVYMELSSLRSEDTAVYYCARG |
| | | | KREGGTEYLRVWGQGTLVTVSS |
| 184 | VH | 29871 | QVQLVQSGAEVKKPGASVKVSCKASGYTFQSYY |
| | | | MHWVRQAPGQGLEWMGKINPSGGSTWYAQKFQG |
| | | | RVTMTRDTSTSTVYMELSSLRSEDTAVYYCARG |
| | | | KREGGTEYLRHWGQGTLVTVSS |
| 185 | VH | 31418 | QVQLVQSGAEVKKPGASVKVSGKASGYTFKSYE |
| | | | MHWVRQAPGQGLEWMGRINPSGGSTWYAQKFQG |
| | | | RVTMTRDTSTSTVYMELSSLRSEDTAVYYCARG |
| | | | KREGGTEYLRHWGQGTLVTVSS |
| 186 | VH | 31431 | QVQLVQSGAEVKKPGASVKVSCKASGYTFTSYQ |
| | | | MHWVRQAPGQGLEWMGRINPSGGSTWYAQKFQG |
| | | | RVTMTRDTSTSTVYMELSSLRSEDTAVYYCARG |
| | | | KREGGTEYLRHWGQGTLVTVSS |
| 187 | VH | 31421 | QVQLVQSGAEVKKPGASVKVSCKASGYTFFSYY |
| | | | MYWVRQAPGQGLEWMGVINPLGGGTSYAQKFQG |
| | | | RVTMTRDTSTSTVYMELSSLRSEDTAVYYCARG |
| | | | KREGGTEYLRHWGQGTLVTVSS |
| 188 | VH | 31429 | QVQLVQSGAEVKKPGASVKVSCKASGYTFVSYF |
| | | | MHWVRQAPGQGLEWMGSINPRGGSTSYAQKFQG |
| | | | RVTMTRDTSTSTVYMELSSLRSEDTAVYYCARG |
| | | | KREGGTEYLRHWGQGTLVTVSS |
| 189 | VH | 29872 | QVQLVQSGAEVKKPGASVKVSCKASGYTFKSYE |
| | | | MHWVRQAPGQGLEWMGRINPSVGSTWYAQKFQG |
| | | | RVTMTRDTSTSTVYMELSSLRSEDTAVYYCARG |
| | | | KREGGTEYLRHWGQGTLVTVSS |
| 190 | VH | 28347 | QVQLVQSGAEVKKPGSSVKVSCKASGGTFSSLA |
| | | | ISWVRQAPGQGLEWMGGIIPIFGTANYAQKFQG |
| | | | RVTITADESTNTAYMELSSLRSEDTAVYYCARG |
| | | | GAKYASTYGMDVWGQGTTVTVSS |
| 191 | VH | 31896 | QVQLVQSGAEVKKPGSSVKVSCKASGGTFSKLA |
| | | | ISWVRQAPGQGLEWMGGIGFGTANYAQKFQGRV |
| | | | TITADESASTAYMELSSLRSEDTAVYYCARGGA |
| | | | KYASTHGMDVWGQGTTVTVSS |
| 192 | VH | 31432 | QVQLVQSGAEVKKPGSSVKVSCKASGGTFSHTA |
| | | | ISWVRQAPGQGLEWMGGILPIGGTANYAQKFQG |
| | | | RVTITADESTSTAYMELSSLRSEDTAVYYCARG |
| | | | GAKYASQLGMDVWGQGTTVTVSS |
| 193 | VH | 31915 | QVQLVQSGAEVKKPGSSVKVSCKASGGIFSSLP |
| | | | ISWVRQAPGQGLEWMGGIGFGTANYAQKFQGRV |
| | | | TITADESTSTAYMELSSLRSEDTAVYYGARGGA |
| | | | KYASKWGMDVWGQGTTVTVSS |
| 194 | VH | 31436 | QVQLVQSGAEVKKPGSSVKVSCHASGGTFSLLA |
| | | | ISWVRQAPGQGLEWMGGILPIAGTANYAQKFQG |
| | | | RVTITADESTSTAYMELSSLRSEDTAVYYCARG |
| | | | GAKYAVGYGMDVWGQGTTVIVSS |
| 195 | VH | 31437 | QVQLVQSGAEVKKPGASVKVSCKASGGTFQSLA |
| | | | ISWVRQAPGQGLEWMGGILPIGGTANYAQKFQG |
| | | | RVTITADESTSTAYMELSSLRSEDTAVYYCARG |
| | | | GAKYAGRYGMDVWGQGTTVTVSS |
| 196 | VH | 31905 | QVQLVQSGAEVKKPGSSVKVSCKASGGTFPSNA |
| | | | ISWVRQAPGQGLEWMGGIGFGTANYAQKFQGRV |
| | | | TITADESTSTAYMELSSLRSEDTAVYYCARGGA |
| | | | KYARTYGMDVWGQGTTVTVSS |
| 197 | VH | 31901 | QVQLVQSGAEVKKPGSSVKVSCKASGGTFSSLP |
| | | | ISWVRQAPGQGLEWMGGIGFGTANYAQKFQGRV |
| | | | TITALESTSTAYMELSSLRSEDTAVYYCARGGA |
| | | | KYAGRYGMDVWGQGTTVTVSS |
| 198 | VH | 29852 | QVQLVQSGAEVKKPGSSVKVSGKASGGTFSAMA |
| | | | ISWVRQAPGQGLEWMGGILPIAGTANYAQKFQG |
| | | | RVTITADESTSTAYMELSSLRSEDTAVYYCARG |
| | | | GAKYASTYGMDVWGQGTTVTVSS |
| 199 | VH | 29851 | QVQLVQSGAEVKKPGSSVKVSCKASGGTFASLA |
| | | | ISWVRQAPGQGLEWMGGILPIFGEANYAQKFQG |
| | | | RVTITADESTSTAYMELSSLRSEDTAVYYCARG |
| | | | GAKYASTYGMDVWGQGTTVTVSS |
| 200 | VH | 29857 | QVQLVQSGAEVKKPGSSVKVSCKASGGTFSWLA |
| | | | ISWVRQAPGQGLEWMGGIIPRGGTANYAQKFQG |
| | | | RVTITADESTSTAYMELSSLRSEDTAVYYCARG |
| | | | GAKYASTYGMDVWGQGTTVTVSS |
| 201 | VH | 27571 | QVQLVQSGAEVKKPGSSVKASCKASGGTFSSYA |
| | | | ISWVRQAPGQGLEWMGSIIPIFGTANYAQKFRG |
| | | | RVTITADESTSTTYMELSSLRSEDTAVYYCARE |
| | | | SGGYRDHRLDVWGQGTMVTVSS |
| 202 | VH | 31861 | QVQLVQSGAEVKKPGSSVKVSCKASGGTFGSYG |
| | | | ISWVRQAPGQGLEWMGSIIPEFGIANYAQKFQG |
| | | | RVTITADESTSTAYMELSSLRSEDTAVYYCARE |
| | | | SGTYRDHRLDVWGQGTMVTVSS |
| 203 | VH | 31873 | QVQLVQSGAEVKKPGSSVKVSCKASGGTFSKYG |
| | | | ISWVRQAPGQGLEWMGSIIPEFGIANYAQKFQG |
| | | | RVTITADESTSTAYMELSSLRSEDTAVYYCARE |
| | | | SGGYRDHRLGVWGQGTMVTVSS |
| 204 | VH | 31393 | QVQLVQSGAEVKKPGSSVKVSCKASGGTFESYG |
| | | | ISWVRQAPGQGLEWMGSIIPEFGIANYAQKFQG |
| | | | RVTITADESTSTTYMELSSLRSEDTAVYYCARE |
| | | | SGGYRDHRLDVWGQGTMVTVSS |
| 205 | VH | 27534 | QVQLVQSGAEVKKPGSSVKVSCKASGGTFSSYA |
| | | | ISWVRQAPGQGLEWMGGIIPIFGTANYAQKFQG |
| | | | RVTITADESTSTAYMELSSLRSEDTAVYYCARD |
| | | | FTDYSSGYSSGWTYWGQGTLVTVSS |
| 206 | VH | 27536 | QVQLVQSGAEVKKPGSSVKVSCKASGGTFSNYA |
| | | | ISWVRQAPGQGLEWMGGIIPIFGTANYAQKFQG |
| | | | RVTITADESTSTAYMELSSLRSEDTAVYYCARD |
| | | | TLYSSGAYYGYNVWGQGTMVTVSS |
| 207 | VH | 27588 | QVQLVQSGAEVKKPGSSVKVSCKASGGTFSNYA |
| | | | ISWVRQAPGQGLEWMGGIIPIFGTANYAQKFQG |
| | | | RVTITADESTSTAYMELSSLRSEDTAVYYCARA |
| | | | KRGYDSYGGVYFDYWGQGTLVTVSS |
| 208 | VH | 27590 | QVQLVQSGAEVKKPGSSVKVSCKASGGTFSNYA |
| | | | ISWVRQAPGQGLEWMGGIIPIFGTANYAQKFQG |
| | | | RVTITADESTSTAYMELSSLRSEDTAVYYCARG |
| | | | PTVTATTSIGTHNWFDPWGQGTLVTVSS |
| 209 | VH | 27597 | QVQLVQSGAEVKKPGSSVKVSCKASGGTFSSYA |
| | | | ISWVRQAPGQGLEWMGSIIPIFGTANYAQKFQG |
| | | | RVTITADESTSTAYMELSSLRSEDTAVYYCARE |
| | | | GRGYDSSRYYKFWFDPWGQGTLVTVSS |
| 210 | VH | 27575 | QVQLVQSGAEVKEPGSSVKVSCKASGGTFSSYA |
| | | | TSWVRQAPGQGLEWMGGIIPISGTANYAQEFQG |
| | | | RVTITADESTSTAYMELSSLRSEDTAVYYCARD |
| | | | GGGYRHHYFDLWGRGTLVTVSS |
| 211 | VH | 27568 | QVQLVQSGAEVKKPGSSVKVPCKASGGTFSSYA |
| | | | ISWVRQAPEQGLEWMGSIIPIFGTANYAQKFQG |
| | | | RVTITADESTSTAYMELSSLRSEDTAVYYCAGE |
| | | | SGGYRDHKLDVWGQGTVVTVSS |
| 212 | VH | 27577 | QVQLVQSGAEVKKPGSSVKVSCKASGGAFSSYA |
| | | | IGWVRQAPGQGLEWMGGIIPTFGTANYAQKFQG |
| | | | RVTITADESTSTAYMELSSLRSEDTAVYYCARD |
| | | | GGGYQHHYFDLWGRGTLVTVSS |
| 213 | VH | 27587 | QVQLVQSGAEVKKPGSSVKVSCKASGGTFSSYA |
| | | | ISWVRQAPGQGLEWMGSIIPIFGTANYAQKFQG |
| | | | RVTITADESTSTAYMELSSLRSEDTAVYYCARE |
| | | | SGGYRDHKLDVWGQGTMVTVSS |
| 214 | VH | 27589 | QVQLVQSGAEVKKPGSSVKVSCKASGGTFSSYA |
| | | | ISWVRQAPGQGLEWMGGIIPIFGTANYAQKFQG |
| | | | RVTITADESTSTAYMELSSLRSEDTAVYYCARD |
| | | | SGYHRHYSDYWGQGTLVTVSS |
| 215 | VH | 27596 | QVQLVQSGAEVKKPGSSVKVSCKASGGTFSSYA |
| | | | ISWVRQAPGQGLEWMGGIIPIFGTANYAQKFQG |
| | | | RVTITADESTSTAYMELSSLRSEDTAVYYCARD |
| | | | PLGIRKHWFDPWGQGTLVTVSS |
| 216 | VH | 27535 | QVQLVQSGAEVKKPGSSVKVSCKASGGTFSSYA |
| | | | ISWVRQAPGQGLEWMGGIIPIFGTANYAQKFQG |
| | | | RVTITADESTSTAYMELSSLRSEDTAVYYCARD |
| | | | TPRWRYHYFDYWGQGTLVTVSS |
| 217 | VH | 27550 | EVQLVESGGGLVKPGGSLRLSCAASGFTFSSYS |
| | | | MNWVRQAPGKGLEWVSSISSSSSYIYYADSVKG |
| | | | RFTISRDNAKNSLYLQMNSLRAEDTAVYYCARE |
| | | | RRGSLALGMDVWGQGTLVTVSS |
| 218 | VH | 27549 | QVQLVESGGGVVQPGRSLRLSCAASGFTFSSYG |
| | | | MNWVRQAPGKGLEWVAVIWYDGSNKYYADSVKG |
| | | | RFTISRDNSKNTLYLQMNSLRAEDTAVYYCARD |
| | | | LGGYSYGEPYYYYYGMDVWGQGTTVTVSS |
| 219 | VL | 27579 | ETVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQYHSYITFG |
| | | | GGTKVEIK |
| 220 | VL | 31895 | EIVLTQSPGTLSLSPGERATLSCRASQSVASSY |
| | | | LAWYQQKPGQAPRLLIYGASNRFITGIPDRESGS |
| | | | GSGTDFTLTISRLEPEDFAVYYCQQYHNAITFG |
| | | | GGTKVEIK |
| 221 | VL | 31891 | EIVLTQSPGTLSLSPGERATLSCRASQSVSSSY |
| | | | LAWYQQKPGQAPRLLIYYASSRAYGIPDRFSGS |
| | | | GSGTDFTLTISRLEPEDFAVYYCQQYHNAITFG |
| | | | GGTKVEIK |
| 222 | VL | 31418 | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQYYFYITFG |
| | | | GGTKVEIK |
| 223 | VL | 31430 | EIVLTQSPGTLSLSPGERATLSCFASQSVSYSY |
| | | | LAWYQQKPGQAPRLLIYGASSRANGIPDRFSGS |
| | | | GSGTDFTLTISRLEPEDFAVYYCQQYHSALTFG |
| | | | GGTKVEIK |
| 224 | VL | 31431 | EIVLTQSPGTLSLSPGERATLSCRASQSVASSY |
| | | | LAWYQQKPGQAPRLLIYGASNRHTGIPDRFSGS |
| | | | GSGTDFTLTISRLEPEDFAVYYCQQYHGGITFG |
| | | | GGTKVEIK |
| 225 | VL | 31421 | EIVLTQSPGTLSLSPGERATLSCKASESVSSSY |
| | | | LAWYQQKPGQAPRLLIYYASSRATGIPDRFSGS |
| | | | GSGTDFTLTISRLEPEDFAVYYCQQYHRRITFG |
| | | | GGTKVEIK |
| 226 | VL | 31429 | EIVLTQSPGTLSLSPGERATLSCRASQYVSSSY |
| | | | LAWYQQKPGQAPRLLIYYASNRATGIPDRFSGS |
| | | | GSGTDFTLTISRLEPEDFAVYYCQQYHSGITFG |
| | | | GGTKVEIK |
| 227 | VL | 28347 | DIVMTQSPDSLAVSLGERATINCKSSQSVLFSS |
| | | | NNKNYLAWYQQKPGQPPKLLIYWASTRESGVPD |
| | | | RFSGSGSGTDFTLTISSLQAEDVAVYYCQQYYL |
| | | | YPLTFGGGTKVEIK |
| 228 | VL | 31896 | DIVMTQSPDSLAVSLGERATINCKSSRSVLFSS |
| | | | NNKNYLAWYQQKPGQPPKLLIYWASTRESGVPD |
| | | | RFSGSGSGTDFTLTISSLQAEDVAVYYCQQYWT |
| | | | YPLTFGGGTKVEIK |
| 229 | VL | 31915 | DIVMTQSPDSLAVSLGERATINCKSSQSVLFSS |
| | | | NNKNYLAWYQQKPGQPPKLLIYWASSRESGVPD |
| | | | RFSGSGSGTDFTLTISSLQAEDVAVYYCQQYWT |
| | | | YPLTFGGGTKVEIK |
| 230 | VL | 31905 | DIVMTQSPDSLAVSLGERATINCKSSKSVLYSN |
| | | | NNKNYLAWYQQKPGQPPKLLIYWASTRQSGVPD |
| | | | RFSGSGSGTDFTLTISSLQAEDVAVYYCQQYLL |
| | | | YPLTFGGGTKVEIK |
| 231 | VL | 31901 | GIVMTQSPDSLAVSLGERATINCKSSQSVLFSS |
| | | | NNKNYLAWYQQKPGQPPKLLIYWASIRASGVPD |
| | | | RFSGSGSGTDFTLTISSLQAEDVAVYYCQQYYL |
| | | | YPLTFGGGTKVEIK |
| 232 | VL | 27571 | EIVMTQSPATLSVSPGERATLSCRASQSVGSNL |
| | | | AWYQQKPGQAPRLLIYGASTRATGIPARFSGSG |
| | | | SGTEFTLTISSLQSEDFAVYYCQQYLIWPLTFG |
| | | | GGTKVEIK |
| 233 | VL | 31861 | EIVMTQSPATLSVSPGERATLSCRASQSVGSNL |
| | | | AWYQQKPGQAPRLLIYGASTRATGIPARFSGSG |
| | | | SGTEFTLTISSLQSEDFAVYYCQQYLLWPLTEG |
| | | | GGTKVEIK |
| 234 | VL | 31873 | EIVMTQSPATLSVSPGERATLSCRASQSVGSNL |
| | | | AWYQQKPGQAPRLLIYGASTRASGIPARFSGSG |
| | | | SGTEFTLTISSLQSEDFAVYYCQQYLLWPLTFG |
| | | | GGTKVEIK |
| 235 | VL | 28337 | DIVMTQSPDSLAVSLGERATINCKSSQSVLFSS |
| | | | NNKNYLAWYQQKPGQPPKLLIYWASTRESGVPD |
| | | | RFSGSGSGTDFTLTISSLQAEDVAVYYCQQFYF |
| | | | YPPTFGGGTKVEIK |
| 236 | VL | 27536 | DIVMTQSPDSLAVSLGERATINCKSSQSVLYSS |
| | | | NNKNYLAWYQQKPGQPPKLLIYWASTRESGVPD |
| | | | RFSGSGSGTDFTLTISSLQAEDVAVYYCQQAYT |
| | | | FPPTFGGGTKVEIK |
| 237 | VL | 27588 | DIQMTQSPSSLSASVGDRVTITCQASQDISNYL |
| | | | NWYQQKPGKAPKLLIYDASNLETGVPSRFSGSG |
| | | | SGTDFTFTISSLQPEDIATYYCQQYYIEPPTFG |
| | | | GGTKVEIK |
| 238 | VL | 27590 | EIVLTQSPATLSLSPGERATLSCRASQSVSSYL |
| | | | AWYQQKPGQAPRLLIYDASNRATGIPARFSGSG |
| | | | SGTDFTLTISSLEPEDFAVYYCQQRNFYPPTFG |
| | | | GGTKVEIK |
| 239 | VL | 27597 | EIVLTQSPATLSLSPGERATLSCRASQSVSSYL |
| | | | AWYQQKPGQAPRLLIYDASNRATGIPARFSGSG |
| | | | SGTDFTLTISSLEPEDFAVYYCQQFVLWPRTFG |
| | | | GGTKVEIK |
| 240 | VL | 27575 | EIVLTQSPATLSLSPGERATLSCRASQSVSRYL |
| | | | AWYQQKPGQAPRLLIYDASNRATGIPARFSGSG |
| | | | SGTDFTLTISSLEPEDFAVYYCQQHVNFPLTFG |
| | | | GGTKVEIK |
| 241 | VL | 27568 | EIVLTQSPATLSLSPGERATLSCRASQSVSSYL |
| | | | AWYQQKPGQAPRLLIYDASNRATGIPARFSGSG |
| | | | SGTDFTLTISSLEPEDFAVYYCQQSVFWPITFG |
| | | | GGTKVEIK |
| 242 | VL | 27577 | EIVMTQSPATLSVSPGERATLSCRASQSVGSNL |
| | | | AWYQQKPGQAPRLLIYGASTRATGIPARFSGSG |
| | | | SGTEFTLTISSLQSEDFAVYYCQQLTKWPLTFG |
| | | | GGTKVEIK |
| 243 | VL | 27587 | EIVLTQSPATLSLSPGERATLSCRASQSVSSYL |
| | | | AWYQQKPGQAPRLLIYDASKRATGIPARFSGSG |
| | | | SGTDFTLTISSLEPEDFAVYYCQQDVLWPLTFG |
| | | | GGTKVEIK |
| 244 | VL | 27589 | DIQMTQSPSTLSASVGDRVTITCRASQSISSWL |
| | | | AWYQQKPGKAPKLLIYKASSLESGVPSRFSGSG |
| | | | SGTEFTLTISSLQPDDFATYYCQQYGLFPITFG |
| | | | GGTKVEIK |
| 245 | VL | 27596 | EIVMTQSPATLSLSPGERATLSCRASQSVSSYL |
| | | | AWYQQKPGQAPRLLIYDASNRATGIPARFSGSG |
| | | | SGTDFTLTISSLEPEDFAVYYCQQHTVWPITFG |
| | | | GGTKVEIK |
| 246 | VL | 27535 | EIVLTQSPATLSLSPGERATLSCRASQSVSSYL |
| | | | AWYQQKPGQAPRLLIYDASKRATGIPARFSGSG |
| | | | SGTDFTLTISSLEPEDFAVYYCQQVLNYPLTFG |
| | | | GGTKVEIK |
| 247 | VL | 27550 | DIQMTQSPSSLSASVGDRVTITCQASQDISNYL |
| | | | NWYQQKPGKAPKLLIYDASNLETGVPSRFSGSG |
| | | | SGTDFTFTISSLQPEDIATYYCQQSYFLPPTFG |
| | | | GGTKVEIK |
| 248 | VL | 27549 | EIVLTQSPGTLSLSPGERATLSCRASQSVSSDY |
| | | | LAWYQQKPGQAPRLLIYGASSRATGIPDRFSGS |
| | | | GSGTDFTLTISRLEPEDFAVYYCQQAHSSPYTF |
| | | | GGGTKVEIK |
| 249 | | hCD39 | MEDTKESNVKTFCSKNILAILGFSSIIAVIALL |
| | | | AVGLTQNKALPENVKYGIVLDAGSSHTSLYIYK |
| | | | WPAEKENDTGVVHQVEECRVKGPGISKFVQKVN |
| | | | EIGIYLTDCMERAREVIPRSQHQETPVYLGATA |
| | | | GMRLLRMESEELADRVLDVVERSLSNYPFDFQG |
| | | | ARIITGQEEGAYGWITINYLLGKESQKTRWFSI |
| | | | VPYETNNQETFGALDLGGASTQVTFVPQNQTIE |
| | | | SPDNALQFRLYGKDYNVYTHSFLCYGKDQALWQ |
| | | | KLAKDIQVASNEILRDPCFHPGYKKVVNVSDLY |
| | | | KTPCTKRFEMTLPFQQFEIQGIGNYQQCHQSIL |
| | | | ELFNTSYCPYSQCAFNGIFLPPLQGDFGAFSAF |
| | | | YFVMKFLNLTSEKVSQEKVTEMMKKFCAQPWEE |
| | | | IKTSYAGVKEKYLSEYCFSGTYILSLLLQGYHF |
| | | | TADSWEHIHFIGKIQGSDAGWTLGYMLNLTNMI |
| | | | PAEQPLSTPLSHSTYVFLMVLFSLVLFTVAIIG |
| | | | LLIFHKPSYFWKDMV |
| 250 | | mCD39 | MEDIKDSKVKRFCSKNILIILGFTSILAVIALI |
| | | | AVGLTQNKPLPENVKYGIVLDAGSSHTNLYIYE |
| | | | WPAEKENDTGVVQQLEECQVKGPGISKYAQKTD |
| | | | EIGAYLAECMELSTELIPTSKHHQTPVYLGATA |
| | | | GMRLLRMESEQSADEVLAAVSTSLKSYPFDFQG |
| | | | AKIITGQEEGAYGWITINYLLGRFTQEQSWLSL |
| | | | ISDSQKQETFGALDLGGASTQITFVPQNSTIES |
| | | | PENSLQFRLYGEDYTVYTHSFLCYGKDQALWQK |
| | | | LAKDIQVSSGGVLKDPCFNPGYEKVVNVSELYG |
| | | | TPCTKRFEKKLPFDQFRIQGTGDYEQCHQSILE |
| | | | LFNNSHCPYSQCAFNGVFLPPLHGSFGAFSAFY |
| | | | FVMDFFKKVAKNSVISQEKMTEITKNFCSKSWE |
| | | | ETKTSYPSVKEKYLSEYCFSGAYILSLLQGYNF |
| | | | TDSSWEQIHFMGKIKDSNAGWTLGYMLNLTNMI |
| | | | PAEQPLSPPLPHSTYIGLMVLFSLLLVAVAITG |
| | | | LFIYSKPSYFWKEAV |
| 251 | | Macaca | MLFDSILSTVGLSKLVSVVSSPAAALSKSNVKT |
| | | fascicularis | FCSKNILAILGESSIIAVIALLAVGLTQNKALP |
| | | cCD39 | ENIKYGIVLDAGSSHISLYTYKWPAEKENDTGV |
| | | | VHQVEECRVKGPGISKYVQKVNEIGIYLTDCME |
| | | | RAREVIPRSQHQETPVYLGATAGMRLLRMESEE |
| | | | LADRVLDVVERSLSNYPFDFQGARIITGQEEGA |
| | | | YGWITINYLLGKFSQKTRWFSIVPYETNNQETF |
| | | | GALDLGGASTQITFVPQNQTTESPDNALQFRLY |
| | | | GKDYNVYTHSFLCYGKDQALWQKLAKDIQVASN |
| | | | EILRDPCFHPGYKKVVNVSDLYKTPCTKRFEMT |
| | | | LPFQQFEIQGIGNYQQCHQSVLELENTSYCPYS |
| | | | QCAFNGIFLPPLQGDFGAFSAFYFVMNFLNLTS |
| | | | EKVSQEKVTEMMKKFCSQPWEEIKTSYAGVKEK |
| | | | YLSEYCFSGTYILSLLLQGYHETADSWEHIHFI |
| | | | GKIQGSDAGWTLGYMLNLTNMIPAEQPLSTPLS |
| | | | HSTYVFLMVLFSLVLVIVAIIGLLIFHKPSYFW |
| | | | KDMV |
| 252 | | hCD39 ECD | TQNKALPENVKYGIVLDAGSSHTSLYIYKWPAE |
| | | | KENDTGVVHQVEECRVKGPGISKFVQKVNEIGI |
| | | | YLTDCMERAREVIPRSQHQETPVYLGATAGMRL |
| | | | LRMESEELADRVLDVVERSLSNYPFDEQGARII |
| | | | TGQEEGAYGWITINYLLGKESQKTRWFSIVPYE |
| | | | TNNQETFGALDLGGASTQVTFVPQNQTIESPDN |
| | | | ALQFRLYGKDYNVYTHSFLCYGKDQALWQKLAK |
| | | | DIQVASNEILRDPCFHPGYKKVVNVSDLYKTPC |
| | | | TKRFEMTLPFQQFEIQGIGNYQQCHQSILELFN |
| | | | TSYCPYSQCAFNGIFLPPLQGDFGAFSAFYFVM |
| | | | KFLNLTSEKVSQEKVTEMMKKFCAQPWEEIKTS |
| | | | YAGVKEKYLSEYCESGTYILSLLLQGYHFTADS |
| | | | WEHIHFIGKIQGSDAGWTLGYMLNLTNMIPAEQ |
| | | | PLSTPLSHSTYVFLMVLFSLVLFTVAIIGLLIF |
| | | | HKPSYFWKDMV |
| 253 | | mCD39 ECD | TQNKPLPENVKYGIVLDAGSSHTNLYIYKWPAE |
| | | | KENDTGVVQQLEECQVKGPGISKYAQKTDEIGA |
| | | | YLAECMELSTELIPTSKHHQTPVYLGATAGMRL |
| | | | LRMESEQSADEVLAAVSTSLKSYPEDFQGAKII |
| | | | TGQEEGAYGWITINYLLGRFTQEQSWLSLISDS |
| | | | QKQETFGALDLGGASTQITFVPQNSTIESPENS |
| | | | LQFRLYGEDYTVYTHSFLCYGKDQALWQKLAKD |
| | | | IQVSSGGVLKDPCFNPGYEKVVNVSELYGTPCT |
| | | | KRFEKKLPFDQFRIQGTGDYEQCHQSILELFNN |
| | | | SHCPYSQCAFNGVFLPPLHGSFGAFSAFYFVMD |
| | | | FFKKVAKNSVISQEKMTEITKNFCSKSWEETKT |
| | | | SYPSVKEKYLSEYCFSGAYILSLLQGYNFTDSS |
| | | | WEQIHFMGKIKDSNAGWTLGYMLNLTNMIPAEQ |
| | | | PLSPPLPHSTYIGLMVLFSLLLVAVAITGLFIY |
| | | | SKPSYFWKEAV |
| 254 | | cCD39 ECD | TQNKALPENIKYGIVLDAGSSHTSLYIYKWPAE |
| | | | KENDTGVVHQVEECRVKGPGISKYVQKVNEIGI |
| | | | YLTDCMERAREVIPRSQHQETPVYLGATAGMRL |
| | | | LRMESEELADRVLDVVERSLSNYPFDFQGARII |
| | | | TGQEEGAYGWITINYLLGKESQKTRWFSIVPYE |
| | | | TNNQETFGALDLGGASTQITFVPQNQTTESPDN |
| | | | ALQFRLYGKDYNVYTHSFLCYGKDQALWQKLAK |
| | | | DIQVASNEILRDPCFHPGYKKVVNVSDLYKTPC |
| | | | TKRFEMTLPFQQFEIQGIGNYQQCHQSVLELFN |
| | | | TSYCPYSQCAFNGIFLPPLQGDFGAFSAFYFVM |
| | | | NELNLTSEKVSQEKVIEMMKKECSQPWEEIKTS |
| | | | YAGVKEKYLSEYCFSGTYILSLLLQGYHFTADS |
| | | | WEHIHFIGKIQGSDAGWTLGYMLNLTNMIPAEQ |
| | | | PLSTPLSHSTYVFLMVLFSLVLVIVAIIGLLIF |
| | | | HKPSYFWKDMV |
| 255 | HC | 31895 | QVQLVQSGAEVKKPGASVKVSCKASGYTFKSYE |
| | | | MHWVRQAPGQGLEWMGRINPSVGSTWYAQKFQG |
| | | | RVTMTRDTSTSTVYMELSSLRSEDTAVYYCARG |
| | | | KREGGTEYLRKWGQGTLVTVSSASTKGPSVFPL |
| | | | APCSRSTSESTAALGCLVKDYEPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLIVDKSRWQEGNVFSCSVM |
| | | | HEALHNHYTQKSLSLSLGK |
| 256 | LC | 31895 | EIVLTQSPGTLSLSPGERATLSCRASQSVASSY |
| | | | LAWYQQKPGQAPRLLIYGASNRHTGIPDRFSGS |
| | | | GSGTDFTLTISRLEPEDFAVYYCQQYHNAITFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 257 | HC | 31415 | QVQLVQSGAEVKKPGASVKVSCKASGYTFTSYQ |
| | | | MHWVRQAPGQGLEWMGRINPSGGSTWYAQKFQG |
| | | | RVTMTRDTSTSTVYMELSSLRSEDTAVYYCARG |
| | | | KREGGTEYLRSWGQGTLVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 258 | LC | 31415 | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQYHSYITFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 259 | HC | 31891 | QVQLVQSGAEVKKPGASVKVSCKASGYIFKSYE |
| | | | MHWVRQAPGQGLEWMGRINPSVGSTWYAQKFQG |
| | | | RVTMTRDTSTSTVYMELSSLRSEDTAVYYCARG |
| | | | KREGGTEYLRVWGQGTLVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 260 | LC | 31891 | EIVLTQSPGTLSLSPGERATLSCRASQSVSSSY |
| | | | LAWYQQKPGQAPRLLIYYASSRAYGIPDRFSGS |
| | | | GSGTDFTLTISRLEPEDFAVYYCQQYHNAITFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 261 | HC | 31418 | QVQLVQSGAEVKKPGASVKVSCKASGYTFKSYE |
| | | | MHWVRQAPGQGLEWMGRINPSGGSTWYAQKFQG |
| | | | RVTMTRDTSTSTVYMELSSLRSEDTAVYYCARG |
| | | | KREGGTEYLRHWGQGTLVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGEPCPPCP |
| | | | APEFLGGPSVFLFPPKPKDILMISRTPEVTCVV |
| | | | VDVSQEDPEVQENWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 262 | LC | 31418 | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQYYFYITFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 263 | HC | 31430 | QVQLVQSGAEVKKPGASVKVSCKASGYTFKSYE |
| | | | MHWVRQAPGQGLEWMGRINPSGGSTWYAQKFQG |
| | | | RVTMTRDTSTSTVYMELSSLRSEDTAVYYCARG |
| | | | KREGGTEYLRHWGQGTLVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 264 | LC | 31430 | EIVLTQSPGTLSLSPGERATLSCEASQSVSYSY |
| | | | LAWYQQKPGQAPRLLIYGASSRANGIPDRFSGS |
| | | | GSGTDFTLTISRLEPEDFAVYYCQQYHSALTFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 265 | HC | 31915 | QVQLVQSGAEVKKPGSSVKVSCKASGGTFSSLP |
| | | | ISWVRQAPGQGLEWMGGIGEGTANYAQKFQGRV |
| | | | TITADESTSTAYMELSSLRSEDTAVYYCARGGA |
| | | | KYASKWGMDVWGQGTTVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVEGFYPSDIAVEWESNGQPENNYKTTPP |
| | | | VLDSDGSFFLYSRLTVDKSRWQEGNVFSCSVMH |
| | | | EALHNHYTQKSLSLSLGK |
| 266 | LC | 31915 | DIVMTQSPDSLAVSLGERATINCKSSQSVLFSS |
| | | | NNKNYLAWYQQKPGQPPKLLIYWASSRESGVPD |
| | | | RFSGSGSGTDFTLTISSLQAEDVAVYYCQQYWT |
| | | | YPLTFGGGTKVEIKRTVAAPSVFIFPPSDEQLK |
| | | | SGTASVVCLLNNFYPREAKVQWKVDNALQSGNS |
| | | | QESVTEQDSKDSTYSLSSTLTLSKADYEKHKVY |
| | | | ACEVTHQGLSSPVTKSFNRGEC |
| 267 | HC | 31905 | QVQLVQSGAEVKKPGSSVKVSCKASGGTFPSNA |
| | | | ISWVRQAPGQGLEWMGGIGFGTANYAQKFQGRV |
| | | | TITADESTSTAYMELSSLRSEDTAVYYCARGGA |
| | | | KYARTYGMDVWGQGTTVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSLGK |
| 268 | LC | 31905 | DIVMTQSPDSLAVSLGERATINCKSSKSVLYSN |
| | | | NNKNYLAWYQQKPGQPPKLLIYWASTRQSGVPD |
| | | | RFSGSGSGTDFTLTISSLQAEDVAVYYCQQYLL |
| | | | YPLTFGGGTKVEIKRTVAAPSVFIFPPSDEQLK |
| | | | SGTASVVCLLNNFYPREAKVQWKVDNALQSGNS |
| | | | QESVTEQDSKDSTYSLSSTLTLSKADYEKHKVY |
| | | | ACEVTHQGLSSPVTKSFNRGEC |
| 269 | HC | 31901 | QVQLVQSGAEVKKPGSSVKVSCKASGGTFSSLP |
| | | | ISWVRQAPGQGLEWMGGIGFGTANYAQKFQGRV |
| | | | TITADESTSTAYMELSSLRSEDTAVYYCARGGA |
| | | | KYAGRYGMDVWGQGTTVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSLGK |
| 270 | LC | 31901 | GIVMTQSPDSLAVSLGERATINCKSSQSVLFSS |
| | | | NNKNYLAWYQQKPGQPPKLLIYWASIRASGVPD |
| | | | RFSGSGSGTDFTLTISSLQAEDVAVYYCQQYYL |
| | | | YPLTFGGGTKVEIKRIVAAPSVFIFPPSDEQLK |
| | | | SGTASVVCLLNNFYPREAKVQWKVDNALQSGNS |
| | | | QESVTEQDSKDSTYSLSSTLTLSKADYEKHKVY |
| | | | ACEVTHQGLSSPVTKSFNRGEC |
| 271 | HC | 31861 | QVQLVQSGAEVKKPGSSVKVSCKASGGTEGSYG |
| | | | ISWVRQAPGQGLEWMGSIIPEFGIANYAQKFQG |
| | | | RVTITADESTSTAYMELSSLRSEDTAVYYCARE |
| | | | SGTYRDHRLDVWGQGTMVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGEPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 272 | LC | 31861 | EIVMTQSPATLSVSPGERATLSCRASQSVGSNL |
| | | | AWYQQKPGQAPRLLIYGASTRATGIPARFSGSG |
| | | | SGTEFTLTISSLQSEDFAVYYCQQYLLWPLTFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 273 | HC | 31873 | QVQLVQSGAEVKKPGSSVKVSCKASGGTFSKYG |
| | | | ISWVRQAPGQGLEWMGSIIPEFGIANYAQKFQG |
| | | | RVTITADESTSTAYMELSSLRSEDTAVYYCARE |
| | | | SGGYRDHRLGVWGQGTMVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDILMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 274 | LC | 31873 | EIVMTQSPATLSVSPGERATLSCRASQSVGSNL |
| | | | AWYQQKPGQAPRLLIYGASTRASGIPARESGSG |
| | | | SGTEFTLTISSLQSEDFAVYYCQQYLLWPLTFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 275 | HC | 31393 | QVQLVQSGAEVKKPGSSVKVSCKASGGTFESYG |
| | | | ISWVRQAPGQGLEWMGSIIPEFGIANYAQKFQG |
| | | | RVTITADESTSTTYMELSSLRSEDTAVYYCARE |
| | | | SGGYRDHRLDVWGQGTMVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDILMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 276 | LC | 31393 | EIVMTQSPATLSVSPGERATLSCRASQSVGSNL |
| | | | AWYQQKPGQAPRLLIYGASTRATGIPARFSGSG |
| | | | SGTEFTLTISSLQSEDFAVYYCQQYLLWPLTFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 277 | HC | 27597 | QVQLVQSGAEVKKPGSSVKVSCKASGGTFSSYA |
| | | | ISWVRQAPGQGLEWMGSIIPIFGTANYAQKFQG |
| | | | RVTITADESTSTAYMELSSLRSEDTAVYYCARE |
| | | | GRGYDSSRYYKFWFDPWGQGTLVTVSSASTKGP |
| | | | SVFPLAPCSRSTSESTAALGCLVKDYFPEPVTV |
| | | | SWNSGALTSGVHTFPAVLQSSGLYSLSSVVTVP |
| | | | SSSLGTKTYTCNVDHKPSNTKVDKRVESKYGPP |
| | | | CPPCPAPEFLGGPSVFLFPPKPKDTLMISRTPE |
| | | | VTCVVVDVSQEDPEVQFNWYVDGVEVHNAKTKP |
| | | | REEQFNSTYRVVSVLTVLHQDWLNGKEYKCKVS |
| | | | NKGLPSSIEKTISKAKGQPREPQVYTLPPSQEE |
| | | | MTKNQVSLTCLVKGFYPSDIAVEWESNGQPENN |
| | | | YKTTPPVLDSDGSFFLYSRLTVDKSRWQEGNVF |
| | | | SCSVMHEALHNHYTQKSLSLSLGK |
| 278 | LC | 27597 | EIVLTQSPATLSLSPGERATLSCRASQSVSSYL |
| | | | AWYQQKPGQAPRLLIYDASNRATGIPARFSGSG |
| | | | SGTDFTLTISSLEPEDFAVYYCQQFVLWPRTFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 279 | HC | 27575 | QVQLVQSGAEVKEPGSSVKVSCKASGGTFSSYA |
| | | | TSWVRQAPGQGLEWMGGIIPISGTANYAQEFQG |
| | | | RVTITADESTSTAYMELSSLRSEDTAVYYCARD |
| | | | GGGYRHHYFDLWGRGTLVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLIVDKSRWQEGNVESCSVM |
| | | | HEALHNHYTQKSLSLSLGK |
| 280 | LC | 27575 | EIVLTQSPATLSLSPGERATLSCRASQSVSRYL |
| | | | AWYQQKPGQAPRLLIYDASNRATGIPARFSGSG |
| | | | SGTDFTLTISSLEPEDFAVYYCQQHVNFPLTFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 281 | HC | 27568 | QVQLVQSGAEVKKPGSSVKVPCKASGGTFSSYA |
| | | | ISWVRQAPEQGLEWMGSIIPIEGTANYAQKFQG |
| | | | RVTITADESTSTAYMELSSLRSEDTAVYYCAGE |
| | | | SGGYRDHKLDVWGQGTVVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 282 | LC | 27568 | EIVLTQSPATLSLSPGERATLSCRASQSVSSYL |
| | | | AWYQQKPGQAPRLLIYDASNRATGIPARFSGSG |
| | | | SGTDFTLTISSLEPEDFAVYYCQQSVFWPITFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 283 | HC | 27577 | QVQLVQSGAEVKKPGSSVKVSCKASGGAFSSYA |
| | | | IGWVRQAPGQGLEWMGGIIPTFGTANYAQKFQG |
| | | | RVTITADESTSTAYMELSSLRSEDTAVYYCARD |
| | | | GGGYQHHYFDLWGRGTLVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 284 | LC | 27577 | EIVMTQSPATLSVSPGERATLSCRASQSVGSNL |
| | | | AWYQQKPGQAPRLLIYGASTRATGIPARFSGSG |
| | | | SGTEFTLTISSLQSEDFAVYYCQQLTKWPLTFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 285 | HC | 27587 | QVQLVQSGAEVKKPGSSVKVSCKASGGTFSSYA |
| | | | ISWVRQAPGQGLEWMGSIIPIFGTANYAQKFQG |
| | | | RVTITADESTSTAYMELSSLRSEDTAVYYCARE |
| | | | SGGYRDHKLDVWGQGTMVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 286 | LC | 27587 | EIVLTQSPATLSLSPGERATLSCRASQSVSSYL |
| | | | AWYQQKPGQAPRLLIYDASKRATGIPARFSGSG |
| | | | SGTDFTLTISSLEPEDFAVYYCQQDVLWPLTFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 287 | HC | 27589 | QVQLVQSGAEVKKPGSSVKVSCKASGGTFSSYA |
| | | | ISWVRQAPGQGLEWMGGIIPIFGTANYAQKFQG |
| | | | RVTITADESTSTAYMELSSLRSEDTAVYYCARD |
| | | | SGYHRHYSDYWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSLGK |
| 288 | LC | 27589 | DIQMTQSPSTLSASVGDRVTITCRASQSISSWL |
| | | | AWYQQKPGKAPKLLIYKASSLESGVPSRFSGSG |
| | | | SGTEFTLTISSLQPDDFATYYCQQYCLFPITFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 289 | HC | 27596 | QVQLVQSCAEVKKPCSSVKVSCKASGGTFSSYA |
| | | | ISWVRQAPGQGLEWMGGIIPIFGTANYAQKFQG |
| | | | RVTITADESTSTAYMELSSLRSEDTAVYYCARD |
| | | | PLGIRKHWFDPWGQGTLVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDTAVEWESNGQPENNYKTTP |
| | | | PVLDSDGSFFLYSRLIVDKSRWQEGNVFSCSVM |
| | | | HEALHNHYTQKSLSLSLGK |
| 290 | LC | 27596 | EIVMTQSPATLSLSPGERATLSCRASQSVSSYL |
| | | | AWYQQKPGQAPRLLIYDASNRATGIPARFSGSG |
| | | | SGTDFTLTISSLEPEDFAVYYCQQHTVWPITFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 291 | HC | 27535 | QVQLVQSGAEVKKPGSSVKVSCKASGGTFSSYA |
| | | | ISWVRQAPGQGLEWMGGIIPIFGTANYAQKEQG |
| | | | RVTITADESTSTAYMELSSLRSEDTAVYYCARD |
| | | | TPRWRYHYFDYWGQGTLVTVSSASTKGPSVFPL |
| | | | APCSRSTSESTAALGCLVKDYEPEPVTVSWNSG |
| | | | ALTSGVHTFPAVLQSSGLYSLSSVVTVPSSSLG |
| | | | TKTYTCNVDHKPSNTKVDKRVESKYGEPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYECKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 292 | LC | 27535 | EIVLTQSPATLSLSPGERATLSCRASQSVSSYL |
| | | | AWYQQKPGQAPRLLIYDASKRATGIPARFSGSG |
| | | | SGTDFTLTISSLEPEDFAVYYCQQVLNYPLTFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 293 | HC | 27550 | EVQLVESGGGLVKPGGSLRLSCAASGFTFSSYS |
| | | | MNWVRQAPGKGLEWVSSISSSSSYIYYADSVKG |
| | | | RFTISRDNAKNSLYLQMNSLRAEDTAVYYCARE |
| | | | RRGSLALGMDVWGQGTLVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKIKPREEQF |
| | | | NSTYRVVSVLTVLHQDWLNGKEYKCKVSNKGLP |
| | | | SSIEKTISKAKGQPREPQVYTLPFSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 294 | LC | 27550 | DIQMTQSPSSLSASVGDRVTITCQASQDISNYL |
| | | | NWYQQKPGKAPKLLIYDASNLETGVPSRFSGSG |
| | | | SGTDFTFTISSLQPEDIATYYCQQSYFLPPTFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 295 | HC | 27549 | QVQLVESGGGVVQPGRSLRLSCAASGFTFSSYG |
| | | | MNWVRQAPGKGLEWVAVIWYDGSNKYYADSVKG |
| | | | RFTISRDNSKNTLYLQMNSLRAEDTAVYYCARD |
| | | | LGGYSYGEPYYYYYGMDVWGQGTTVTVSSASTK |
| | | | GPSVFPLAPCSRSTSESTAALGCLVKDYFPEPV |
| | | | TVSWNSGALTSGVHTFPAVLQSSGLYSLSSVVT |
| | | | VPSSSLGTKTYTCNVDHKPSNTKVDKRVESKYG |
| | | | PPCPPCPAPEFLGGPSVFLFPPKPKDTLMISRT |
| | | | PEVTCVVVDVSQEDPEVQFNWYVDGVEVHNAKT |
| | | | KPREEQFNSTYRVVSVLTVLHQDWLNGKEYKCK |
| | | | VSNKGLPSSIEKTISKAKGQPREPQVYTLPPSQ |
| | | | EEMTKNQVSLTCLVKGFYPSDIAVEWESNGQPE |
| | | | NNYKTTPPVLDSDGSFFLYSRLTVDKSRWQEGN |
| | | | VFSCSVMHEALHNHYTQKSLSLSLGK |
| 296 | LC | 27549 | EIVLTQSPGTLSLSPGERATLSCRASQSVSSDY |
| | | | LAWYQQKPGQAPRLLIYGASSRATGIPDRFSGS |
| | | | GSGTDFTLTISRLEPEDFAVYYCQQAHSSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 297 | HC | 31414 | QVQLVQSGAEVKKPGASVKVSCKASGYTFKSYE |
| | | | MHWVRQAPGQGLEWMGRINPSVGSTWYAQKFQG |
| | | | RVTMTRDTSTSTVYMELSSLRSEDTAVYYCARG |
| | | | KREGGTEYLRNWGQGTLVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSLGK |
| 298 | LC | 31414 | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQYHSYITFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

What is claimed is:

1. An antigen binding protein that binds specifically to a human CD39 (hCD39) and is capable of 1, 2, 3, 4, 5, 6, 7, 8, 9, 10, 11, 12, 13, 14, 15, 16, 17, or 18 of the following:
a) inhibiting binding of CD39 to ATP;
b) inhibiting conversion by CD39 of ATP to ADP and/or ADP to AMP;
c) decreasing affinity of CD39 for ATP or ADP;
d) inhibiting or impeding release of ADP or AMP from CD39;
e) impeding or inhibiting CD39 processivity;
f) inhibiting platelet aggregation;
g) decreasing levels of phosphate, ADP, AMP, and/or adenosine and/or increasing levels of ATP;
h) increasing T effector cell function;
i) decreasing the number of regulatory T cells in tissues or in circulation;
j) suppressing regulatory T cells or regulatory T cell activity;
k) increasing B cell function;
l) increasing antigen presenting cell function;
m) inhibiting CD39 function on tumor cells;
n) inhibiting processing of at least one of phospho-antigen from phosphorylated isoprenoid, phosphorylated vitamin B metabolite, and/or phosphorylated riboflavin;
o) decreasing or preventing activation of phospho antigen specific T cells selected from MAIT cells and γδ T cells;
p) inhibiting angiogenesis;
q) increasing proliferation of stimulated CD4⁺ and CD8⁺ T cells;
r) increasing stimulated PBMC Secretion of INF-γ, TNF-α, IL-2 and/or IL-1β.

2. The antigen binding protein of claim 1, wherein the antigen binding protein has 1, 2, 3, 4, 5, 6, or 7 of the following characteristics:
a) is a monoclonal antibody;
b) is a human antibody, a humanized antibody, or a chimeric antibody;
c) is a bispecific antibody, a multi-specific antibody, a diabody, or a multivalent antibody;
d) is of the IgG1, IgG2, IgG3, IgG4, or IgM type;
e) is an antigen-binding antibody fragment;
f) is a Fab fragment, a Fab′ fragment, a F(ab′)2 fragment, or an Fv fragment; and/or
g) is a single chain antibody, a single domain antibody, or a nanobody.

3. A pharmaceutical composition comprising an effective amount of an antibody which binds to hCD39 and has 1, 2, 3, 4, 5, 6, 7, 8, 9, 10, 11, 12, 13, 14, 15, or 16 of the following of characteristics:
a) blocks or decreases hydrolysis of ATP to ADP and/or ADP to AMP as determined by at least one of: (i) a decreased phosphate release (Pi), (ii) an increase in ATP levels, and (iii) a decrease of ADP, AMP, and/or adenosine levels,
b) increases T effector cell activity;
c) suppresses regulatory T cell or decreases a regulatory T cell activity;
d) decreases number of regulatory T cells in tissues or in circulation;
e) increases B cell function;
f) increases antigen presenting cell function;
g) inhibits CD39 function on tumor cells;
h) blocks or inhibits processing of at least one of phospho-antigen from a phosphorylated isoprenoid, phosphorylated vitamin B metabolite, and phosphorylated riboflavin;
i) decreases or prevents activation of phospho antigen specific T cells selected from MAIT cells and γδ T cells;
k) inhibits angiogenesis;
l) decreases affinity for ATP and/or ADP;
m) inhibits release of ADP or AMP from CD39;
n) impedes or inhibits CD39 processivity;
o) inhibits platelet aggregation;
p) increases proliferation of stimulated CD4⁺ and CD8⁺ T cells;
r) increases stimulated PBMC Secretion of INF-γ, TNF-α, IL-2, and/or IL-1β.

4. A pharmaceutical composition comprising the antigen-binding protein of claim 1 or claim 2.

5. The pharmaceutical composition of claim 4, further comprising an effective amount of at least one of the following
a) an anti-PD-1 antibody,
b) an anti-PD-L1 antibody,
c) an anti-CD73 antibody,
d) an anti-CD38 antibody,
e) an anti-A2A receptor antibody,
f) an anti-A2B receptor antibody,
g) an anti-A2A/A2B dual receptor antibody, or any combination thereof, or
h) a small molecule inhibitor,
or a combination thereof.

6. The pharmaceutical composition of claim 4 or claim 5, further comprising one or both of
a) an antibody to an inhibitory receptor or ligand and/or
b) an antibody to a costimulatory receptor or ligand,
or a combination thereof.

7. The pharmaceutical composition of claim 6, wherein
(i) the inhibitory receptor or ligand is at least one of CTLA-4, PD-L2, LAG-3, Tim3, neuritin, BTLA, CECAM-1, CECAM-5, VISTA, LAIR1, CD160, 2B4, TGF-R, HHLA2, ILT2, ILT3, ILT4, HLA-G, HLA-C, and/or a Killer-cell immunoglobulin-like receptor (KIR) and/or
(ii) the costimulatory receptor or ligand is at least one of OX40, CD2, CD27, CDS, ICAM-1, LFA-1, ICOS (CD278), 4-1BB (CD137), GITR, CD28, CD30, CD40, BAFFR, HVEM, CD7, LIGHT, NKG2C, SLAMF7, NKp80, CD160, B7-H3, and/or CD83.

8. The pharmaceutical composition of claim 7, wherein the costimulatory receptor or ligand LFA-1 further comprises an LFA-1 β-chain CD18 and/or an LFA-1 α-chain CD11a.

9. The antigen binding protein of claim 1, wherein the antigen binding protein has one or more of the following characteristics:
a) binds to a human CD39 polypeptide or a variant thereof with a KD of less than about 20 nM;
b) binds to a cyno CD39 polypeptide or a variant thereof with a KD of less than about 200 nM;
c) binds to a murine CD39 polypeptide or a variant thereof with a KD of less than about 200 nM; or
d) a combination of at least 2 of a), b), and c).

10. An antigen binding protein that competes or is capable of competing for binding to human CD39 with a reference antigen binding protein, wherein the reference antigen binding protein is the antigen binding protein of claim 1.

11. An antigen binding protein that binds to or is capable of competing for binding to human CD39 with a reference antigen binding protein, wherein the reference antigen binding protein binds to an epitope at positions 143-158 or 274-277 of SEQ ID NO: 249 on a human CD39 polypeptide, including, but not limited to, D150, E153, R154 or N99 alone or in combination with D150, E153, R154 or any combination thereof.

12. The antigen binding protein of claim 10 or claim 11, wherein the antigen binding protein and the reference antibody cross-compete or are capable of cross-competing for binding to a human CD39.

13. The antigen binding protein of claim 1, comprising a human heavy chain constant region or fragment or a variant thereof, wherein the constant region variant comprises up to 20 conservatively modified amino acid substitutions from any sequence set forth SEQ ID NOs: 179-218.

14. An isolated antibody molecule capable of binding to human CD39 (hCD39), comprising a heavy chain variable region (VH) and a light chain variable region (VL), VH and/or VL comprising 1, 2, 3, 4, 5, or 6 of:
a) a VHCDR1 having the sequence set forth in SEQ ID NOs: 1-45,
b) a VHCDR2 having the sequence set forth in SEQ ID NOs: 46-81,
c) a VHCDR3 having the sequence set forth in SEQ ID NOs: 82-109,
d) a VLCDR1 having the sequence set forth in SEQ ID NOs: 110-124,
e) a VLCDR2 having the sequence set forth in SEQ ID NOs: 125-140, and
f) a VLCDR3 having the sequence set forth in SEQ ID NOs: 141-166.

15. An isolated antibody molecule capable of binding to human CD39 (hCD39), comprising a heavy chain variable region (VH) and a light chain variable region (VL), the VH comprising,
a) a VHCDR1 having a sequence set forth in SEQ ID NOs: 1-45,
b) a VHCDR2 having a sequence set forth in SEQ ID NOs: 46-81, and
c) a VHCDR3 having a sequence set forth in SEQ ID NOs: 82-109; and the V_{L} comprising,
a) a VLCDR1 having a sequence set forth in SEQ ID NO: 110-124,
b) a VLCDR2 having a sequence set forth in SEQ ID NO: 125-140, and
c) a VLCDR3 having a sequence set forth in SEQ ID NO: 141-166.

16. An isolated nucleic acid encoding an antigen binding protein according to claim 1, claim 14, or claim 15.

17. An expression vector comprising the nucleic acid according to claim 16.

18. A prokaryotic or eukaryotic host cell comprising the vector of claim 17.

19. An oncolytic virus encoding the nucleic acid of either of claim 16 or 17.

20. A method for the production of a recombinant protein comprising the steps of expressing a nucleic acid according to claim 16 in a prokaryotic or eukaryotic host cell and recovering the protein from the cell or the cell culture supernatant.

21. A method for treatment of a subject suffering from cancer, a chronic infection, or from an inflammatory disease, comprising the step of administering to the subject a pharmaceutical composition comprising an effective amount of the antigen binding protein of claim 1 or the pharmaceutical composition of claim 3.

22. The method of claim 21, wherein the cancer is a solid cancer.

23. The method of claim 21, wherein the cancer is a hematological cancer.

24. A method for modulating immune system function in a subject in need thereof, comprising the step of contacting a population of immune cells of the subject with a pharmaceutical composition comprising an effective amount of the antigen binding protein of claim 1, under conditions such that the immune system is modulated.

25. A method for inducing or enhancing an immune response in a subject in need thereof, comprising the step of administering to the subject a pharmaceutical composition comprising an antigen binding protein, wherein the immune response is generated against a tumor antigen.

26. The method of claim 24 or claim 25, wherein the subject is a human subject.

27. The method of claim 25, wherein the antigen binding protein comprises a bispecific antibody or a complexing antigen binding protein.

28. The method of claim 27, wherein the antigen binding protein, the bispecific antibody, or the complexing antigen binding protein is administered in an amount sufficient to achieve 1, 2, 3, 4, 5, 6, or 7 of the following in the subject:
a) reduction of CD39 ATPase activity in a target cell population;
b) reduction of regulatory T cells suppression of activity of effector T cells;
c) reduction of levels of regulatory T cells;
d) activation of effector T cells;
e) induction or enhancement of effector T cell proliferation;
f) inhibition of tumor growth; and/or
g) induction of tumor regression.

29. The method of claim 28, wherein the target cell population comprises T cells, B cells, monocytes, macrophages, dendritic cells, myeloid-derived suppressor cells, and/or tumor cells.

30. The method of claim 25, wherein the method further comprises one or more of the following
a) administering chemotherapy;
b) administering radiation therapy; and/or
c) administering one or more additional therapeutic agents.

31. The method of claim 30, wherein the one or more additional therapeutic agents comprise one or more immunostimulatory agents.

32. The method of claim 31, wherein the one or more immunostimulatory agents comprise an antagonist to an inhibitory receptor of an immune cell.

33. The method of claim 32, wherein the inhibitory receptor is at least one of CTLA-4, PD-1, PD-L1, PD-L2, LAG-3, Tim3, neuritin, BTLA, CECAM-1, CECAM-5, VISTA, LAIR1, CD160, 2B4, TGF-R, and/or a Killer-cell immunoglobulin-like receptor (KIR).

34. The method of claim 31, wherein the one or more immunostimulatory agents comprise an agonist of a co-stimulatory receptor of an immune cell.

35. The method of claim 34, wherein the co-stimulatory receptor is OX40, CD2, CD27, CDS, ICAM-1, LFA-1, ICOS (CD278), 4-1BB (CD137), GITR, CD28, CD30, CD40, BAFFR, HVEM, CD7, LIGHT, NKG2C, SLAMF7, NKp80, CD160, B7-H3, or a CD83 ligand.

36. The method of claim 35, wherein the costimulatory receptor or ligand LFA-1 further comprises an LFA-1 β-chains CD18 and/or an LFA-1 α-chain CD11a.

37. The method of claim 31, wherein the one or more immunostimulatory agents comprise a cytokine.

38. The method of claim 37, wherein the cytokine is at least one of IL-2, IL-5, IL-7, IL-12, IL-15, and/or IL-21.

39. The method of claim 31, wherein the one or more immunostimulatory agents comprise an oncolytic virus.

40. The method of claim 39, wherein the oncolytic virus is a Herpes simplex virus, a Vesicular stomatitis virus, an adenovirus, a Newcastle disease virus, a vaccinia virus, or a maraba virus.

41. The method of claim 31, wherein the one or more immunostimulatory agents comprise a chimeric antigen engineered T cell.

42. The method of claim 31, wherein the one or more immunostimulatory agents comprise a bi- or multispecific T cell directed antibody.

43. The method of claim 25, wherein the one or more additional therapeutic agents comprise at least one trap for an immune suppressive agent.

44. The method of claim 43, wherein the at least one trap for an immune suppressive agent comprises at least one of an anti-TGF-beta antibody, a TGFb receptor trap, an anti-IL-10 antibody and/or anti-IL-10 receptor trap, and/or an anti-IL-35 antibody trap and/or an anti-IL-35 receptor trap.

45. The method of any one of any of claims 3-44, wherein administration of the pharmaceutical composition results in induction or enhancement of proliferation of a T-effector cell, or modulation of I-kappaB and/or NF-κB in the T cell, or modulation of CD39 activity in the T cell, or T cell receptor induced signaling in a T-effector cell, or a combination thereof.

46. A method of screening for a test compound comprising an antigen binding protein of claim 1 capable of inhibiting an activity of CD39, comprising the steps of:
contacting a test sample containing CD39 with a test compound;
comparing the activity of the test sample to a control sample;
whereby a decrease in the activity of CD39 in the test sample compared to the control sample identifies the compound as one that inhibits the activity of CD39.

47. The method of claim 46, wherein the control sample comprises a sample not contacted with a test compound.

48. An isolated antibody molecule capable of binding to human CD39 (hCD39), comprising a heavy chain variable region (VH) and a light chain variable region (VL), VH comprising at least one of:
a) a VHCDR1 having an amino acid sequence that is at least 90% identical to the sequence set forth in SEQ ID NOs: 1-45,
b) a VHCDR2 having an amino acid sequence that is at least 90% identical to the sequence set forth in SEQ ID NOs: 46-81, and
c) a VHCDR3 having an amino acid sequence that is at least 90% identical to the sequence set forth in SEQ ID NOs: 82-109; and
VL comprising at least one of:
a) a VLCDR1 having an amino acid sequence that is at least 90% identical to the sequence set forth in SEQ ID NOs: 110-124,
b) a VLCDR2 having an amino acid sequence that is at least 90% identical to the sequence set forth in SEQ ID NOs: 125-140, and
c) a VLCDR3 having an amino acid sequence that is at least 90% identical to the sequence set forth in SEQ ID NOs 141-166.

49. An isolated antibody molecule capable of binding to human CD39 (hCD39), comprising a heavy chain variable region (VH) and a light chain variable region (VL),
VH comprising at least one of:
a) a VHCDR1 having an amino acid sequence that is homologous to the sequence set forth in SEQ ID NOs: 1-45,
b) a VHCDR2 having an amino acid sequence that is homologous to the sequence set forth in SEQ ID NOs: 46-81, and
c) a VHCDR3 having an amino acid sequence that is homologous to the sequence set forth in SEQ ID NOs: 82-109; and
VL comprising at least one of:
a) a VLCDR1 having an amino acid sequence that is homologous to the sequence set forth in SEQ ID NOs: 110-124,
b) a VLCDR2 having an amino acid sequence that is homologous to the sequence set forth in SEQ ID NOs: 125-140, and
c) a VLCDR3 having an amino acid sequence that is homologous to the sequence set forth in SEQ ID NOs: 141-166.

50. An isolated antibody molecule capable of binding to human CD39 (hCD39), comprising a heavy chain and a light chain, the heavy chain comprising one or more molecules having a sequence consisting of one of SEQ ID NO: 255, SEQ ID NO: 257, SEQ ID NO: 259, SEQ ID NO: 261, SEQ ID NO: 263, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, or SEQ ID NO: 295 and the light chain comprising one or more molecules having a sequence consisting of one of SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, or SEQ ID NO: 296.

51. An isolated antibody molecule capable of binding to human CD39 (hCD39), comprising a heavy chain and a light chain,
a) the heavy chain comprising one or more molecules, each molecule having a sequence consisting of SEQ ID NO: 255 and the light chain comprising one or more, each molecule having a sequence consisting of SEQ ID NO: 256;
b) the heavy chain comprising one or more molecules, each molecule having a sequence consisting of SEQ ID NO: 257 and the light chain comprising one or more molecules, each molecule having a sequence consisting of SEQ ID NO: 258;
c) the heavy chain comprising one or more molecules, each molecule having a sequence consisting of SEQ ID NO: 259 and the light chain comprising one or more molecules, each molecule having a sequence consisting of SEQ ID NO: 260;
d) the heavy chain comprising one or more molecules, each molecule having a sequence consisting of SEQ ID NO: 261 and the light chain comprising one or more molecules, each molecule having a sequence consisting of SEQ ID NO: 262;
c) the heavy chain comprising one or more molecules, each molecule having a sequence consisting of SEQ ID NO: 263 and the light chain comprising one or more molecules, each molecule having a sequence consisting of SEQ ID NO: 264;
f) the heavy chain comprising one or more molecules, each molecule having a sequence consisting of SEQ ID NO: 265 and the light chain comprising one or more molecules, each molecule having a sequence consisting of SEQ ID NO: 266;
g) the heavy chain comprising one or more molecules, each molecule having a sequence consisting of SEQ ID NO: 267 and the light chain comprising one or more molecules, each molecule having a sequence consisting of SEQ ID NO: 268;
h) the heavy chain comprising one or more molecules, each molecule having a sequence consisting of SEQ ID NO: 269 and the light chain comprising one or more molecules, each molecule having a sequence consisting of SEQ ID NO: 270;
i) the heavy chain comprising one or more molecules, each molecule having a sequence consisting of SEQ ID NO: 271 and the light chain comprising one or more molecules, each molecule having a sequence consisting of SEQ ID NO: 272;
j) the heavy chain comprising one or more molecules, each molecule having a sequence consisting of SEQ ID NO: 273 and the light chain comprising one or more molecules, each molecule having a sequence consisting of SEQ ID NO: 274;
k) the heavy chain comprising one or more molecules, each molecule having a sequence consisting of SEQ ID NO: 275 and the light chain comprising one or more molecules, each molecule having a sequence consisting of SEQ ID NO: 276;
l) the heavy chain comprising one or more molecules, each molecule having a sequence consisting of SEQ ID NO: 277 and the light chain comprising one or more molecules, each molecule having a sequence consisting of SEQ ID NO: 278
m) the heavy chain comprising one or more molecules, each molecule having a sequence consisting of SEQ ID NO: 279 and the light chain comprising one or more molecules, each molecule having a sequence consisting of SEQ ID NO: 280;
n) the heavy chain comprising one or more molecules, each molecule having a sequence consisting of SEQ ID NO: 281 and the light chain comprising one or more molecules, each molecule having a sequence consisting of SEQ ID NO: 282;
o) the heavy chain comprising one or more molecules, each molecule having a sequence consisting of SEQ ID NO: 283 and the light chain comprising one or more molecules, each molecule having a sequence consisting of SEQ ID NO: 284;
p) the heavy chain comprising one or more molecules, each molecule having a sequence consisting of SEQ ID NO: 285 and the light chain comprising one or more molecules, each molecule having a sequence consisting of SEQ ID NO: 286;
q) the heavy chain comprising one or more molecules, each molecule having a sequence consisting of SEQ ID NO: 287 and the light chain comprising one or more molecules, each molecule having a sequence consisting of SEQ ID NO: 288;
r) the heavy chain comprising one or more molecules, each molecule having a sequence consisting of SEQ ID NO: 289 and the light chain comprising one or more molecules, each molecule having a sequence consisting of SEQ ID NO: 290;
s) the heavy chain comprising one or more molecules, each molecule having a sequence consisting of SEQ ID NO: 291 and the light chain comprising one or more molecules, each molecule having a sequence consisting of SEQ ID NO: 292;
t) the heavy chain comprising one or more molecules, each molecule having a sequence consisting of SEQ ID NO: 293 and the light chain comprising one or more molecules, each molecule having a sequence consisting of SEQ ID NO: 294; or
u) the heavy chain comprising one or more molecules, each molecule having a sequence consisting of SEQ ID NO: 295 and the light chain comprising one or more molecules, each molecule having a sequence consisting of SEQ ID NO: 296.

52. An isolated nucleic acid encoding an antigen binding protein according to any one of claims 48-51.

53. An isolated antibody molecule capable of binding to human CD39 (hCD39), comprising a heavy chain variable region (VH) and a light chain variable region (VL), VH and/or VL comprising 1, 2, 3, 4, 5, or 6 of:
a) a VHCDR1 sequence comprising:
(i) a Kabat CDRH1 sequence defined by the consensus sequence S-Y-Δ₃-M-Δ₅ (SEQ ID NOS: 25-29 and 44-45), where Δ₃ is E, F, Q, or Y and Δ₅ is H or Y;
(ii) a Kabat CDR-H1 sequence defined by the consensus sequence θ₁-θ₂-θ₃-I-S (SEQ ID NOS: 30-37), where θ₁ is A, H, K, L, S, or W; θ₂ is L, M, N, or T; and θ₃ is A or P;
(iii) a Kabat CDR-H1 sequence defined by the consensus sequence η₁-Y-η₃-I-S SEQ ID NOS: 38-41), where η₁ is S, K, N, or R and η₃ is A or G;
(iv) a Chothia CDR-H1 sequence defined by the consensus sequence G-Y-T-F-Ω₅-S-Y (SEQ ID NOS: 1-2 and 4-6), where Ω₅ is T, K, Q, F, or V;
(v) a Chothia CDR-H1 sequence defined by the consensus sequence G-G-T-F-ν₅-ν₆-Y (SEQ ID NOS: 17-22 and 24), where ν₅ is S, G, or E and ν₆ is S, K, R, or S; or
(vi) a Chothia CDR-H1 consensus sequence defined by the consensus sequence G-G-T-F-κ₅-κ₆-κ₇ (SEQ ID NOS: 7-16), where κ₅ is S, Q, P, or A; κ₆ is S, K, H, L, A, or W; and κ₇ is Y, L, T, N, or M,
b) a VHCDR2 sequence comprising:
(i) a Kabat CDR-H2 sequence defined by the consensus sequence ε₁-I-N-P-ε₅-ε₆-G-S-T-ε₁₀-Y-A-Q-K-F-Q-G (SEQ ID NOS: 63-66 and 68), where ε₁ is K, S, R, or V; ε₅ is L, R, or S; ε₆ is G or V; and ε₁₀ is S or W;
(ii) a Kabat CDR-H2 sequence defined by the consensus sequence G-I-α₃-α₄-α₅-α₆-G-T-A-N-Y-A-Q-K-F-Q-G (SEQ ID NOS: 69-72), where α₃ is I or L or is absent; α₄ is P or is absent; and α₅ is I, G, or R; and α₆ is A, F, or G;
(iii) a Kabat CDR-H2 sequence defined by the consensus sequence β₁-I-I-P-β₅-β₆-G-β₈-A-N-Y-A-Q-K-F-G-Q (SEQ ID NOS: 74 and 76-79), where β₁ is S or G; β₅ is I, E, S, or T; β₆ is F, I, or S; and β₈ is I or T;
(iv) a Chothia CDR-H2 sequence defined by the consensus sequence N-P-ε₅-ε₆-G-S-T (SEQ ID NOS: 46-48), where ε₅ is L, R, or S and ε₆ is G or V;
(v) a Chothia CDR-H2 sequence defined by the consensus sequence α₃-α₄-α₅-α₆-G-T-A (SEQ ID NOS: 51-54), where α₃ is I or L or is absent; α₄ is P or is absent; and α₅ is I, G, or R; and α₆ is A, F, or G; or
(vi) a Chothia CDR-H2 sequence defined by the consensus sequence I-P-β₅-β₆-G-β₈-A (SEQ ID NOS: 56-60), where β₅ is I, E, S, or T; β₆ is F, I, or S; and β₈ is I or T,
c) a VHCDR3 sequence comprising:
(i) a CDR-H3 sequence defined by the consensus sequence G-K-R-E-G-G-T-E-Y-L-R-γ₁₂ (SEQ ID NOS: 82-86), where γ₁₂ is H, K, S, N, or V;
(ii) a CDR-H3 sequence defined by the consensus sequence E-S-G-Φ₄-Y-R-D-H-R-L-Φ₁₁-V (SEQ ID NOS: 94-96), where Φ₄ is G or T and Φ₁₁ is D or G; or
(iii) a CDR-H3 sequence defined by the consensus sequence G-G-A-K-Y-A-∃₇-∃₈-∃₉-G-M-D-V (SEQ ID NOS: 87-93), where is, V, G or R; ∃₈ T, Q, K, G, or R; and ∃₉ is Y, H, L, or W,
d) a VLCDR1 sequence comprising:
(i) a CDR-L1 sequence defined by the consensus sequence ϕ₁-A-S-ϕ₄-ϕ₅-V-ϕ₇-ϕ₈-ϕ₉-Y-L-A (SEQ ID NOS: 1101-114), where ϕ₁ is E, K, or R; ϕ₄ is Q or E; ϕ₅ is S or Y; ϕ₇ is S or A; ϕ₈ is S or Y; and ϕ₉ is D or S;
(ii) a CDR-L1 sequence defined by the consensus sequence σ₁-A-S-Q-σ₅-σ₆-σ₇-σ₈-σ₉-L-σ₁₁ (SEQ ID NOS: 118 and 120-123), where σ₁ is Q or R1; σ₅ is D or S; σ₆ is I or V; σ₇ is G or S; σ₈ is N, R, or S; σ₉ is N, Y, or W; and σ₁₁ is A or N; or
(iii) a CDR-L1 sequence defined by the consensus sequence K-S-S-Γ₄-S-V-L-Γ₈-S-Γ₁₀-N-N-K-N-Y-L-A (SEQ ID NOS: 115-117), where Γ₄ is Q, R or K; Γ₈ is F or Y; and Γ₁₀ is S or N,
e) a VLCDR2 sequence comprising:
(i) a CDR-L2 sequence defined by the consensus sequence ψ₁-A-S-ψ₄-R-ψ₆-ψ₇ (SEQ ID NOS: 125-136), where ψ₁ is G or Y, ψ₄ is S or N; ψ₆ is A or H; and ψ₇ is T, Y, or N;
(ii) a CDR-L2 sequence defined by the consensus sequence D-A-S-χ₄-R-A-T (SEQ ID NOS: 138 and 139), where £₄ is N or K; or
(iii) a CDR-L2 sequence defined by the consensus sequence W-A-S-T-R-σ₆-S (SEQ ID NOS: 131 and 133-134), where σ₆ is A, E, or Q, and
f) a VLCDR3 sequence comprising:
(i) a CDR-L3 sequence defined by the consensus sequence Q-Q-Y-π₄-π₅-π₆-π₇-T (SEQ ID NOS: 141-147), where π₄ is G, H, or Y; π₅ is S, N, F, G, or R; π₆ is S, Y, A, G, or R; and π₇ is P, I, or L;
(ii) a CDR-L3 sequence defined by consensus sequence Q-Q-λ₃-λ₄-λ₅-λ₆-P-T (SEQ ID NOS: 148-150), where λ₃ is R, F, H, S, L, D, Y, or V; λ₄ is S, V, T, G, L, Y, or N; λ₅ is N, L, F, K, or V; and λ₆ is W, F, Y, or L;
(iii) a CDR-L3 sequence defined by the consensus sequence Q-Q-Y-ρ₃-ρ₄-W-P-L-T (SEQ ID NOS: 151 and 152), where ρ₃ is N or L and ρ₄ is N or L; or
(iv) a CDR-L3 sequence defined by the consensus sequence Q-Q-ω₃-ω₄-ω₅-ω₆-P-ω₈-T (SEQ ID NOS: 153-156), where ω₃ is Y or F; ω₄ is Y or W; ω₅ is S, L, T, or F; ω₆ is T, Y, or F; and ω₈ is L or P.


SEQUENCE LISTING (296 sequences)

SEQ ID NO: 1 - protein, 7 aa, synthetic construct
  REGION 1..7: note = Synthetic: CDR-H1, Chothia
GYTFTSY

SEQ ID NO: 2 - protein, 7 aa, synthetic construct
  REGION 1..7: note = Synthetic: CDR-H1, Chothia
GYTFKSY

SEQ ID NO: 3 - protein, 7 aa, synthetic construct
  REGION 1..7: note = Synthetic: CDR-H1, Chothia
GYIFKSY

SEQ ID NO: 4 - protein, 7 aa, synthetic construct
  REGION 1..7: note = Synthetic: CDR-H1, Chothia
GYTFQSY

SEQ ID NO: 5 - protein, 7 aa, synthetic construct
  REGION 1..7: note = Synthetic: CDR-H1, Chothia
GYTFFSY

SEQ ID NO: 6 - protein, 7 aa, synthetic construct
  REGION 1..7: note = Synthetic: CDR-H1, Chothia
GYTFVSY

SEQ ID NO: 7 - protein, 10 aa, synthetic construct
  REGION 1..10: note = Synthetic: CDR-H1, Chothia
GGTFSSLAIS

SEQ ID NO: 8 - protein, 10 aa, synthetic construct
  REGION 1..10: note = Synthetic: CDR-H1, Chothia
GGTFSKLAIS

SEQ ID NO: 9 - protein, 7 aa, synthetic construct
  REGION 1..7: note = Synthetic: CDR-H1, Chothia
GGTFSHT

SEQ ID NO: 10 - protein, 7 aa, synthetic construct
  REGION 1..7: note = Synthetic: CDR-H1, Chothia
GGTFSSL

SEQ ID NO: 11 - protein, 7 aa, synthetic construct
  REGION 1..7: note = Synthetic: CDR-H1, Chothia
GGTFSLL

SEQ ID NO: 12 - protein, 7 aa, synthetic construct
  REGION 1..7: note = Synthetic: CDR-H1, Chothia
GGTFQSL

SEQ ID NO: 13 - protein, 7 aa, synthetic construct
  REGION 1..7: note = Synthetic: CDR-H1, Chothia
GGTFPSN

SEQ ID NO: 14 - protein, 7 aa, synthetic construct
  REGION 1..7: note = Synthetic: CDR-H1, Chothia
GGTFSAM

SEQ ID NO: 15 - protein, 7 aa, synthetic construct
  REGION 1..7: note = Synthetic: CDR-H1, Chothia
GGTFASL

SEQ ID NO: 16 - protein, 7 aa, synthetic construct
  REGION 1..7: note = Synthetic: CDR-H1, Chothia
GGTFSWL

SEQ ID NO: 17 - protein, 7 aa, synthetic construct
  REGION 1..7: note = Synthetic: CDR-H1, Chothia
GGTFSSY

SEQ ID NO: 18 - protein, 7 aa, synthetic construct
  REGION 1..7: note = Synthetic: CDR-H1, Chothia
GGTFGSY

SEQ ID NO: 19 - protein, 7 aa, synthetic construct
  REGION 1..7: note = Synthetic: CDR-H1, Chothia
GGTFSKY

SEQ ID NO: 20 - protein, 7 aa, synthetic construct
  REGION 1..7: note = Synthetic: CDR-H1, Chothia
GGTFGRY

SEQ ID NO: 21 - protein, 7 aa, synthetic construct
  REGION 1..7: note = Synthetic: CDR-H1, Chothia
GGTFESY

SEQ ID NO: 22 - protein, 7 aa, synthetic construct
  REGION 1..7: note = Synthetic: CDR-H1, Chothia
GGTFSNY

SEQ ID NO: 23 - protein, 7 aa, synthetic construct
  REGION 1..7: note = Synthetic: CDR-H1, Chothia
GGAFSSY

SEQ ID NO: 24 - protein, 7 aa, synthetic construct
  REGION 1..7: note = Synthetic: CDR-H1, Chothia
GFTFSSY

SEQ ID NO: 25 - protein, 5 aa, synthetic construct
  REGION 1..5: note = Synthetic: CDR-H1, Kabat
SYYMH

SEQ ID NO: 26 - protein, 5 aa, synthetic construct
  REGION 1..5: note = Synthetic: CDR-H1, Kabat
SYEMH

SEQ ID NO: 27 - protein, 5 aa, synthetic construct
  REGION 1..5: note = Synthetic: CDR-H1, Kabat
SYQMH

SEQ ID NO: 28 - protein, 5 aa, synthetic construct
  REGION 1..5: note = Synthetic: CDR-H1, Kabat
SYYMY

SEQ ID NO: 29 - protein, 5 aa, synthetic construct
  REGION 1..5: note = Synthetic: CDR-H1, Kabat
SYFMH

SEQ ID NO: 30 - protein, 5 aa, synthetic construct
  REGION 1..5: note = Synthetic: CDR-H1, Kabat
SLAIS

SEQ ID NO: 31 - protein, 5 aa, synthetic construct
  REGION 1..5: note = Synthetic: CDR-H1, Kabat
KLAIS

SEQ ID NO: 32 - protein, 5 aa, synthetic construct
  REGION 1..5: note = Synthetic: CDR-H1, Kabat
HTAIS

SEQ ID NO: 33 - protein, 5 aa, synthetic construct
  REGION 1..5: note = Synthetic: CDR-H1, Kabat
SLPIS

SEQ ID NO: 34 - protein, 5 aa, synthetic construct
  REGION 1..5: note = Synthetic: CDR-H1, Kabat
LLAIS

SEQ ID NO: 35 - protein, 5 aa, synthetic construct
  REGION 1..5: note = Synthetic: CDR-H1, Kabat
SNAIS

SEQ ID NO: 36 - protein, 5 aa, synthetic construct
  REGION 1..5: note = Synthetic: CDR-H1, Kabat
AMAIS

SEQ ID NO: 37 - protein, 5 aa, synthetic construct
  REGION 1..5: note = Synthetic: CDR-H1, Kabat
WLAIS

SEQ ID NO: 38 - protein, 5 aa, synthetic construct
  REGION 1..5: note = Synthetic: CDR-H1, Kabat
SYAIS

SEQ ID NO: 39 - protein, 5 aa, synthetic construct
  REGION 1..5: note = Synthetic: CDR-H1, Kabat
SYGIS

SEQ ID NO: 40 - protein, 5 aa, synthetic construct
  REGION 1..5: note = Synthetic: CDR-H1, Kabat
KYGIS

SEQ ID NO: 41 - protein, 5 aa, synthetic construct
  REGION 1..5: note = Synthetic: CDR-H1, Kabat
NYAIS

SEQ ID NO: 42 - protein, 5 aa, synthetic construct
  REGION 1..5: note = Synthetic: CDR-H1, Kabat
SYATS

SEQ ID NO: 43 - protein, 5 aa, synthetic construct
  REGION 1..5: note = Synthetic: CDR-H1, Kabat
SYAIG

SEQ ID NO: 44 - protein, 5 aa, synthetic construct
  REGION 1..5: note = Synthetic: CDR-H1, Kabat
SYSMN

SEQ ID NO: 45 - protein, 5 aa, synthetic construct
  REGION 1..5: note = Synthetic: CDR-H1, Kabat
SYGMN

SEQ ID NO: 46 - protein, 7 aa, synthetic construct
  REGION 1..7: note = Synthetic: CDR-H2, Chothia
NPSGGST

SEQ ID NO: 47 - protein, 6 aa, synthetic construct
  REGION 1..6: note = Synthetic: CDR-H2, Chothia
NPSVGS

SEQ ID NO: 48 - protein, 6 aa, synthetic construct
  REGION 1..6: note = Synthetic: CDR-H2, Chothia
NPSGGS

SEQ ID NO: 49 - protein, 6 aa, synthetic construct
  REGION 1..6: note = Synthetic: CDR-H2, Chothia
NPLGGG

SEQ ID NO: 50 - protein, 6 aa, synthetic construct
  REGION 1..6: note = Synthetic: CDR-H2, Chothia
NPRGGS

SEQ ID NO: 51 - protein, 6 aa, synthetic construct
  REGION 1..6: note = Synthetic: CDR-H2, Chothia
IPIFGT

SEQ ID NO: 52 - protein, 4 aa, synthetic construct
  REGION 1..4: note = Synthetic: CDR-H2, Chothia
GFGT

SEQ ID NO: 53 - protein, 6 aa, synthetic construct
  REGION 1..6: note = Synthetic: CDR-H2, Chothia
LPIGGT

SEQ ID NO: 54 - protein, 6 aa, synthetic construct
  REGION 1..6: note = Synthetic: CDR-H2, Chothia
LPIAGT

SEQ ID NO: 55 - protein, 6 aa, synthetic construct
  REGION 1..6: note = Synthetic: CDR-H2, Chothia
LPIFGE

SEQ ID NO: 56 - protein, 6 aa, synthetic construct
  REGION 1..6: note = Synthetic: CDR-H2, Chothia
IPRGGT

SEQ ID NO: 57 - protein, 6 aa, synthetic construct
  REGION 1..6: note = Synthetic: CDR-H2, Chothia
IPEFGI

SEQ ID NO: 58 - protein, 6 aa, synthetic construct
  REGION 1..6: note = Synthetic: CDR-H2, Chothia
IPSIGT

SEQ ID NO: 59 - protein, 6 aa, synthetic construct
  REGION 1..6: note = Synthetic: CDR-H2, Chothia
IPISGT

SEQ ID NO: 60 - protein, 6 aa, synthetic construct
  REGION 1..6: note = Synthetic: CDR-H2, Chothia
IPTFGT

SEQ ID NO: 61 - protein, 6 aa, synthetic construct
  REGION 1..6: note = Synthetic: CDR-H2, Chothia
SSSSSY

SEQ ID NO: 62 - protein, 6 aa, synthetic construct
  REGION 1..6: note = Synthetic: CDR-H2, Chothia
WYDGSN

SEQ ID NO: 63 - protein, 17 aa, synthetic construct
  REGION 1..17: note = Synthetic: CDR-H2, Kabat
VINPSGGSTSYAQKFQG

SEQ ID NO: 64 - protein, 17 aa, synthetic construct
  REGION 1..17: note = Synthetic: CDR-H2, Kabat
RINPSVGSTWYAQKFQG

SEQ ID NO: 65 - protein, 17 aa, synthetic construct
  REGION 1..17: note = Synthetic: CDR-H2, Kabat
RINPSGGSTWYAQKFQG

SEQ ID NO: 66 - protein, 17 aa, synthetic construct
  REGION 1..17: note = Synthetic: CDR-H2, Kabat
KINPSGGSTWYAQKFQG

SEQ ID NO: 67 - protein, 17 aa, synthetic construct
  REGION 1..17: note = Synthetic: CDR-H2, Kabat
VINPLGGGTSYAQKFQG

SEQ ID NO: 68 - protein, 17 aa, synthetic construct
  REGION 1..17: note = Synthetic: CDR-H2, Kabat
SINPRGGSTSYAQKFQG

SEQ ID NO: 69 - protein, 17 aa, synthetic construct
  REGION 1..17: note = Synthetic: CDR-H2, Kabat
GIIPIFGTANYAQKFQG

SEQ ID NO: 70 - protein, 15 aa, synthetic construct
  REGION 1..15: note = Synthetic: CDR-H2, Kabat
GIGFGTANYAQKFQG

SEQ ID NO: 71 - protein, 17 aa, synthetic construct
  REGION 1..17: note = Synthetic: CDR-H2, Kabat
GILPIGGTANYAQKFQG

SEQ ID NO: 72 - protein, 17 aa, synthetic construct
  REGION 1..17: note = Synthetic: CDR-H2, Kabat
GILPIAGTANYAQKFQG

SEQ ID NO: 73 - protein, 17 aa, synthetic construct
  REGION 1..17: note = Synthetic: CDR-H2, Kabat
GILPIFGEANYAQKFQG

SEQ ID NO: 74 - protein, 17 aa, synthetic construct
  REGION 1..17: note = Synthetic: CDR-H2, Kabat
GIIPRGGTANYAQKFQG

SEQ ID NO: 75 - protein, 17 aa, synthetic construct
  REGION 1..17: note = Synthetic: CDR-H2, Kabat
SIIPIFGTANYAQKFRG

SEQ ID NO: 76 - protein, 17 aa, synthetic construct
  REGION 1..17: note = Synthetic: CDR-H2, Kabat
SIIPEFGIANYAQKFQG

SEQ ID NO: 77 - protein, 17 aa, synthetic construct
  REGION 1..17: note = Synthetic: CDR-H2, Kabat
SIIPIFGTANYAQKFQG

SEQ ID NO: 78 - protein, 17 aa, synthetic construct
  REGION 1..17: note = Synthetic: CDR-H2, Kabat
GIIPISGTANYAQEFQG

SEQ ID NO: 79 - protein, 17 aa, synthetic construct
  REGION 1..17: note = Synthetic: CDR-H2, Kabat
GIIPTFGTANYAQKFQG

SEQ ID NO: 80 - protein, 17 aa, synthetic construct
  REGION 1..17: note = Synthetic: CDR-H2, Kabat
SISSSSSYIYYADSVKG

SEQ ID NO: 81 - protein, 17 aa, synthetic construct
  REGION 1..17: note = Synthetic: CDR-H2, Kabat
VIWYDGSNKYYADSVKG

SEQ ID NO: 82 - protein, 12 aa, synthetic construct
  REGION 1..12: note = Synthetic: CDR-H3
GKREGGTEYLRH

SEQ ID NO: 83 - protein, 12 aa, synthetic construct
  REGION 1..12: note = Synthetic: CDR-H3
GKREGGTEYLRK

SEQ ID NO: 84 - protein, 12 aa, synthetic construct
  REGION 1..12: note = Synthetic: CDR-H3
GKREGGTEYLRS

SEQ ID NO: 85 - protein, 12 aa, synthetic construct
  REGION 1..12: note = Synthetic: CDR-H3
GKREGGTEYLRN

SEQ ID NO: 86 - protein, 12 aa, synthetic construct
  REGION 1..12: note = Synthetic: CDR-H3
GKREGGTEYLRV

SEQ ID NO: 87 - protein, 13 aa, synthetic construct
  REGION 1..13: note = Synthetic: CDR-H3
GGAKYASTYGMDV

SEQ ID NO: 88 - protein, 13 aa, synthetic construct
  REGION 1..13: note = Synthetic: CDR-H3
GGAKYASTHGMDV

SEQ ID NO: 89 - protein, 13 aa, synthetic construct
  REGION 1..13: note = Synthetic: CDR-H3
GGAKYASQLGMDV

SEQ ID NO: 90 - protein, 13 aa, synthetic construct
  REGION 1..13: note = Synthetic: CDR-H3
GGAKYASKWGMDV

SEQ ID NO: 91 - protein, 13 aa, synthetic construct
  REGION 1..13: note = Synthetic: CDR-H3
GGAKYAVGYGMDV

SEQ ID NO: 92 - protein, 13 aa, synthetic construct
  REGION 1..13: note = Synthetic: CDR-H3
GGAKYAGRYGMDV

SEQ ID NO: 93 - protein, 13 aa, synthetic construct
  REGION 1..13: note = Synthetic: CDR-H3
GGAKYARTYGMDV

SEQ ID NO: 94 - protein, 12 aa, synthetic construct
  REGION 1..12: note = Synthetic: CDR-H3
ESGGYRDHRLDV

SEQ ID NO: 95 - protein, 12 aa, synthetic construct
  REGION 1..12: note = Synthetic: CDR-H3
ESGTYRDHRLDV

SEQ ID NO: 96 - protein, 12 aa, synthetic construct
  REGION 1..12: note = Synthetic: CDR-H3
ESGGYRDHRLGV

SEQ ID NO: 97 - protein, 15 aa, synthetic construct
  REGION 1..15: note = Synthetic: CDR-H3
DFTDYSSGYSSGWTY

SEQ ID NO: 98 - protein, 14 aa, synthetic construct
  REGION 1..14: note = Synthetic: CDR-H3
DTLYSSGAYYGYNV

SEQ ID NO: 99 - protein, 15 aa, synthetic construct
  REGION 1..15: note = Synthetic: CDR-H3
AKRGYDSYGGVYFDY

SEQ ID NO: 100 - protein, 18 aa, synthetic construct
  REGION 1..18: note = Synthetic: CDR-H3
GPTVTATTSIGTHNWFDP

SEQ ID NO: 101 - protein, 28 aa, synthetic construct
  REGION 1..28: note = Synthetic: CDR-H3
EGRGYDSSRYYKFWFDPWGQGTLVTVSS

SEQ ID NO: 102 - protein, 12 aa, synthetic construct
  REGION 1..12: note = Synthetic: CDR-H3
DGGGYRHHYFDL

SEQ ID NO: 103 - protein, 12 aa, synthetic construct
  REGION 1..12: note = Synthetic: CDR-H3
ESGGYRDHKLDV

SEQ ID NO: 104 - protein, 12 aa, synthetic construct
  REGION 1..12: note = Synthetic: CDR-H3
DGGGYQHHYFDL

SEQ ID NO: 105 - protein, 11 aa, synthetic construct
  REGION 1..11: note = Synthetic: CDR-H3
DSGYHRHYSDY

SEQ ID NO: 106 - protein, 12 aa, synthetic construct
  REGION 1..12: note = Synthetic: CDR-H3
DPLGIRKHWFDP

SEQ ID NO: 107 - protein, 12 aa, synthetic construct
  REGION 1..12: note = Synthetic: CDR-H3
DTPRWRYHYFDY

SEQ ID NO: 108 - protein, 12 aa, synthetic construct
  REGION 1..12: note = Synthetic: CDR-H3
ERRGSLALGMDV

SEQ ID NO: 109 - protein, 19 aa, synthetic construct
  REGION 1..19: note = Synthetic: CDR-H3
DLGGYSYGEPYYYYYGMDV

SEQ ID NO: 110 - protein, 12 aa, synthetic construct
  REGION 1..12: note = Synthetic: CDR-L1
RASQSVSSSYLA

SEQ ID NO: 111 - protein, 12 aa, synthetic construct
  REGION 1..12: note = Synthetic: CDR-L1
RASQSVASSYLA

SEQ ID NO: 112 - protein, 12 aa, synthetic construct
  REGION 1..12: note = Synthetic: CDR-L1
EASQSVSYSYLA

SEQ ID NO: 113 - protein, 12 aa, synthetic construct
  REGION 1..12: note = Synthetic: CDR-L1
KASESVSSSYLA

SEQ ID NO: 114 - protein, 12 aa, synthetic construct
  REGION 1..12: note = Synthetic: CDR-L1
RASQYVSSSYLA

SEQ ID NO: 115 - protein, 17 aa, synthetic construct
  REGION 1..17: note = Synthetic: CDR-L1
KSSQSVLFSSNNKNYLA

SEQ ID NO: 116 - protein, 17 aa, synthetic construct
  REGION 1..17: note = Synthetic: CDR-L1
KSSRSVLFSSNNKNYLA

SEQ ID NO: 117 - protein, 17 aa, synthetic construct
  REGION 1..17: note = Synthetic: CDR-L1
KSSKSVLYSNNNKNYLA

SEQ ID NO: 118 - protein, 11 aa, synthetic construct
  REGION 1..11: note = Synthetic: CDR-L1
RASQSVGSNLA

SEQ ID NO: 119 - protein, 17 aa, synthetic construct
  REGION 1..17: note = Synthetic: CDR-L1
KSSQSVLYSSNNKNYLA

SEQ ID NO: 120 - protein, 11 aa, synthetic construct
  REGION 1..11: note = Synthetic: CDR-L1
QASQDISNYLN

SEQ ID NO: 121 - protein, 11 aa, synthetic construct
  REGION 1..11: note = Synthetic: CDR-L1
RASQSVSSYLA

SEQ ID NO: 122 - protein, 11 aa, synthetic construct
  REGION 1..11: note = Synthetic: CDR-L1
RASQSVSRYLA

SEQ ID NO: 123 - protein, 11 aa, synthetic construct
  REGION 1..11: note = Synthetic: CDR-L1
RASQSISSWLA

SEQ ID NO: 124 - protein, 12 aa, synthetic construct
  REGION 1..12: note = Synthetic: CDR-L1
RASQSVSSDYLA

SEQ ID NO: 125 - protein, 7 aa, synthetic construct
  REGION 1..7: note = Synthetic: CDR-L2
GASSRAT

SEQ ID NO: 126 - protein, 7 aa, synthetic construct
  REGION 1..7: note = Synthetic: CDR-L2
GASNRHT

SEQ ID NO: 127 - protein, 7 aa, synthetic construct
  REGION 1..7: note = Synthetic: CDR-L2
YASSRAY

SEQ ID NO: 128 - protein, 7 aa, synthetic construct
  REGION 1..7: note = Synthetic: CDR-L2
GASSRAN

SEQ ID NO: 129 - protein, 7 aa, synthetic construct
  REGION 1..7: note = Synthetic: CDR-L2
YASSRAT

SEQ ID NO: 130 - protein, 7 aa, synthetic construct
  REGION 1..7: note = Synthetic: CDR-L2
YASNRAT

SEQ ID NO: 131 - protein, 7 aa, synthetic construct
  REGION 1..7: note = Synthetic: CDR-L2
WASTRES

SEQ ID NO: 132 - protein, 7 aa, synthetic construct
  REGION 1..7: note = Synthetic: CDR-L2
WASSRES

SEQ ID NO: 133 - protein, 7 aa, synthetic construct
  REGION 1..7: note = Synthetic: CDR-L2
WASTRQS

SEQ ID NO: 134 - protein, 7 aa, synthetic construct
  REGION 1..7: note = Synthetic: CDR-L2
WASTRAS

SEQ ID NO: 135 - protein, 7 aa, synthetic construct
  REGION 1..7: note = Synthetic: CDR-L2
GASTRAT

SEQ ID NO: 136 - protein, 7 aa, synthetic construct
  REGION 1..7: note = Synthetic: CDR-L2
GASTRAS

SEQ ID NO: 137 - protein, 7 aa, synthetic construct
  REGION 1..7: note = Synthetic: CDR-L2
DASNLET

SEQ ID NO: 138 - protein, 7 aa, synthetic construct
  REGION 1..7: note = Synthetic: CDR-L2
DASNRAT

SEQ ID NO: 139 - protein, 7 aa, synthetic construct
  REGION 1..7: note = Synthetic: CDR-L2
DASKRAT

SEQ ID NO: 140 - protein, 7 aa, synthetic construct
  REGION 1..7: note = Synthetic: CDR-L2
KASSLES

SEQ ID NO: 141 - protein, 8 aa, synthetic construct
  REGION 1..8: note = Synthetic: CDR-L3
QQYHSYIT

SEQ ID NO: 142 - protein, 8 aa, synthetic construct
  REGION 1..8: note = Synthetic: CDR-L3
QQYHNAIT

SEQ ID NO: 143 - protein, 8 aa, synthetic construct
  REGION 1..8: note = Synthetic: CDR-L3
QQYYFYIT

SEQ ID NO: 144 - protein, 8 aa, synthetic construct
  REGION 1..8: note = Synthetic: CDR-L3
QQYHSALT

SEQ ID NO: 145 - protein, 8 aa, synthetic construct
  REGION 1..8: note = Synthetic: CDR-L3
QQYHGGIT

SEQ ID NO: 146 - protein, 8 aa, synthetic construct
  REGION 1..8: note = Synthetic: CDR-L3
QQYHRRIT

SEQ ID NO: 147 - protein, 8 aa, synthetic construct
  REGION 1..8: note = Synthetic: CDR-L3
QQYHSGIT

SEQ ID NO: 148 - protein, 9 aa, synthetic construct
  REGION 1..9: note = Synthetic: CDR-L3
QQYYLYPLT

SEQ ID NO: 149 - protein, 9 aa, synthetic construct
  REGION 1..9: note = Synthetic: CDR-L3
QQYWTYPLT

SEQ ID NO: 150 - protein, 9 aa, synthetic construct
  REGION 1..9: note = Synthetic: CDR-L3
QQYLLYPLT

SEQ ID NO: 151 - protein, 9 aa, synthetic construct
  REGION 1..9: note = Synthetic: CDR-L3
QQYLIWPLT

SEQ ID NO: 152 - protein, 9 aa, synthetic construct
  REGION 1..9: note = Synthetic: CDR-L3
QQYLLWPLT

SEQ ID NO: 153 - protein, 9 aa, synthetic construct
  REGION 1..9: note = Synthetic: CDR-L3
QQFYFFPPT

SEQ ID NO: 154 - protein, 9 aa, synthetic construct
  REGION 1..9: note = Synthetic: CDR-L3
QQAYTFPPT

SEQ ID NO: 155 - protein, 9 aa, synthetic construct
  REGION 1..9: note = Synthetic: CDR-L3
QQYYIFPPT

SEQ ID NO: 156 - protein, 9 aa, synthetic construct
  REGION 1..9: note = Synthetic: CDR-L3
QQRNFYPPT

SEQ ID NO: 157 - protein, 9 aa, synthetic construct
  REGION 1..9: note = Synthetic: CDR-L3
QQFVLWPRT

SEQ ID NO: 158 - protein, 9 aa, synthetic construct
  REGION 1..9: note = Synthetic: CDR-L3
QQHVNFPLT

SEQ ID NO: 159 - protein, 9 aa, synthetic construct
  REGION 1..9: note = Synthetic: CDR-L3
QQSVFWPIT

SEQ ID NO: 160 - protein, 9 aa, synthetic construct
  REGION 1..9: note = Synthetic: CDR-L3
QQLTKWPLT

SEQ ID NO: 161 - protein, 9 aa, synthetic construct
  REGION 1..9: note = Synthetic: CDR-L3
QQDVLWPLT

SEQ ID NO: 162 - protein, 9 aa, synthetic construct
  REGION 1..9: note = Synthetic: CDR-L3
QQYGLFPIT

SEQ ID NO: 163 - protein, 9 aa, synthetic construct
  REGION 1..9: note = Synthetic: CDR-L3
QQHTVWPIT

SEQ ID NO: 164 - protein, 9 aa, synthetic construct
  REGION 1..9: note = Synthetic: CDR-L3
QQVLNYPLT

SEQ ID NO: 165 - protein, 9 aa, synthetic construct
  REGION 1..9: note = Synthetic: CDR-L3
QQSYFLPPT

SEQ ID NO: 166 - protein, 9 aa, synthetic construct
  REGION 1..9: note = Synthetic: CDR-L3
QQAHSSPYT

SEQ ID NO: 167 - protein, 22 aa, synthetic construct
  REGION 1..22: note = Synthetic: Leader for scFV, scFv-Fc, Leader
MKYLLPTAAAGLLLLAAQPAMA

SEQ ID NO: 168 - protein, 15 aa, synthetic construct
  REGION 1..15: note = Synthetic: Linker for scFV, scFV-FC, Linker
GGGGSGGGGSGGGGS

SEQ ID NO: 169 - protein, 11 aa, synthetic construct
  REGION 1..11: note = Synthetic: C-Term Tag for scFV, scFV-FC, C-Term Tag
GPGGQHHHHHH

SEQ ID NO: 170 - protein, 231 aa, synthetic construct
  REGION 1..231: note = Synthetic: C-Term Tag for scFv, scFV-FC, C-Term Tag
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 276 aa, synthetic construct
  REGION 1..276: note = Synthetic: scFv, 29872
MKYLLPTAAAGLLLLAAQPAMAQVQLVQSGAEVKEPGASVKVSCKAPGYTFTSYYMHWVRQAPGQGLEWMGVINPSGGSTSYAQKFQGRVTMTRDTSTSTVYMELSSLRSEDTAVYYCARGKREGGTEYLRHWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYHSYITFGGGTKVEIKGPGGQHHHHHH

SEQ ID NO: 172 - protein, 276 aa, synthetic construct
  REGION 1..276: note = Synthetic: scFv, 31895
MKYLLPTAAAGLLLLAAQPAMAQVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRKWGQGTLVTVSSGGGGSGGGGSGGGGSEIVLTQSPGTLSLSPGERATLSCRASQSVASSYLAWYQQKPGQAPRLLIYGASNRHTGIPDRFSGSGSGTDFTLTISRLEPEDFAVYYCQQYHNAITFGGGTKVEIKGPGGQHHHHHH

SEQ ID NO: 173 - protein, 276 aa, synthetic construct
  REGION 1..276: note = Synthetic: scFv, 31414
MKYLLPTAAAGLLLLAAQPAMAQVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRNWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYHSYITFGGGTKVEIKGPGGQHHHHHH

SEQ ID NO: 174 - protein, 281 aa, synthetic construct
  REGION 1..281: note = Synthetic: scFv, 31905
MKYLLPTAAAGLLLLAAQPAMAQVQLVQSGAEVKKPGSSVKVSCKASGGTFPSNAISWVRQAPGQGLEWMGGIGFGTANYAQKFQGRVTITADESTSTAYMELSSLRSEDTAVYYCARGGAKYARTYGMDVWGQGTTVTVSSGGGGSGGGGSGGGGSDIVMTQSPDSLAVSLGERATINCKSSKSVLYSNNNKNYLAWYQQKPGQPPKLLIYWASTRQSGVPDRFSGSGSGTDFTLTISSLQAEDVAVYYCQQYLLYPLTFGGGTKVEIKGPGGQHHHHHH

SEQ ID NO: 175 - protein, 496 aa, synthetic construct
  REGION 1..496: note = Synthetic: scFv-Fc, 29872
MKYLLPTAAAGLLLLAAQPAMAQVQLVQSGAEVKEPGASVKVSCKAPGYTFTSYYMHWVRQAPGQGLEWMGVINPSGGSTSYAQKFQGRVTMTRDTSTSTVYMELSSLRSEDTAVYYCARGKREGGTEYLRHWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYHSYITFGGGTKVEIKPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 496 aa, synthetic construct
  REGION 1..496: note = Synthetic: scFv-Fc, 31895
MKYLLPTAAAGLLLLAAQPAMAQVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRKWGQGTLVTVSSGGGGSGGGGSGGGGSEIVLTQSPGTLSLSPGERATLSCRASQSVASSYLAWYQQKPGQAPRLLIYGASNRHTGIPDRFSGSGSGTDFTLTISRLEPEDFAVYYCQQYHNAITFGGGTKVEIKPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 496 aa, synthetic construct
  REGION 1..496: note = Synthetic: scFv-Fc, 31414
MKYLLPTAAAGLLLLAAQPAMAQVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRNWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYHSYITFGGGTKVEIKPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 501 aa, synthetic construct
  REGION 1..501: note = Synthetic: scFv-Fc, 31905
MKYLLPTAAAGLLLLAAQPAMAQVQLVQSGAEVKKPGSSVKVSCKASGGTFPSNAISWVRQAPGQGLEWMGGIGFGTANYAQKFQGRVTITADESTSTAYMELSSLRSEDTAVYYCARGGAKYARTYGMDVWGQGTTVTVSSGGGGSGGGGSGGGGSDIVMTQSPDSLAVSLGERATINCKSSKSVLYSNNNKNYLAWYQQKPGQPPKLLIYWASTRQSGVPDRFSGSGSGTDFTLTISSLQAEDVAVYYCQQYLLYPLTFGGGTKVEIKPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 121 aa, synthetic construct
  REGION 1..121: note = Synthetic: VH, 27579
QVQLVQSGAEVKEPGASVKVSCKAPGYTFTSYYMHWVRQAPGQGLEWMGVINPSGGSTSYAQKFQGRVTMTRDTSTSTVYMELSSLRSEDTAVYYCARGKREGGTEYLRHWGQGTLVTVSS

SEQ ID NO: 180 - protein, 121 aa, synthetic construct
  REGION 1..121: note = Synthetic: VH, 31895
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRKWGQGTLVTVSS

SEQ ID NO: 181 - protein, 121 aa, synthetic construct
  REGION 1..121: note = Synthetic: VH, 31415
QVQLVQSGAEVKKPGASVKVSCKASGYTFTSYQMHWVRQAPGQGLEWMGRINPSGGSTWYAQKFQGRVTMTRDTSTSTVYMELSSLRSEDTAVYYCARGKREGGTEYLRSWGQGTLVTVSS

SEQ ID NO: 182 - protein, 121 aa, synthetic construct
  REGION 1..121: note = Synthetic: VH, 31414
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRNWGQGTLVTVSS

SEQ ID NO: 183 - protein, 121 aa, synthetic construct
  REGION 1..121: note = Synthetic: VH, 31891
QVQLVQSGAEVKKPGASVKVSCKASGYIFKSYEMHWVRQAPGQGLEWMGRINPSVGSTWYAQKFQGRVTMTRDTSTSTVYMELSSLRSEDTAVYYCARGKREGGTEYLRVWGQGTLVTVSS

SEQ ID NO: 184 - protein, 121 aa, synthetic construct
  REGION 1..121: note = Synthetic: VH, 29871
QVQLVQSGAEVKKPGASVKVSCKASGYTFQSYYMHWVRQAPGQGLEWMGKINPSGGSTWYAQKFQGRVTMTRDTSTSTVYMELSSLRSEDTAVYYCARGKREGGTEYLRHWGQGTLVTVSS

SEQ ID NO: 185 - protein, 121 aa, synthetic construct
  REGION 1..121: note = Synthetic: VH, 31418
QVQLVQSGAEVKKPGASVKVSCKASGYTFKSYEMHWVRQAPGQGLEWMGRINPSGGSTWYAQKFQGRVTMTRDTSTSTVYMELSSLRSEDTAVYYCARGKREGGTEYLRHWGQGTLVTVSS

SEQ ID NO: 186 - protein, 121 aa, synthetic construct
  REGION 1..121: note = Synthetic: VH, 31431
QVQLVQSGAEVKKPGASVKVSCKASGYTFTSYQMHWVRQAPGQGLEWMGRINPSGGSTWYAQKFQGRVTMTRDTSTSTVYMELSSLRSEDTAVYYCARGKREGGTEYLRHWGQGTLVTVSS

SEQ ID NO: 187 - protein, 121 aa, synthetic construct
  REGION 1..121: note = Synthetic: VH, 31421
QVQLVQSGAEVKKPGASVKVSCKASGYTFFSYYMYWVRQAPGQGLEWMGVINPLGGGTSYAQKFQGRVTMTRDTSTSTVYMELSSLRSEDTAVYYCARGKREGGTEYLRHWGQGTLVTVSS

SEQ ID NO: 188 - protein, 121 aa, synthetic construct
  REGION 1..121: note = Synthetic: VH, 31429
QVQLVQSGAEVKKPGASVKVSCKASGYTFVSYFMHWVRQAPGQGLEWMGSINPRGGSTSYAQKFQGRVTMTRDTSTSTVYMELSSLRSEDTAVYYCARGKREGGTEYLRHWGQGTLVTVSS

SEQ ID NO: 189 - protein, 121 aa, synthetic construct
  REGION 1..121: note = Synthetic: VH, 29872
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRHWGQGTLVTVSS

SEQ ID NO: 190 - protein, 122 aa, synthetic construct
  REGION 1..122: note = Synthetic: VH, 28347
QVQLVQSGAEVKKPGSSVKVSCKASGGTFSSLAISWVRQAPGQGLEWMGGIIPIFGTANYAQKFQGRVTITADESTNTAYMELSSLRSEDTAVYYCARGGAKYASTYGMDVWGQGTTVTVSS

SEQ ID NO: 191 - protein, 120 aa, synthetic construct
  REGION 1..120: note = Synthetic: VH, 31896
QVQLVQSGAEVKKPGSSVKVSCKASGGTFSKLAISWVRQAPGQGLEWMGGIGFGTANYAQKFQGRVTITADESASTAYMELSSLRSEDTAVYYCARGGAKYASTHGMDVWGQGTTVTVSS

SEQ ID NO: 192 - protein, 122 aa, synthetic construct
  REGION 1..122: note = Synthetic: VH, 31432
QVQLVQSGAEVKKPGSSVKVSCKASGGTFSHTAISWVRQAPGQGLEWMGGILPIGGTANYAQKFQGRVTITADESTSTAYMELSSLRSEDTAVYYCARGGAKYASQLGMDVWGQGTTVTVSS

SEQ ID NO: 193 - protein, 120 aa, synthetic construct
  REGION 1..120: note = Synthetic: VH, 31915
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYASKWGMDVWGQGTTVTVSS

SEQ ID NO: 194 - protein, 122 aa, synthetic construct
  REGION 1..122: note = Synthetic: VH, 31436
QVQLVQSGAEVKKPGSSVKVSCKASGGTFSLLAISWVRQAPGQGLEWMGGILPIAGTANYAQKFQGRVTITADESTSTAYMELSSLRSEDTAVYYCARGGAKYAVGYGMDVWGQGTTVTVSS

SEQ ID NO: 195 - protein, 122 aa, synthetic construct
  REGION 1..122: note = Synthetic: VH, 31437
QVQLVQSGAEVKKPGASVKVSCKASGGTFQSLAISWVRQAPGQGLEWMGGILPIGGTANYAQKFQGRVTITADESTSTAYMELSSLRSEDTAVYYCARGGAKYAGRYGMDVWGQGTTVTVSS

SEQ ID NO: 196 - protein, 120 aa, synthetic construct
  REGION 1..120: note = Synthetic: VH, 31905
QVQLVQSGAEVKKPGSSVKVSCKASGGTFPSNAISWVRQAPGQGLEWMGGIGFGTANYAQKFQGRVTITADESTSTAYMELSSLRSEDTAVYYCARGGAKYARTYGMDVWGQGTTVTVSS

SEQ ID NO: 197 - protein, 120 aa, synthetic construct
  REGION 1..120: note = Synthetic: VH, 31901
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYAGRYGMDVWGQGTTVTVSS

SEQ ID NO: 198 - protein, 122 aa, synthetic construct
  REGION 1..122: note = Synthetic: VH, 29852
QVQLVQSGAEVKKPGSSVKVSCKASGGTFSAMAISWVRQAPGQGLEWMGGILPIAGTANYAQKFQGRVTITADESTSTAYMELSSLRSEDTAVYYCARGGAKYASTYGMDVWGQGTTVTVSS

SEQ ID NO: 199 - protein, 122 aa, synthetic construct
  REGION 1..122: note = Synthetic: VH, 29851
QVQLVQSGAEVKKPGSSVKVSCKASGGTFASLAISWVRQAPGQGLEWMGGILPIFGEANYAQKFQGRVTITADESTSTAYMELSSLRSEDTAVYYCARGGAKYASTYGMDVWGQGTTVTVSS

SEQ ID NO: 200 - protein, 122 aa, synthetic construct
  REGION 1..122: note = Synthetic: VH, 29857
QVQLVQSGAEVKKPGSSVKVSCKASGGTFSWLAISWVRQAPGQGLEWMGGIIPRGGTANYAQKFQGRVTITADESTSTAYMELSSLRSEDTAVYYCARGGAKYASTYGMDVWGQGTTVTVSS

SEQ ID NO: 201 - protein, 121 aa, synthetic construct
  REGION 1..121: note = Synthetic: VH, 27571
QVQLVQSGAEVKKPGSSVKASCKASGGTFSSYAISWVRQAPGQGLEWMGSIIPIFGTANYAQKFRGRVTITADESTSTTYMELSSLRSEDTAVYYCARESGGYRDHRLDVWGQGTMVTVSS

SEQ ID NO: 202 - protein, 121 aa, synthetic construct
  REGION 1..121: note = Synthetic: VH, 31861
QVQLVQSGAEVKKPGSSVKVSCKASGGTFGSYGISWVRQAPGQGLEWMGSIIPEFGIANYAQKFQGRVTITADESTSTAYMELSSLRSEDTAVYYCARESGTYRDHRLDVWGQGTMVTVSS

SEQ ID NO: 203 - protein, 121 aa, synthetic construct
  REGION 1..121: note = Synthetic: VH, 31873
QVQLVQSGAEVKKPGSSVKVSCKASGGTFSKYGISWVRQAPGQGLEWMGSIIPEFGIANYAQKFQGRVTITADESTSTAYMELSSLRSEDTAVYYCARESGGYRDHRLGVWGQGTMVTVSS

SEQ ID NO: 204 - protein, 121 aa, synthetic construct
  REGION 1..121: note = Synthetic: VH, 31393
QVQLVQSGAEVKKPGSSVKVSCKASGGTFESYGISWVRQAPGQGLEWMGSIIPEFGIANYAQKFQGRVTITADESTSTTYMELSSLRSEDTAVYYCARESGGYRDHRLDVWGQGTMVTVSS

SEQ ID NO: 205 - protein, 124 aa, synthetic construct
  REGION 1..124: note = Synthetic: VH, 27534
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDFTDYSSGYSSGWTYWGQGTLVTVSS

SEQ ID NO: 206 - protein, 123 aa, synthetic construct
  REGION 1..123: note = Synthetic: VH, 27536
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCARDTLYSSGAYYGYNVWGQGTMVTVSS

SEQ ID NO: 207 - protein, 124 aa, synthetic construct
  REGION 1..124: note = Synthetic: VH, 27588
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCARAKRGYDSYGGVYFDYWGQGTLVTVSS

SEQ ID NO: 208 - protein, 127 aa, synthetic construct
  REGION 1..127: note = Synthetic: VH, 27590
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCARGPTVTATTSIGTHNWFDPWGQGTLVTVSS

SEQ ID NO: 209 - protein, 126 aa, synthetic construct
  REGION 1..126: note = Synthetic: VH, 27597
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCAREGRGYDSSRYYKFWFDPWGQGTLVTVSS

SEQ ID NO: 210 - protein, 121 aa, synthetic construct
  REGION 1..121: note = Synthetic: VH, 27575
QVQLVQSGAEVKEPGSSVKVSCKASGGTFSSYATSWVRQAPGQGLEWMGGIIPISGTANYAQEFQGRVTITADESTSTAYMELSSLRSEDTAVYYCARDGGGYRHHYFDLWGRGTLVTVSS

SEQ ID NO: 211 - protein, 121 aa, synthetic construct
  REGION 1..121: note = Synthetic: VH, 27568
QVQLVQSGAEVKKPGSSVKVPCKASGGTFSSYAISWVRQAPEQGLEWMGSIIPIFGTANYAQKFQGRVTITADESTSTAYMELSSLRSEDTAVYYCAGESGGYRDHKLDVWGQGTVVTVSS

SEQ ID NO: 212 - protein, 121 aa, synthetic construct
  REGION 1..121: note = Synthetic: VH, 27577
QVQLVQSGAEVKKPGSSVKVSCKASGGAFSSYAIGWVRQAPGQGLEWMGGIIPTFGTANYAQKFQGRVTITADESTSTAYMELSSLRSEDTAVYYCARDGGGYQHHYFDLWGRGTLVTVSS

SEQ ID NO: 213 - protein, 121 aa, synthetic construct
  REGION 1..121: note = Synthetic: VH, 27587
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARESGGYRDHKLDVWGQGTMVTVSS

SEQ ID NO: 214 - protein, 120 aa, synthetic construct
  REGION 1..120: note = Synthetic: VH, 27589
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDSGYHRHYSDYWGQGTLVTVSS

SEQ ID NO: 215 - protein, 121 aa, synthetic construct
  REGION 1..121: note = Synthetic: VH, 27596
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPLGIRKHWFDPWGQGTLVTVSS

SEQ ID NO: 216 - protein, 121 aa, synthetic construct
  REGION 1..121: note = Synthetic: VH, 27535
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDTPRWRYHYFDYWGQGTLVTVSS

SEQ ID NO: 217 - protein, 121 aa, synthetic construct
  REGION 1..121: note = Synthetic: VH, 27550
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARERRGSLALGMDVWGQGTLVTVSS

SEQ ID NO: 218 - protein, 128 aa, synthetic construct
  REGION 1..128: note = Synthetic: VH, 27549
QVQLVESGGGVVQPGRSLRLSCAASGFTFSSYGMNWVRQAPGKGLEWVAVIWYDGSNKYYADSVKGRFTISRDNSKNTLYLQMNSLRAEDTAVYYCARDLGGYSYGEPYYYYYGMDVWGQGTTVTVSS

SEQ ID NO: 219 - protein, 107 aa, synthetic construct
  REGION 1..107: note = Synthetic: VL, 27579
EIVLTQSPGTLSLSPGERATLSCRASQSVSSSYLAWYQQKPGQAPRLLIYGASSRATGIPDRFSGSGSGTDFTLTISRLEPEDFAVYYCQQYHSYITFGGGTKVEIK

SEQ ID NO: 220 - protein, 107 aa, synthetic construct
  REGION 1..107: note = Synthetic: VL, 31895
EIVLTQSPGTLSLSPGERATLSCRASQSVASSYLAWYQQKPGQAPRLLIYGASNRHTGIPDRFSGSGSGTDFTLTISRLEPEDFAVYYCQQYHNAITFGGGTKVEIK

SEQ ID NO: 221 - protein, 107 aa, synthetic construct
  REGION 1..107: note = Synthetic: VL, 31891
EIVLTQSPGTLSLSPGERATLSCRASQSVSSSYLAWYQQKPGQAPRLLIYYASSRAYGIPDRFSGSGSGTDFTLTISRLEPEDFAVYYCQQYHNAITFGGGTKVEIK

SEQ ID NO: 222 - protein, 107 aa, synthetic construct
  REGION 1..107: note = Synthetic: VL, 31418
EIVLTQSPGTLSLSPGERATLSCRASQSVSSSYLAWYQQKPGQAPRLLIYGASSRATGIPDRFSGSGSGTDFTLTISRLEPEDFAVYYCQQYYFYITFGGGTKVEIK

SEQ ID NO: 223 - protein, 107 aa, synthetic construct
  REGION 1..107: note = Synthetic: VL, 31430
EIVLTQSPGTLSLSPGERATLSCEASQSVSYSYLAWYQQKPGQAPRLLIYGASSRANGIPDRFSGSGSGTDFTLTISRLEPEDFAVYYCQQYHSALTFGGGTKVEIK

SEQ ID NO: 224 - protein, 107 aa, synthetic construct
  REGION 1..107: note = Synthetic: VL, 31431
EIVLTQSPGTLSLSPGERATLSCRASQSVASSYLAWYQQKPGQAPRLLIYGASNRHTGIPDRFSGSGSGTDFTLTISRLEPEDFAVYYCQQYHGGITFGGGTKVEIK

SEQ ID NO: 225 - protein, 107 aa, synthetic construct
  REGION 1..107: note = Synthetic: VL, 31421
EIVLTQSPGTLSLSPGERATLSCKASESVSSSYLAWYQQKPGQAPRLLIYYASSRATGIPDRFSGSGSGTDFTLTISRLEPEDFAVYYCQQYHRRITFGGGTKVEIK

SEQ ID NO: 226 - protein, 107 aa, synthetic construct
  REGION 1..107: note = Synthetic: VL, 31429
EIVLTQSPGTLSLSPGERATLSCRASQYVSSSYLAWYQQKPGQAPRLLIYYASNRATGIPDRFSGSGSGTDFTLTISRLEPEDFAVYYCQQYHSGITFGGGTKVEIK

SEQ ID NO: 227 - protein, 113 aa, synthetic construct
  REGION 1..113: note = Synthetic: VL, 28347
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYLYPLTFGGGTKVEIK

SEQ ID NO: 228 - protein, 113 aa, synthetic construct
  REGION 1..113: note = Synthetic: VL, 31896
DIVMTQSPDSLAVSLGERATINCKSSRSVLFSSNNKNYLAWYQQKPGQPPKLLIYWASTRESGVPDRFSGSGSGTDFTLTISSLQAEDVAVYYCQQYWTYPLTFGGGTKVEIK

SEQ ID NO: 229 - protein, 113 aa, synthetic construct
  REGION 1..113: note = Synthetic: VL, 31915
DIVMTQSPDSLAVSLGERATINCKSSQSVLFSSNNKNYLAWYQQKPGQPPKLLIYWASSRESGVPDRFSGSGSGTDFTLTISSLQAEDVAVYYCQQYWTYPLTFGGGTKVEIK

SEQ ID NO: 230 - protein, 113 aa, synthetic construct
  REGION 1..113: note = Synthetic: VL, 31905
DIVMTQSPDSLAVSLGERATINCKSSKSVLYSNNNKNYLAWYQQKPGQPPKLLIYWASTRQSGVPDRFSGSGSGTDFTLTISSLQAEDVAVYYCQQYLLYPLTFGGGTKVEIK

SEQ ID NO: 231 - protein, 113 aa, synthetic construct
  REGION 1..113: note = Synthetic: VL, 31901
GIVMTQSPDSLAVSLGERATINCKSSQSVLFSSNNKNYLAWYQQKPGQPPKLLIYWASTRASGVPDRFSGSGSGTDFTLTISSLQAEDVAVYYCQQYYLYPLTFGGGTKVEIK

SEQ ID NO: 232 - protein, 107 aa, synthetic construct
  REGION 1..107: note = Synthetic: VL, 27571
EIVMTQSPATLSVSPGERATLSCRASQSVGSNLAWYQQKPGQAPRLLIYGASTRATGIPARFSGSGSGTEFTLTISSLQSEDFAVYYCQQYLIWPLTFGGGTKVEIK

SEQ ID NO: 233 - protein, 107 aa, synthetic construct
  REGION 1..107: note = Synthetic: VL, 31861
EIVMTQSPATLSVSPGERATLSCRASQSVGSNLAWYQQKPGQAPRLLIYGASTRATGIPARFSGSGSGTEFTLTISSLQSEDFAVYYCQQYLLWPLTFGGGTKVEIK

SEQ ID NO: 234 - protein, 107 aa, synthetic construct
  REGION 1..107: note = Synthetic: VL, 31873
EIVMTQSPATLSVSPGERATLSCRASQSVGSNLAWYQQKPGQAPRLLIYGASTRASGIPARFSGSGSGTEFTLTISSLQSEDFAVYYCQQYLLWPLTFGGGTKVEIK

SEQ ID NO: 235 - protein, 113 aa, synthetic construct
  REGION 1..113: note = Synthetic: VL, 28337
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQFYFYPPTFGGGTKVEIK

SEQ ID NO: 236 - protein, 113 aa, synthetic construct
  REGION 1..113: note = Synthetic: VL, 27536
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQAYTFPPTFGGGTKVEIK

SEQ ID NO: 237 - protein, 107 aa, synthetic construct
  REGION 1..107: note = Synthetic: VL, 27588
DIQMTQSPSSLSASVGDRVTITCQASQDISNYLNWYQQKPGKAPKLLIYDASNLETGVPSRFSGSGSGTDFTFTISSLQPEDIATYYCQQYYIFPPTFGGGTKVEIK

SEQ ID NO: 238 - protein, 107 aa, synthetic construct
  REGION 1..107: note = Synthetic: VL, 27590
EIVLTQSPATLSLSPGERATLSCRASQSVSSYLAWYQQKPGQAPRLLIYDASNRATGIPARFSGSGSGTDFTLTISSLEPEDFAVYYCQQRNFYPPTFGGGTKVEIK

SEQ ID NO: 239 - protein, 107 aa, synthetic construct
  REGION 1..107: note = Synthetic: VL, 27597
EIVLTQSPATLSLSPGERATLSCRASQSVSSYLAWYQQKPGQAPRLLIYDASNRATGIPARFSGSGSGTDFTLTISSLEPEDFAVYYCQQFVLWPRTFGGGTKVEIK

SEQ ID NO: 240 - protein, 107 aa, synthetic construct
  REGION 1..107: note = Synthetic: VL, 27575
EIVLTQSPATLSLSPGERATLSCRASQSVSRYLAWYQQKPGQAPRLLIYDASNRATGIPARFSGSGSGTDFTLTISSLEPEDFAVYYCQQHVNFPLTFGGGTKVEIK

SEQ ID NO: 241 - protein, 107 aa, synthetic construct
  REGION 1..107: note = Synthetic: VL, 27568
EIVLTQSPATLSLSPGERATLSCRASQSVSSYLAWYQQKPGQAPRLLIYDASNRATGIPARFSGSGSGTDFTLTISSLEPEDFAVYYCQQSVFWPITFGGGTKVEIK

SEQ ID NO: 242 - protein, 107 aa, synthetic construct
  REGION 1..107: note = Synthetic: VL, 27577
EIVMTQSPATLSVSPGERATLSCRASQSVGSNLAWYQQKPGQAPRLLIYGASTRATGIPARFSGSGSGTEFTLTISSLQSEDFAVYYCQQLTKWPLTFGGGTKVEIK

SEQ ID NO: 243 - protein, 107 aa, synthetic construct
  REGION 1..107: note = Synthetic: VL, 27587
EIVLTQSPATLSLSPGERATLSCRASQSVSSYLAWYQQKPGQAPRLLIYDASKRATGIPARFSGSGSGTDFTLTISSLEPEDFAVYYCQQDVLWPLTFGGGTKVEIK

SEQ ID NO: 244 - protein, 107 aa, synthetic construct
  REGION 1..107: note = Synthetic: VL, 27589
DIQMTQSPSTLSASVGDRVTITCRASQSISSWLAWYQQKPGKAPKLLIYKASSLESGVPSRFSGSGSGTEFTLTISSLQPDDFATYYCQQYGLFPITFGGGTKVEIK

SEQ ID NO: 245 - protein, 107 aa, synthetic construct
  REGION 1..107: note = Synthetic: VL, 27596
EIVMTQSPATLSLSPGERATLSCRASQSVSSYLAWYQQKPGQAPRLLIYDASNRATGIPARFSGSGSGTDFTLTISSLEPEDFAVYYCQQHTVWPITFGGGTKVEIK

SEQ ID NO: 246 - protein, 107 aa, synthetic construct
  REGION 1..107: note = Synthetic: VL, 27535
EIVLTQSPATLSLSPGERATLSCRASQSVSSYLAWYQQKPGQAPRLLIYDASKRATGIPARFSGSGSGTDFTLTISSLEPEDFAVYYCQQVLNYPLTFGGGTKVEIK

SEQ ID NO: 247 - protein, 107 aa, synthetic construct
  REGION 1..107: note = Synthetic: VL, 27550
DIQMTQSPSSLSASVGDRVTITCQASQDISNYLNWYQQKPGKAPKLLIYDASNLETGVPSRFSGSGSGTDFTFTISSLQPEDIATYYCQQSYFLPPTFGGGTKVEIK

SEQ ID NO: 248 - protein, 108 aa, synthetic construct
  REGION 1..108: note = Synthetic: VL, 27549
EIVLTQSPGTLSLSPGERATLSCRASQSVSSDYLAWYQQKPGQAPRLLIYGASSRATGIPDRFSGSGSGTDFTLTISRLEPEDFAVYYCQQAHSSPYTFGGGTKVEIK

SEQ ID NO: 249 - protein, 510 aa, synthetic construct
  REGION 1..510: note = Synthetic: hCD39
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 250 - protein, 510 aa, synthetic construct
  REGION 1..510: note = Synthetic: mCD39
MEDIKDSKVKRFCSKNILIILGFTSILAVIALIAVGLTQNKPLPENVKYGIVLDAGSSHTNLYIYKWPAEKENDTGVVQQLEECQVKGPGISKYAQKTDEIGAYLAECMELSTELIPTSKHHQTPVYLGATAGMRLLRMESEQSADEVLAAVSTSLKSYPFDFQGAKIITGQEEGAYGWITINYLLGRFTQEQSWLSLISDSQKQETFGALDLGGASTQITFVPQNSTIESPENSLQFRLYGEDYTVYTHSFLCYGKDQALWQKLAKDIQVSSGGVLKDPCFNPGYEKVVNVSELYGTPCTKRFEKKLPFDQFRIQGTGDYEQCHQSILELFNNSHCPYSQCAFNGVFLPPLHGSFGAFSAFYFVMDFFKKVAKNSVISQEKMTEITKNFCSKSWEETKTSYPSVKEKYLSEYCFSGAYILSLLQGYNFTDSSWEQIHFMGKIKDSNAGWTLGYMLNLTNMIPAEQPLSPPLPHSTYIGLMVLFSLLLVAVAITGLFIYSKPSYFWKEAV

SEQ ID NO: 251 - protein, 532 aa, Macaca fascicularis
  REGION 1..532: note = misc_feature - Macaca fascicularis cCD39
MLFDSILSTVGLSKLVSVVSSPAAALSKSNVKTFCSKNILAILGFSSIIAVIALLAVGLTQNKALPENIKYGIVLDAGSSHTSLYIYKWPAEKENDTGVVHQVEECRVKGPGISKYVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQITFVPQNQTTESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSVLELFNTSYCPYSQCAFNGIFLPPLQGDFGAFSAFYFVMNFLNLTSEKVSQEKVTEMMKKFCSQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLVIVAIIGLLIFHKPSYFWKDMV

SEQ ID NO: 252 - protein, 473 aa, synthetic construct
  REGION 1..473: note = Synthetic: hCD39 ECD
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 253 - protein, 473 aa, synthetic construct
  REGION 1..473: note = Synthetic: mCD39 ECD
TQNKPLPENVKYGIVLDAGSSHTNLYIYKWPAEKENDTGVVQQLEECQVKGPGISKYAQKTDEIGAYLAECMELSTELIPTSKHHQTPVYLGATAGMRLLRMESEQSADEVLAAVSTSLKSYPFDFQGAKIITGQEEGAYGWITINYLLGRFTQEQSWLSLISDSQKQETFGALDLGGASTQITFVPQNSTIESPENSLQFRLYGEDYTVYTHSFLCYGKDQALWQKLAKDIQVSSGGVLKDPCFNPGYEKVVNVSELYGTPCTKRFEKKLPFDQFRIQGTGDYEQCHQSILELFNNSHCPYSQCAFNGVFLPPLHGSFGAFSAFYFVMDFFKKVAKNSVISQEKMTEITKNFCSKSWEETKTSYPSVKEKYLSEYCFSGAYILSLLQGYNFTDSSWEQIHFMGKIKDSNAGWTLGYMLNLTNMIPAEQPLSPPLPHSTYIGLMVLFSLLLVAVAITGLFIYSKPSYFWKEAV

SEQ ID NO: 254 - protein, 473 aa, synthetic construct
  REGION 1..473: note = Synthetic: cCD39 ECD
TQNKALPENIKYGIVLDAGSSHTSLYIYKWPAEKENDTGVVHQVEECRVKGPGISKYVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQITFVPQNQTTESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSVLELFNTSYCPYSQCAFNGIFLPPLQGDFGAFSAFYFVMNFLNLTSEKVSQEKVTEMMKKFCSQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLVIVAIIGLLIFHKPSYFWKDMV

SEQ ID NO: 255 - protein, 448 aa, synthetic construct
  REGION 1..448: note = Synthetic: G4, 31895
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRKWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 256 - protein, 214 aa, synthetic construct
  REGION 1..214: note = Synthetic: LC, 31895
EIVLTQSPGTLSLSPGERATLSCRASQSVASSYLAWYQQKPGQAPRLLIYGASNRHTGIPDRFSGSGSGTDFTLTISRLEPEDFAVYYCQQYHNAITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 372 aa, synthetic construct
  REGION 1..372: note = Synthetic: G4, 31415
QVQLVQSGAEVKKPGASVKVSCKASGYTFTSYQMHWVRQAPGQGLEWMGRINPSGGSTWYAQKFQGRVTMTRDTSTSTVYMELSSLRSEDTAVYYCARGKREGGTEYLRSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 258 - protein, 214 aa, synthetic construct
  REGION 1..214: note = Synthetic: LC, 31415
EIVLTQSPGTLSLSPGERATLSCRASQSVSSSYLAWYQQKPGQAPRLLIYGASSRATGIPDRFSGSGSGTDFTLTISRLEPEDFAVYYCQQYHSYITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 259 - protein, 372 aa, synthetic construct
  REGION 1..372: note = Synthetic: G4, 31891
QVQLVQSGAEVKKPGASVKVSCKASGYIFKSYEMHWVRQAPGQGLEWMGRINPSVGSTWYAQKFQGRVTMTRDTSTSTVYMELSSLRSEDTAVYYCARGKREGGTEYLRVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 260 - protein, 214 aa, synthetic construct
  REGION 1..214: note = Synthetic: LC, 31891
EIVLTQSPGTLSLSPGERATLSCRASQSVSSSYLAWYQQKPGQAPRLLIYYASSRAYGIPDRFSGSGSGTDFTLTISRLEPEDFAVYYCQQYHNAITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 261 - protein, 372 aa, synthetic construct
  REGION 1..372: note = Synthetic: G4, 31418
QVQLVQSGAEVKKPGASVKVSCKASGYTFKSYEMHWVRQAPGQGLEWMGRINPSGGSTWYAQKFQGRVTMTRDTSTSTVYMELSSLRSEDTAVYYCARGKREGGTEYLRHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 262 - protein, 214 aa, synthetic construct
  REGION 1..214: note = Synthetic: LC, 31418
EIVLTQSPGTLSLSPGERATLSCRASQSVSSSYLAWYQQKPGQAPRLLIYGASSRATGIPDRFSGSGSGTDFTLTISRLEPEDFAVYYCQQYYFYITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 263 - protein, 372 aa, synthetic construct; identical to SEQ ID NO: 261
  REGION 1..372: note = Synthetic: G4, 31430

SEQ ID NO: 264 - protein, 214 aa, synthetic construct
  REGION 1..214: note = Synthetic: LC, 31430
EIVLTQSPGTLSLSPGERATLSCEASQSVSYSYLAWYQQKPGQAPRLLIYGASSRANGIPDRFSGSGSGTDFTLTISRLEPEDFAVYYCQQYHSALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 371 aa, synthetic construct
  REGION 1..371: note = Synthetic: G4, 31915
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYASKWGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 266 - protein, 220 aa, synthetic construct
  REGION 1..220: note = Synthetic: LC, 31915
DIVMTQSPDSLAVSLGERATINCKSSQSVLFSSNNKNYLAWYQQKPGQPPKLLIYWASSRESGVPDRFSGSGSGTDFTLTISSLQAEDVAVYYCQQYWTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 371 aa, synthetic construct
  REGION 1..371: note = Synthetic: G4, 31905
QVQLVQSGAEVKKPGSSVKVSCKASGGTFPSNAISWVRQAPGQGLEWMGGIGFGTANYAQKFQGRVTITADESTSTAYMELSSLRSEDTAVYYCARGGAKYARTYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 268 - protein, 220 aa, synthetic construct
  REGION 1..220: note = Synthetic: LC, 31905
DIVMTQSPDSLAVSLGERATINCKSSKSVLYSNNNKNYLAWYQQKPGQPPKLLIYWASTRQSGVPDRFSGSGSGTDFTLTISSLQAEDVAVYYCQQYLLYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 371 aa, synthetic construct
  REGION 1..371: note = Synthetic: G4, 31901
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYAGRYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 270 - protein, 220 aa, synthetic construct
  REGION 1..220: note = Synthetic: LC, 31901
GIVMTQSPDSLAVSLGERATINCKSSQSVLFSSNNKNYLAWYQQKPGQPPKLLIYWASTRASGVPDRFSGSGSGTDFTLTISSLQAEDVAVYYCQQYYLYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 372 aa, synthetic construct
  REGION 1..372: note = Synthetic: G4, 31861
QVQLVQSGAEVKKPGSSVKVSCKASGGTFGSYGISWVRQAPGQGLEWMGSIIPEFGIANYAQKFQGRVTITADESTSTAYMELSSLRSEDTAVYYCARESGTYRDHRLDVWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 272 - protein, 214 aa, synthetic construct
  REGION 1..214: note = Synthetic: LC, 31861
EIVMTQSPATLSVSPGERATLSCRASQSVGSNLAWYQQKPGQAPRLLIYGASTRATGIPARFSGSGSGTEFTLTISSLQSEDFAVYYCQQYLLWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 273 - protein, 372 aa, synthetic construct
  REGION 1..372: note = Synthetic: G4, 31873
QVQLVQSGAEVKKPGSSVKVSCKASGGTFSKYGISWVRQAPGQGLEWMGSIIPEFGIANYAQKFQGRVTITADESTSTAYMELSSLRSEDTAVYYCARESGGYRDHRLGVWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 274 - protein, 214 aa, synthetic construct
  REGION 1..214: note = Synthetic: LC, 31873
EIVMTQSPATLSVSPGERATLSCRASQSVGSNLAWYQQKPGQAPRLLIYGASTRASGIPARFSGSGSGTEFTLTISSLQSEDFAVYYCQQYLLWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 275 - protein, 372 aa, synthetic construct
  REGION 1..372: note = Synthetic: G4, 31393
QVQLVQSGAEVKKPGSSVKVSCKASGGTFESYGISWVRQAPGQGLEWMGSIIPEFGIANYAQKFQGRVTITADESTSTTYMELSSLRSEDTAVYYCARESGGYRDHRLDVWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 276 - protein, 214 aa, synthetic construct; identical to SEQ ID NO: 272
  REGION 1..214: note = Synthetic: LC, 31393

SEQ ID NO: 277 - protein, 377 aa, synthetic construct
  REGION 1..377: note = Synthetic: G4, 27597
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCAREGRGYDSSRYYKFWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 278 - protein, 214 aa, synthetic construct
  REGION 1..214: note = Synthetic: LC, 27597
EIVLTQSPATLSLSPGERATLSCRASQSVSSYLAWYQQKPGQAPRLLIYDASNRATGIPARFSGSGSGTDFTLTISSLEPEDFAVYYCQQFVLWPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 279 - protein, 372 aa, synthetic construct
  REGION 1..372: note = Synthetic: G4, 27575
QVQLVQSGAEVKEPGSSVKVSCKASGGTFSSYATSWVRQAPGQGLEWMGGIIPISGTANYAQEFQGRVTITADESTSTAYMELSSLRSEDTAVYYCARDGGGYRHHYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 280 - protein, 214 aa, synthetic construct
  REGION 1..214: note = Synthetic: LC, 27575
EIVLTQSPATLSLSPGERATLSCRASQSVSRYLAWYQQKPGQAPRLLIYDASNRATGIPARFSGSGSGTDFTLTISSLEPEDFAVYYCQQHVNFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 281 - protein, 372 aa, synthetic construct
  REGION 1..372: note = Synthetic: G4, 27568
QVQLVQSGAEVKKPGSSVKVPCKASGGTFSSYAISWVRQAPEQGLEWMGSIIPIFGTANYAQKFQGRVTITADESTSTAYMELSSLRSEDTAVYYCAGESGGYRDHKLDVWGQGTVVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 282 - protein, 214 aa, synthetic construct
  REGION 1..214: note = Synthetic: LC, 27568
EIVLTQSPATLSLSPGERATLSCRASQSVSSYLAWYQQKPGQAPRLLIYDASNRATGIPARFSGSGSGTDFTLTISSLEPEDFAVYYCQQSVFWPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - protein, 372 aa, synthetic construct
  REGION 1..372: note = Synthetic: G4, 27577
QVQLVQSGAEVKKPGSSVKVSCKASGGAFSSYAIGWVRQAPGQGLEWMGGIIPTFGTANYAQKFQGRVTITADESTSTAYMELSSLRSEDTAVYYCARDGGGYQHHYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 284 - protein, 214 aa, synthetic construct
  REGION 1..214: note = Synthetic: LC, 27577
EIVMTQSPATLSVSPGERATLSCRASQSVGSNLAWYQQKPGQAPRLLIYGASTRATGIPARFSGSGSGTEFTLTISSLQSEDFAVYYCQQLTKWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 285 - protein, 372 aa, synthetic construct
  REGION 1..372: note = Synthetic: G4, 27587
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARESGGYRDHKLDVWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 286 - protein, 214 aa, synthetic construct
  REGION 1..214: note = Synthetic: LC, 27587
EIVLTQSPATLSLSPGERATLSCRASQSVSSYLAWYQQKPGQAPRLLIYDASKRATGIPARFSGSGSGTDFTLTISSLEPEDFAVYYCQQDVLWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - protein, 371 aa, synthetic construct
  REGION 1..371: note = Synthetic: G4, 27589
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDSGYHRHYSDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 288 - protein, 214 aa, synthetic construct
  REGION 1..214: note = Synthetic: LC, 27589
DIQMTQSPSTLSASVGDRVTITCRASQSISSWLAWYQQKPGKAPKLLIYKASSLESGVPSRFSGSGSGTEFTLTISSLQPDDFATYYCQQYGLFPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 289 - protein, 372 aa, synthetic construct
  REGION 1..372: note = Synthetic: G4, 27596
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPLGIRKHWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 290 - protein, 214 aa, synthetic construct
  REGION 1..214: note = Synthetic: LC, 27596
EIVMTQSPATLSLSPGERATLSCRASQSVSSYLAWYQQKPGQAPRLLIYDASNRATGIPARFSGSGSGTDFTLTISSLEPEDFAVYYCQQHTVWPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 291 - protein, 372 aa, synthetic construct
  REGION 1..372: note = Synthetic: G4, 27535
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDTPRWRYHYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 292 - protein, 214 aa, synthetic construct
  REGION 1..214: note = Synthetic: LC, 27535
EIVLTQSPATLSLSPGERATLSCRASQSVSSYLAWYQQKPGQAPRLLIYDASKRATGIPARFSGSGSGTDFTLTISSLEPEDFAVYYCQQVLNYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 293 - protein, 372 aa, synthetic construct
  REGION 1..372: note = Synthetic: G4, 27550
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARERRGSLALGMDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 294 - protein, 214 aa, synthetic construct
  REGION 1..214: note = Synthetic: LC, 27550
DIQMTQSPSSLSASVGDRVTITCQASQDISNYLNWYQQKPGKAPKLLIYDASNLETGVPSRFSGSGSGTDFTFTISSLQPEDIATYYCQQSYFLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 379 aa, synthetic construct
  REGION 1..379: note = Synthetic: G4, 27549
QVQLVESGGGVVQPGRSLRLSCAASGFTFSSYGMNWVRQAPGKGLEWVAVIWYDGSNKYYADSVKGRFTISRDNSKNTLYLQMNSLRAEDTAVYYCARDLGGYSYGEPYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKG

SEQ ID NO: 296 - protein, 215 aa, synthetic construct
  REGION 1..215: note = Synthetic: LC, 27549
EIVLTQSPGTLSLSPGERATLSCRASQSVSSDYLAWYQQKPGQAPRLLIYGASSRATGIPDRFSGSGSGTDFTLTISRLEPEDFAVYYCQQAHSSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC